(19) United States
(12) Patent Application Publication
(10) Pub. No.: US 2026/0207615 A1
(43) Pub. Date: Jul. 23, 2026

(54) FLUOROPYRIDOPYRIMIDINE AND FLUOROQUINAZOLINE DERIVATIES AND METHODS OF USE THEREOF

(71) Applicant: Frontier Medicines Corporation, South San Francisco, CA (US)

(72) Inventors: Snahel PATEL, Foster City, CA (US); Philip A. GERKEN, San Francisco, CA (US); Irina DOTSENKO, San Mateo, CA (US); Monika J. WILLIAMS, Woodside, CA (US)

(21) Appl. No.: 19/142,110

(22) PCT Filed: Dec. 22, 2023
(86) PCT No.: PCT/US2023/085854

§ 371 Date: Jun. 20, 2025

Related U.S. Application Data
(60) Provisional application No. 63/435,121, filed on Dec. 23, 2022.
(60) Provisional application No. 63/464,187, filed on May 4, 2023.
(60) Provisional application No. 63/465,444, filed on May 10, 2023.
(60) Provisional application No. 63/531,234, filed on Aug. 7, 2023.

(51) Int. Cl. A61K 31/5386; A61K 31/517; A61K 31/519; A61K 31/5377; A61K 31/55; A61P 35/00; C07D 519/00

(52) U.S. Cl. CPC A61K 31/5386; A61K 31/517; A61K 31/519; A61K 31/5377; A61K 31/55; A61P 35/00; C07D 519/00

(57) ABSTRACT

The present disclosure provides compounds and methods useful in the treatment and suppression of cancer, for example, useful for treating or suppressing cancers characterized by KRAS G12C. Also provided are pharmaceutical compositions containing such compounds and processes for preparing such compounds.

### CROSS-REFERENCE TO RELATED APPLICATIONS

This application claims priority to and benefit of U.S. Provisional Patent Application No. 63/435,121, filed on Dec. 23, 2022; U.S. Provisional Patent Application No. 63/464,187, filed on May 4, 2023; U.S. Provisional Patent Application No. 63/465,444, filed on May 10, 2023; and U.S. Provisional Patent Application No. 63/531,234, filed on Aug. 7, 2023; the disclosures of each of which are hereby incorporated herein by reference in their entirety.

### FIELD OF THE DISCLOSURE

The present disclosure provides compounds useful in treating or suppressing cancer, and in particular, useful in treating or suppressing cancers characterized by the KRAS G12C mutant. Also provided are pharmaceutical formulations containing such compounds, processes for preparing such compounds, and methods of using such compounds in the treatment or suppression of cancers.

### BACKGROUND

KRAS is a molecular switch. Under normal physiological conditions, the protein is bound to guanosine diphosphate (GDP) in the “off state.” In response to signaling through receptor tyrosine kinases (RTKs) such as EGFR, the GDP is exchanged to guanosine triphosphate (GTP) in a process facilitated by guanine nucleotide exchange factors (GEFs) such as SOS. The GTP-bound form of KRAS is in the “on state,” and interacts with proteins such as RAF and PI3K to promote downstream signaling that leads to cell proliferation and survival. KRAS can slowly hydrolyze GTP back to GDP, thus returning to the off-state, in a process facilitated by GAPs (GTPase-activating Proteins).

KRAS mutations are found in approximately 30% of all human cancers, and are highly prevalent among three of the deadliest forms of cancer:pancreatic (95%), colorectal (45%), and lung (35%). Together, these cancers occur in more than 200,000 patients annually in the US alone. One particular mutation, a glycine to cysteine substitution at position 12 (G12C), occurs in more than 40,000 patients per year. The KRAS G12C mutation impairs hydrolysis of GTP to GDP, thus trapping KRAS in the on-state and promoting cancer cell proliferation.

The cysteine residue of G12C provides an opportunity to develop targeted covalent drugs for this mutant KRAS. Early clinical trial results for KRAS G12C inhibitors AMG 510 and MRTX849 have shown encouraging results for non-small cell lung cancer (NSCLC), but the data are less compelling for colorectal cancer (CRC). Moreover, even in cases where patients respond to initial treatment, there are signs that the response may be limited in duration and that resistance could arise rapidly.

Most inhibitors of KRAS mutants bind preferentially to the GDP-bound form of the protein. For example, Amgen KRAS inhibitor AMG 510 and Mirati KRAS inhibitor MRTX849 react with the GDP-bound form of KRAS G12C at least 1000-fold more rapidly than with the GTP-bound form of the protein. One form of resistance that has been observed is for cancer cells to increase signaling through RTKs, thus increasing the amount of GTP-bound KRAS, which is less affected by current inhibitors. Thus, creating a molecule that could bind to and inhibit both the GDP- and GTP-bound forms of KRAS could have substantial utility.

What is needed are compounds useful in the treatment of cancer, such as cancers characterized by KRAS G12C. What is further needed are compounds useful in the treatment of cancers characterized by KRAS G12C, wherein the compounds bind to and inhibit both the inactive GDP- and activated GTP-bound forms of KRAS. What is further needed are compounds useful in the treatment of cancers characterized by KRAS G12C, wherein the compound has improved inhibition of the GTP-bound form of KRAS G12C.

### SUMMARY

In one aspect, the invention provides a compound of Formula (I), Formula (II), Formula (III) or Formula (IV):

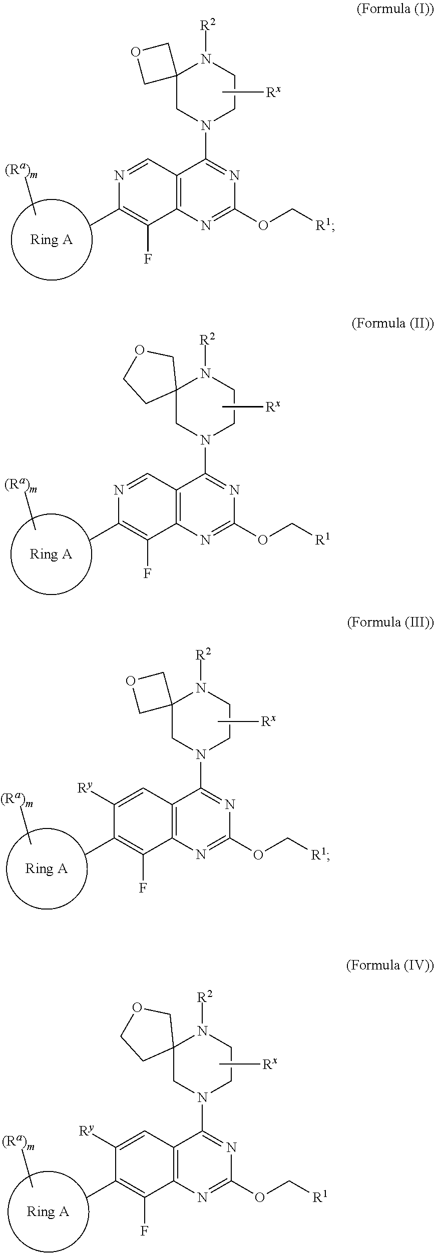

or a salt thereof; and/or an isotopologue thereof; wherein:

Ring A is a 6-10 membered aryl or a 5-10 membered heteroaryl;
each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
m is 0, 1, 2 or 3;
R¹ is

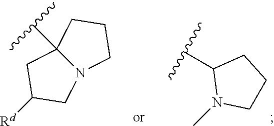

R^{d} is H or F;
R² is

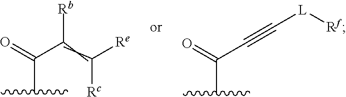

R^{b} is —H or —F;
R^{c} is —H;
R^{e} is —H, —Y—R^{e1}, —Y—R^{e2}, or —Y—R^{e3};
Y is —(C(R^{Y1})(R^{Y2}))ₓ—;
x is 0, 1 or 2 provided that when R^{e} is Y—R^{e3}, x is not 0;
R^{Y1} and R^{Y2} in each occurrence are independently selected from —H and —CH₃;
R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
L is —(C(R^{L1})(L^{L2}))_{z}—;
z is 0, 1 or 2;
R^{L1} and R^{L2} in each occurrence are independently selected from —H and —CH₃;
R^{f} is a 4-10 membered heterocycle which is substituted with 0, 1, 2, 3 or 4 substituents independently selected from halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl;
R^{x} is selected from —H and C₁-C₄ alkyl; and
R^{y} is selected from halo and C₁-C₄ haloalkyl.

In an embodiment, the compound is of Formula (I).

In an embodiment, the compound is of Formula (II).

In an embodiment, the compound is of Formula (III).

In an embodiment, the compound is of Formula (IV).

In some embodiments, including any of the embodiments in the preceding paragraphs, the compound is selected from the group consisting of the compounds of Table 1; and all salts and isotopologues thereof.

In another aspect provided is a pharmaceutical formulation comprising a compound as described herein, including but not limited to a compound described in the preceding paragraphs, and a pharmaceutically acceptable carrier, wherein when the compound is a salt, the salt is a pharmaceutically acceptable salt.

In another aspect provided is a method of treating or suppressing cancer comprising: administering a therapeutically effective amount of a compound as described herein, including but not limited to a compound described in the preceding paragraphs, or a pharmaceutical formulation, including but not limited to the pharmaceutical formulation described in the preceding paragraphs, to a subject in need thereof, wherein when the compound is a salt, the salt is a pharmaceutically acceptable salt. In some embodiments, the cancer is selected from the group consisting of: lung, colorectal, pancreatic, bile duct, thyroid, gall bladder, uterine, mesothelioma, cervical, and bladder cancers. In some embodiments, the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the method is for treating the cancer. In some embodiments, including any of the foregoing embodiments, the method is for suppressing the cancer. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, the method further comprises administering to the subject a therapeutically effective amount of an additional chemotherapeutic agent.

In another aspect provided is the use of a compound as described herein, including but not limited to any of the foregoing embodiments, as a medicament. In another aspect is the use of a compound as described herein, including but not limited to any of the foregoing embodiments, for treating or suppressing cancer. In another aspect is the use of a compound as described herein, including but not limited to any of the foregoing embodiments, in the manufacture of a medicament for use in treating or suppressing cancer. In some embodiments, including any of the foregoing embodiments, the use is for treating the cancer. In some embodiments, including any of the foregoing embodiments, the use is for suppressing the cancer.

In another aspect provided is a compound as described herein, including but not limited to any of the foregoing embodiments for use in the manufacturing of a medicament for treating or suppressing cancer. In another aspect is a compound as described herein, including but not limited to any of the foregoing embodiments, for use in treating or suppressing cancer. In another aspect is the compound as described herein, including but not limited to any of the foregoing embodiments, for use in the manufacture of a medicament for treating or suppressing cancer. In some embodiments, including any of the foregoing embodiments, the use is for treating the cancer. In some embodiments, including any of the foregoing embodiments, the use is for suppressing the cancer.

It is to be understood that the description of compounds, compositions, formulations, and methods of treatment described herein include “comprising”, “consisting of”, and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

Additional embodiments, features, and advantages of the present disclosure will be apparent from the following detailed description and through practice of the present disclosure.

### DETAILED DESCRIPTION

Provided herein are compounds useful in treating cancer, and methods of using such compounds for treating cancer. In some embodiments, the compounds are useful in treating cancers characterized by KRAS G12C. In some embodiments, the compounds advantageously inhibit both the inactive GDP- and activated GTP-bound forms of KRAS G12C. In some embodiments, the compounds advantageously have improved inhibition of the GTP-bound form of KRAS G12C.

### Definitions

The abbreviations used herein have their conventional meaning within the chemical and biological arts, unless otherwise specified.

It is to be understood that descriptions of compound structures, including possible substitutions, are limited to those which are chemically possible.

Reference to “about” a value or parameter herein includes (and describes) variations that are directed to that value or parameter per se. For example, description referring to “about X” includes description of “X”. As used herein, and unless otherwise specified, the terms “about” and “approximately,” when used in connection with temperatures, doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 15%, within 10%, within 5%, within 4%, within 3%, within 2%, within 1%, or within 0.5% of the specified dose, amount, or weight percent.

The terms “a” and “an,” as used in herein mean one or more, unless context clearly dictates otherwise.

The terms “subject,” “individual,” and “patient” mean an individual organism, preferably a vertebrate, more preferably a mammal, most preferably a human. Examples of patients include humans, livestock such as cows, goats, sheep, pigs, and rabbits, and companion animals such as dogs, cats, and horses. In some embodiments, the subject has been identified or diagnosed as having a cancer or tumor having a KRAS G12C mutation (e.g., as determined using a regulatory agency-approved, e.g., FDA-approved, assay or kit).

“Treating” a disorder with the compounds and methods discussed herein is defined as administering one or more of the compounds discussed herein, with or without additional therapeutic agents, in order to reduce or eliminate either the disorder or one or more symptoms of the disorder, or to retard the progression of the disorder or of one or more symptoms of the disorder, or to reduce the severity of the disorder or of one or more symptoms of the disorder.

“Suppression” of a disorder with the compounds and methods discussed herein is defined as administering one or more of the compounds discussed herein, with or without additional therapeutic agents, in order to suppress the clinical manifestation of the disorder, or to suppress the manifestation of adverse symptoms of the disorder. The distinction between treatment and suppression is that treatment occurs after adverse symptoms of the disorder are manifest in a subject, while suppression occurs before adverse symptoms of the disorder are manifest in a subject. Suppression may be partial, substantially total, or total. In some embodiments, genetic screening can be used to identify patients at risk of the disorder. The compounds and methods disclosed herein can then be administered to asymptomatic patients at risk of developing the clinical symptoms of the disorder, in order to suppress the appearance of any adverse symptoms.

“Therapeutic use” of the compounds discussed herein is defined as using one or more of the compounds discussed herein to treat or suppress a disorder, as defined herein. A “therapeutically effective amount” of a compound is an amount of the compound, which, when administered to a subject, is sufficient to reduce or eliminate either the disorder or one or more symptoms of the disorder, or to retard the progression of the disorder or of one or more symptoms of the disorder, or to reduce the severity of the disorder or of one or more symptoms of the disorder, or to suppress the clinical manifestation of a disorder, or to suppress the manifestation of adverse symptoms of a disorder. A therapeutically effective amount can be given in one or more administrations.

A “KRAS G12C mediated cancer” is used interchangeably herein with a “cancer characterized by KRAS G12C”, and indicates that the cancer comprises cells which contain the KRAS G12C mutant.

While the compounds described herein can occur and can be used as the neutral (non-salt) compound, the description is intended to embrace all salts of the compounds described herein, as well as methods of using such salts of the compounds. In some embodiments, the salts of the compounds comprise pharmaceutically acceptable salts.

A “pharmaceutically acceptable salt” of a compound means a salt that is pharmaceutically acceptable to humans and/or animals, and which, upon administration, retains at least some of the desired pharmacological activity of the parent compound. Such salts include: (a) acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (b) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Additional information on suitable pharmaceutically acceptable salts can be found in Remington's Pharmaceutical Sciences, 17th ed., Mack Publishing Company, Easton, PA, 1985, which is incorporated herein by reference in its entirety.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

“Isotopologue” refers herein to a compound which differs in its isotopic composition from its “natural” isotopic composition. “Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition. The description of compounds herein also includes all isotopologues, in some embodiments, partially deuterated or perdeuterated analogs, of all compounds herein. “Isotopically enriched” may also refer to a compound containing at least one atom having an isotopic composition other than the natural isotopic composition of that atom. “Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Alkyl” means a linear, branched, or a combination thereof, saturated monovalent hydrocarbon radical having the defined number of carbons. For example, C₁-C₄ alkyl includes e.g., methyl, ethyl, propyl, 2-propyl, butyl, and the like.

“Alkylene” means a linear, branched, or a combination thereof, saturated divalent hydrocarbon radical having the defined number of carbons. For example, C₁-C₄ alkylene includes e.g., methylene, ethylene, propylene, 1-methylpropylene, 2-methylpropylene, butylene, and the like. “C₀ alkylene” means a bond. For example, C₀-C₂ alkylene includes a bond, methylene, ethylene, and the like.

“Alkynyl” means a linear or branched monovalent hydrocarbon radical having one or more triple bonds and the defined number of carbons. For example, C₂-C₄ alkyne includes e.g., ethynyl, propynyl, 2-propynyl, butynyl, and the like.

“Alkoxy” means an —OR radical where R is alkyl as defined above, or a —R′OR″ radical where R′ is an alkylene and and R″ is an alkyl group as defined above where the defined number of alkyl carbons in the alkoxy group are equal to the total number of carbons in R′ and R″. For example, C₁-C₄ alkoxy indicates e.g., methoxy, ethoxy, propoxy, 2-propoxy, n-, iso-, tert-butoxy, methoxymethyl, ethoxymethyl, propoxymethyl, isopropoxymethyl, and the like. In some embodiments, alkoxy is a —OR radical. In some embodiments, alkoxy is a —R′OR″ radical. In some embodiments, when a nitrogen is substituted with an alkoxy group, the alkoxy group is not linked to the nitrogen via the oxygen or a carbon that is immediately adjacent to the oxygen in the alkoxy group. For example, the alkoxy-substituted nitrogen is not N—OR or N—CH₂—O—R″.

“Alkoxyalkoxy” means an —OR radical where R is alkoxy as defined above, provided that the attachment point of R is not an oxygen atom, or a —R′OR″ radical where R′ is an alkylene and and R″ is an alkoxy group as defined above, provided that the the attachment point of R″ is not an oxygen atom, where the defined number of alkyl carbons in the alkoxyalkoxy group are equal to the total number of carbons in R′ and R″. For example, C₁-C₆ alkoxyalkoxy indicates e.g., —OCH₂OCH₃, —OCH₂CH₂OCH₃, —OCH₂CH₂OCH₃, —CH₂OCH₂OCH₃, —CH₂OCH₂CH₂OCH₃, —CH₂OCH₂CH₂OCH₂CH₃, —CH₂CH₂OCH₂CH₂OCH₂CH₃ and the like. In some embodiments, alkoxyalkoxy is a —OR radical. In some embodiments, alkoxyalkoxy is a —R′OR″ radical. In some embodiments, when a nitrogen is substituted with an alkoxyalkoxy group, the alkoxyalkoxy group is not linked to the nitrogen via the oxygen or a carbon that is immediately adjacent to the oxygen in the alkoxyalkoxy group. For example, the alkoxyalkoxy-substituted nitrogen is not N—OR or N—CH₂—O—R″.

“Aminoalkyl” means an —NHR radical where R is alkyl as defined above, or a —NRR′ radical where R and R′ are alkyl groups as defined above, or an —R″NH₂ radical where R″ is an alkylene group as defined above, or an —R″NHR radical where R″ is an alkylene group as defined above and R′ is an alkyl group as defined above, or a —R″NRR′ radical where where R″ is an alkylene group as defined above and R and R′ are alkyl groups as defined above, where the defined number of alkyl carbons in the aminoalkyl group is equal to the total number of carbons in R, R′ and R″ as applicable. For example, C₁-C₆ aminoalkyl indicates e.g., —NHCH₃, —NHCH₂CH₃, —NHCH₂ (CH₃)₂, —N(CH₃)₂, —N(CH₃)CH₂CH₃, —(CH₂CH₃)₂, —CH₂NH₂, —CH₂CH₂NH₂, —CH₂NHCH₃, —CH₂N(CH₃)₂, —CH₂CH₂NHCH₃, —CH₂CH₂N(CH₃)₂ and the like. In some embodiments, aminoalkyl is an —NHR radical. In some embodiments, aminoalkyl is an —NRR′ radical. In some embodiments, an aminoalkyl is an —R″NH₂ radical. In some embodiments, aminoalkyl is a —R″NHR radical. In some embodiments, aminoalkyl is a —R″NRR′ radical. In some embodiments, when an oxygen is substituted with an aminoalkyl group, the aminoalkyl group is not linked to the oxygen via the nitrogen or a carbon that is immediately adjacent to the nitrogen in the aminoalkyl group. For example, the aminoalkyl-substituted oxygen is not O—NR or O—CH₂—NHR.

“Cycloalkyl” means a monocyclic saturated monovalent hydrocarbon radical having the defined number of carbon atoms. For example, C₃-C₆ cycloalkyl includes cyclopropyl, cyclobutyl, cyclopentyl, and cyclohexyl.

“Cycloalkylene” means a monocyclic saturated divalent hydrocarbon radical having the defined number of carbon atoms. For example, C₃-C₆ cycloalkylene includes cyclopropylene, cyclobutylene, cyclopentylene, and cyclohexylene.

“Halo” means fluoro, chloro, bromo, or iodo. In some embodiments, halo is fluoro or chloro.

“Haloalkyl” means an alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means an —OR^{a}′ radical where R^{a}′ is haloalkyl as defined above, or a —R^{b}′OR″ radical where R^{b}′ and R^{c}′ are alkyl or haloalkyl groups as defined above where the defined number of alkyl carbons in the haloalkoxy group are equal to the total number of carbons in R^{b}′ and R^{c}′. Halo atom(s) may be present in R^{b}′, or R^{c}′, or both, provided that at least one of R^{b}′ and R^{c}′ comprises a halo atom. For example, C₁-C₄ haloalkoxy indicates e.g., —OCF₃, —OCHF₂, —CH₂OCF₃, —CH₂CH(F)CH₂OCH₃, —CH₂CH(F)CH₂OCHF₂, and the like. In some embodiments, haloalkoxy is a —OR^{a}′ radical. In some embodiments, haloalkoxy is a —R^{b}′OR″ radical. When all of the halo atom(s) in the haloalkoxy group are fluoro, it can be referred to in this Application as fluoroalkoxy. In some embodiments, when a nitrogen is substituted with a haloalkoxy group, the haloalkoxy group is not linked to the nitrogen via the oxygen or a carbon that is immediately adjacent to the oxygen in the haloalkoxy group. For example, the haloalkoxy-substituted nitrogen is not N—OR^{a}′ or N—C(H)ₙ(X)ₘ—O—R″.

“Hydroxyalkyl” means an alkyl radical as defined above, which is substituted with one or more hydroxyl (—OH) groups, e.g., one to three hydroxyl groups, e.g., —CH₂OH, —CH₂CH₂OH, —C(OH)(CH₃)₂, —CH(OH)CH₃ and the like.

A “heterocyclic group”, unless otherwise specified, means a saturated or partially unsaturated cyclic group comprising 3-12 ring atoms, in which 1-4 ring atoms are heteroatoms independently selected from the group consisting of nitrogen, oxygen, and sulfur, the remaining rings being C. The sulfur group may be present either as —S— or as —S(O)₂—. Unless otherwise specified, the heterocyclic group includes single as well as multiple ring systems including fused, bridged, and spiro ring systems. In some embodiments, the heterocyclic group is a single ring. In some embodiments, the heterocyclic group comprises two fused rings. In some embodiments, the heterocyclic group comprises two spiro rings. In some embodiments, the heterocyclic group comprises a bridged ring system.

A “carbocyclic group”, unless otherwise specified, means a saturated or partially unsaturated cyclic group comprising 3-12 ring atoms, in which the ring atoms are C. Unless otherwise specified, the carbocyclic group includes single as well as multiple ring systems including fused, bridged, and spiro ring systems. In some embodiments, the carbocyclic group is a single ring. In some embodiments, the carbocyclic group comprises two fused rings. In some embodiments, the carbocyclic group comprises two spiro rings. In some embodiments, the carbocyclic group comprises a bridged ring system.

“Heteroaryl” means a monovalent monocyclic or bicyclic aromatic radical of 5 to 10 ring atoms, unless otherwise stated, where one or more (in some embodiments, one, two, or three) ring atoms are heteroatom(s) independently selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

A “spiro” cycloalkyl group indicates that the cycloalkyl group is linked to the remaining portion of the compound through a spiro linkage. For example, a cyclohexyl group that is substituted with a “spiro C₃-C₄ cycloalkyl” group indicates:

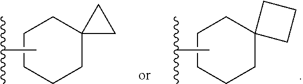

“In need of treatment” as used herein means the patient is being treated by a physician or other caregiver after diagnoses of the disease, or a determination that the patient is at risk for developing the disease. In some embodiments, the patient has been diagnosed as having a KRAS G12C mediated cancer. In some embodiments, the patient has been determined to be at risk of developing a KRAS G12C mediated cancer.

“Administration”, “administer” and the like, as they apply to, for example, a patient, cell, tissue, organ, or biological fluid, refer to contact of, for example, a compound of Formula (I), Formula (II), Formula (III) or Formula (IV), or a pharmaceutically acceptable salt and/or isotopologue thereof, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Optional” or “optionally” means that the subsequently described event or circumstance may but need not occur, and that the description includes instances where the event or circumstance occurs and instances in which it does not.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule or a tablet having a fixed ratio of active ingredients or in multiple, separate capsules or tablets for each active ingredient. In addition, such administration also encompasses use of each type of therapeutic agent in a sequential manner. In either case, the treatment regimen will provide beneficial effects of the drug combination in treating the conditions or disorders described herein.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (cis/trans), E/Z isomers, enantiomers, diastereomers, and mixtures thereof in any ratio including racemic mixtures, salts and solvates of the compounds described herein, as well as methods of making such compounds. Any compound described herein may also be referred to as a drug.

Provided herein are compounds of Formula (I), Formula (II), Formula (III) and Formula (IV). Unless the context requires otherwise, reference throughout this specification to “a compound of Formula (I) and/or a compound of Formula (II) and/or a compound of Formula (III) and/or a Compound of Formula (IV)” or “compounds of Formula (I) and/or compounds of Formula (II) and/or compounds of Formula (III) and/or compounds of Formula (IV)” refers to all embodiments of Formula (I) Formula (II), Formula (III) and Formula (IV), including, for example, compounds of Formulas (I-a), (I-b), (I-c), (II-a), (II-b), (II-c) as well as the compounds of Table 1. In some embodiments, provided are compounds of Formula (I) and/or Formula (II) and/or Formula (III) and/or Formula (IV) or pharmaceutically acceptable salts thereof. In some embodiments, the compounds of Formula (I) and/or Formula (II) and/or Formula (III) and/or Formula (IV) are provided as pharmaceutically acceptable salts. In some embodiments, the compounds of Formula (I) and/or Formula (II) and/or Formula (III) and/or Formula (IV) are provided as the corresponding free base (i.e., are not salts).

In an embodiment, provided is a compound of Formula (I), Formula (II), Formula (III) or Formula (IV):

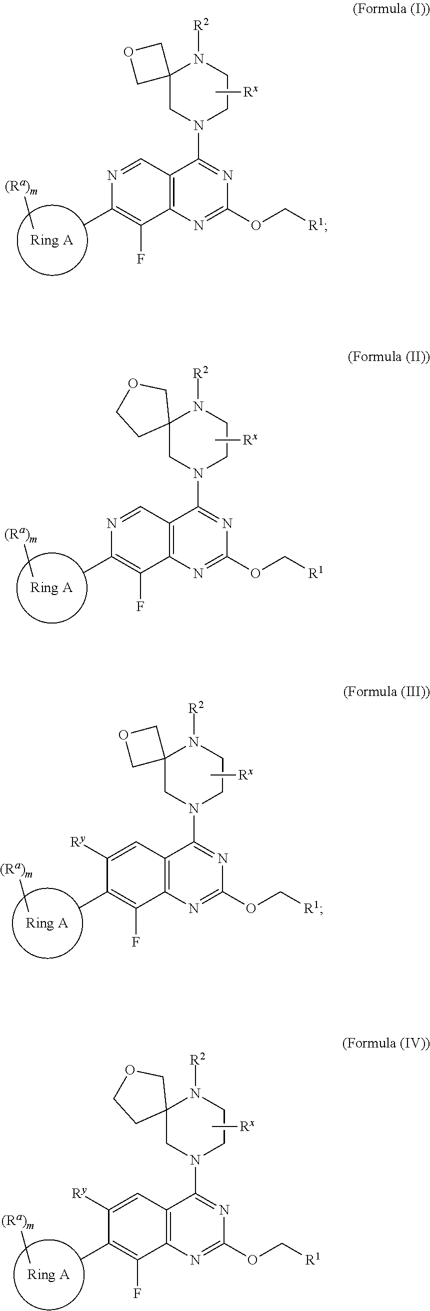

or a salt thereof; and/or an isotopologue thereof; wherein:
Ring A is a 6-10 membered aryl or a 5-10 membered heteroaryl;
each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
m is 0, 1, 2 or 3;
R¹ is

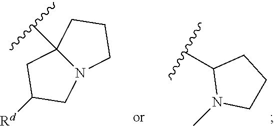

R^{d} is H or F;
R² is

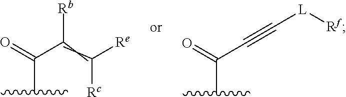

R^{b} is —H or —F;
R^{c} is —H;
R^{e} is —H, —Y—R^{e1}, —Y—R^{e2}, or —Y—R^{e3};
Y is —(C(R^{Y1})(R^{Y2}))ₓ—;
x is 0, 1 or 2 provided that when R^{e} is Y—R^{e3}, x is not 0;
R^{Y1} and R^{Y2} in each occurrence are independently selected from —H and —CH₃;
R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
L is —(C(R^{L1})(L^{L2}))_{z}—;
z is 0, 1 or 2;
R^{L1} and R^{L2} in each occurrence are independently selected from —H and —CH₃;
R^{f} is a 4-10 membered heterocycle which is substituted with 0, 1, 2, 3 or 4 substituents independently selected from halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl;
R^{x} is selected from —H and C₁-C₄ alkyl; and
R^{y} is selected from halo and C₁-C₄ haloalkyl.

In an embodiment, provided is a compound of Formula (I), Formula (II), Formula (III) or Formula (IV) or a salt thereof; and/or an isotopologue thereof, wherein Ring A, m, R¹, R², R^{x}, and R^{y} are as defined in any of the embodiments described herein.

In an embodiment, the compound is of Formula (I).

In an embodiment, the compound is of Formula (II).

In an embodiment, the compound is of Formula (III).

In an embodiment, the compound is of Formula (IV).

In an embodiment, provided is a compound of Formula (I-a):

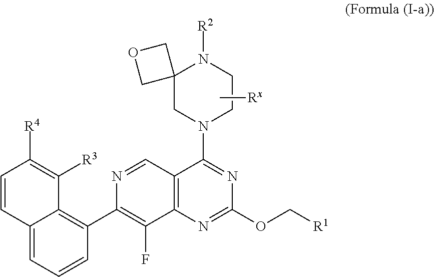

or a salt thereof; and/or an isotopologue thereof; wherein R¹, R², R³, R⁴ and R^{x} are as defined herein.

In an embodiment, provided is a compound of Formula (I-b):

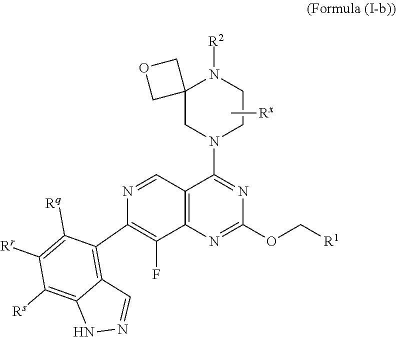

or a salt thereof; and/or an isotopologue thereof; wherein R¹, R², R^{x} R^{q}, R^{r}, R^{s} and R^{x} are as defined herein.

In an embodiment, provided is a compound of Formula (I-c):

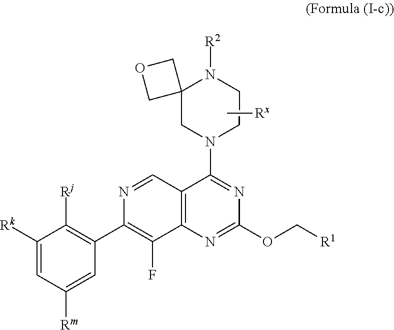

or a salt thereof; and/or an isotopologue thereof; wherein R¹, R², R^{x}, R^{j}, R^{k}, R^{m} and R^{x} are as defined herein.

In an embodiment, provided is a compound of Formula (II-a):

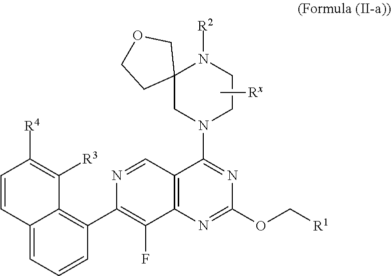

or a salt thereof; and/or an isotopologue thereof; wherein R¹, R², R³, R⁴ and R^{x} are as defined herein.

In an embodiment, provided is a compound of Formula (II-b):

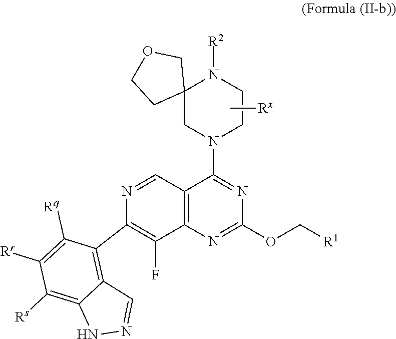

or a salt thereof; and/or an isotopologue thereof; wherein R¹, R², R^{x}, R^{q}, R^{r}, R^{s} and R^{x} are as defined herein.

In an embodiment, provided is a compound of Formula (II-c):

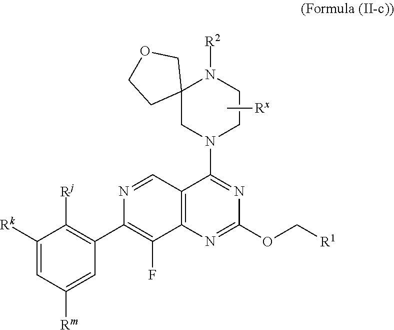

or a salt thereof; and/or an isotopologue thereof; wherein R¹, R², R^{x}, Rⁱ, R^{k}, R^{m} and R^{x} are as defined herein.

In an embodiment the stereochemistry of the spirotetrahydrofuran ring in Formula (II), (IV), (II-a), (II-b) and (II-c) is R. In an embodiment the stereochemistry of the spirotetrahydrofuran ring in Formula (II), (IV), (II-a), (II-b) and (II-c) is S.

As generally defined herein, Ring A is a 6-10 membered aryl or a 5-10 membered heteroaryl.

In an embodiment, Ring A is selected from a 6-10 membered aryl and a 9-10 membered bicyclic heteroaryl having 1, 2 or 3 heteroatoms independently selected from N, O and S.

In an embodiment, Ring A is selected from the group consisting of naphthalenyl, phenyl, benzothiazolyl, isoquinolinyl, indazolyl and pyridinyl. In an embodiment, Ring A is selected from the group consisting of naphthalenyl, phenyl, isoquinolinyl, indazolyl and pyridinyl. In an embodiment, Ring A is selected from the group consisting of naphthalenyl, phenyl, benzothiazolyl and indazolyl. In an embodiment, Ring A is selected from the group consisting of naphthalenyl, phenyl and indazolyl. In an embodiment, Ring A is selected from the group consisting of naphthalenyl and phenyl.

In an embodiment, Ring A is naphthalenyl. In an embodiment, Ring A is phenyl. In an embodiment, Ring A is indazolyl. In an embodiment ring A is benzothiazolyl (e.g., benzo[d]thiazolyl).

In an embodiment, Ring A is selected from the group consisting of naphthalen-1-yl, phenyl, isoquinolin-1-yl, benzo[d]thiazol-4-yl, indazol-3-yl, indazol-4-yl and pyridin-1-yl. In an embodiment, Ring A is selected from the group consisting of naphthalen-1-yl, phenyl, isoquinolin-1-yl, indazol-4-yl and pyridin-1-yl. In an embodiment, Ring A is selected from the group consisting of naphthalen-1-yl, phenyl, and indazol-4-yl. In an embodiment, Ring A is selected from the group consisting of naphthalen-1-yl and phenyl. In an embodiment, Ring A is naphthalen-1-yl. In an embodiment, Ring A is phenyl. In an embodiment, Ring A is indazol-4-yl.

In an embodiment, Ring A is selected from the group consisting of:

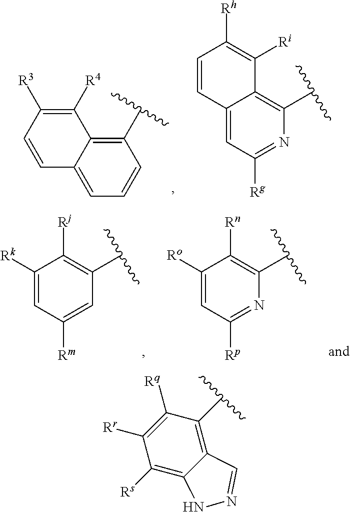

wherein each R³, R⁴, R^{h}, Rⁱ, R^{j}, R^{k}, Rⁿ, R^{o}, R^{q}, R^{s}, R^{g}, R^{m} and R^{p} is as defined herein.

In an embodiment, Ring A is selected from the group consisting of:

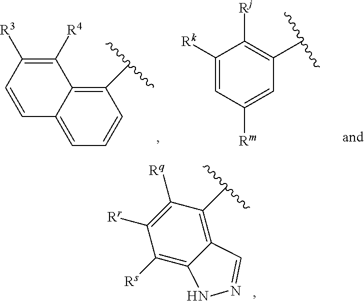

wherein each R³, R⁴, R^{q}, R^{s}, and R^{m} is as defined herein.

In an embodiment, Ring A is selected from the group consisting of:

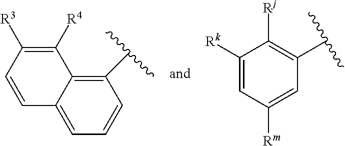

wherein each R³, R⁴, R^{h}, Rⁱ, R^{j}, R^{k}, and R^{m} is as defined herein. In an embodiment, Ring A is

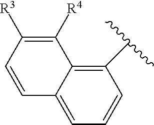

wherein each R³ and R⁴ is as defined herein. In an embodiment, Ring A is

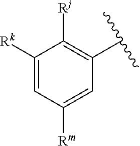

wherein each R^{j}, R^{k}, and R^{m} is as defined herein. In an embodiment, Ring A is

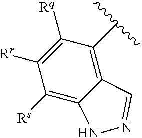

wherein each R⁹, R^{r} and R^{s} is as defined herein. In an embodiment, Ring A is

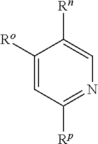

wherein each Rⁿ, R^{o} and R^{p} is as defined herein.

In an embodiment, Ring A is selected from:

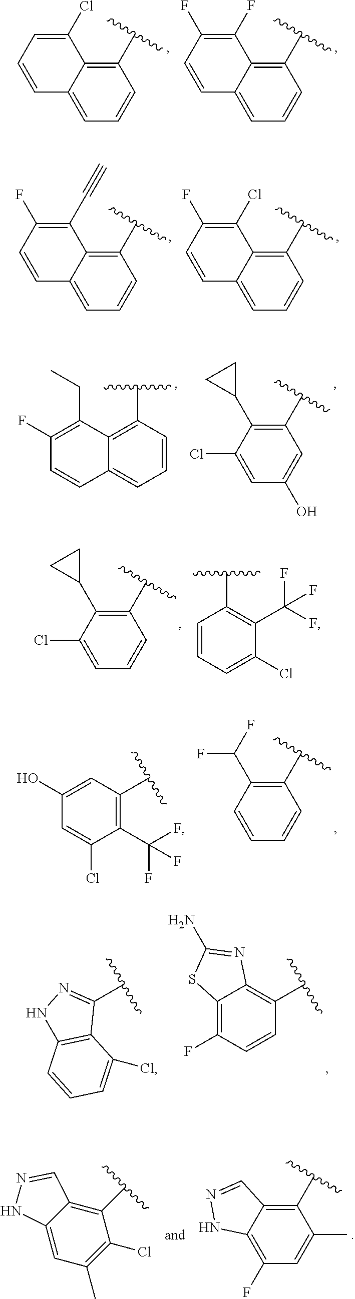

In an embodiment, Ring A is selected from:

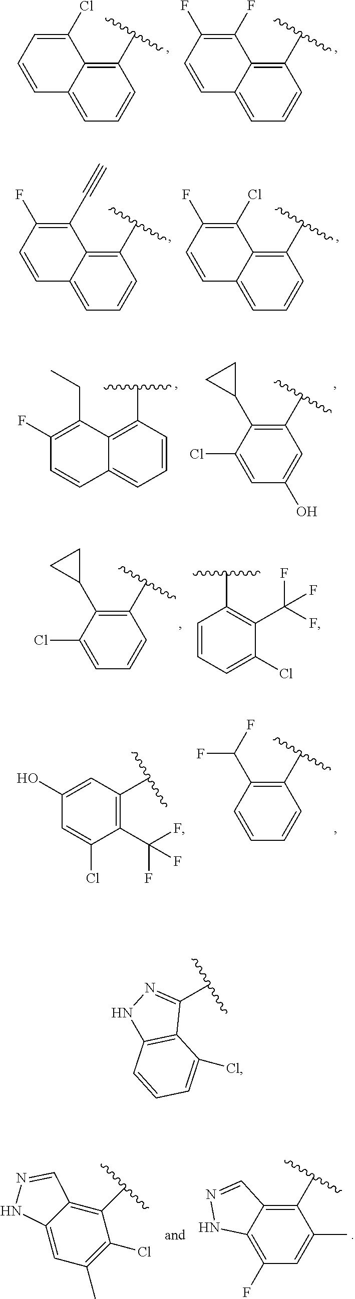

In an embodiment, Ring A is selected from:

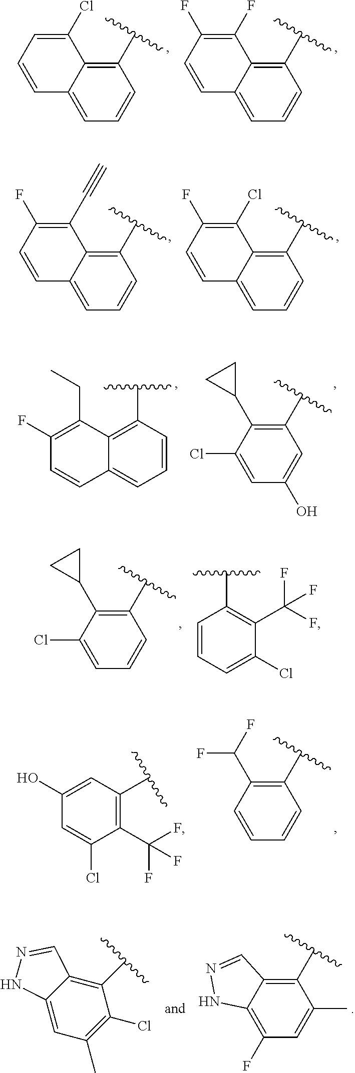

In an embodiment, Ring A is selected from:

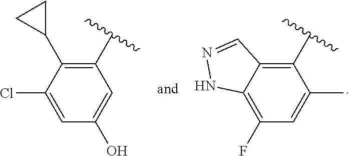 [SMILES: *c1cc(O)cc(Cl)c1C1CC1 ; *c1c(C)cc(F)c2[nH]ncc12]

In an embodiment, Ring A is

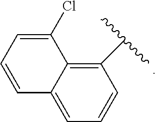 [SMILES: *c1cccc2cccc(Cl)c12]

In an embodiment, Ring A is

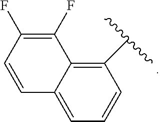 [SMILES: *c1cccc2ccc(F)c(F)c12]

In an embodiment, Ring A is

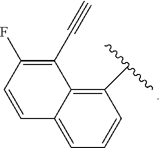 [SMILES: *c1cccc2ccc(F)c(C#C)c12]

In an embodiment, Ring A is

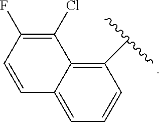 [SMILES: *c1cccc2ccc(F)c(Cl)c12]

In an embodiment, Ring A is

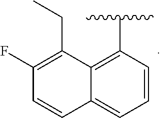 [SMILES: *c1cccc2ccc(F)c(CC)c12 ; "."]

In an embodiment, Ring A is

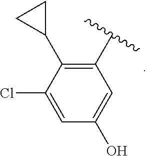 [SMILES: *c1cc(O)cc(Cl)c1C1CC1]

In an embodiment, Ring A is

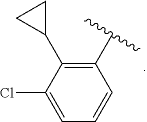 [SMILES: *c1cccc(Cl)c1C1CC1]

In an embodiment, Ring A is

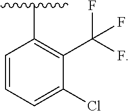 [SMILES: *c1cccc(Cl)c1C(F)(F)F]

In an embodiment, Ring A is

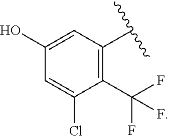 [SMILES: *c1cc(O)cc(Cl)c1C(F)(F)F]

In an embodiment, Ring A is

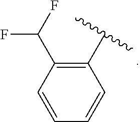 [SMILES: *c1ccccc1C(F)F]

In an embodiment, Ring A is

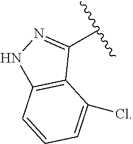 [SMILES: *c1n[nH]c2cccc(Cl)c12]

In an embodiment, Ring A is

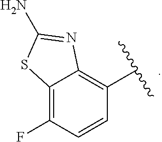 [SMILES: *c1ccc(F)c2sc(N)nc12]

In an embodiment, Ring A is

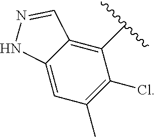 [SMILES: *c1c(Cl)c(C)cc2[nH]ncc12]

In an embodiment, Ring A is

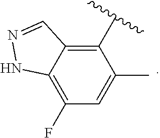 [SMILES: *c1c(C)cc(F)c2[nH]ncc12]

As generally defined herein, each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

In an embodiment, each R^{a} is independently selected from halo, —OH, C₁-C₄ alkyl, C₃-C₄ cycloalkyl, C₁-C₄ haloalkyl and C₂-C₃ alkynyl.

In an embodiment, each R^{a} is independently selected from halo and C₁-C₄ alkyl. In an embodiment, each R^{a} is independently selected from halo and C₃-C₄ cycloalkyl. In an embodiment, each R^{a} is independently selected from halo and C₁-C₄ haloalkyl. In an embodiment, each R^{a} is independently selected from halo and C₂-C₃ alkynyl. In an embodiment, each R^{a} is independently selected from halo and —NH₂. In an embodiment, each R^{a} is independently selected from —F, —Cl, —OH, —NH₂, -Me, -Et, -cyclopropyl, —CF₃ and —C═CH. In an embodiment, each R^{a} is independently selected from OH, —Cl and -cyclopropyl. In an embodiment, each R^{a} is independently selected from —F, and —NH₂. In an embodiment, each R^{a} is independently halo.

In an embodiment, each R^{a} is independently selected from —F, —Cl, —OH, -Me, -Et, -cyclopropyl, —CF₃ and —C═CH. In an embodiment, each R^{a} is independently selected from —F, —Cl, -Me and -Et. In an embodiment, each R^{a} is independently selected from —F, —Cl and -cyclopropyl. In an embodiment, each R^{a} is independently selected from —F, —Cl and —CF₃. In an embodiment, each R^{a} is independently selected from —F, —Cl and —C═CH. In an embodiment, each R^{a} is independently selected from —F and —Cl. In an embodiment, each R^{a} is —F. In an embodiment, each R^{a} is —Cl. In an embodiment, each R^{a} is —OH. In an embodiment, each R^{a} is -Me. In an embodiment, each R^{a} is -Et. In an embodiment, each R^{a} is -cyclopropyl. In an embodiment, each R^{a} is —CF₃. In an embodiment, each R^{a} is —C═CH.

As generally defined herein, m is 0, 1, 2 or 3. In an embodiment, m is 1, 2 or 3. In an embodiment, m is 1 or 2. In an embodiment, m is 2 or 3. In an embodiment, m is 1. In an embodiment, m is 2. In an embodiment, m is 3.

As generally defined herein, each R³ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R³ is independently selected from the group consisting of hydrogen, halo, C₁-C₄ alkyl, C₃-C₄ cycloalkyl, C₁-C₄ haloalkyl and C₂-C₃ alkynyl. In an embodiment, each R³ is independently selected from the group consisting of hydrogen, halo and C₁-C₄ alkyl. In an embodiment, each R³ is independently selected from the group consisting of hydrogen, halo and C₃-C₄ cycloalkyl. In an embodiment, each R³ is independently selected from the group consisting of hydrogen, halo and C₁-C₄ haloalkyl. In an embodiment, each R³ is independently selected from the group consisting of hydrogen, halo and C₂-C₃ alkynyl. In an embodiment, R³ is selected from halo and C₁-C₄ alkyl. In an embodiment, R³ is selected from halo and C₂-C₃ alkynyl. In an embodiment, each R³ is independently selected from the group consisting of hydrogen and halo. In an embodiment, each R³ is independently halo. In an embodiment, each R³ is independently selected from the group consisting of —H, —F, —Cl, -Me, -Et, -cyclopropyl, —CF₃ and —C═CH. In an embodiment, each R³ is independently selected from the group consisting of —H, —F, —Cl, -Me and -Et. In an embodiment, each R³ is independently selected from the group consisting of —H, —F, —Cl and -cyclopropyl. In an embodiment, each R³ is independently selected from the group consisting of —H, —F, —Cl and —CF₃. In an embodiment, each R³ is independently selected from the group consisting of —H, —F, —Cl and —C═CH. In an embodiment, each R³ is independently selected from the group consisting of —H, —F and —Cl. In an embodiment, R³ is selected from —F, —Cl, -Et, and —C═CH. In an embodiment, R³ is selected from —F, —Cl, and -Et. In an embodiment, R³ is selected from —F, —Cl and —C═CH. In an embodiment, each R³ is independently selected from the group consisting of —F and —Cl. In an embodiment, R³ is —F. In an embodiment, R³ is —Cl. In an embodiment, R³ is -Et. In an embodiment, R³ is —C═CH.

As generally defined herein, each R⁴ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R⁴ is independently selected from the group consisting of hydrogen, halo, C₁-C₄ alkyl, C₃-C₄ cycloalkyl, C₁-C₄ haloalkyl and C₂-C₃ alkynyl. In an embodiment, each R⁴ is independently selected from the group consisting of hydrogen, halo and C₁-C₄ alkyl. In an embodiment, each R⁴ is independently selected from the group consisting of hydrogen, halo and C₃-C₄ cycloalkyl. In an embodiment, each R⁴ is independently selected from the group consisting of hydrogen, halo and C₁-C₄ haloalkyl. In an embodiment, each R⁴ is independently selected from the group consisting of hydrogen, halo and C₂-C₃ alkynyl. In an embodiment, each R⁴ is independently selected from the group consisting of hydrogen and halo. In an embodiment, each R⁴ is independently halo. In an embodiment, each R⁴ is independently selected from the group consisting of —H, —F, —Cl, -Me, -Et, -cyclopropyl, —CF₃ and —C═CH. In an embodiment, each R⁴ is independently selected from the group consisting of —H, —F, —Cl, -Me and -Et. In an embodiment, each R⁴ is independently selected from the group consisting of —H, —F, —Cl and -cyclopropyl. In an embodiment, each R⁴ is independently selected from the group consisting of —H, —F, —Cl and —CF₃. In an embodiment, each R⁴ is independently selected from the group consisting of —H, —F, —Cl and —C═CH. In an embodiment, each R⁴ is independently selected from the group consisting of —H, —F and —Cl. In an embodiment, each R⁴ is independently selected from the group consisting of —F and —Cl. In an embodiment, R⁴ is selected from —H and —F. In an embodiment, R⁴ is —H. In an embodiment, R⁴ is —F.

As generally defined herein, each R^{h} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{h} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{h} is independently selected from the group consisting of hydrogen, halo and C₁-C₄ alkyl. In an embodiment, each R^{h} is independently selected from the group consisting of hydrogen, halo and C₃-C₄ cycloalkyl. In an embodiment, each R^{h} is independently selected from the group consisting of hydrogen, halo and C₁-C₄ haloalkyl. In an embodiment, each R^{h} is independently selected from the group consisting of hydrogen, halo and C₂-C₃ alkynyl. In an embodiment, each R^{h} is independently selected from the group consisting of hydrogen and halo. In an embodiment, each R^{h} is independently halo. In an embodiment, each R^{h} is independently selected from the group consisting of —H, —F, —Cl, -Me, -Et, -cyclopropyl, —CF₃ and —C═CH. In an embodiment, each R^{h} is independently selected from the group consisting of —H, —F, —Cl, -Me and -Et. In an embodiment, each R^{h} is independently selected from the group consisting of —H, —F, —Cl and -cyclopropyl. In an embodiment, each R^{h} is independently selected from the group consisting of —H, —F, —Cl and —CF₃. In an embodiment, each R^{h} is independently selected from the group consisting of —H, —F, —Cl and —C═CH. In an embodiment, each R^{h} is independently selected from the group consisting of —H, —F and —Cl. In an embodiment, each R^{h} is independently selected from the group consisting of —F and —Cl.

As generally defined herein, each R¹ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R¹ is independently selected from the group consisting of hydrogen, halo, C₁-C₄ alkyl, C₃-C₄ cycloalkyl, C₁-C₄ haloalkyl and C₂-C₃ alkynyl. In an embodiment, each R¹ is independently selected from the group consisting of hydrogen, halo and C₁-C₄ alkyl. In an embodiment, each R¹ is independently selected from the group consisting of hydrogen, halo and C₃-C₄ cycloalkyl. In an embodiment, each R¹ is independently selected from the group consisting of hydrogen, halo and C₁-C₄ haloalkyl. In an embodiment, each R¹ is independently selected from the group consisting of hydrogen, halo and C₂-C₃ alkynyl. In an embodiment, each R¹ is independently selected from the group consisting of hydrogen and halo. In an embodiment, each R¹ is independently halo. In an embodiment, each Rⁱ is independently selected from the group consisting of —H, —F, —Cl, -Me, -Et, -cyclopropyl, —CF₃ and —C═CH. In an embodiment, each R¹ is independently selected from the group consisting of —H, —F, —Cl, -Me and -Et. In an embodiment, each R¹ is independently selected from the group consisting of —H, —F, —Cl and -cyclopropyl. In an embodiment, each R¹ is independently selected from the group consisting of —H, —F, —Cl and —CF₃. In an embodiment, each R¹ is independently selected from the group consisting of —H, —F, —Cl and —C═CH. In an embodiment, each R¹ is independently selected from the group consisting of —H, —F and —Cl. In an embodiment, each R¹ is independently selected from the group consisting of —F and —Cl.

As generally defined herein, each R^{j} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{j} is independently selected from the group consisting of hydrogen, halo, C₁-C₄ alkyl, C₃-C₄ cycloalkyl, C₁-C₄ haloalkyl and C₂-C₃ alkynyl. In an embodiment, each R¹ is independently selected from the group consisting of hydrogen, halo and C₁-C₄ alkyl. In an embodiment, each R¹ is independently selected from the group consisting of hydrogen, halo and C₃-C₄ cycloalkyl. In an embodiment, each R¹ is independently selected from the group consisting of hydrogen, halo and C₁-C₄ haloalkyl. In an embodiment, each R¹ is independently selected from the group consisting of hydrogen, halo and C₂-C₃ alkynyl. In an embodiment, each R^{j} is independently selected from the group consisting of hydrogen and halo. In an embodiment, R^{j} is selected from C₃-C₄ cycloalkyl and C₁-C₄ haloalkyl. In an embodiment, each R^{j} is independently halo. In an embodiment, each R^{j} is independently selected from the group consisting of —H, —F, —Cl, -Me, -Et, -cyclopropyl, —CF₃ and —C═CH. In an embodiment, each R¹ is independently selected from the group consisting of —H, —F, —Cl, -Me and -Et. In an embodiment, each R^{j} is independently selected from the group consisting of —H, —F, —Cl and -cyclopropyl. In an embodiment, each R¹ is independently selected from the group consisting of —H, —F, —Cl and —CF₃. In an embodiment, each R¹ is independently selected from the group consisting of —H, —F, —Cl and —C═CH. In an embodiment, each R^{j} is independently selected from the group consisting of —H, —F and —Cl. In an embodiment, each R¹ is independently selected from the group consisting of —F and —Cl. In an embodiment, R^{j} is selected from cyclopropyl, —CHF₂ and —CF₃. In an embodiment, R^{j} is cyclopropyl. In an embodiment, R^{j} is —CHF₂. In an embodiment, R^{j} is —CF₃.

As generally defined herein, each R^{k} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{k} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{k} is independently selected from the group consisting of hydrogen, halo and C₁-C₄ alkyl. In an embodiment, each R^{k} is independently selected from the group consisting of hydrogen, halo and C₃-C₄ cycloalkyl. In an embodiment, each R^{k} is independently selected from the group consisting of hydrogen, halo and C₁-C₄ haloalkyl. In an embodiment, each R^{k} is independently selected from the group consisting of hydrogen, halo and C₂-C₃ alkynyl. In an embodiment, each R^{k} is independently selected from the group consisting of hydrogen and halo. In an embodiment, each R^{k} is independently halo. In an embodiment, each R^{k} is independently selected from the group consisting of —H, —F, —Cl, -Me, -Et, -cyclopropyl, —CF₃ and —C═CH. In an embodiment, each R^{k} is independently selected from the group consisting of —H, —F, —Cl, -Me and -Et. In an embodiment, each R^{k} is independently selected from the group consisting of —H, —F, —Cl and -cyclopropyl. In an embodiment, each R^{k} is independently selected from the group consisting of —H, —F, —Cl and —CF₃. In an embodiment, each R^{k} is independently selected from the group consisting of —H, —F, —Cl and —C═CH. In an embodiment, each R^{k} is independently selected from the group consisting of —H, —F and —Cl. In an embodiment, each R^{k} is independently selected from the group consisting of —F and —Cl. In an embodiment, R^{k} is selected from —H and —Cl. In an embodiment, R^{k} is —H. In an embodiment, R^{k} is —Cl.

As generally defined herein, each Rⁿ is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each Rⁿ is independently selected from the group consisting of hydrogen, halo, C₁-C₄ alkyl, C₃-C₄ cycloalkyl, C₁-C₄ haloalkyl and C₂-C₃ alkynyl. In an embodiment, each Rⁿ is independently selected from the group consisting of hydrogen, halo and C₁-C₄ alkyl. In an embodiment, each Rⁿ is independently selected from the group consisting of hydrogen, halo and C₃-C₄ cycloalkyl. In an embodiment, each Rⁿ is independently selected from the group consisting of hydrogen, halo and C₁-C₄ haloalkyl. In an embodiment, each Rⁿ is independently selected from the group consisting of hydrogen, halo and C₂-C₃ alkynyl. In an embodiment, each Rⁿ is independently selected from the group consisting of hydrogen and halo. In an embodiment, each Rⁿ is independently halo. In an embodiment, each Rⁿ is independently selected from the group consisting of —H, —F, —Cl, -Me, -Et, -cyclopropyl, —CF₃ and —C═CH. In an embodiment, each Rⁿ is independently selected from the group consisting of —H, —F, —Cl, -Me and -Et. In an embodiment, each Rⁿ is independently selected from the group consisting of —H, —F, —Cl and -cyclopropyl. In an embodiment, each Rⁿ is independently selected from the group consisting of —H, —F, —Cl and —CF₃. In an embodiment, each Rⁿ is independently selected from the group consisting of —H, —F, —Cl and —C═CH. In an embodiment, each Rⁿ is independently selected from the group consisting of —H, —F and —Cl. In an embodiment, each Rⁿ is independently selected from the group consisting of —F and —Cl.

As generally defined herein, each R^{o} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{o} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{o} is independently selected from the group consisting of hydrogen, halo and C₁-C₄ alkyl. In an embodiment, each R^{o} is independently selected from the group consisting of hydrogen, halo and C₃-C₄ cycloalkyl. In an embodiment, each R^{o} is independently selected from the group consisting of hydrogen, halo and C₁-C₄ haloalkyl. In an embodiment, each R^{o} is independently selected from the group consisting of hydrogen, halo and C₂-C₃ alkynyl. In an embodiment, each R^{o} is independently selected from the group consisting of hydrogen and halo. In an embodiment, each R^{o} is independently halo. In an embodiment, each R^{o} is independently selected from the group consisting of —H, —F, —Cl, -Me, -Et, -cyclopropyl, —CF₃ and —C═CH. In an embodiment, each R^{o} is independently selected from the group consisting of —H, —F, —Cl, -Me and -Et. In an embodiment, each R^{o} is independently selected from the group consisting of —H, —F, —Cl and -cyclopropyl. In an embodiment, each R^{o} is independently selected from the group consisting of —H, —F, —Cl and —CF₃. In an embodiment, each R^{o} is independently selected from the group consisting of —H, —F, —Cl and —C═CH. In an embodiment, each R^{o} is independently selected from the group consisting of —H, —F and —Cl. In an embodiment, each R^{o} is independently selected from the group consisting of —F and —Cl.

As generally defined herein, each R^{a} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{a} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{a} is independently selected from the group consisting of hydrogen, halo and C₁-C₄ alkyl. In an embodiment, each R^{a} is independently selected from the group consisting of hydrogen, halo and C₃-C₄ cycloalkyl. In an embodiment, each R^{a} is independently selected from the group consisting of hydrogen, halo and C₁-C₄ haloalkyl. In an embodiment, each R^{a} is independently selected from the group consisting of hydrogen, halo and C₂-C₃ alkynyl. In an embodiment, each R^{a} is independently selected from the group consisting of hydrogen and halo. In an embodiment, R^{a} is selected from the group consisting of halo and C₁-C₄ alkyl.

In an embodiment, each R^{a} is independently halo. In an embodiment, each R^{a} is independently selected from the group consisting of —H, —F, C₁, -Me, -Et, -cyclopropyl, —CF₃ and —C═CH. In an embodiment, each R^{a} is independently selected from the group consisting of —H, —F, —Cl, -Me and -Et. In an embodiment, each R^{a} is independently selected from the group consisting of —H, —F, —Cl and -cyclopropyl. In an embodiment, each R^{a} is independently selected from the group consisting of —H, —F, —Cl and —CF₃. In an embodiment, each R^{a} is independently selected from the group consisting of —H, —F, —Cl and —C═CH. In an embodiment, each R^{a} is independently selected from the group consisting of —H, —F and —Cl. In an embodiment, each R^{a} is independently selected from the group consisting of —F and —Cl. In an embodiment, each R^{a} is selected from the group consisting of hydrogen, —F, —Cl, and -Me. In an embodiment, each R^{a} is selected from the group consisting of —Cl, and -Me. In an embodiment, each R^{a} is —Cl. In an embodiment, each R^{a} is -Me.

As generally defined herein, each R^{T} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{r} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{r} is independently selected from the group consisting of hydrogen, halo and C₁-C₄ alkyl. In an embodiment, each R^{r} is independently selected from the group consisting of hydrogen, halo and C₃-C₄ cycloalkyl. In an embodiment, each R^{T} is independently selected from the group consisting of hydrogen, halo and C₁-C₄ haloalkyl. In an embodiment, each R^{r} is independently selected from the group consisting of hydrogen, halo and C₂-C₃ alkynyl. In an embodiment, each R^{T} is independently selected from the group consisting of hydrogen and halo. In an embodiment, R^{r} is selected from the group consisting of hydrogen and C₁-C₄ alkyl. In an embodiment, each R^{T} is independently halo. In an embodiment, each R^{r} is independently selected from the group consisting of —H, —F, —Cl, -Me, -Et, -cyclopropyl, —CF₃ and —C═CH. In an embodiment, each R^{r} is independently selected from the group consisting of —H, —F, —Cl, -Me and -Et. In an embodiment, each R^{r} is independently selected from the group consisting of —H, —F, —Cl and -cyclopropyl. In an embodiment, each R^{T} is independently selected from the group consisting of —H, —F, —Cl and —CF₃. In an embodiment, each R^{T} is independently selected from the group consisting of —H, —F, —Cl and —C═CH. In an embodiment, each R^{r} is independently selected from the group consisting of —H, —F and —Cl. In an embodiment, each R^{T} is independently selected from the group consisting of —F and —Cl. In an embodiment, R^{r} is selected from the group consisting of —H and -Me. In an embodiment, R^{r} is —H. In an embodiment, R^{r} is -Me.

As generally defined herein, each R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{s} is independently selected from the group consisting of hydrogen, halo, C₁-C₄ alkyl, C₃-C₄ cycloalkyl, C₁-C₄ haloalkyl and C₂-C₃ alkynyl. In an embodiment, each R^{s} is independently selected from the group consisting of hydrogen, halo and C₁-C₄ alkyl. In an embodiment, each R^{s} is independently selected from the group consisting of hydrogen, halo and C₃-C₄ cycloalkyl. In an embodiment, each R^{s} is independently selected from the group consisting of hydrogen, halo and C₁-C₄ haloalkyl. In an embodiment, each R^{s} is independently selected from the group consisting of hydrogen, halo and C₂-C₃ alkynyl. In an embodiment, each R^{s} is independently selected from the group consisting of hydrogen and halo. In an embodiment, each R^{s} is independently halo. In an embodiment, each R^{s} is independently selected from the group consisting of —H, —F, —Cl, -Me, -Et, -cyclopropyl, —CF₃ and —C═CH. In an embodiment, each R^{s} is independently selected from the group consisting of —H, —F, —Cl, -Me and -Et. In an embodiment, each R^{s} is independently selected from the group consisting of —H, —F, —Cl and -cyclopropyl. In an embodiment, each R^{s} is independently selected from the group consisting of —H, —F, —Cl and —CF₃. In an embodiment, each R^{s} is independently selected from the group consisting of —H, —F, —Cl and —C═CH. In an embodiment, each R^{s} is independently selected from the group consisting of —H, —F and —Cl. In an embodiment, each R^{s} is independently selected from the group consisting of —F and —Cl. In an embodiment, R^{s} is selected from the group consisting of —H and —F. In an embodiment, R^{s} is —H. In an embodiment, R^{s} is —F.

As generally defined herein, each R& is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R& is independently selected from the group consisting of —H and —OH. In an embodiment, each R& is independently —H. In an embodiment, each R& is independently —OH.

As generally defined herein, each R^{m} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{m} is independently selected from the group consisting of —H and —OH. In an embodiment, each R^{m} is independently —H. In an embodiment, each R^{m} is independently —OH.

As generally defined herein, each R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. In an embodiment, each R^{p} is independently selected from the group consisting of —H and —OH. In an embodiment, each R^{p} is independently —H. In an embodiment, each R^{p} is independently —OH.

As generally defined herein, R¹ is

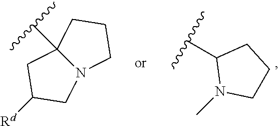

wherein R^{d} is as defined herein. In an embodiment, R¹ is selected from

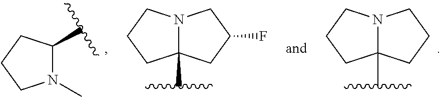

In an embodiment, R¹ is

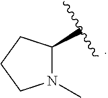 [SMILES: *[C@@H]1CCCN1C]

In an embodiment, R¹ is

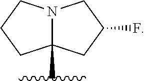 [SMILES: *[C@@]12CCCN1C[C@H](F)C2]

In an embodiment, R¹ is

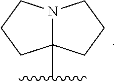 [SMILES: *C12CCCN1CCC2]

As generally defined herein, R^{d} is H or F. In an embodiment, R^{d} is H. In an embodiment, R^{d} is F.

As generally defined herein, R² is

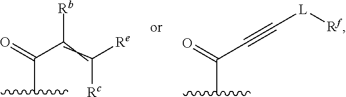

wherein R^{b}, R^{c}, R^{e}, L and R^{f} are as defined herein.

In an embodiment, R² is

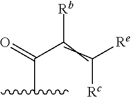

wherein R^{b}, R^{c} R^{e} are as defined herein. In an embodiment, R² is

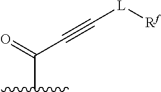

wherein L and R^{f} are as defined herein.

In an embodiment, R² is selected from the group consisting of:

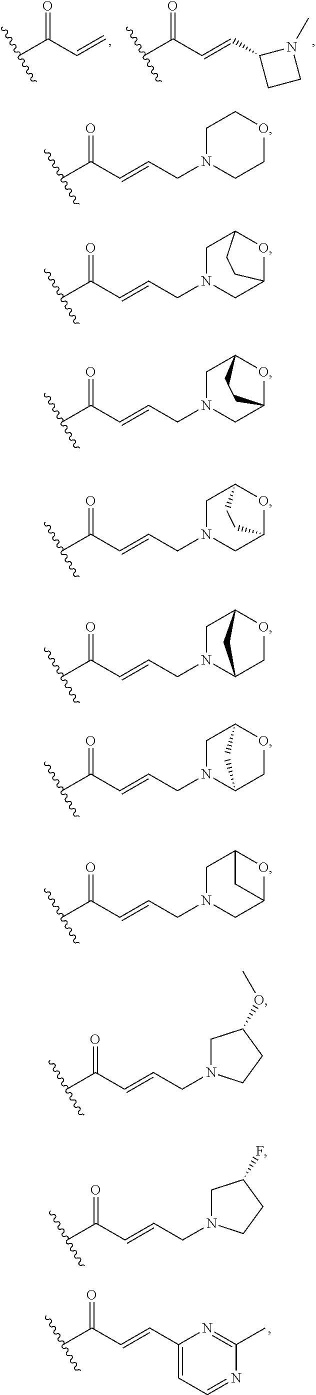

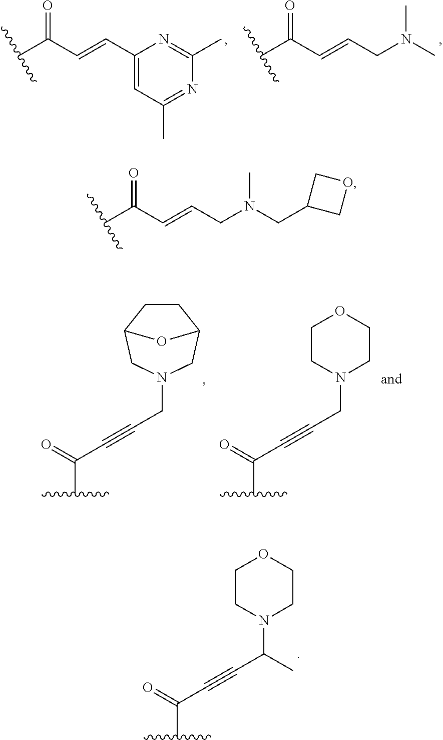

In an embodiment, R² is

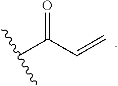 [SMILES: *C(=O)C=C]

In an embodiment, R² is

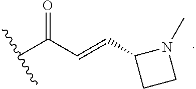 [SMILES: *C(=O)/C=C/[C@H]1CCN1C]

In an embodiment, R² is

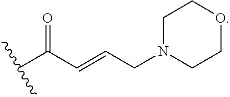 [SMILES: *C(=O)/C=C/CN1CCOCC1]

In an embodiment, R² is

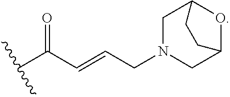 [SMILES: *C(=O)/C=C/CN1CC2CCC(C1)O2]

In an embodiment, R² is

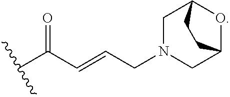 [SMILES: *C(=O)/C=C/CN1C[C@H]2CC[C@@H](C1)O2]

In an embodiment, R² is

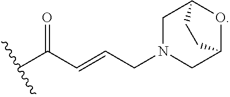 [SMILES: *C(=O)/C=C/CN1C[C@H]2CC[C@@H](C1)O2]

In an embodiment, R² is

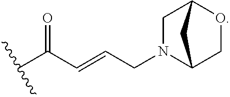 [SMILES: *C(=O)/C=C/CN1C[C@H]2C[C@@H]1CO2]

In an embodiment, R² is

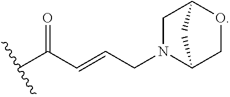 [SMILES: *C(=O)/C=C/CN1C[C@@H]2C[C@H]1CO2]

In an embodiment, R² is

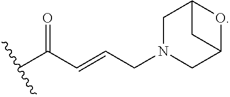 [SMILES: *C(=O)/C=C/CN1CC2CC(C1)O2]

In an embodiment, R² is

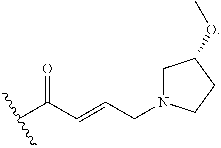 [SMILES: *C(=O)/C=C/CN1CC[C@@H](OC)C1]

In an embodiment, R² is

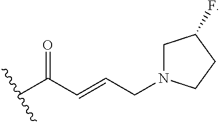 [SMILES: *C(=O)/C=C/CN1CC[C@@H](F)C1]

In an embodiment, R² is

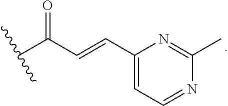 [SMILES: *C(=O)/C=C/c1ccnc(C)n1]

In an embodiment, R² is

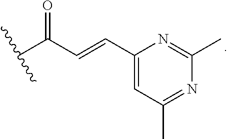 [SMILES: *C(=O)/C=C/c1cc(C)nc(C)n1]

In an embodiment, R² is

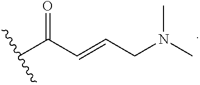 [SMILES: *C(=O)/C=C/CN(C)C]

In an embodiment, R² is

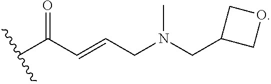 [SMILES: *C(=O)/C=C/CN(C)CC1COC1]

In an embodiment, R² is

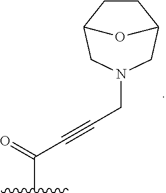 [SMILES: *C(=O)C#CCN1CC2CCC(C1)O2]

In an embodiment, R² is

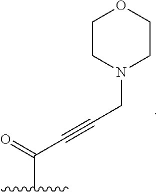 [SMILES: *C(=O)C#CCN1CCOCC1]

In an embodiment, R² is

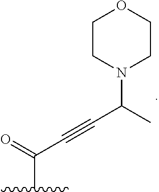 [SMILES: *C(=O)C#CC(C)N1CCOCC1]

As generally defined herein, R^{b} is —H or —F. In an embodiment, R^{b} is —H. In an embodiment, R^{b} is —F.

As generally defined herein, R^{c} is —H.

As generally defined herein, R^{e} is —H, —Y—R^{e1}, —Y—R^{e2}, or —Y—R^{e3}, wherein Y, R^{e1}, R^{e2} and R^{e3} are as defined herein. In an embodiment, R^{e} is selected from —H, —R^{e1}, —CH₂—R^{e1}, —CH(CH₃)—R^{e1}, —R^{e2}, CH₂—R^{e3}, CH(CH₃)—R^{e3} wherein Y, R^{e1}, R^{e2} and R^{e3} are as defined herein. In an embodiment, R^{e} is selected from —H, —R^{e1}, CH₂—R^{e1}, —R^{e2} and —CH₂—R^{e3} wherein Y, R^{e1}, R^{e2} and R^{e3} are as defined herein.

In an embodiment, R^{e} is selected from —R^{e1}, —CH₂—R^{e1} and —CH(CH₃)—R^{e1} wherein R^{e1} is as defined herein. In an embodiment, R^{e} is selected from —R^{e1} and —CH₂—R^{e1} wherein R^{e1} is as defined herein. In an embodiment, R^{e} is —R^{e1} wherein R^{e1} is as defined herein. In an embodiment, R^{e} is —CH₂—R^{e1} wherein R^{e1} is as defined herein. In an embodiment, R^{e} is —CH(CH₃)—R^{e1} wherein R^{e1} is as defined herein.

In an embodiment, R^{e} is selected from

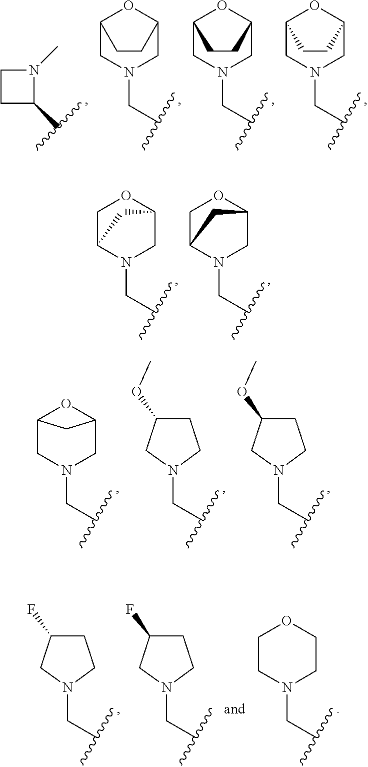

In an embodiment, R^{e} is

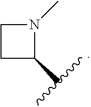 [SMILES: *[C@H]1CCN1C]

In an embodiment, R^{e} is

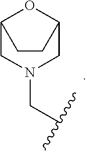 [SMILES: *CN1CC2CCC(C1)O2]

In an embodiment, R^{e} is

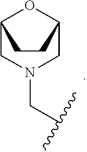 [SMILES: *CN1C[C@H]2CC[C@@H](C1)O2]

In an embodiment, R^{e} is

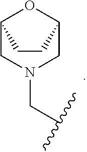 [SMILES: *CN1C[C@H]2CC[C@@H](C1)O2]

In an embodiment, R^{e} is

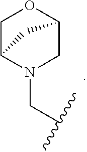 [SMILES: *CN1C[C@H]2C[C@@H]1CO2]

In an embodiment, R^{e} is

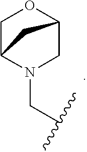 [SMILES: *CN1C[C@@H]2C[C@H]1CO2]

In an embodiment, R^{e} is

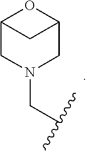 [SMILES: *CN1CC2CC(C1)O2]

In an embodiment, R^{e} is

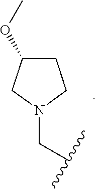 [SMILES: *CN1CC[C@@H](OC)C1]

In an embodiment, R^{e} is

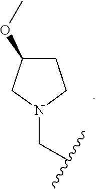 [SMILES: *CN1CC[C@H](OC)C1]

In an embodiment, R^{e} is

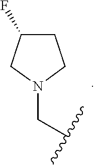 [SMILES: *CN1CC[C@@H](F)C1]

In an embodiment, R^{e} is

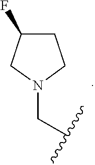 [SMILES: *CN1CC[C@H](F)C1]

In an embodiment, R^{e} is

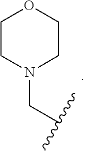 [SMILES: *CN1CCOCC1]

In an embodiment, R^{e} is selected from —R^{e2}, —CH₂—R^{e2} and —CH(CH₃)—R^{e2} wherein R^{e2} is as defined herein. In an embodiment, R^{e} is selected from —R^{e2} and —CH₂—R^{e2} wherein R^{e2} is as defined herein. In an embodiment, R^{e} is —R^{e2} wherein R^{e2} is as defined herein. In an embodiment, R^{e} is —CH₂—R^{e2} wherein R^{e2} is as defined herein. In an embodiment, R^{e} is —CH(CH₃)—R^{e2} wherein R^{e2} is as defined herein.

In an embodiment, R^{e} is selected from the group consisting of

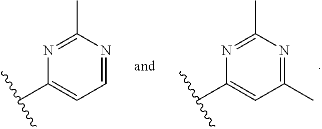

In an embodiment, R^{e} is

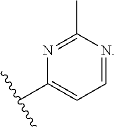 [SMILES: *c1ccnc(C)n1]

In an embodiment, R^{e} is

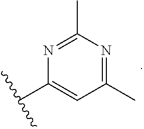 [SMILES: *c1cc(C)nc(C)n1]

In an embodiment, R^{e} is selected from —CH₂—R^{e3} and —CH(CH₃)—R^{e3} wherein R^{e3} is as defined herein. In an embodiment, R^{e} is —CH₂—R^{e3} wherein R^{e3} is as defined herein. In an embodiment, R^{e} is —CH(CH₃)—R^{e3} wherein R^{e3} is as defined herein.

In an embodiment, R^{e} is selected from

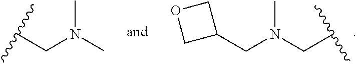

In an embodiment, R^{e} is

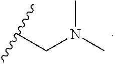 [SMILES: *CN(C)C]

In an embodiment, R^{e} is

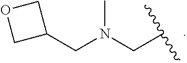 [SMILES: *CN(C)CC1COC1]

In an embodiment, R^{e} is —H.

In an embodiment, R^{e} is selected from the group consisting of: —H.

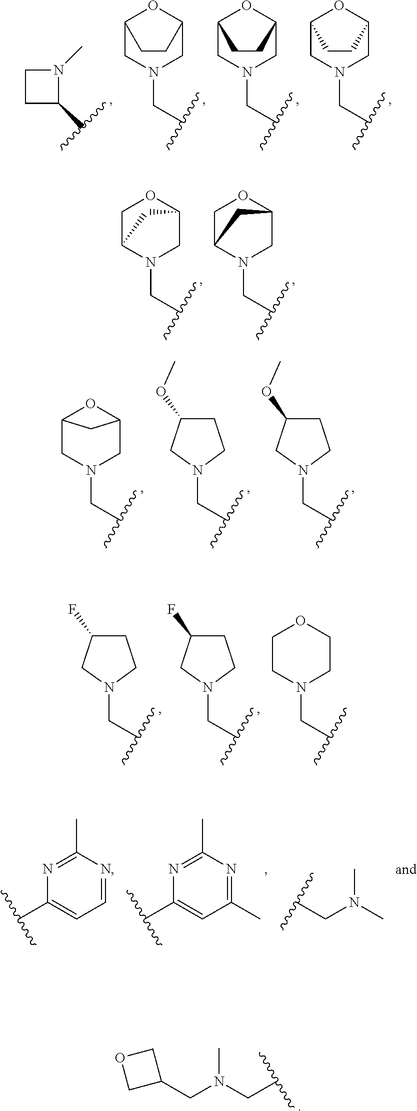

As generally defined herein, Y is —(C(R^{Y1})(R^{Y2}))ₓ—, wherein R^{Y1}, R^{Y2} and x are as defined herein. In an embodiment, Y is selected from a bond, —CH₂—, —CH(CH₃)—, —C(CH₃)₂—, —CH₂CH₂—, —CH(CH₃)CH₂— and —CH₂CH(CH₃)—. In an embodiment, Y is selected from a bond, —CH₂— and —CH(CH₃)—. In an embodiment, Y is selected from a bond and —CH₂—. In an embodiment, Y is a bond. In an embodiment, Y is —CH₂—. In an embodiment, Y is —CH(CH₃)—.

As generally defined herein, x is 0, 1 or 2, provided that when R^{e} is Y—R^{e3}, x is not 0. In an embodiment, x is 0 or 1. In an embodiment, x is 1 or 2. In an embodiment, x is 0. In an embodiment, x is 0 or 1. In an embodiment, x is 1. In an embodiment, x is 2.

As generally defined herein, each R^{Y1} is independently selected from —H and —CH₃. In an embodiment, each R^{Y1} is —H. In an embodiment, at least one R^{Y1} is -Me. In an embodiment, two R^{Y1} on the same carbon are -Me.

As generally defined herein, each R^{Y2} is independently selected from —H and —CH₃. In an embodiment, each R^{Y2} is —H. In an embodiment, at least one R^{Y2} is -Me. In an embodiment, two R^{Y2} on the same carbon are -Me.

As generally defined herein, R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy.

In an embodiment, R^{e1} is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the monocyclic heterocycle of R^{e1} is substituted with 0 or 1 instance of C₁-C₄ alkyl. In an embodiment, the monocyclic heterocycle of R^{e1} is substituted with 0 or 1 instance of methyl.

In an embodiment, R^{e1} is selected from azetidinyl, pyrrolidinyl and morpholinyl substituted with 0 or 1 instance of methyl. In an embodiment, R^{e1} is azetidinyl substituted with 0 or 1 instance of methyl. In an embodiment, R^{e1} is N-methyl azetidinyl.

In an embodiment, the attachment point for the monocyclic heterocycle is on a carbon atom.

In an embodiment, R^{e1} is

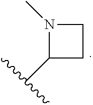 [SMILES: *C1CCN1C]

In an embodiment, R^{e1} is

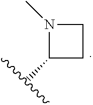 [SMILES: *[C@H]1CCN1C]

In an embodiment, R^{e1} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the attachment point for R^{e1} is the nitrogen atom of the heterocycle.

In an embodiment, R^{e1} is selected from the group consisting of:

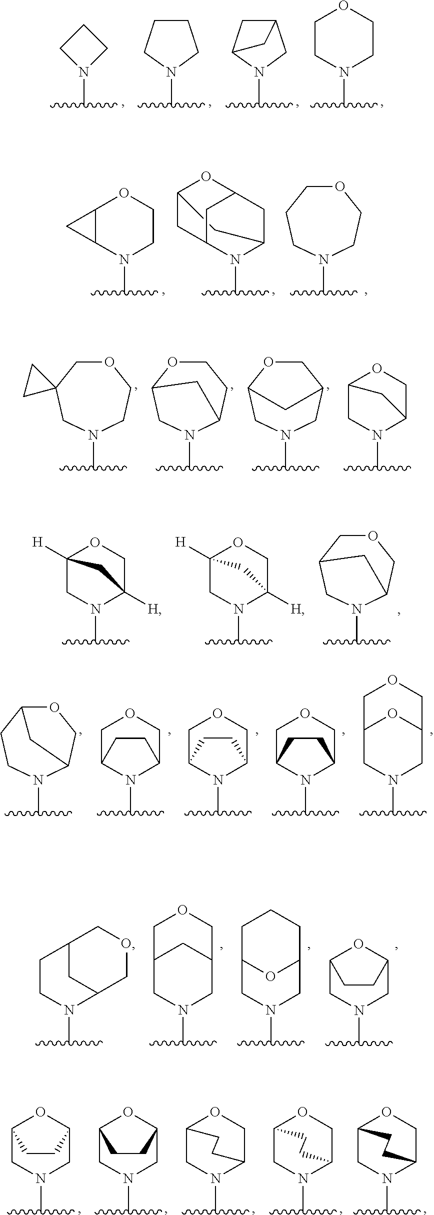

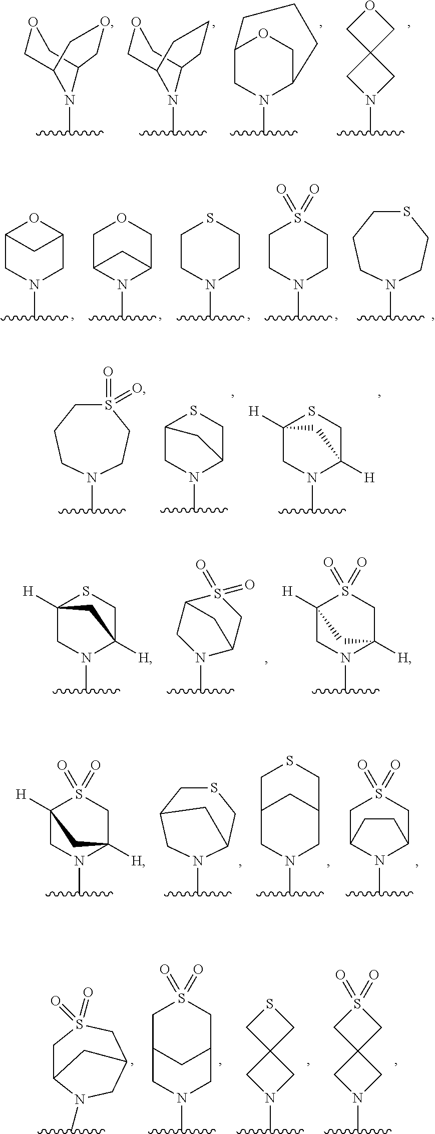

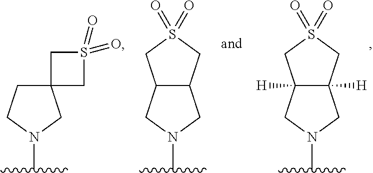

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from

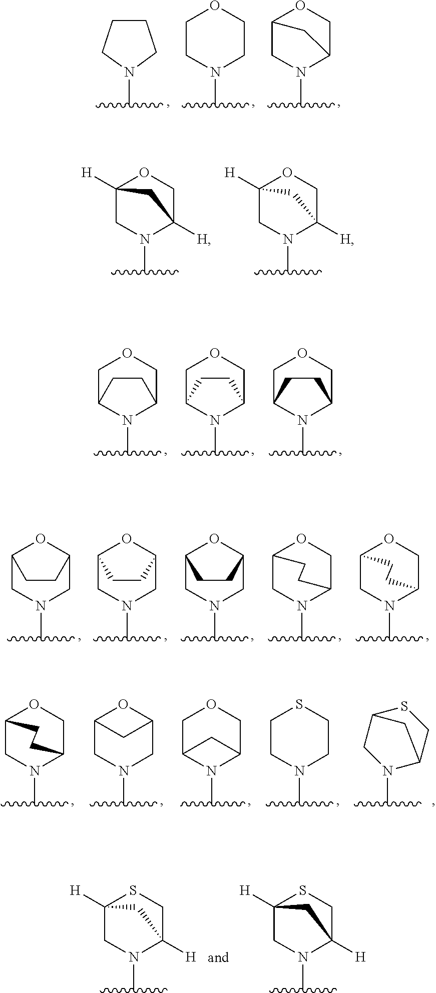

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is selected from

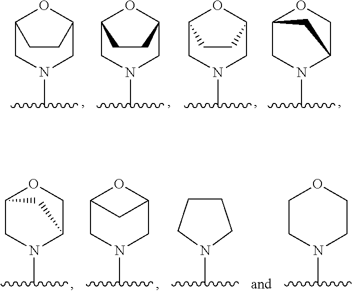

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{e1} is

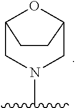 [SMILES: *N1CC2CCC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

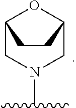 [SMILES: *N1C[C@H]2CC[C@@H](C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

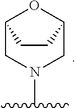 [SMILES: *N1C[C@H]2CC[C@@H](C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

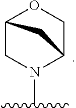 [SMILES: *N1C[C@H]2C[C@@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

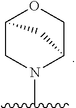 [SMILES: *N1C[C@@H]2C[C@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

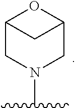 [SMILES: *N1CC2CC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

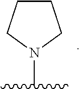 [SMILES: *N1CCCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{e1} is

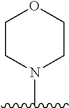 [SMILES: *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, the 4-10 membered heterocycle of R^{e1} is substituted with 0, 1 or 2 substituents independently selected from —F, —OMe and -Me. In an embodiment, the 4-10 membered heterocycle of R^{e1} is unsubstituted.

In an embodiment, R^{e1} is selected from the group consisting of:

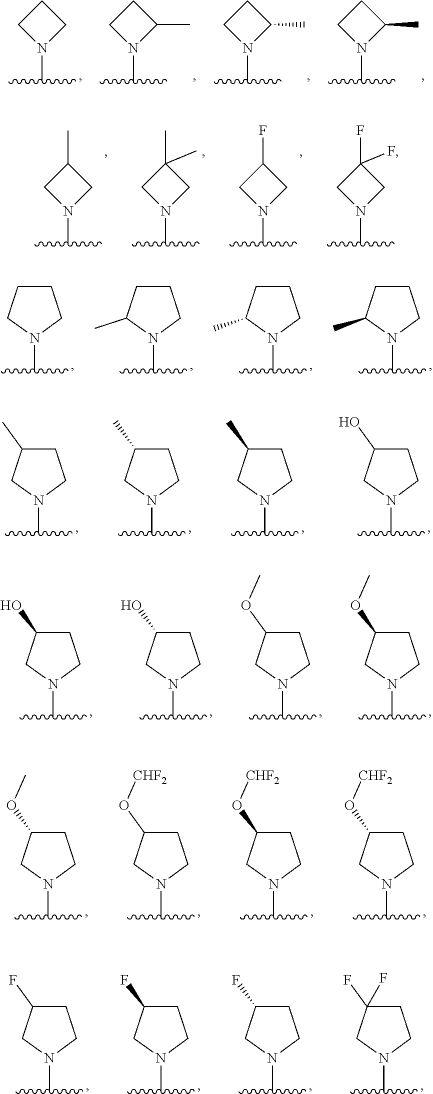

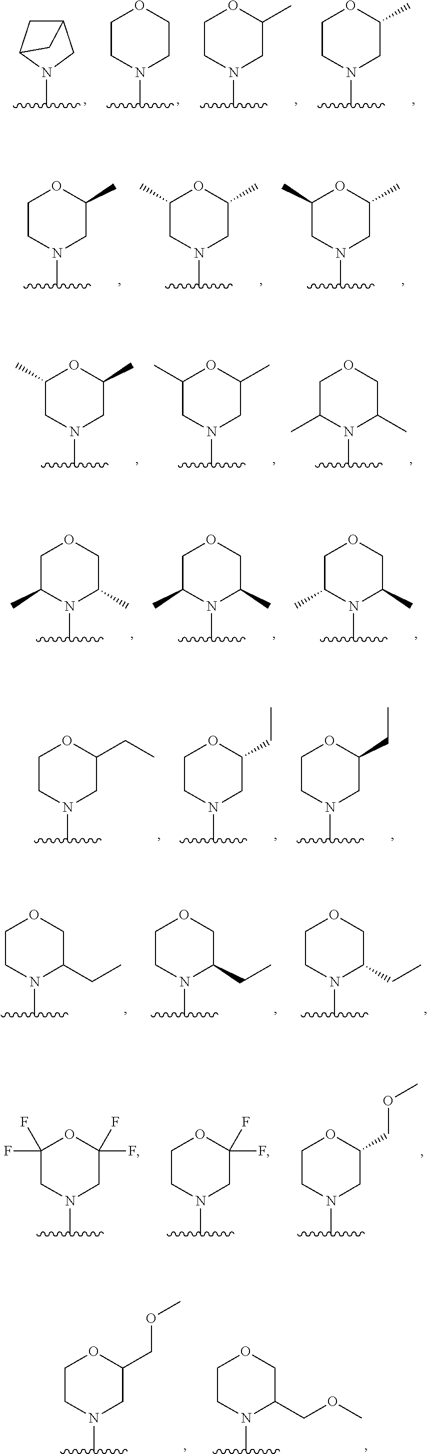

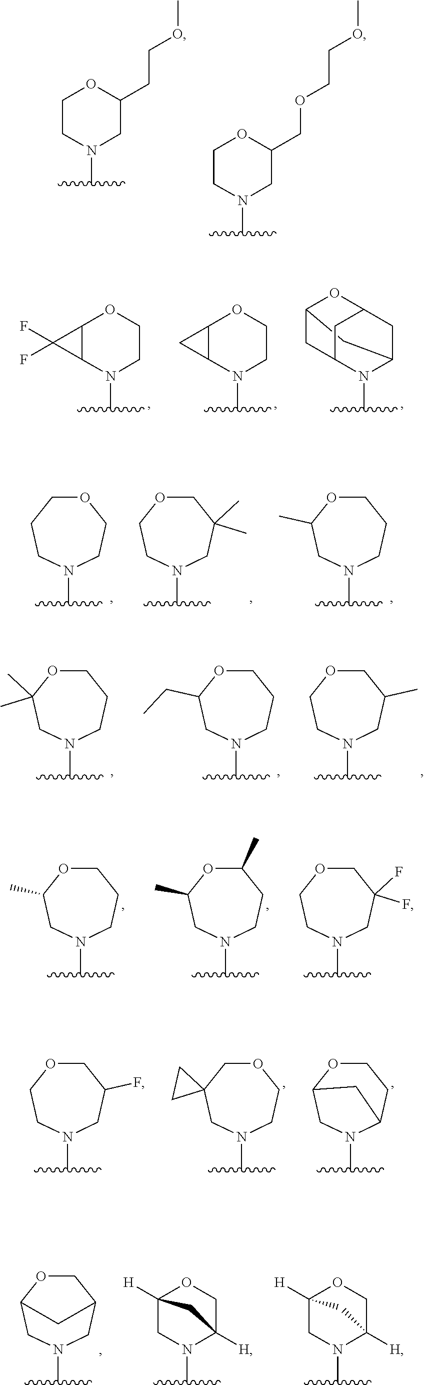

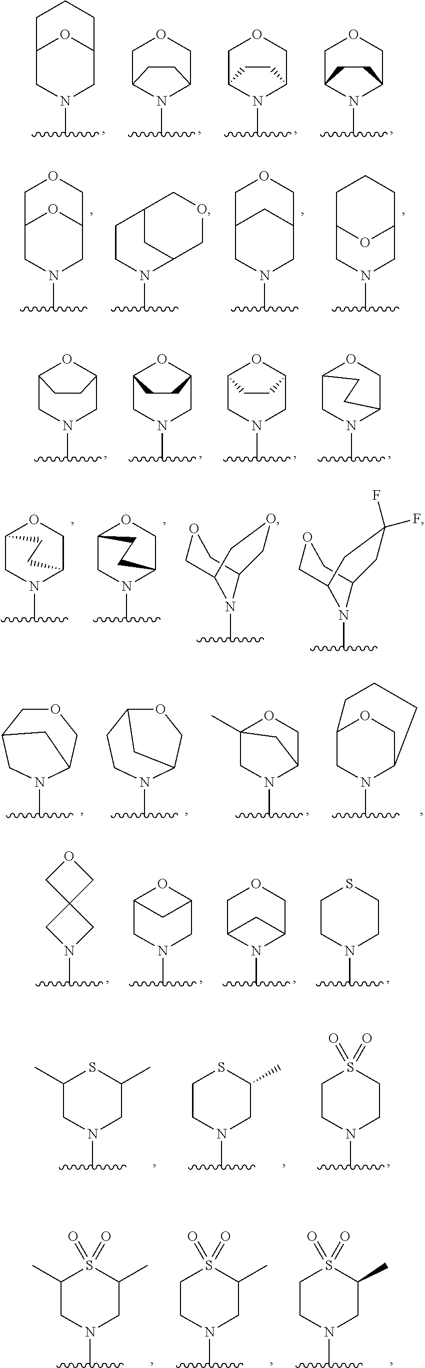

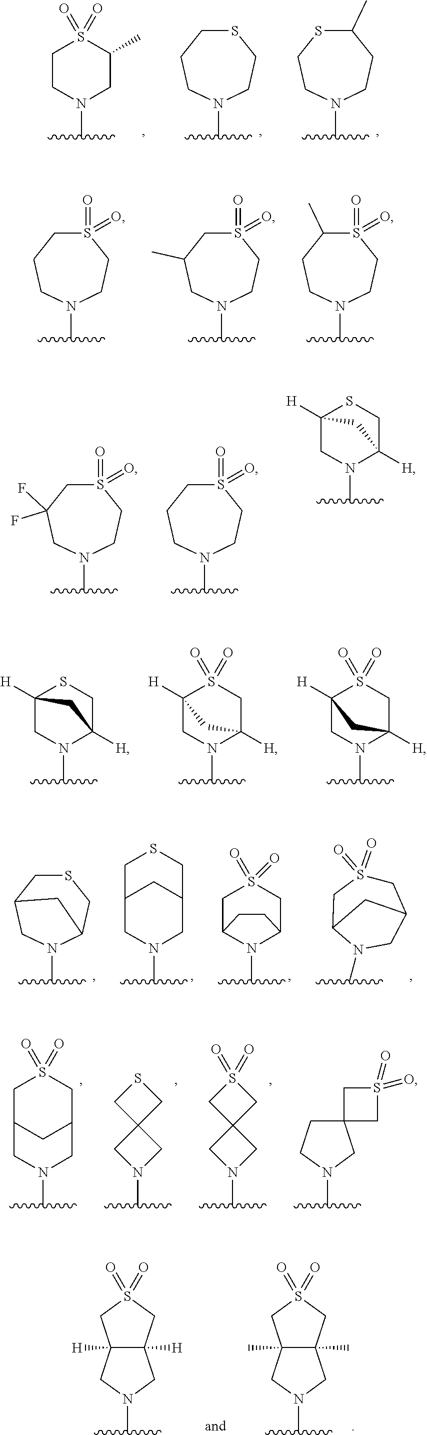

In an embodiment, R^{e1} is selected from

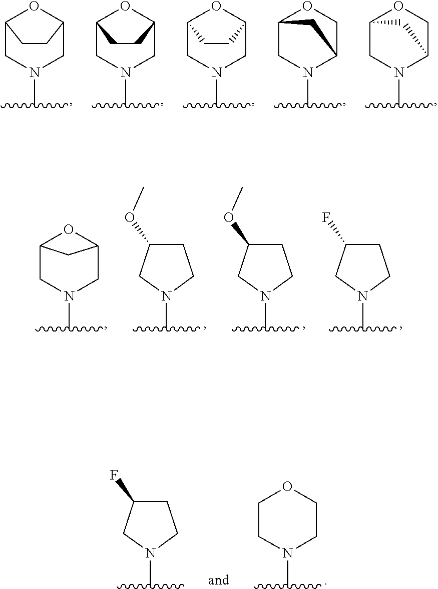

In an embodiment, R^{e1} is unsubstituted

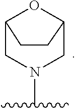 [SMILES: *N1CC2CCC(C1)O2]

In an embodiment, R^{e1} is unsubstituted

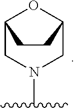 [SMILES: *N1C[C@H]2CC[C@@H](C1)O2]

In an embodiment, R^{e1} is unsubstituted

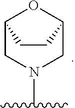 [SMILES: *N1C[C@H]2CC[C@@H](C1)O2]

In an embodiment, R^{e1} is unsubstituted

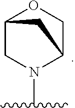 [SMILES: *N1C[C@H]2C[C@@H]1CO2]

In an embodiment, R^{e1} is unsubstituted

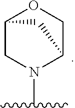 [SMILES: *N1C[C@@H]2C[C@H]1CO2]

In an embodiment, R^{e1} is unsubstituted

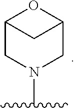 [SMILES: *N1CC2CC(C1)O2]

In an embodiment, R^{e1} is

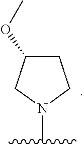 [SMILES: *N1CC[C@@H](OC)C1]

In an embodiment, R^{e1} is

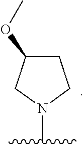 [SMILES: *N1CC[C@H](OC)C1]

In an embodiment, R^{e1} is

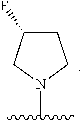 [SMILES: *N1CC[C@@H](F)C1]

In an embodiment, R^{e1} is

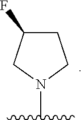 [SMILES: *N1CC[C@H](F)C1]

In an embodiment, R^{e1} is unsubstituted

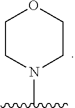 [SMILES: *N1CCOCC1]

As generally defined herein, R^{e2} is a 5-6 membered heteroaryl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl.

In an embodiment, R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of:

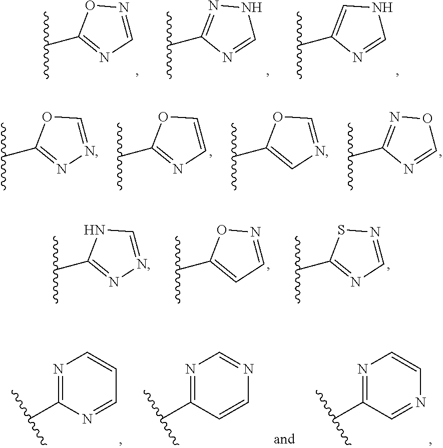

each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is a 6 membered heteroaryl group substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is pyrimidinyl or pyridazinyl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is pyrimidinyl or pyridazinyl substituted with 0, 1 or 2 instances of C₁-C₄ alkyl. In an embodiment, R^{e2} is pyrimidinyl substituted with 0, 1 or 2 instances of C₁-C₄ alkyl. In an embodiment, R^{e2} is pyridazinyl substituted with 0, 1 or 2 instances of C₁-C₄ alkyl. In an embodiment, R^{e2} is pyrimidinyl or pyridazinyl substituted with 0, 1 or 2 instances of -Me. In an embodiment, R^{e2} is pyrimidinyl substituted with 0, 1 or 2 instances of -Me. In an embodiment, R^{e2} is pyridazinyl substituted with 0, 1 or 2 instances of -Me.

In an embodiment, R^{e2} is selected from the group consisting of:

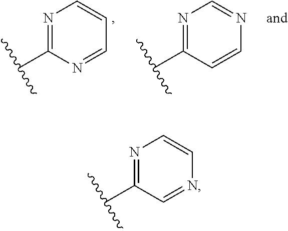 [SMILES: *c1ncccn1 ; *c1ccncn1 ; "and" ; *c1cnccn1]

substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

In an embodiment, R^{e2} is selected from the group consisting of

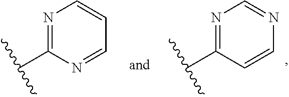 [SMILES: *c1ncccn1 ; *c1ccncn1]

substituted with 0, 1 or 2 instances of C₁-C₄ alkyl. In an embodiment, R^{e2} is

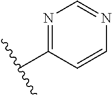 [SMILES: *c1ccncn1]

substituted with 0, 1 or 2 instances of C₁-C₄ alkyl. In an embodiment, R^{e2} is

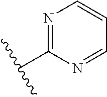 [SMILES: *c1ncccn1]

substituted with 0, 1 or 2 instances of C₁-C₄ alkyl. In an embodiment, R^{e2} is

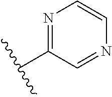 [SMILES: *c1cnccn1]

substituted with 0, 1 or 2 instances of C₁-C₄ alkyl. In an embodiment, R^{e2} is selected from the group consisting of

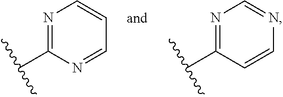 [SMILES: *c1ncccn1 ; "and" ; *c1ccncn1]

substituted with 0, 1 or 2 instances of -Me.

In an embodiment, R^{e2} is

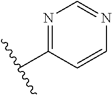 [SMILES: *c1ccncn1]

substituted with 0, 1 or 2 instances of -Me. In an embodiment, R^{e2} is

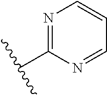 [SMILES: *c1ncccn1]

substituted with 0, 1 or 2 instances of -Me. In an embodiment, R^{e2} is

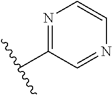 [SMILES: *c1cnccn1]

substituted with 0, 1 or 2 instances of -Me.

In an embodiment, R^{e2} is selected from the group consisting of

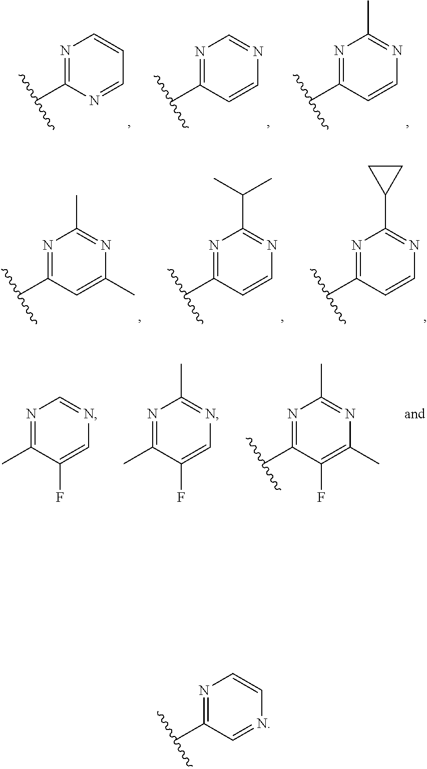

In an embodiment, R^{e2} is selected from the group consisting of

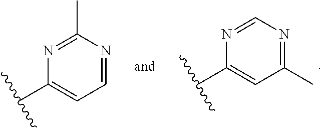

In an embodiment, R^{e2} is

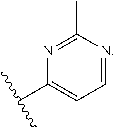 [SMILES: *c1ccnc(C)n1]

In an embodiment, R^{e2} is

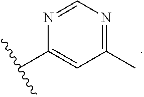 [SMILES: *c1cc(C)ncn1]

As generally defined herein, R^{e3} is —NR³¹R³². In an embodiment, R^{e3} is selected from

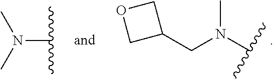

In an embodiment, R^{e3} is

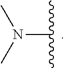 [SMILES: *N(C)C]

In an embodiment, R^{e3} is

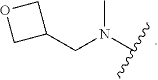 [SMILES: *N(C)CC1COC1]

As generally defined herein, R³¹ is selected from the group consisting of hydrogen, C₁-C₄ alkyl, C₁-C₄ alkoxy, C₁-C₄ haloalkoxy, and C₁-C₄ alkyl substituted with a 3-6 membered heterocycle. In an embodiment, R³¹ is C₁-C₄ alkyl. In an embodiment, R³¹ is -Me.

As generally defined herein, R³² is selected from the group consisting of hydrogen, C₁-C₄ alkyl, C₁-C₄ alkoxy, C₁-C₄ haloalkoxy, and C₁-C₄ alkyl substituted with a 3-6 membered heterocycle. In an embodiment, R³² is C₁-C₄ alkyl or C₁-C₄ alkyl substituted with a 3-6 membered heterocycle. In an embodiment, R³² is C₁-C₄ alkyl substituted with a 3-6 membered heterocycle. In an embodiment, R³² is C₁-C₄ alkyl substituted with a heterocycle selected from azetidine, oxetane, pyrrolidine, tetrahydrofuran, piperidine, tetrahydropyran and morpholine. In an embodiment, R³² is C₁-C₄ alkyl substituted with oxetane. In an embodiment, R³² is

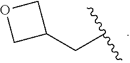 [SMILES: *CC1COC1]

In an embodiment, R³² is C₁-C₄ alkyl. In an embodiment, R³² is Me.

As generally defined herein, L is —(C(R^{L1})(L^{L2}))_{z}—, wherein L¹, L² and z are as defined herein. In an embodiment, L is selected from a bond, —CH₂—, —CH(CH₃)—, —C(CH₃)₂—, —CH₂CH₂—, —CH(CH₃)CH₂— and —CH₂CH(CH₃)—. In an embodiment, L is selected from a bond, —CH₂— and —CH(CH₃)—. In an embodiment, L is selected from a bond and —CH₂—. In an embodiment, L is a bond. In an embodiment, L is —CH₂—. In an embodiment, L is —CH(CH₃)—.

As generally defined herein, z is 0, 1 or 2. In an embodiment, z is 0 or 1. In an embodiment, z is 1 or 2. In an embodiment, z is 0. In an embodiment, z is 0 or 1. In an embodiment, z is 1. In an embodiment, z is 2.

As generally defined herein, R^{L1} is independently selected from —H and —CH₃. In an embodiment, each R^{L1} is —H. In an embodiment, at least one R^{L1} is -Me.

As generally defined herein, R^{L2} is independently selected from —H and —CH₃. In an embodiment, each R^{L2} is —H. In an embodiment, at least one R^{L2} is -Me.

In an embodiment, one R^{L1} and one R^{L2} on the same carbon are both-Me.

As generally defined herein, R^{f} is a 4-10 membered heterocycle which is substituted with 0, 1, 2, 3 or 4 substituents independently selected from halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl.

In an embodiment, R^{f} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the attachment point for R^{f} is the nitrogen atom of the heterocycle.

In an embodiment, R^{f} is selected from the group consisting of:

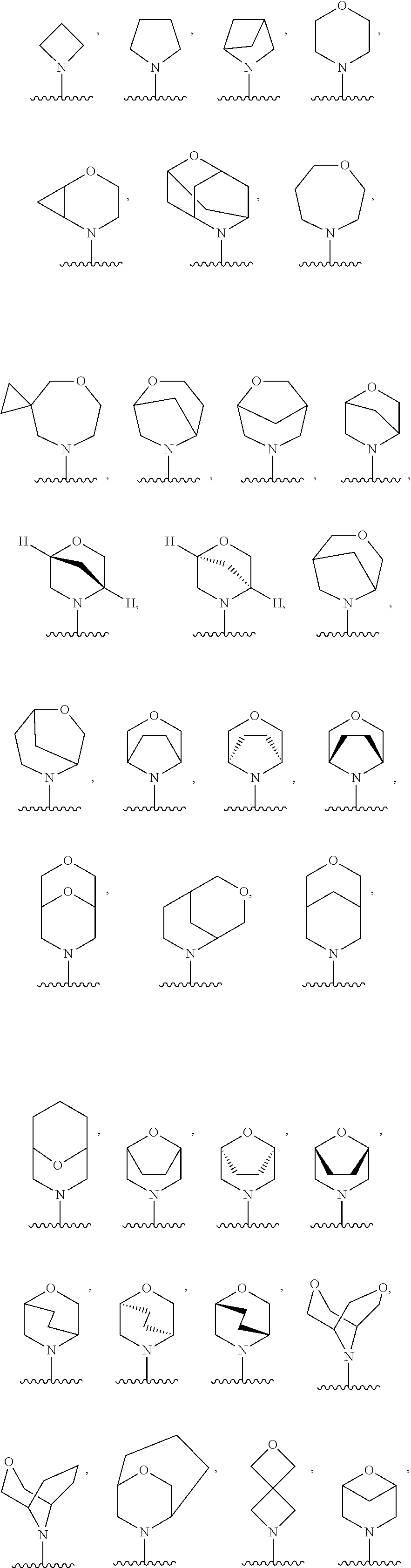

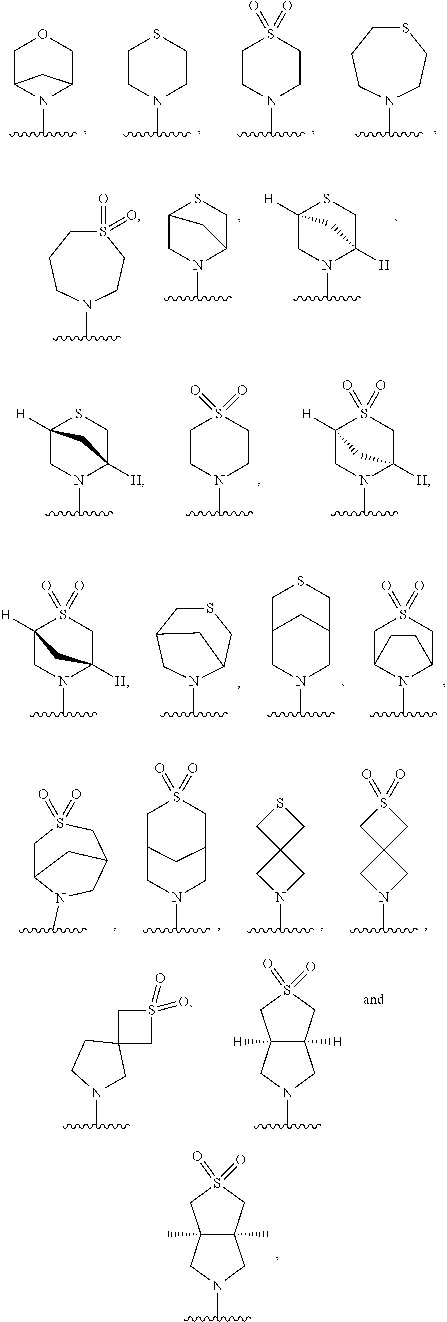

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is selected from

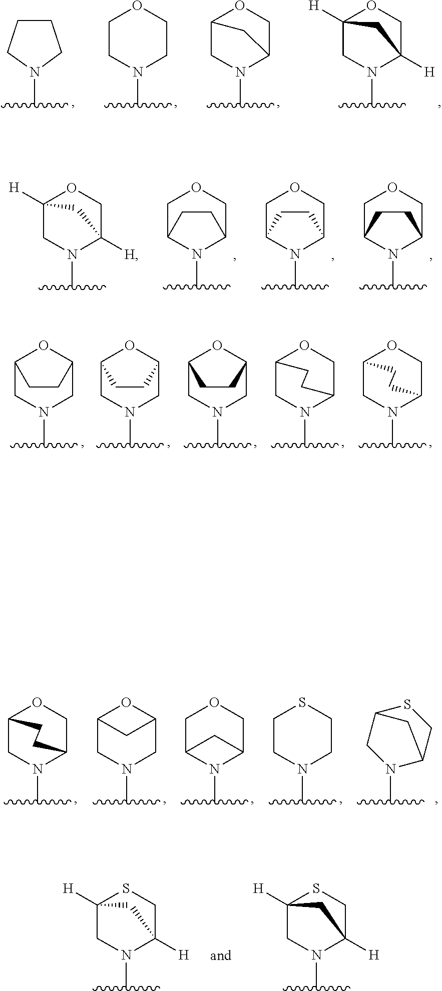

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, R^{f} is selected from

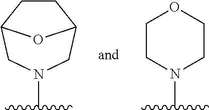 [SMILES: *N1CC2CCC(C1)O2 ; *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{f} is

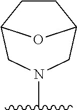 [SMILES: *N1CC2CCC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy. In an embodiment, R^{f} is

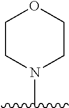 [SMILES: *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

In an embodiment, the 4-10 membered heterocycle of R^{f} is substituted with 0, 1 or 2 substituents independently selected from —F, —OMe and -Me. In an embodiment, the 4-10 membered heterocycle of R^{f} is unsubstituted.

In an embodiment, R^{f} is selected from the group consisting of:

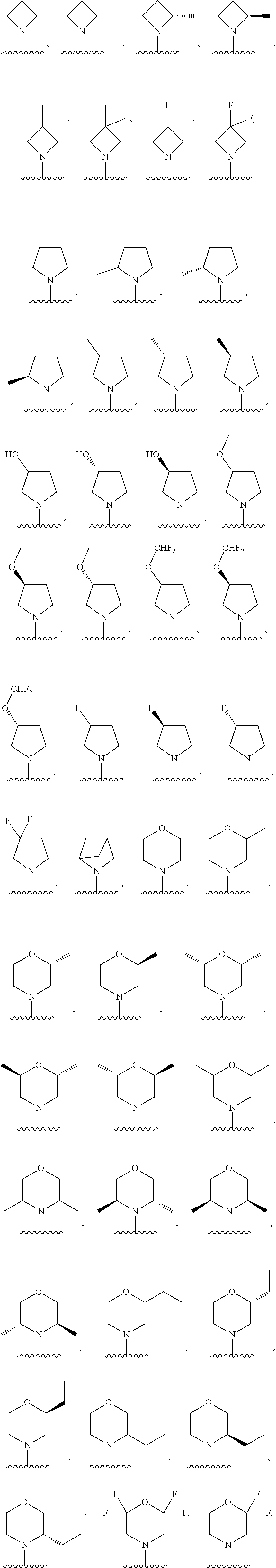

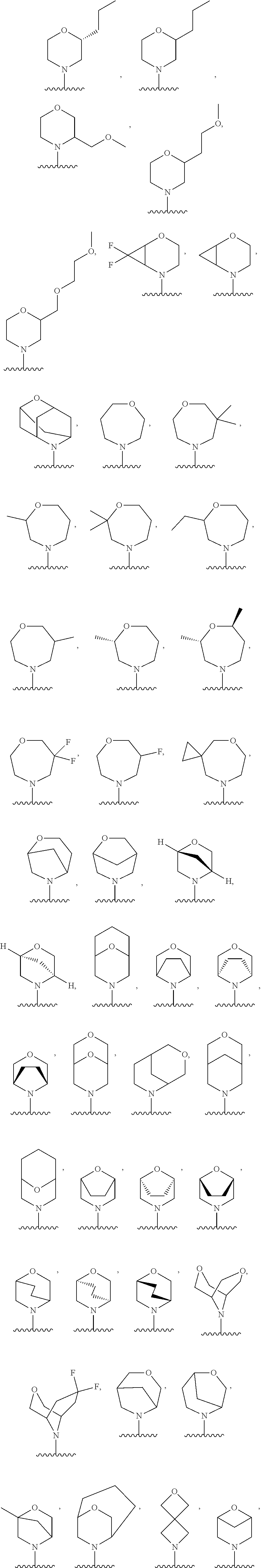

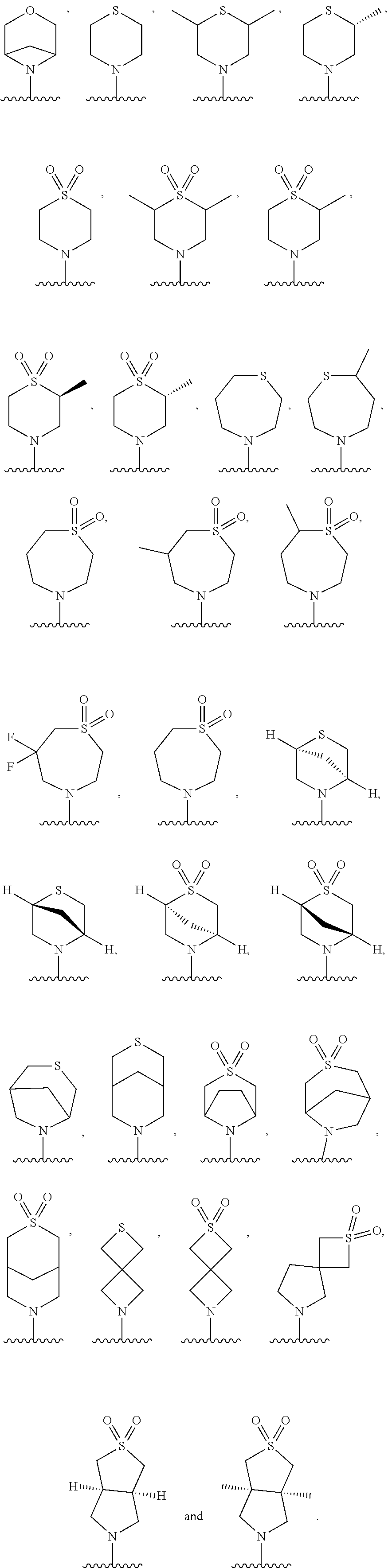

In an embodiment, R^{f} is unsubstituted

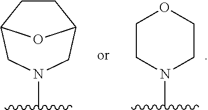 [SMILES: *N1CC2CCC(C1)O2 ; *N1CCOCC1]

In an embodiment, R^{f} is unsubstituted

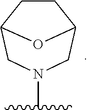 [SMILES: *N1CC2CCC(C1)O2]

In an embodiment R^{f} is unsubstituted

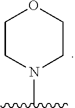 [SMILES: *N1CCOCC1]

As generally defined herein, R^{x} is selected from —H and C₁-C₄ alkyl. In an embodiment, R^{x} is selected from —H and -Me. In an embodiment, R^{x} is -Me. In an embodiment, R^{x} is —H.

As generally defined herein, R^{y} is selected from halo and C₁-C₄ haloalkyl. In an embodiment, R^{y} is selected from —Cl and —CF₃. In an embodiment, R^{y} is —Cl. In an embodiment, R^{y} is —CF₃.

In an embodiment, the compound is selected from the compounds of Table 1 or a salt thereof; and/or an isotopologue thereof. In an embodiment, the compound is not a salt.

In an embodiment, the compound is a salt. In an embodiment, the salt is a formate salt. In an embodiment, the salt is a trifluoroacetate salt. In an embodiment, the salt is a pharmaceutically acceptable salt.

Unless otherwise indicated, the absolute stereochemistry of all chiral atoms is as depicted.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

In some variations, any of the compounds described herein, such as a compound of Formula (I), Formula (II), Formula (III), or Formula (IV), or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Any formula given herein, such as Formula (I), Formula (II), Formula (III), or Formula (IV), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of, provided herein can be combined with every other variation or embodiment of Ring A, R^{a}, R^{b}, R^{c}, R^{d}, R^{e}, R^{e1}, R^{e2}, R^{e3}, R^{f}, R^{g}, R^{h}, Rⁱ, R^{j}, R^{k}, R^{L1}, R^{L2}, R^{m}, Rⁿ, R^{o}, R^{p}, R^{q}, R^{r}, R^{s}, R^{t}, R^{x}, R^{y}, R^{Y1}, R^{Y2}, R¹, R², R³, R⁴, R³¹, R³², or z, provided herein can be combined with every other variation or embodiment of Ring A, R^{a}, R^{b}, R^{c}, R^{d}, R^{e}, R^{e1}, R^{e2}, R^{e3}, R^{f}, R^{g}, R^{h}, Rⁱ, R^{j}, R^{k}, R^{L1}, R^{L2}, R^{m}, Rⁿ, R^{o}, R^{p}, R^{q}, R^{r}, R^{s}, R^{t}, R^{x}, R^{y}, R^{Y1}, R^{Y2}, R¹, R², R³, R⁴, R³¹, R³², or z, as if each combination had been individually and specifically described.

As used herein, when any variable occurs more than one time in a chemical formula, its definition on each occurrence is independent of its definition at every other occurrence.

### Methods for Treatment of Cancer

The compounds of Formula (I), Formula (II), Formula (III) and Formula (IV) and pharmaceutically acceptable salts and/or isotopologues thereof, including embodiments thereof disclosed herein, are useful for the treatment of cancer, which include but are not limited to, various types of cancer including e.g. lung, colorectal, pancreatic, bile duct, thyroid, gall bladder, uterine, mesothelioma, cervical, and bladder cancers. More particularly, cancers that may be treated by the compounds of Formula (I), Formula (II), Formula (III) and Formula (IV), and pharmaceutically acceptable salts and/or isotopologues thereof, including embodiments thereof disclosed herein, include, but are not limited to cancers such as glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cell myeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cell malignancies, pediatric neuroblastoma, and melanoma. In some embodiments, including any of the foregoing embodiments, the cancer is a KRAS G12C mediated cancer. In some embodiments, including any of the foregoing embodiments, the subject has been diagnosed as having a KRAS G12C mediated cancer. In some embodiments, including any of the foregoing embodiments, the subject has been determined to be at risk of developing a KRAS G12C mediated cancer.

In some embodiments, including any of the foregoing embodiments, the subject and/or the cancer is resistant or refractory to treatment with certain KRAS inhibitors (e.g., G12C KRAS inhibitors).

The compounds of Formula (I), Formula (II), Formula (III) and Formula (IV), and pharmaceutically acceptable salts and/or isotopologues thereof, including embodiments thereof disclosed herein, may be used for methods for inhibiting KRAS G12C in a cell, by contacting the cell in which inhibition of KRAS G12C activity is desired with an amount of the compound effective to inhibit KRAS G12C activity. Inhibition may be partial or total. In some embodiments, the contacting is in vitro. In some embodiments, the contacting is in vivo.

### Testing

The compounds of Formula (I), Formula (II), Formula (III) and Formula (IV) and pharmaceutically acceptable salts and/or isotopologues thereof, including embodiments thereof disclosed herein, may be tested by, for example, methods described in the Examples below, or by known and generally accepted cell and/or animal models.

The ability of compounds of Formula (I), Formula (II), Formula (III) and Formula (IV), and pharmaceutically acceptable salts and/or isotopologues thereof, to inhibit activity of the GTP-bound form of KRAS G12C can be tested using methods such as the in vitro assay described in Examples B-1 and B-2 below. Example B-1 describes determining, for various compounds, the half-maximal inhibition (IC₅₀) of KRAS G12C loaded with GTP analogue GMPPNP from binding to cRaf, as the Ras-binding domain (RBD). Example B-2 describes determining, for various compounds, the half-maximal inhibition (IC₅₀) of KRAS G12C loaded with GTP analogue GMPPNP from binding to PI3Kα, as the Ras-binding domain (RBD). Example B-3 describes testing compounds for the ability to inhibit cell viability in MCF10A G12C/A59G mutant, which abrogates GTPase activity, thus preventing hydrolysis of GTP to GDP.

### Pharmaceutical Compositions

In general, the compounds of Formula (I), Formula (II), Formula (III) and Formula (IV), and pharmaceutically acceptable salts and/or isotopologues thereof, of this disclosure (also may be referred to herein as “compounds” or “compounds of this disclosure”) will be administered in a therapeutically effective amount by any of the accepted modes of administration for agents that serve similar utilities. Therapeutically effective amounts of compounds of this disclosure may range from about 0.01 to about 500 mg per kg patient body weight per day, which can be administered in single or multiple doses. In some embodiments, a suitable dosage level may be from about 0.1 to about 250 mg/kg per day; or about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of a compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of this disclosure in combination with at least one pharmaceutically acceptable excipient. Acceptable excipients are non-toxic, aid administration, and do not adversely affect the therapeutic benefit of the compound of this disclosure. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds of the disclosure may be administered topically, that is by non-systemic administration. This includes the application of the compounds externally to the epidermis or the buccal cavity and the instillation of such compounds into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt. %) basis, from about 0.01-99.99 wt. % of a compound of this disclosure based on the total formulation, with the balance being one or more suitable pharmaceutical excipients. For example, the compound is present at a level of about 1-80 wt. %.

### Combinations and Combination Therapies

The compounds of this disclosure may be used in combination with one or more other drugs in the treatment of diseases or conditions for which compounds of this disclosure or the other drugs may have utility. Such other drug(s) may be administered contemporaneously or sequentially with a compound of the present disclosure. When a compound of this disclosure is used contemporaneously with one or more other drugs, a pharmaceutical composition in unit dosage form containing such other drugs and the compound of the present disclosure is contemplated. However, the combination therapy may also include therapies in which the compound of this disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other drugs, in addition to a compound of the present disclosure.

The above combinations include combinations of a compound of this disclosure not only with one other drug, but also with two or more other active drugs. Likewise, a compound of this disclosure may be used in combination with other drugs that are used in the prevention, treatment, control, amelioration, or reduction of risk of the diseases or conditions for which a compound of this disclosure is useful. Such other drugs may be administered contemporaneously or sequentially with a compound of the present disclosure. When a compound of this disclosure is used contemporaneously with one or more other drugs, a pharmaceutical composition containing such other drugs in addition to the compound of this disclosure can be used. Accordingly, the pharmaceutical compositions of the present disclosure also include those that also contain one or more other active ingredients, in addition to a compound of this disclosure. The weight ratio of the compound of this disclosure to the second active ingredient may be varied and will depend upon the effective dose of each ingredient. Generally, a therapeutically effective dose of each will be used.

Where the subject in need is suffering from or at risk of suffering from cancer, the subject can be treated with a compound of this disclosure in any combination with one or more other anti-cancer agents.

In some embodiments, the compounds of the present disclosure are used in combination with a CDK 4/6 inhibitor. Examples of CDK 4/6 inhibitors suitable for the provided compositions and methods include, but are not limited to, abemaciclib (N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine); palbociclib (6-acetyl-8-cyclopentyl-5-methyl-2-((5-(piperazin-1-yl)pyridin-2-yl)amino)-pyrido[2,3-d]pyrimidin-7(8H)-one) and ribociclib (7-cyclopentyl-N,N-dimethyl-2-((5-(piperazin-1-yl)pyridin-2-yl)amino)-7H-pyrrolo[2,3-d]pyrimidine-6-carboxamide) whereas the CDK 4/6 inhibitor trilaciclib (2′-((5-(piperazin-1-yl)pyridin-2-yl)amino)-7′,8′-dihydro-6′H-spiro-[cyclohexane-1,9′-pyrazino[1′,2′:1,5]pyrrolo[2,3-d]pyrimidin]-6′-one) is in late stage clinical trials. Another CDK 4/6 inhibitor useful in the methods herein is the CDK 2/4/6 inhibitor PF-06873600 (pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In another embodiment the compounds of the present disclosure are used in combination with Raf family kinase inhibitors. Examples of Raf family kinase inhibitors suitable for the provided compositions and methods include, but are not limited to, encorafenib (LGX818): methyl(S)-(1-((4-(3-(5-chloro-2-fluoro-3-(methylsulfonamido)phenyl)-1-isopropyl-1H-pyrazol-4-yl)pyrimidin-2-yl)amino) propan-2-yl) carbamate; PLX-8394: N-(3-(5-(2-cyclopropylpyrimidin-5-yl)-3a, 7a-dihydro-1H-pyrrolo[2,3-b]pyridine-3-carbonyl)-2,4-difluorophenyl)-3-fluoropyrrolidine-1-sulfonamide; Raf-709: N-(2-methyl-5′-morpholino-6′-((tetrahydro-2H-pyran-4-yl)oxy)-[3,3′-bipyridin]-5-yl)-3-(trifluoromethyl)benzamide; LXH254: N-(3-(2-(2-hydroxyethoxy)-6-morpholinopyridin-4-yl)-4-methylphenyl)-2-(trifluoromethyl) isonicotinamide; Sorafenib: 4-(4-(3-(4-chloro-3-(trifluoromethyl)phenyl)ureido)phenoxy)-N-methylpicolinamide; LY3009120:1-(3,3-dimethylbutyl)-3-(2-fluoro-4-methyl-5-(7-methyl-2-(methylamino)pyrido-[2,3-d]pyrimidin-6-yl)phenyl) urea; Lifirafenib (BGB-283); 5-(((1R,1aS,6bS)-1-(6-(trifhioro-methyl)-1H-benzo[d]imidazol-2-yl)-1a, 6b-dihydro-1H-cyclopropa[b]benzofuran-5-yl)methyl)-3,4-dihydro-1,8-naphthyridin-2 (1H)-one; Tak-632: N-(7-cyano-6-(4-fluoro-3-(2-(3-(trifluoromethyl)-phenyl) acetamido) phenoxy)benzo[d]thiazol-2-yl)cyclopropanecarboxamide; CEP-32496:1-(3-((6,7-dimethoxyquinazolin-4-yl)oxy)phenyl)-3-(5-(1,1,1-trifluoro-2-methylpropan-2-yl) isoxazol-3-yl) urea; CCT196969:1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl) urea; and R05126766: N-[3-fluoro-4-[4-methyl-2-oxo-7-(2-pyrimidinyloxy)-2H-1-benzopyran-3-yl]methyl]-2-pyridinyl]-N′-methylsulfamide.

In another embodiment the compounds of the present disclosure are used in combination with Src family kinases. Examples of Src family kinase inhibitors suitable for the provided compositions and methods include, but are not limited to, Dasatinib (N-(2-chloro-6-methylphenyl)-2-((6-(4-(2-hydroxyethyl) piperazin-1-yl)-2-methylpyrimidin-4-yl)amino) thiazole-5-carboxamide); Ponatinib (3-(imidazo[1,2-b]pyridazin-3-ylethynyl)-4-methyl-N-(4-((4-methylpiperazin-1-yl)methyl)-3-(trifluoromethyl)phenyl)benzamide); Vandetanib (N-(4-bromo-2-fluorophenyl)-6-methoxy-7-((1-methylpiperidin-4-yl)methoxy) quinazolin-4-amine); Bosutinib (4-((2,4-dichloro-5-methoxyphenyl)amino)-6-methoxy-7-(3-(4-methylpiperazin-1-yl)-propoxy) quinoline-3-carbonitrile); Saracatinib (N-(5-chlorobenzo[d][1,3]dioxol-4-yl)-7-(2-(4-methylpiperazin-1-yl) ethoxy)-5-((tetrahydro-2H-pyran-4-yl)oxy) quinazolin-4-amine); KX2-391 (N-benzyl-2-(5-(4-(2-morpholinoethoxy)phenyl)pyridin-2-yl) acetamide); SU6656 ((Z)-N,N-dimethyl-2-oxo-3-((4,5,6,7-tetrahydro-1H-indol-2-yl)methylene) indoline-5-sulfonamide); PP1 (1-(tert-butyl)-3-(p-tolyl)-1H-pyrazolo[3,4-d]pyrimidin-4-amine); WH-4-023 (2,6-dimethylphenyl(2,4-dimethoxyphenyl)(2-((4-(4-methylpiperazin-1-yl)phenyl)amino)pyrimidin-4-yl) carbamate) and KX-01 (N-benzyl-2-(5-(4-(2-morpholinoethoxy)phenyl)pyridin-2-yl) acetamide). In one embodiment, the Src inhibitor is Dasatinib. In one embodiment, the Src inhibitor is Saracatinib. In one embodiment, the Src inhibitor is Ponatinib. In one embodiment, the Src inhibitor is Vandetanib. In one embodiment, the Src inhibitor is KX-01.

In another embodiment the compounds of the present disclosure are used in combination with a SHP-2 inhibitor which include, but are not limited to SHP-099 (6-(4-amino-4-methylpiperidin-1-yl)-3-(2,3-dichlorophenyl) pyrazine-2-amine dihydrochloride), RMC-4550 (3 (3S,4S)-(4-amino-3-methyl-2-oxa-8-azaspiro[4.5]decan-8-yl)-6-(2,3-dichlorophenyl) pyrazin-2-yl) methanol), RMC-4360 (Revolution Medicines), TN0155 (Novartis), BBP-398 (BridgeBio), and ERAS-601 (Erasca).

In another embodiment the compounds of the present disclosure are used in combination with an mTOR inhibitor. Examples of mTOR inhibitors suitable for the provided compositions and methods include, but are not limited to, Everolimus, Rapamycin, Zotarolimus (ABT-578), ridaforolimus (Deforolimus; MK-8669), Sapanisertib (INK128; 5-(4-amino-1-isopropyl-1H-pyrazolo[3,4-d]pyrimidin-3-yl)benzo[d]oxazol-2-amine), Torin-1; 1-(4-(4-propionylpiperazin-1-yl)-3-(trifluoromethyl)cyclohexyl)-9-(quinolin-3-yl)benzo[h][1,6]naphthyridin-2 (1H)-one, dactolisib (BEZ235); 2-methyl-2-(4-(3-methyl-2-oxo-8-(quinolin-3-yl)-2,3-dihydro-1H-imidazo[4,5-c]quinolin-1-yl)phenyl) propanenitrile, buparlisib (5-(2,6-dimorpholin-4-ylpyrimidin-4-yl)-4-(trifluoromethyl)pyridin-2-amine); GDC-0941 (pictilisib); 4-[2-(1H-indazol-4-yl)-6-[(4-methylsulfonylpiperazin-1-yl)methyl]thieno[3,2-d]pyrimidin-4-yl]morpholine); GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl) urea), VS-5584 (SB2343)(5-(8-methyl-2-morpholin-4-yl-9-propan-2-ylpurin-6-yl)pyrimidin-2-amine) and vistusertib (AZD-2014; 3-(2,4-bis((S)-3-methylmorpholino)pyrido-[2,3-d]pyrimidin-7-yl)-N-methylbenzamide).

In another embodiment the compounds of the present disclosure are used in combination with a pan ErbB family inhibitor. In one embodiment the KRAS and pan ErbB family inhibitors are the only active agents in the provided compositions and methods. In one embodiment, the pan ErbB family inhibitor is an irreversible inhibitor. Examples of irreversible pan ErbB family inhibitors suitable for the provided compositions and methods include, but are not limited to, Afatinib; Dacomitinib; Canertinib; Poziotinib, AV 412 (N-4-([3-(chloro-4-fluorophenyl)amino]-7-[3-methyl-3-(4-methyl-1-piperazin-1-butyn-1-yl]-6-quinazolinyl]-2-prepenamide); PF 6274484 N-4-([3-(chloro-4-fluorophenyl)amino]-7-methoxy-6-quinazolinyl]-2-propenamide) and HKI 357 N-(2 (E)-N-[[4-[[3-chloro-4-[(fluorophenyl)methoxy]phenyl]amino]-3-cyano-7-ethoxy-6-quinolinyl]-4-(dimethylamino)-2-butenamide). In another embodiment, the pan ErbB family inhibitor is a reversible inhibitor. Examples of reversible pan ErbB family inhibitors suitable for the provided compositions and methods include, but are not limited to erlotinib, gefitinib, sapitinib; varlitinib; TAK-285 (N-[2-[4-[3-chloro-4-[3-(trifluoromethyl) phenoxy]phenylamino]-5H-pyrrolo[3,2-d]pyrimidin-5-yl]ethyl]-3-hydroxy-3-methylbutanamide); AEE788 (S)-(6-(4-((4-ethylpiperazin-1-ylmethyl)phenyl]-N-(1-phenylethyl)-7H-pyrrolo[2,3-d]pyrimidin-4-amine); tarloxotinib 3-[N-[4-(3-bromo-4-chlorophenylamino)-pyrido[3,4-d]pyrimidin-6-yl]carbamoyl]-N,N-dimethyl-N-(1-methyl-4-nitro-1H-imidazol-5-ylmethyl)-2 (E)-propen-1-aminium bromide); BMS 599626 ((3S)-3-morpholinylmethyl-[4-[1-[(3-fluorophenyl)methyl]-1H-indazol-5-yl]amino]-5-methylpurrolo[2,1-f][1,2,4]triazine-6-yl]carbamate dihydrochloride); and GW 583340 (N-[3-chloro-4-(3-fluorobenzyloxy)phenyl]-6-[2-[2-(methylsulfonyl)ethylaminomethyl]thiazol-4-yl]quinazolin-4-amine dihydrochloride).

In one embodiment, the pan ErbB family inhibitor is a combination of an EGFR inhibitor and a HER2 inhibitor, wherein the EGFR inhibitor and the HER2 inhibitor are a combination of two of: AG 1478 (N-(3-chlorophenyl)-6,7-dimethoxyquinazolin-4-amine hydrochloride); AG 555 ((E)-2-cyano-3-(3,4-dihydoxyphenyl)-N-(3-phenylpropyl)-2-propenamide); AG 556 ((E)-2-cyano-3-(3,4-dihydroxyphenyl)-N-(4-phenylbutyl)-2-propenamide; AG 825 (E-3-[3-benzothiazol-2-ylsulfanylmethyl)-4-hydroxy-5-methoxyphenyl]-2-cyano-2-propenamide); CP 724714 (2-methoxy-N-[(2E)-3-[4-[3-methyl-4-(6-methylpyridin-3-yloxy)phenylamino]quinazolin-6-yl]-2-propen-1-yl]acetamide; BIBU 1361 (N-(3-chloro-4-fluorophenyl)-6-[4-(diethylaminomethyl)-piperidin-1-yl]pyrimido[5,4-d]pyrimidin-4-amine dihydrochloride); BIBU 1382; (N⁸-(3-chloro-4-fluorophenyl)-N²-(1-methyl-4-piperidinyl)pyrimidino[5,4-d]pyrimidin-4-amine dihydrochloride), JNJ 28871063 (5E-4-amino-6-[4-(benzyloxy)-3-chlorophenylamino]-pyrimidine-5-carbaldehyde N-[2-(4-morpholinyl)ethyl]oxime hydrochloride); PD 153035 (4-(3-bromophenylamino)-6,7-dimethoxyquinazoline hydrochloride); and PD 158780 (N⁴-(3-bromophenyl)-N⁶-methyl-pyrido[3,4-d]pyrimidine-4,6-diamine).

In one embodiment, the pan ErbB family inhibitor is an anti-EGFR antibody, an anti-HER2 antibody or combination of an anti-EGFR antibody and anti-HER2 antibody. Antibodies, including monoclonal antibodies, antibody conjugates and bispecific antibodies, targeting EGFR and/or HER2 are well known and several antibodies are commercially available for research and human clinical use. Examples of anti-EGFR antibodies suitable for the provided compositions and methods include necitumumab, panitumumab and cetuximab. Examples of anti-HER2 antibodies suitable for the provided compositions and methods include, pertuzumab, trastuzumab, and trastuzumab emtansine.

In some embodiments, the compounds of the present disclosure are used in combination with an immune checkpoint inhibitor. Examples of immune checkpoint inhibitors suitable for the provided compositions and methods include, but are not limited to, PD-1, PD-L1, CTLA-4, and LAG-3 inhibitors, such as Pembrolizumab (Keytruda®), Nivolumab (Opdivo®), Cemiplimab (Libtayo®), Atezolizumab (Tecentriq®), Avelumab (Bavencio®), Durvalumab (Imfinzi™), Ipilimumab (Yervoy®), Relatlimab, Opdualag, and Dostarlimab (Jemperli).

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### Enumerated Embodiments

The following enumerated embodiments are representative of some aspects of the invention.

### Embodiment 1

A compound of Formula (I), Formula (II), Formula (III) or Formula (IV)

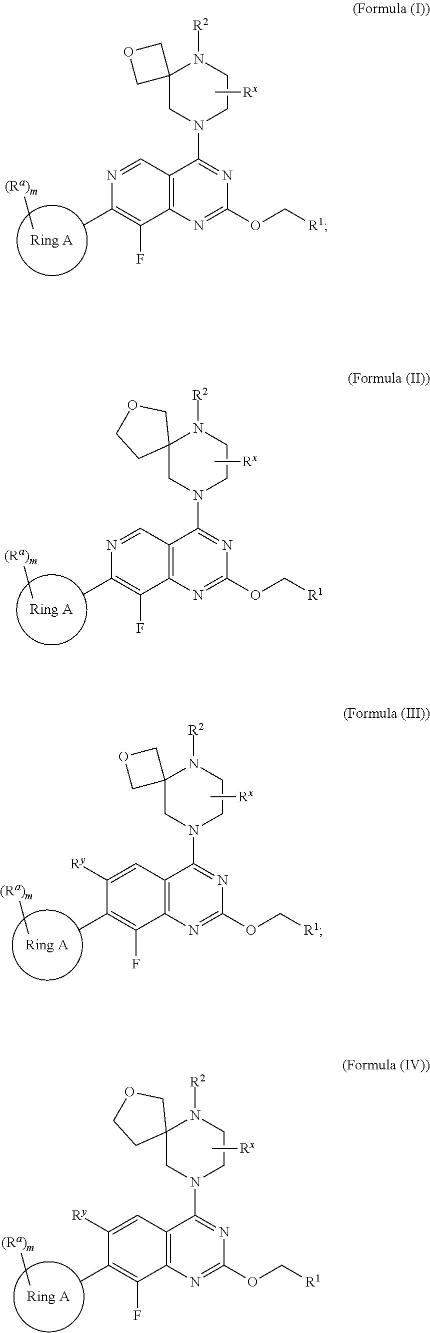

or a salt thereof; and/or an isotopologue thereof; wherein:
Ring A is a 6-10 membered aryl or a 5-10 membered heteroaryl;
each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
m is 0, 1, 2 or 3;
R¹ is

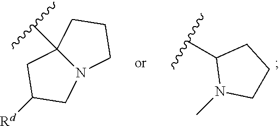

R^{d} is H or F;
R² is

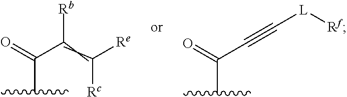

R^{b} is —H or —F;
R^{c} is —H;
R^{e} is —H, —Y—R^{e1}, —Y—R^{e2}, or —Y—R^{e3};
Y is —(C(R^{Y1})(R^{Y2}))ₓ—;
x is 0, 1 or 2 provided that when R^{e} is Y—R^{e3}, x is not 0;
R^{Y1} and R^{Y2} in each occurrence are independently selected from —H and —CH₃;
R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
L is —(C(R^{L1})(L^{L2}))_{z}—;
z is 0, 1 or 2;
R^{L1} and R^{L2} in each occurrence are independently selected from —H and —CH₃;
R^{f} is a 4-10 membered heterocycle which is substituted with 0, 1, 2, 3 or 4 substituents independently selected from halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl;
R^{x} is selected from —H and C₁-C₄ alkyl; and
R^{y} is selected from halo and C₁-C₄ haloalkyl.

### Embodiment 2

A compound of Formula (I) or Formula (II)

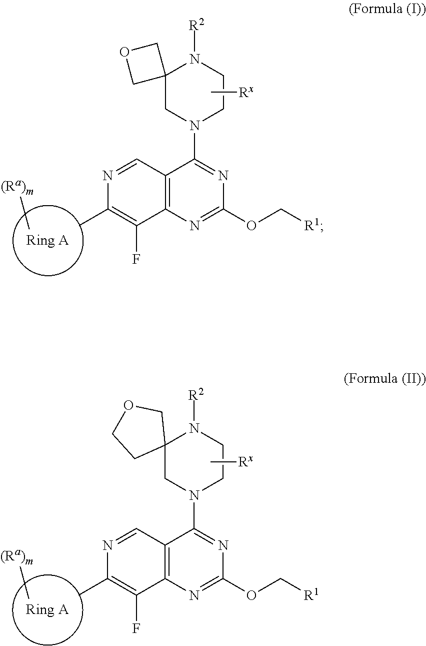

or a salt thereof; and/or an isotopologue thereof; wherein:
Ring A is a 6-10 membered aryl or a 5-10 membered heteroaryl;
each R^{a} is independently selected from halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
m is 0, 1, 2 or 3;
R¹ is

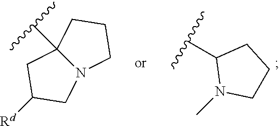

R^{d} is H or F;
R² is

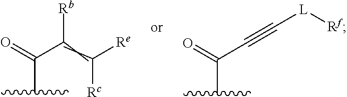

R^{b} is —H or —F;
R^{c} is —H;
R^{e} is —H, —Y—R^{e1}, —Y—R^{e2}, or —Y—R^{e3};
Y is —(C(R^{Y1})(R^{Y2}))ₓ—;
x is 0, 1 or 2 provided that when R^{e} is Y—R^{e3}, x is not 0;
R^{Y1} and R^{Y2} in each occurrence are independently selected from —H and —CH₃;
R^{e1} is a 4-10 membered heterocycle which is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
L is —(C(R^{L1})(L^{L2}))_{z}—;
z is 0, 1 or 2;
R^{L1} and R^{L2} in each occurrence are independently selected from —H and —CH₃;

R^{f} is a 4-10 membered heterocycle which is substituted with 0, 1, 2, 3 or 4 substituents independently selected from halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl; and
R^{x} is selected from —H and C₁-C₄ alkyl.

### Embodiment 3

The compound of embodiment 1, wherein the compound is of Formula (I) or Formula (II).

### Embodiment 4

The compound of embodiment 1, wherein the compound is of Formula (III) or Formula (IV).

### Embodiment 5

The compound of embodiment 1, wherein the compound is of Formula (I) or Formula (III).

### Embodiment 6

The compound of embodiment 1, wherein the compound is of Formula (II) or Formula (IV).

### Embodiment 7

The compound of embodiment 1, wherein the compound is of Formula (I).

### Embodiment 8

The compound of embodiment 1, wherein the compound is of Formula (II).

### Embodiment 9

The compound of embodiment 1, wherein the compound is of Formula (III).

### Embodiment 10

The compound of embodiment 1, wherein the compound is of Formula (IV).

### Embodiment 11

The compound of any one of embodiments 1-10, wherein Ring A is selected from a 6-10 membered aryl and a 9-10 membered bicyclic heteroaryl having 1, 2 or 3 heteroatoms independently selected from N, O and S.

### Embodiment 12

The compound of any one of embodiments 1-10, wherein Ring A is selected from the group consisting of naphthalenyl, phenyl, benzothiazolyl, isoquinolinyl, indazolyl and pyridinyl.

### Embodiment 13

The compound of any one of embodiments 1-10, wherein Ring A is selected from the group consisting of naphthalenyl, phenyl, isoquinolinyl, indazolyl and pyridinyl.

### Embodiment 14

The compound of any one of embodiments 1-10, wherein Ring A is selected from the group consisting of naphthalenyl, phenyl, benzothiazolyl and indazolyl.

### Embodiment 15

The compound of any one of embodiments 1-10, wherein Ring A is selected from the group consisting of naphthalenyl, phenyl and indazolyl.

### Embodiment 16

The compound of any one of embodiments 1-10, wherein Ring A is selected from the group consisting of naphthalenyl and phenyl.

### Embodiment 17

The compound of any one of embodiments 1-10, wherein Ring A is naphthalenyl.

### Embodiment 18

The compound of any one of embodiments 1-10, wherein Ring A is phenyl.

### Embodiment 19

The compound of any one of embodiments 1-10, wherein Ring A is benzothiazolyl.

### Embodiment 20

The compound of any one of embodiments 1-10, wherein Ring A is indazolyl.

### Embodiment 21

The compound of any one of embodiments 1-10, wherein Ring A is selected from the group consisting of naphthalen-1-yl, phenyl, isoquinolin-1-yl, benzo[d]thiazol-4-yl, indazol-3-yl, indazol-4-yl and pyridin-1-yl.

### Embodiment 22

The compound of any one of embodiments 1-10, wherein Ring A is selected from the group consisting of naphthalen-1-yl, phenyl, isoquinolin-1-yl, indazol-4-yl and pyridin-1-yl.

### Embodiment 23

The compound of any one of embodiments 1-10, wherein Ring A is selected from the group consisting of naphthalen-1-yl, phenyl, benzo[d]thiazol-4-yl, indazol-3-yl, and indazol-4-yl.

### Embodiment 24

The compound of any one of embodiments 1-10, wherein Ring A is selected from the group consisting of naphthalen-1-yl, phenyl, and indazol-4-yl.

### Embodiment 25

The compound of any one of embodiments 1-10, wherein Ring A is selected from the group consisting of naphthalen-1-yl and phenyl.

### Embodiment 26

The compound of any one of embodiments 1-10, wherein Ring A is naphthalen-1-yl.

### Embodiment 27

The compound of any one of embodiments 1-10, wherein Ring A is phenyl.

### Embodiment 28

The compound of any one of embodiments 1-10, wherein Ring A is benzo[d]thiazol-4-yl.

### Embodiment 29

The compound of any one of embodiments 1-10, wherein Ring A is indazol-3-yl.

### Embodiment 30

The compound of any one of embodiments 1-10, wherein Ring A is indazol-4-yl.

### Embodiment 31

The compound of any one of embodiments 1-30, wherein each R^{a} is independently selected from halo, —OH, —NH₂, C₁-C₄ alkyl, C₃-C₄ cycloalkyl, C₁-C₄ haloalkyl, and C₂-C₃ alkynyl.

### Embodiment 32

The compound of any one of embodiments 1-30, wherein each R^{a} is independently selected from halo, —OH, C₁-C₄ alkyl, C₃-C₄ cycloalkyl, C₁-C₄ haloalkyl and C₂-C₃ alkynyl.

### Embodiment 33

The compound of any one of embodiments 1-30, wherein each R^{a} is independently selected from halo and C₁-C₄ alkyl.

### Embodiment 34

The compound of any one of embodiments 1-30, wherein each R^{a} is independently selected from halo and C₃-C₄ cycloalkyl.

### Embodiment 35

The compound of any one of embodiments 1-30, wherein each R^{a} is independently selected from halo and C₁-C₄ haloalkyl.

### Embodiment 36

The compound of any one of embodiments 1-30, wherein each R^{a} is independently selected from halo and C₂-C₃ alkynyl.

### Embodiment 37

The compound of any one of embodiments 1-30, wherein each R^{a} is independently selected from halo and —NH₂.

### Embodiment 38

The compound of any one of embodiments 1-30, wherein each R^{a} is independently halo.

### Embodiment 39

The compound of any one of embodiments 1-30, wherein each R^{a} is independently selected from —F, —Cl, —OH, —NH₂, -Me, -Et, -cyclopropyl, —CF₃ and —C═CH.

### Embodiment 40

The compound of any one of embodiments 1-30, wherein each R^{a} is independently selected from —F, —Cl, —OH, -Me, -Et, cyclopropyl, —CF₃ and —C═CH.

### Embodiment 41

The compound of any one of embodiments 1-30, wherein each R^{a} is independently selected from —F, —Cl, -Me and -Et.

### Embodiment 42

The compound of any one of embodiments 1-30, wherein each R^{a} is independently selected from —OH, —Cl and -cyclopropyl.

### Embodiment 43

The compound of any one of embodiments 1-30, wherein each R^{a} is independently selected from —F, and —NH₂.

### Embodiment 44

The compound of any one of embodiments 1-30, wherein each R^{a} is independently selected from —F, —Cl and -cyclopropyl.

### Embodiment 45

The compound of any one of embodiments 1-30, wherein each R^{a} is independently selected from —F, —Cl and —CF₃.

### Embodiment 46

The compound of any one of embodiments 1-30, wherein each R^{a} is independently selected from —F, —Cl and —C═CH.

### Embodiment 47

The compound of any one of embodiments 1-30, wherein each R^{a} is independently selected from —F and —Cl.

### Embodiment 48

The compound of any one of embodiments 1-47, wherein m is 1, 2 or 3.

### Embodiment 49

The compound of any one of embodiments 1-47, wherein m is 1 or 2.

### Embodiment 50

The compound of any one of embodiments 1-47, wherein m is 2 or 3.

### Embodiment 51

The compound of any one of embodiments 1-47, wherein m is 1.

### Embodiment 52

The compound of any one of embodiments 1-47, wherein m is 2.

### Embodiment 53

The compound of any one of embodiments 1-47, wherein m is 3.

### Embodiment 54A

The compound of any one of embodiments 1-10, wherein Ring A is selected from the group consisting of:

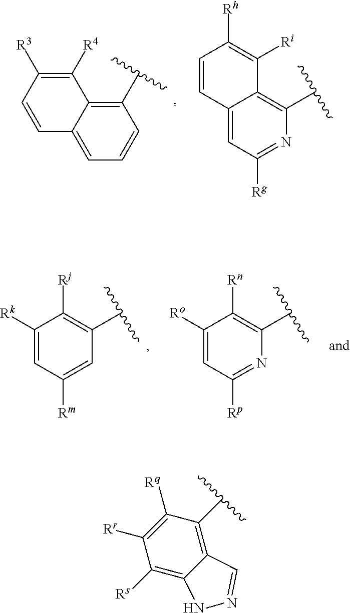

wherein:
each R³, R⁴, R^{h}, Rⁱ, R^{j}, R^{k}, Rⁿ, R^{o}, R^{q}, R^{r} and R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
each R^{g}, R^{m} and R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

### Embodiment 54B

The compound of any one of embodiments 1-10, wherein Ring A is selected from the group consisting of:

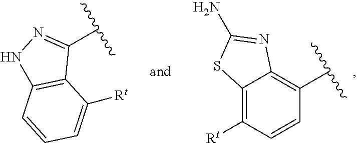

wherein R^{t} is halo.

### Embodiment 54C

The compound of embodiment 54B, or a slat thereof, and/or an isotopologue thereof, wherein R^{t} is —F or —Cl.

### Embodiment 55

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen, halo, C₁-C₄ alkyl, C₃-C₄ cycloalkyl, C₁-C₄ haloalkyl and C₂-C₃ alkynyl.

### Embodiment 56

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen, halo and C₁-C₄ alkyl.

### Embodiment 57

The compound of any embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen, halo and C₃-C₄ cycloalkyl.

### Embodiment 58

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen, halo and C₁-C₄ haloalkyl.

### Embodiment 59

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen, halo and C₂-C₃ alkynyl.

### Embodiment 60

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of hydrogen and halo.

### Embodiment 61

The compound of embodiment 54, wherein each R³, R⁴, R^{h}, Rⁱ, R^{j}, R^{k}, Rⁿ, R^{o}, R^{q}, R^{r} and R^{s} is independently halo.

### Embodiment 62

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl, -Me, -Et, -cyclopropyl, —CF₃ and —C═CH.

### Embodiment 63

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl, -Me and -Et.

### Embodiment 64

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl and -cyclopropyl.

### Embodiment 65

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl and —CF₃.

### Embodiment 66

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl and —C═CH.

### Embodiment 67

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —H, —F and —Cl.

### Embodiment 68

The compound of embodiment 54, wherein each R³, R⁴, R^{h}, Rⁱ, R^{j}, R^{k}, Rⁿ, R^{o}, R^{q}, R^{r} and R^{s} is independently selected from the group consisting of —F and —Cl.

### Embodiment 69

The compound of any one of embodiments 54 to 68, wherein each R^{g}, R^{m} and R^{p} is independently selected from the group consisting of —H and —OH.

### Embodiment 70

The compound of any one of embodiments 54 to 68, wherein each R^{g}, R^{m} and R^{p} is independently —H.

### Embodiment 71

The compound of any one of embodiments 54 to 68, wherein each R^{g}, R^{m} and R^{p} is independently —OH.

### Embodiment 72

The compound of any one of embodiments 54 to 71, wherein Ring A is selected from the group consisting of:

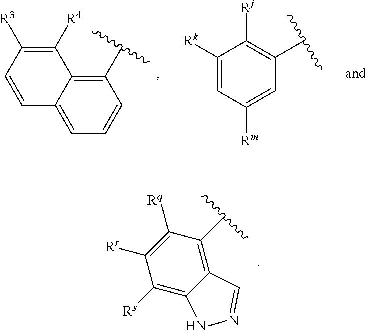

### Embodiment 73

The compound of any one of embodiments 54 to 71, wherein Ring A is selected from the group consisting of:

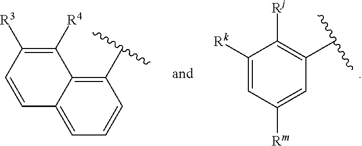

### Embodiment 74

The compound of any one of embodiments 54 to 71, wherein Ring A is

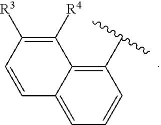

### Embodiment 75

The compound of any one of embodiments 54 to 71, wherein Ring A is

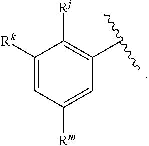

### Embodiment 76

The compound of any one of embodiments 54 to 71, wherein Ring A is

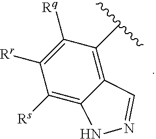

### Embodiment 77

The compound any one of embodiments 54 to 71, wherein Ring A is

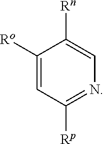

### Embodiment 78

The compound of any one of embodiments 1-10, wherein Ring A is selected from:

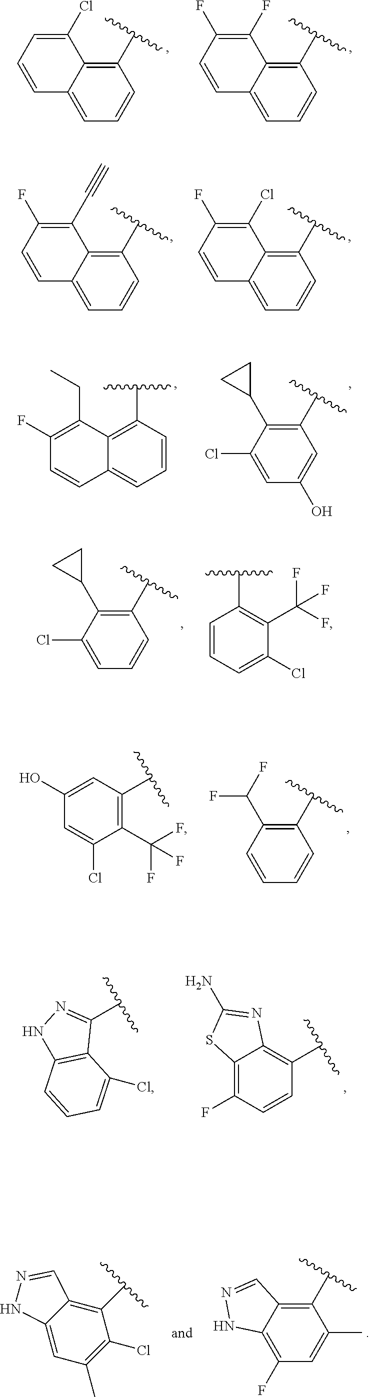

### Embodiment 79

The compound of any one of embodiments 1-10, wherein Ring A is selected from:

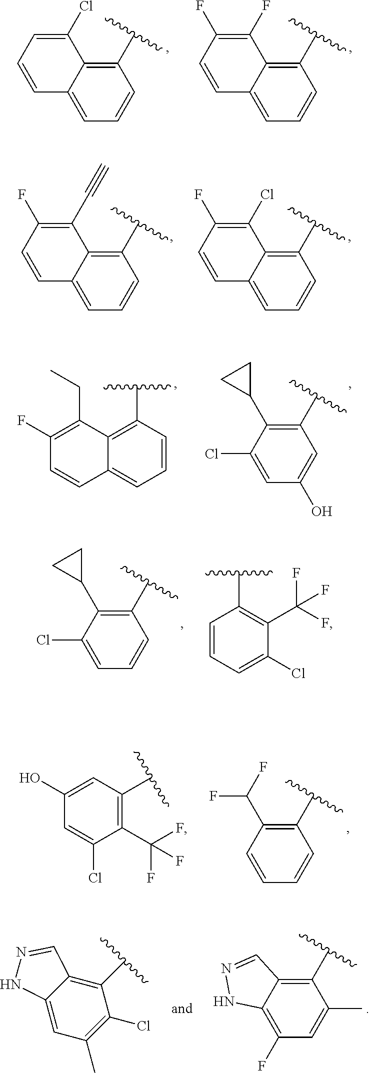

### Embodiment 80

The compound of any one of embodiments 1-10, wherein Ring A is selected from:

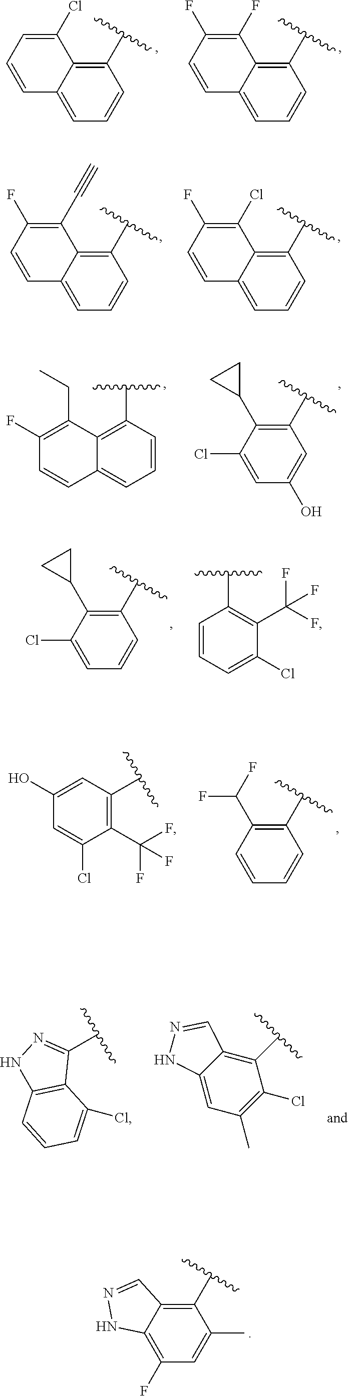

### Embodiment 81

The compound of any one of embodiments 1-10, wherein Ring A is selected from:

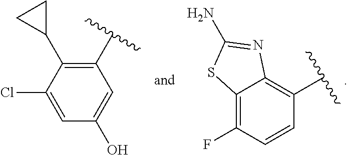 [SMILES: *c1cc(O)cc(Cl)c1C1CC1 ; *c1ccc(F)c2sc(N)nc12]

### Embodiment 82

The compound of any one of embodiments 1-10, wherein Ring A is

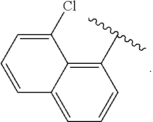 [SMILES: *c1cccc2cccc(Cl)c12]

### Embodiment 83

The compound of any one of embodiments 1-10, wherein Ring A is

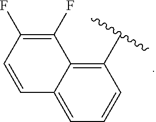 [SMILES: *c1cccc2ccc(F)c(F)c12]

### Embodiment 84

The compound of any one of embodiments 1-10, wherein Ring A is

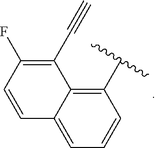 [SMILES: *c1cccc2ccc(F)c(C#C)c12]

### Embodiment 85

The compound of any one of embodiments 1-10, wherein Ring A is

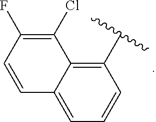 [SMILES: *c1cccc2ccc(F)c(Cl)c12]

### Embodiment 86

The compound of any one of embodiments 1-10, wherein Ring A is

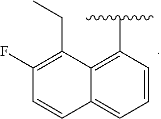 [SMILES: *c1cccc2ccc(F)c(CC)c12 ; "."]

### Embodiment 87

The compound of any one of embodiments 1-10, wherein Ring A is

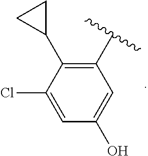 [SMILES: *c1cc(O)cc(Cl)c1C1CC1]

### Embodiment 88

The compound of any one of embodiments 1-10, wherein Ring A is

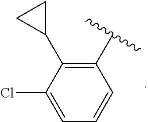 [SMILES: *c1cccc(Cl)c1C1CC1]

### Embodiment 89

The compound of any one of embodiments 1-10, wherein Ring A is

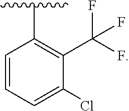 [SMILES: *c1cccc(Cl)c1C(F)(F)F]

### Embodiment 90

The compound of any one of embodiments 1-10, wherein Ring A is

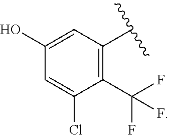 [SMILES: *c1cc(O)cc(Cl)c1C(F)(F)F]

### Embodiment 91

The compound of any one of embodiments 1-10, wherein Ring A is

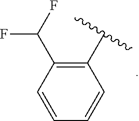 [SMILES: *c1ccccc1C(F)F]

### Embodiment 92

The compound of any one of embodiments 1-10, wherein Ring A is

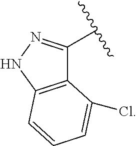 [SMILES: *c1n[nH]c2cccc(Cl)c12]

### Embodiment 93

The compound of any one of embodiments 1-10, wherein Ring A is

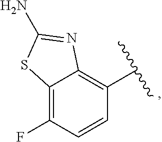 [SMILES: *c1ccc(F)c2sc(N)nc12]

### Embodiment 94

The compound of any one of embodiments 1-10, wherein Ring A is

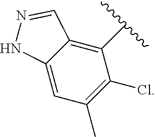 [SMILES: *c1c(Cl)c(C)cc2[nH]ncc12]

### Embodiment 95

The compound of any one of embodiments 1-10, wherein Ring A is

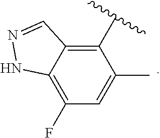 [SMILES: *c1c(C)cc(F)c2[nH]ncc12]

### Embodiment 96

The compound of embodiment 1, wherein the compound is of Formula (I-a) or Formula (II-a):

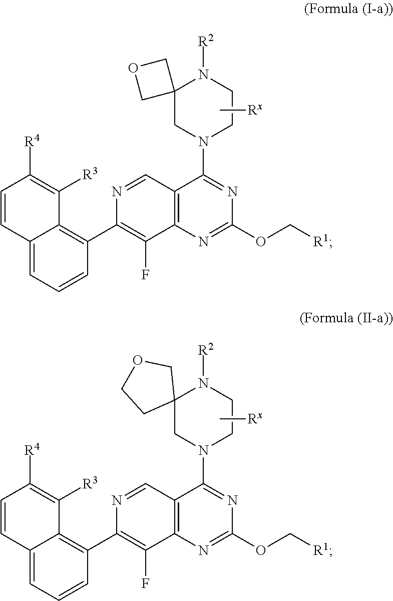

or a salt thereof; and/or an isotopologue thereof; wherein:
R³ is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
R⁴ is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl.

### Embodiment 97

The compound of embodiment 96, wherein the compound is of Formula (I-a).

### Embodiment 98

The compound of embodiment 96, wherein the compound is of Formula (II-a).

### Embodiment 99

The compound of any one of embodiments 54, 72-74 and 96-98, wherein R³ is selected from halo, C₁-C₄ alkyl, and C₂-C₃ alkynyl.

### Embodiment 100

The compound of any one of embodiments 54, 72-74 and 96-98, wherein R³ is selected from halo and C₁-C₄ alkyl.

### Embodiment 101

The compound of any one of embodiments 54, 72-74 and 96-98, wherein R³ is selected from halo and C₂-C₃ alkynyl.

### Embodiment 102

The compound of any one of embodiments 54, 72-74 and 96-98, wherein R³ is halo.

### Embodiment 103

The compound of any one of embodiments 54, 72-74 and 96-98, wherein R³ is selected from —F, —Cl, -Et, and —C═CH.

### Embodiment 104

The compound of any one of embodiments 54, 72-74 and 96-98, wherein R³ is selected from —F, —Cl, and -Et.

### Embodiment 105

The compound of any one of embodiments 54, 72-74 and 96-98, wherein R³ is selected from —F, —Cl and —C═CH.

### Embodiment 106

The compound of any one of embodiments 54, 72-74 and 96-98, wherein R³ is selected from —F and —Cl.

### Embodiment 107

The compound of any one of embodiments 54, 72-74 and 96-98, wherein R³ is —F.

### Embodiment 108

The compound of any one of embodiments 54, 72-74 and 96-98, wherein R³ is —Cl.

### Embodiment 109

The compound of any one of embodiments 54, 72-74 and 96-98, wherein

R³ is -Et.

### Embodiment 110

The compound of any one of embodiments 54, 72-74 and 96-98, wherein R³ is —C═CH.

### Embodiment 111

The compound of any one of embodiments 54, 72-74 and 96-110, wherein R⁴ is selected from hydrogen and halo.

### Embodiment 112

The compound of any one of embodiments 54, 72-74 and 96-110, wherein R⁴ is selected from —H and —F.

### Embodiment 113

The compound of any one of embodiments 54, 72-74 and 96-110, wherein R⁴ is —H.

### Embodiment 114

The compound of any one of embodiments 54, 72-74 and 96-110, wherein R⁴ is —F.

### Embodiment 115

The compound of embodiment 1, wherein the compound is of Formula (I-b) or Formula (II-b):

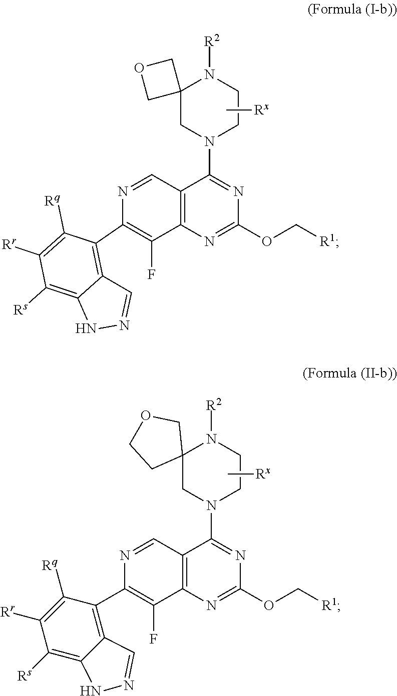

or a salt thereof; and/or an isotopologue thereof; wherein:
each R⁹, R^{r} and R^{s} is independently selected from the group consisting of hydrogen, halo, C₁-C₄ alkyl, C₁-C₄ alkoxy, C₁-C₄ haloalkyl, C₁-C₄ haloalkoxy and C₂-C₃ alkynyl.

### Embodiment 116

The compound of embodiment 115, wherein the compound is of Formula (I-b).

### Embodiment 117

The compound of embodiment 115, wherein the compound is of Formula (II-b).

### Embodiment 118

The compound of any one of embodiments 54, 72, 76 and 97-117, wherein R^{q} is selected from the group consisting of hydrogen, halo and C₁-C₄ alkyl.

### Embodiment 119

The compound of any one of embodiments 54, 72, 76 and 97-117, wherein R^{q} is selected from the group consisting of halo and C₁-C₄ alkyl.

### Embodiment 120

The compound of any one of embodiments 54, 72, 76 and 97-117, wherein R^{q} is selected from the group consisting of hydrogen, —F, —Cl, and -Me.

### Embodiment 121

The compound of any one of embodiments 54, 72, 76 and 97-117, wherein R^{q} is selected from the group consisting of —Cl, and -Me.

### Embodiment 122

The compound of any one of embodiments 54, 72, 76 and 97-117, wherein R^{q} is —Cl.

### Embodiment 123

The compound of any one of embodiments 54, 72, 76 and 97-117, wherein R^{q} is -Me.

### Embodiment 124

The compound of any one of embodiments 54, 72, 76 and 97-123, wherein R^{f} is selected from the group consisting of hydrogen and C₁-C₄ alkyl.

### Embodiment 125

The compound of any one of embodiments 54, 72, 76 and 97-123, wherein R^{f} is selected from the group consisting of —H and -Me.

### Embodiment 126

The compound of any one of embodiments 54, 72, 76 and 97-123, wherein R^{f} is —H.

### Embodiment 127

The compound of any one of embodiments 54, 72, 76 and 97-123, wherein R^{f} is -Me.

### Embodiment 128

The compound of any one of embodiments 54, 72, 76 and 97-127, wherein R^{s} is selected from the group consisting of hydrogen and halo.

### Embodiment 129

The compound of any one of embodiments 54, 72, 76 and 97-127, wherein R^{s} is selected from the group consisting of —H and —F.

### Embodiment 130

The compound of any one of embodiments 54, 72, 76 and 97-127, wherein R^{s} is —H.

### Embodiment 131

The compound of any one of embodiments 54, 72, 76 and 97-127, wherein R^{s} is —F.

### Embodiment 132

The compound of embodiment 1, wherein the compound is of Formula (I-c) or Formula (II-c):

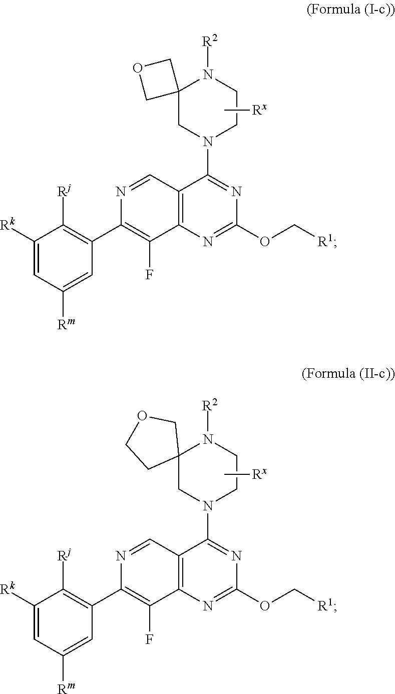

or a salt thereof; and/or an isotopologue thereof; wherein:
R^{j} is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
R^{k} is selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
R^{m} is selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl. Embodiment 133. The compound of embodiment 132, wherein the compound is of Formula (I-c).

### Embodiment 134

The compound of embodiment 132, wherein the compound is of Formula (II-c).

### Embodiment 135

The compound of any one of embodiments 54, 72, 73, 75, 99-114 and 116-134, wherein R^{J} is selected from C₃-C₄ cycloalkyl and C₁-C₄ haloalkyl.

### Embodiment 136

The compound of any one of embodiments 54, 72, 73, 75, 99-114 and 118-134, wherein R^{J} is selected from cyclopropyl, —CHF₂ and —CF₃.

### Embodiment 137

The compound of any one of embodiments 54, 72, 73, 75, 99-114 and 118-134, wherein R^{j} is cyclopropyl.

### Embodiment 138

The compound of any one of embodiments 54, 72, 73, 75, 99-114 and 118-134, wherein R^{j} is —CHF₂.

### Embodiment 139

The compound of any one of embodiments 54, 72, 73, 75, 99-114 and 118-134, wherein R^{j} is —CF₃.

### Embodiment 140

The compound of any one of embodiments 54, 72, 73, 75, 99-114 and 118-139, wherein R^{k} is selected from hydrogen and halo.

### Embodiment 141

The compound of any one of embodiments 54, 72, 73, 75, 99-114 and 118-139, wherein R^{k} is selected from —H and —Cl.

### Embodiment 142

The compound of any one of embodiments 54, 72, 73, 75, 99-114 and 118-139, wherein R^{k} is —H.

### Embodiment 143

The compound of any one of embodiments 54, 72, 73, 75, 99-114 and 118-139, wherein R^{k} is —Cl.

### Embodiment 144

The compound of any one of embodiments 54, 72, 73, 75, 99-114 and 118-143, wherein R^{m} is selected from —H and —OH.

### Embodiment 145

The compound of any one of embodiments 54, 72, 73, 75, 99-114 and 118-143, wherein R^{m} is —H.

### Embodiment 146

The compound of any one of embodiments 54, 72, 73, 75, 99-114 and 116-143, wherein R^{m} is —OH.

### Embodiment 147

The compound of any one of embodiments 1-146, wherein R^{d} is H.

### Embodiment 148

The compound of any one of embodiments 1-146, wherein R^{d} is F.

### Embodiment 149

The compound of any one of embodiments 1-146, wherein R¹ is selected from

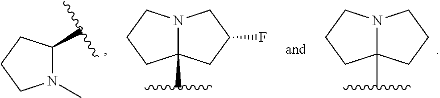

### Embodiment 150

The compound of any one of embodiments 1-146, wherein R¹ is

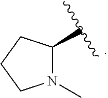 [SMILES: *[C@@H]1CCCN1C]

### Embodiment 151

The compound of any one of embodiments 1-146, wherein R¹ is

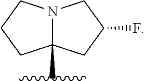 [SMILES: *[C@@]12CCCN1C[C@H](F)C2]

### Embodiment 152

The compound of any one of embodiments 1-146, wherein R¹ is

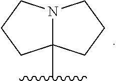 [SMILES: *C12CCCN1CCC2]

### Embodiment 153

The compound of any one of embodiments 1-152, wherein R² is

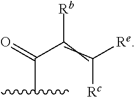

### Embodiment 154

The compound of any one of embodiments 1-153, wherein R^{b} is —H.

### Embodiment 155

The compound of any one of embodiments 1-153, wherein R^{b} is —F.

### Embodiment 156

The compound of any one of embodiments 1-155, wherein each R^{Y1} is —H.

### Embodiment 157

The compound of any one of embodiments 1-155, wherein at least one R^{Y1} is -Me.

### Embodiment 158

The compound of any one of embodiments 1-157, wherein each R^{Y2} is —H.

### Embodiment 159

The compound of any one of embodiments 1-157, wherein at least one R^{Y2} is -Me.

### Embodiment 160

The compound of any one of embodiments 1-159, wherein x is 0 or 1.

### Embodiment 161

The compound of any one of embodiments 1-159, wherein x is 1 or 2.

### Embodiment 162

The compound of any one of embodiments 1-159, wherein x is 0.

### Embodiment 163

The compound of any one of embodiments 1-159, wherein x is 0 or 1.

### Embodiment 164

The compound of any one of embodiments 1-155, wherein Y is selected from a bond, —CH₂—, —CH(CH₃)—, —C(CH₃)₂—, —CH₂CH₂—, —CH(CH₃)CH₂— and —CH₂CH(CH₃)—.

### Embodiment 165

The compound of any one of embodiments 1-155, wherein Y is selected from a bond, —CH₂— and —CH(CH₃)—.

### Embodiment 166

The compound of any one of embodiments 1-155, wherein Y is selected from a bond and —CH₂—.

### Embodiment 167

The compound of any one of embodiments 1-155, wherein Y is a bond.

### Embodiment 168

The compound of any one of embodiments 1-155, wherein Y is —CH₂—.

### Embodiment 169

The compound of any one of embodiments 1-155, wherein Y is —CH(CH₃)—.

### Embodiment 170

The compound of any one of embodiments 1-155, wherein R^{e} is selected from —H, —R^{e1}, —CH₂—R^{e1}, CH(CH₃)—R^{e1}, —R^{e2}, —CH₂—R^{e3}, —CH(CH₃)—R^{e3}.

### Embodiment 171

The compound of any one of embodiments 1-155, wherein R^{e} is selected from —H, —R^{e1}, —CH₂—R^{e1}, —R^{e2} and —CH₂—R^{e3}.

### Embodiment 172

The compound of any one of embodiments 1-155, wherein R^{e} is selected from —R^{e1}, —CH₂—R^{e1} and —CH(CH₃)—R^{e1}.

### Embodiment 173

The compound of any one of embodiments 1-155, wherein R^{e} is selected from —R^{e1} and —CH₂—R^{e1}.

### Embodiment 174

The compound of any one of embodiments 1-155, wherein R^{e} is —R^{e1} Embodiment 175. The compound of any one of embodiments 1-155, wherein R^{e} is —CH₂—R^{e1}.

### Embodiment 176

The compound of any one of embodiments 1-155, wherein R^{e} is —CH(CH₃)—R^{e1}.

### Embodiment 177

The compound of any one of embodiments 1-176, wherein R^{e1} is a 4-7 membered monocyclic heterocycle containing a nitrogen atom as the only heteroatom or containing one nitrogen atom and one oxygen atom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 178

The compound of any one of embodiments 1-176, wherein R^{e1} is a 4-7 membered monocyclic heterocycle containing a nitrogen atom as the only heteroatom, wherein the monocyclic heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 179

The compound of embodiment 177 or 178 wherein the monocyclic heterocycle of R^{e1} is substituted with 0 or 1 instance of C₁-C₄ alkyl.

### Embodiment 180

The compound of embodiment 177 or 178, wherein the monocyclic heterocycle of R^{e1} is substituted with 0 or 1 instance of methyl.

### Embodiment 181

The compound of embodiment 177, wherein R^{e1} is selected from azetidinyl, pyrrolidinyl and morpholinyl substituted with 0 or 1 instance of methyl.

### Embodiment 182

The compound of embodiment 177 or 178, wherein R^{e1} is azetidinyl substituted with 0 or 1 instance of methyl.

### Embodiment 183

The compound of embodiment 177 or 178, wherein R^{e1} is N-methyl azetidinyl.

### Embodiment 184

The compound of any one of embodiments 177-183, wherein the attachment point for the monocyclic heterocycle is on a carbon atom.

### Embodiment 185

The compound of embodiment 184 wherein R^{e1} is

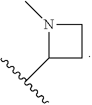 [SMILES: *C1CCN1C]

### Embodiment 186

The compound of embodiment 184 wherein R^{e1} is

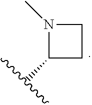 [SMILES: *[C@H]1CCN1C]

### Embodiment 187

The compound of any one of embodiments 1-176, wherein R^{e1} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 188

The compound of embodiment 187, wherein R^{e1} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 189

The compound of embodiment 187, wherein R^{e1} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 190

The compound of embodiment 187, wherein R^{e1} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 191

The compound of embodiment 187, wherein R^{e1} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 192

The compound of embodiment 187, wherein R^{e1} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 193

The compound of embodiment 187, wherein R^{e1} is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 194

The compound of embodiment 187, wherein R^{e1} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 195

The compound of any one of embodiments 187-194, wherein the attachment point for R^{e1} is the nitrogen atom of the heterocycle.

### Embodiment 196

The compound of embodiment 195, wherein R^{e1} is selected from the group consisting of:

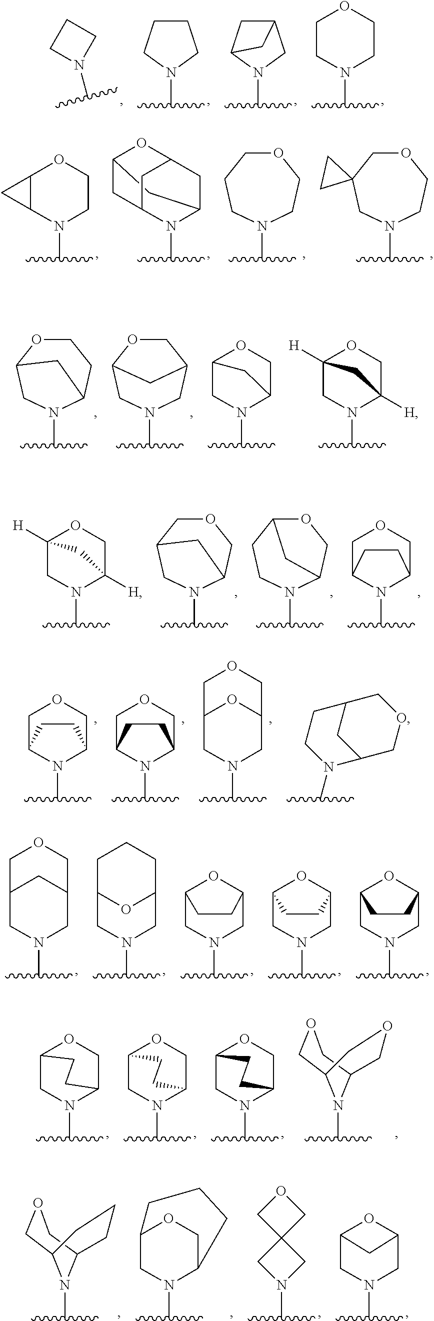

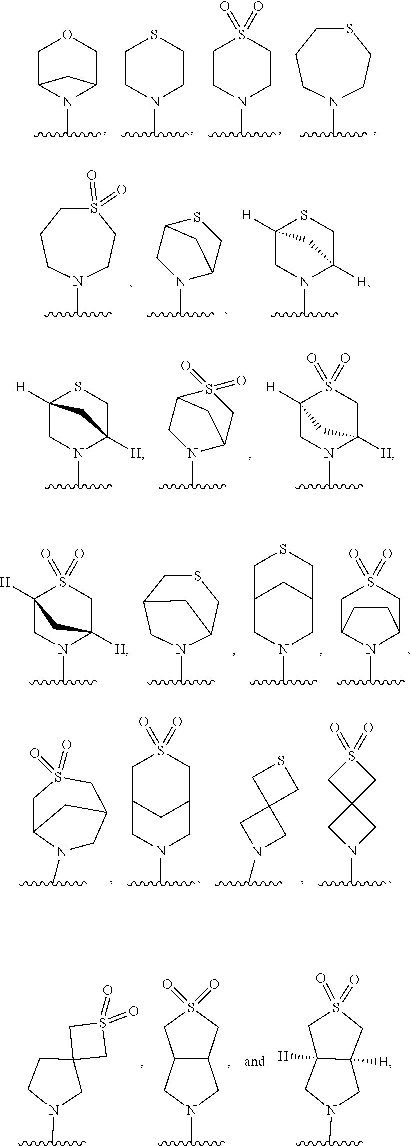

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 197

The compound of embodiment 195, wherein R^{e1} is selected from

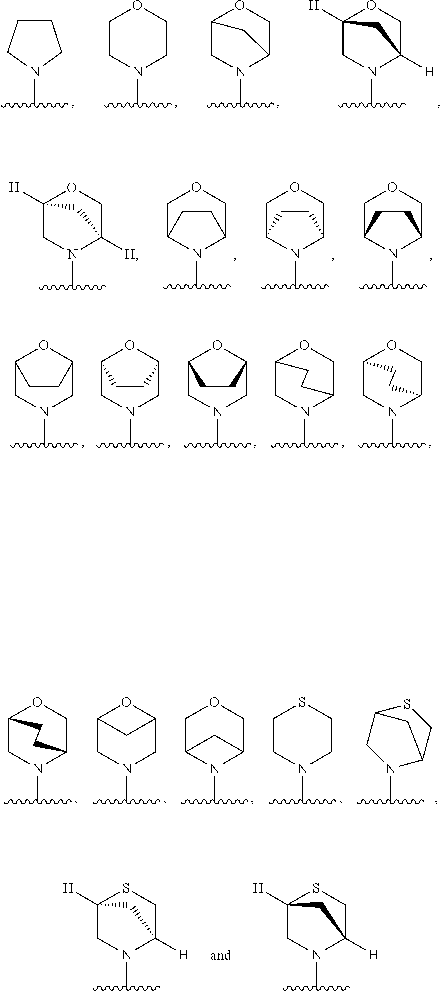

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 198

The compound of embodiment 195, wherein R^{e1} is selected from

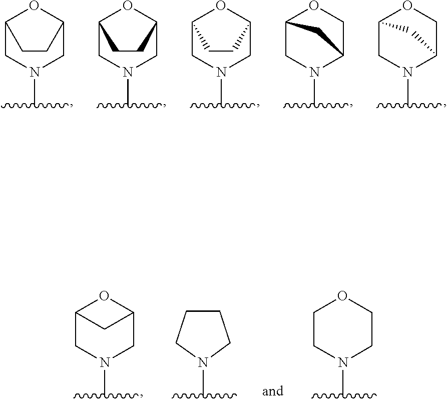

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 199

The compound of embodiment 195, wherein R^{e1} is

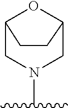 [SMILES: *N1CC2CCC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 200

The compound of embodiment 195, wherein R^{e1} is

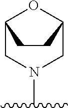 [SMILES: *N1C[C@H]2CC[C@@H](C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 201

The compound of embodiment 195, wherein R^{e1} is

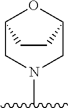 [SMILES: *N1C[C@H]2CC[C@@H](C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 202

The compound of embodiment 195, wherein R^{e1} is

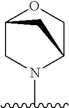 [SMILES: *N1C[C@H]2C[C@@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 203

The compound of embodiment 195, wherein R^{e1} is

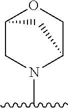 [SMILES: *N1C[C@@H]2C[C@H]1CO2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 204

The compound of embodiment 195, wherein R^{e1} is

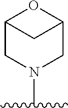 [SMILES: *N1CC2CC(C1)O2]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 205

The compound of embodiment 195, wherein R^{e1} is

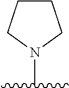 [SMILES: *N1CCCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 206

The compound of embodiment 195, wherein R^{e1} is

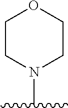 [SMILES: *N1CCOCC1]

substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 207

The compound of any one of embodiments 187-206, wherein the 4-10 membered heterocycle of R^{e1} is substituted with 0, 1 or 2 substituents independently selected from —F, —OMe and -Me.

### Embodiment 208

The compound of any one of embodiments 187-206, wherein the 4-10 membered heterocycle of R^{e1} is unsubstituted.

### Embodiment 209

The compound of embodiment 195, wherein R^{e1} is selected from the group consisting of:

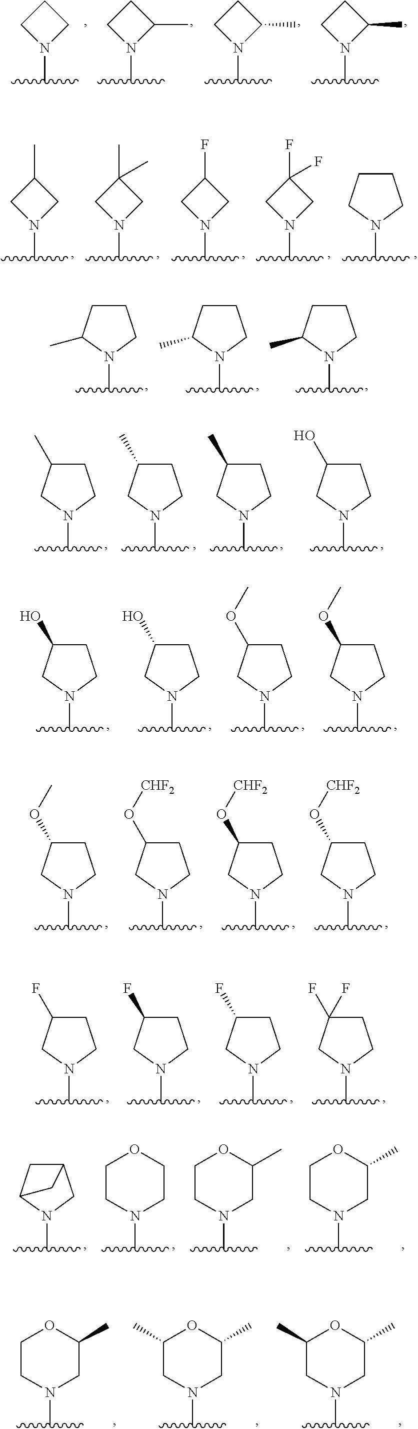

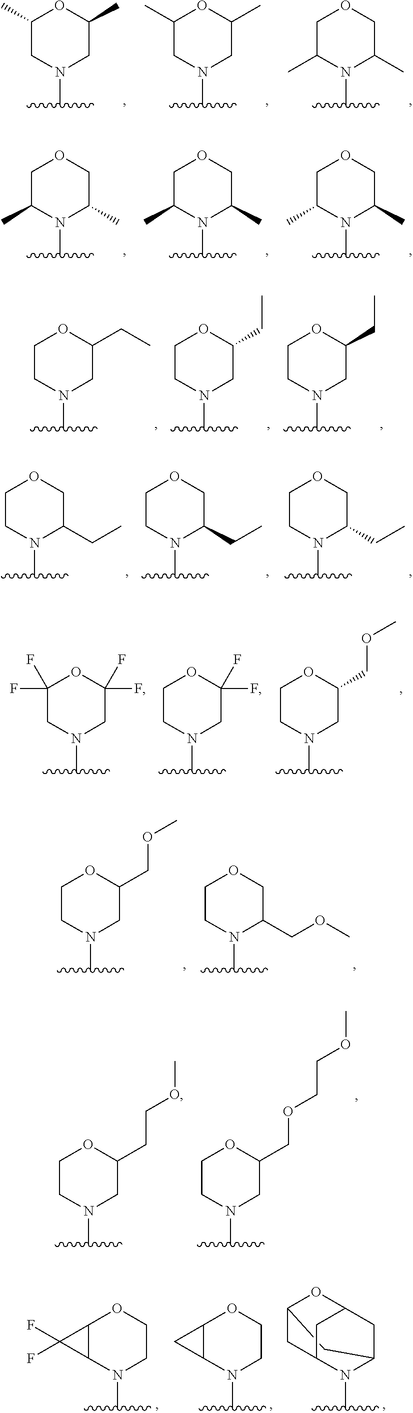

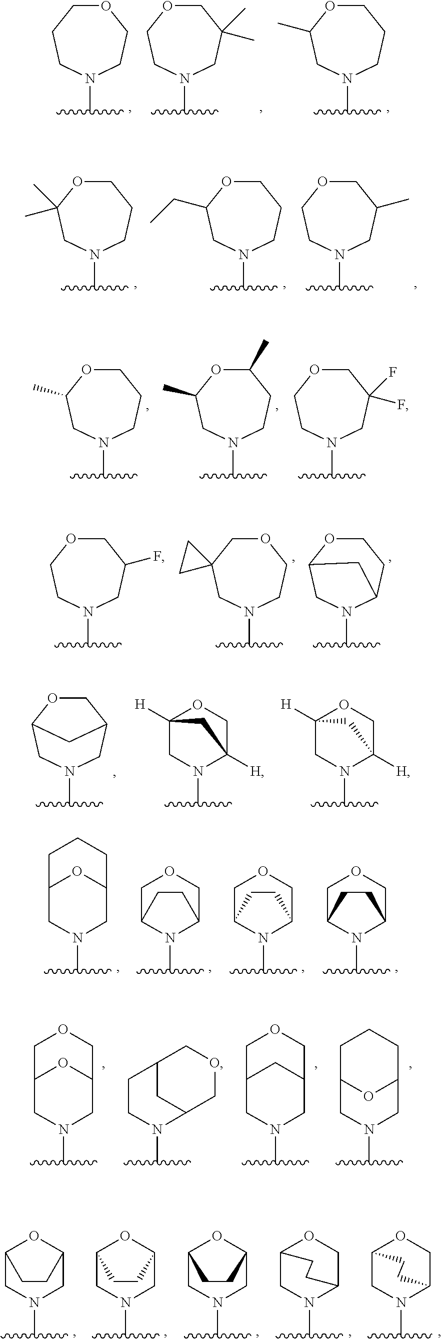

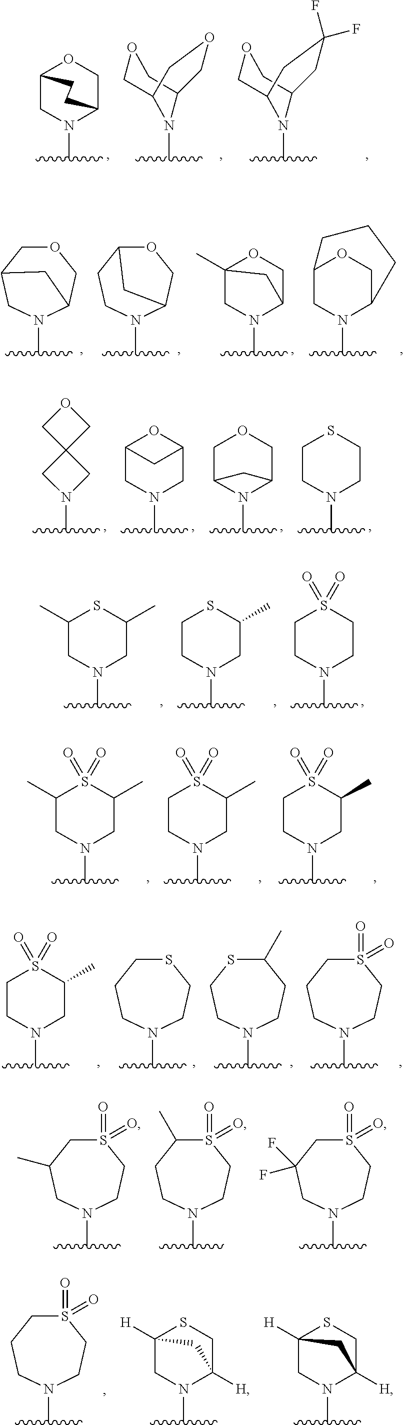

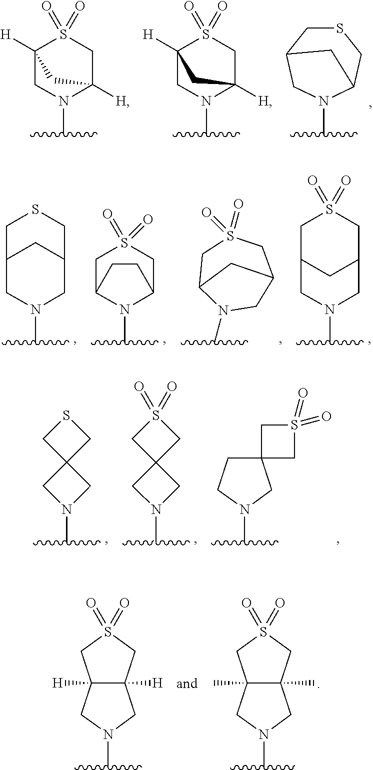

### Embodiment 210

The compound of embodiment 195, wherein R^{e1} is selected from

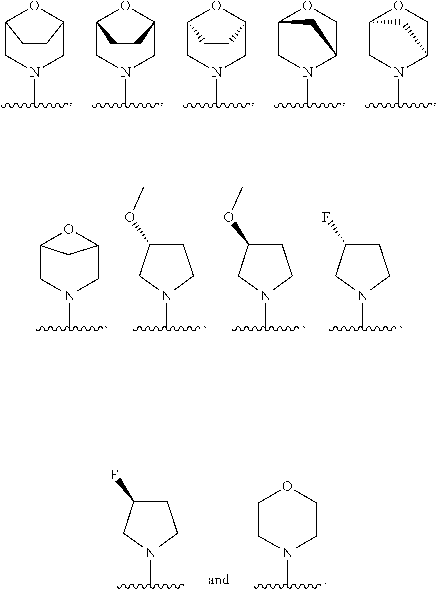

### Embodiment 211

The compound of embodiment 195, wherein R^{e1} is unsubstituted

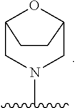 [SMILES: *N1CC2CCC(C1)O2]

### Embodiment 212

The compound of embodiment 195, wherein R^{e1} is unsubstituted

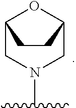 [SMILES: *N1C[C@H]2CC[C@@H](C1)O2]

### Embodiment 213

The compound of embodiment 195, wherein R^{e1} is unsubstituted

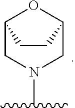 [SMILES: *N1C[C@H]2CC[C@@H](C1)O2]

### Embodiment 214

The compound of embodiment 195, wherein R^{e1} is unsubstituted

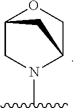 [SMILES: *N1C[C@H]2C[C@@H]1CO2]

### Embodiment 215

The compound of embodiment 195, wherein R^{e1} is unsubstituted

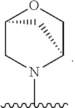 [SMILES: *N1C[C@@H]2C[C@H]1CO2]

### Embodiment 216

The compound of embodiment 195, wherein R^{e1} is unsubstituted

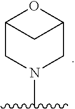 [SMILES: *N1CC2CC(C1)O2]

### Embodiment 217

The compound of embodiment 195, wherein R^{e1} is

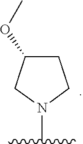 [SMILES: *N1CC[C@@H](OC)C1]

### Embodiment 218

The compound of embodiment 195, wherein R^{e1} is

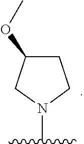 [SMILES: *N1CC[C@H](OC)C1]

### Embodiment 219

The compound of embodiment 195, wherein R^{e1} is

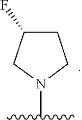 [SMILES: *N1CC[C@@H](F)C1]

### Embodiment 220

The compound of embodiment 195, wherein R^{e1} is

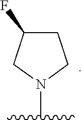 [SMILES: *N1CC[C@H](F)C1]

### Embodiment 221

The compound of embodiment 195, wherein R^{e1} is unsubstituted

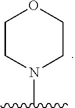 [SMILES: *N1CCOCC1]

### Embodiment 222

The compound of any one of embodiments 1-155, wherein R^{e} is selected from

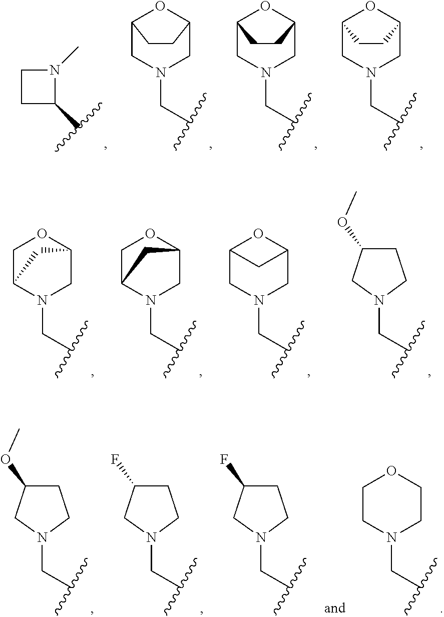

### Embodiment 223

The compound of any one of embodiments 1-155, wherein R^{e} is

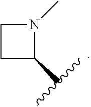 [SMILES: *[C@H]1CCN1C]

### Embodiment 224

The compound of any one of embodiments 1-155, wherein R^{e} is

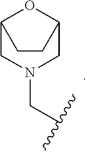 [SMILES: *CN1CC2CCC(C1)O2]

### Embodiment 225

The compound of any one of embodiments 1-155, wherein R^{e} is

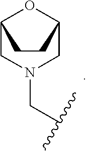 [SMILES: *CN1C[C@H]2CC[C@@H](C1)O2]

### Embodiment 226

The compound of any one of embodiments 1-155, wherein R^{e} is

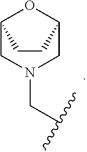 [SMILES: *CN1C[C@H]2CC[C@@H](C1)O2]

### Embodiment 227

The compound of any one of embodiments 1-155, wherein R^{e} is

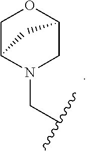 [SMILES: *CN1C[C@H]2C[C@@H]1CO2]

### Embodiment 228

The compound of any one of embodiments 1-155, wherein R^{e} is

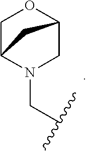 [SMILES: *CN1C[C@@H]2C[C@H]1CO2]

### Embodiment 229

The compound of any one of embodiments 1-155, wherein R^{e} is

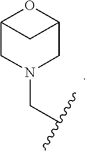 [SMILES: *CN1CC2CC(C1)O2]

### Embodiment 230

The compound of any one of embodiments 1-155, wherein R^{e} is

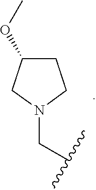 [SMILES: *CN1CC[C@@H](OC)C1]

### Embodiment 231

The compound of any one of embodiments 1-155, wherein R^{e} is

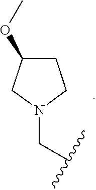 [SMILES: *CN1CC[C@H](OC)C1]

### Embodiment 232

The compound of any one of embodiments 1-155, wherein R^{e} is

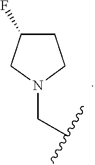 [SMILES: *CN1CC[C@@H](F)C1]

### Embodiment 233

The compound of any one of embodiments 1-155, wherein R^{e} is

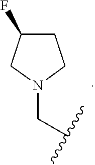 [SMILES: *CN1CC[C@H](F)C1]

### Embodiment 234

The compound of any one of embodiments 1-155, wherein R^{e} is

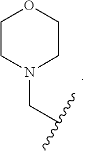 [SMILES: *CN1CCOCC1]

### Embodiment 235

The compound of any one of embodiments 1-155, wherein R^{e} is selected from —R^{e2}, —CH₂—R^{e2} and —CH(CH₃)—R^{e2}.

### Embodiment 236

The compound of any one of embodiments 1-155, wherein R^{e} is selected from —R^{e2} and —CH₂—R^{e2}.

### Embodiment 237

The compound of any one of embodiments 1-155, wherein R^{e} is —R^{e2}.

### Embodiment 238

The compound of any one of embodiments 1-155, wherein R^{e} is —CH₂—R^{e2}.

### Embodiment 239

The compound of any one of embodiments 1-155, wherein R^{e} is —CH(CH₃)—R^{e2}.

### Embodiment 240

The compound of any one of embodiments 1-155 and 235-239, wherein R^{e2} is a 5-6 membered heteroaryl group containing at least one nitrogen atom, wherein the attachment point for the heteroaryl group is a carbon atom group and wherein the heteroaryl is substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 241

The compound of any one of embodiments 1-155 and 235-240, wherein R^{e2} is selected from the group consisting of pyrimidinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 242

The compound of any one of embodiments 1-155 and 235-240, wherein R^{e2} is selected from the group consisting of:

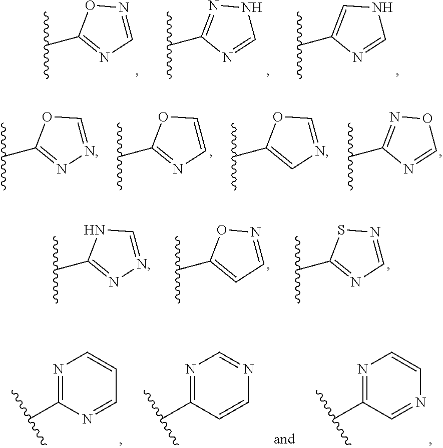

each substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 243

The compound of any one of embodiments 1-155 and 235-240, wherein R^{e2} is a 6 membered heteroaryl group substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 244

The compound of embodiment 243, wherein R^{e2} is pyrimidinyl or pyridazinyl substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 245

The compound of embodiment 243, wherein R^{e2} is pyrimidinyl or pyridazinyl substituted with 0, 1 or 2 instances of C₁-C₄ alkyl.

### Embodiment 246

The compound of embodiment 243, wherein R^{e2} is pyrimidinyl substituted with 0, 1 or 2 instances of C₁-C₄ alkyl.

### Embodiment 247

The compound of embodiment 243, wherein R^{e2} is pyridazinyl substituted with 0, 1 or 2 instances of C₁-C₄ alkyl.

### Embodiment 248

The compound of embodiment 243, wherein R^{e2} is pyrimidinyl or pyridazinyl substituted with 0, 1 or 2 instances of -Me.

### Embodiment 249

The compound of embodiment 243, wherein R^{e2} is pyrimidinyl substituted with 0, 1 or 2 instances of -Me.

### Embodiment 250

The compound of embodiment 243, wherein R^{e2} is pyridazinyl substituted with 0, 1 or 2 instances of -Me.

### Embodiment 251

The compound of embodiment 244, wherein R^{e2} is selected from the group consisting of:

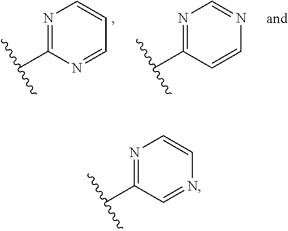 [SMILES: *c1ncccn1 ; *c1ccncn1 ; "and" ; *c1cnccn1]

substituted with 0, 1 or 2 substituents independently selected from halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

### Embodiment 252

The compound of embodiment 244, wherein R^{e2} is selected from the group consisting of

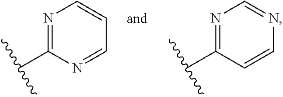 [SMILES: *c1ncccn1 ; "and" ; *c1ccncn1]

substituted with 0, 1 or 2 instances of C₁-C₄ alkyl.

### Embodiment 253

The compound of embodiment 244, wherein R^{e2} is

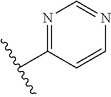 [SMILES: *c1ccncn1]

substituted with 0, 1 or 2 instances of C₁-C₄ alkyl.

### Embodiment 254

The compound of embodiment 244, wherein R^{e2} is

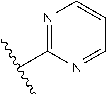 [SMILES: *c1ncccn1]

substituted with 0, 1 or 2 instances of C₁-C₄ alkyl.

### Embodiment 255

The compound of embodiment 244, wherein R^{e2} is

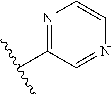 [SMILES: *c1cnccn1]

substituted with 0, 1 or 2 instances of C₁-C₄ alkyl.

### Embodiment 256

The compound of embodiment 244, wherein R^{e2} is selected from the group consisting of

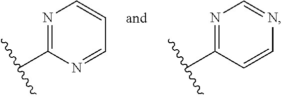 [SMILES: *c1ncccn1 ; "and" ; *c1ccncn1]

substituted with 0, 1 or 2 instances of -Me.

### Embodiment 257

The compound of embodiment 244, wherein R^{e2} is

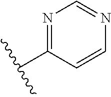 [SMILES: *c1ccncn1]

substituted with 0, 1 or 2 instances of -Me.

### Embodiment 258

The compound of embodiment 244, wherein R^{e2} is

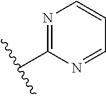 [SMILES: *c1ncccn1]

substituted with 0, 1 or 2 instances of -Me.

### Embodiment 259

The compound of embodiment 244, wherein R^{e2} is

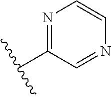 [SMILES: *c1cnccn1]

substituted with 0, 1 or 2 instances of -Me.

### Embodiment 260

The compound of embodiment 244, wherein R^{e2} is selected from the group consisting of

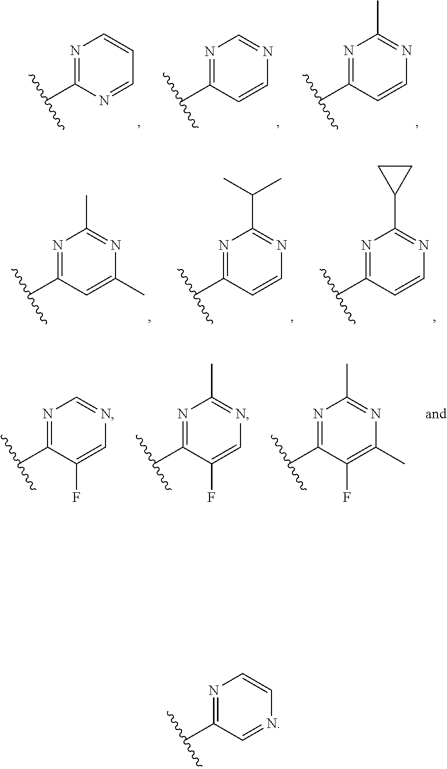

### Embodiment 261

The compound of embodiment 244, wherein R^{e2} is selected from the group consisting of

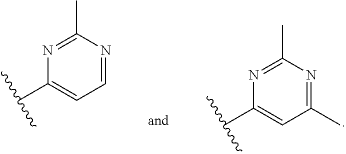

### Embodiment 262

The compound of embodiment 244, wherein R^{e2} is

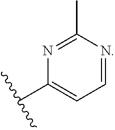 [SMILES: *c1ccnc(C)n1]

### Embodiment 263

The compound of embodiment 244, wherein R^{e2} is

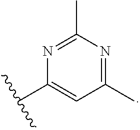 [SMILES: *c1cc(C)nc(C)n1]

### Embodiment 264

The compound of embodiment 244, wherein R^{e} is selected from the group consisting of

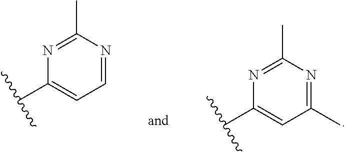

### Embodiment 265

The compound of embodiment 244, wherein R^{e} is

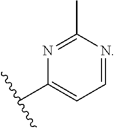 [SMILES: *c1ccnc(C)n1]

### Embodiment 266

The compound of embodiment 244, wherein R^{e} is

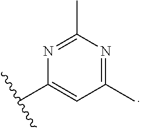 [SMILES: *c1cc(C)nc(C)n1]

### Embodiment 267

The compound of any one of embodiments 1-155, wherein R^{e} is selected from —CH₂—R^{e3} and —CH(CH₃)—R^{e3}.

### Embodiment 268

The compound of any one of embodiments 1-155, wherein R^{e} is —CH₂—R^{e3}.

### Embodiment 269

The compound of any one of embodiments 1-155, wherein R^{e} is —CH(CH₃)—R^{e3}.

### Embodiment 270

The compound of any one of embodiments 1-155 and 267-269, wherein R^{e3} is —NR³¹R³², wherein R³¹ and R³² are independently selected from the group consisting of C₁-C₄ alkyl, C₁-C₄ alkoxy, C₁-C₄ haloalkoxy, and C₁-C₄ alkyl substituted with a 3-6 membered heterocycle containing 1-2 atoms independently selected from N, O and S, including S(O)₂.

### Embodiment 271

The compound of any one of embodiments 1-155 and 267-270, wherein R³¹ is C₁-C₄ alkyl.

### Embodiment 272

The compound of any one of embodiments 1-155 and 267-270, wherein R³¹ is -Me.

### Embodiment 273

The compound of any one of embodiments 1-155 and 267-272, wherein R³² is C₁-C₄ alkyl or C₁-C₄ alkyl substituted with a 3-6 membered heterocycle.

### Embodiment 274

The compound of any one of embodiments 1-155 and 267-272, wherein R³² is C₁-C₄ alkyl substituted with a 3-6 membered heterocycle.

### Embodiment 275

The compound of any one of embodiments 1-155 and 267-274, wherein R³² is C₁-C₄ alkyl substituted with a heterocycle selected from azetidine, oxetane, pyrrolidine, tetrahydrofuran, piperidine, tetrahydropyran and morpholine.

### Embodiment 276

The compound of any one of embodiments 1-155 and 267-274, wherein R³² is C₁-C₄ alkyl substituted with oxetane.

### Embodiment 277

The compound of any one of embodiments 1-155 and 267-274, wherein R³² is

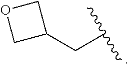 [SMILES: *CC1COC1]

### Embodiment 278

The compound of any one of embodiments 1-155 and 267-272, wherein R³² is C₁-C₄ alkyl.

### Embodiment 279

The compound of any one of embodiments 1-155 and 267-272, wherein R³² is Me.

### Embodiment 280

The compound of any one of embodiments 1-155 and 267-269, wherein R^{e3} is selected from

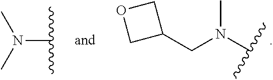

### Embodiment 281

The compound of any one of embodiments 1-155 and 267-269, wherein R^{e3} is

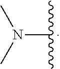 [SMILES: *N(C)C]

### Embodiment 282

The compound of any one of embodiments 1-155 and 267-269, wherein R^{e3} is

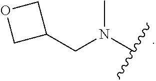 [SMILES: *N(C)CC1COC1]

### Embodiment 283

The compound of any one of embodiments 1-155 and 267-269, wherein R^{e} is selected from

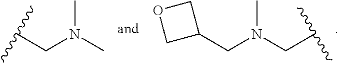

### Embodiment 284

The compound of any one of embodiments 1-155 and 267-269, wherein R^{e} is

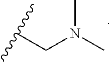 [SMILES: *CN(C)C]

### Embodiment 285

The compound of any one of embodiments 1-155 and 267-269, wherein R^{e} is

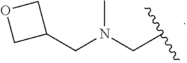 [SMILES: *CN(C)CC1COC1]

### Embodiment 286

The compound of any one of embodiments 1-155, wherein R^{e} is —H.

### Embodiment 287

The compound of any one of embodiments 1-155, wherein R^{e} is selected from the group consisting of: —H,

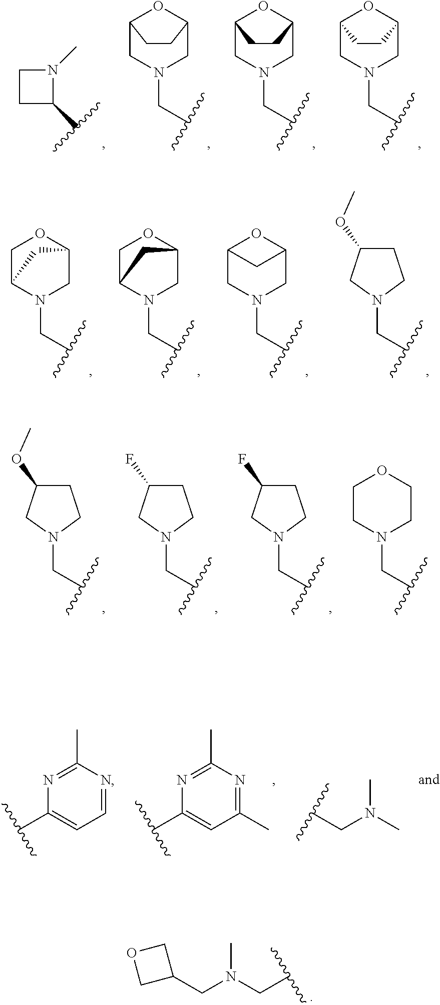

### Embodiment 288

The compound of any one of embodiments 1-152, wherein R² is

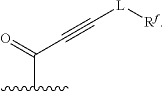

### Embodiment 289

The compound of any one of embodiments 1-152 and 288, wherein each R^{L1} is —H.

### Embodiment 290

The compound of any one of embodiments 1-152 and 288, wherein at least one R^{L1} is -Me.

### Embodiment 291

The compound of any one of embodiments 1-152 and 288-290, wherein each R¹² is —H.

### Embodiment 292

The compound of any one of embodiments 1-152 and 288-290, wherein at least one R^{L2} is -Me.

### Embodiment 293

The compound of any one of embodiments 1-152 and 288-292, wherein z is 0 or 1.

### Embodiment 294

The compound of any one of embodiments 1-152 and 288-292, wherein z is 1 or 2.

### Embodiment 295

The compound of any one of embodiments 1-152 and 288-292, wherein z is 0 or 1.

### Embodiment 296

The compound of any one of embodiments 1-152 and 288-292, wherein z is 0 or 1.

### Embodiment 297

The compound of any one of embodiments 1-152 and 288, wherein L is selected from a bond, —CH₂—, —CH(CH₃)—, —C(CH₃)₂—, —CH₂CH₂—, —CH(CH₃)CH₂— and —CH₂CH(CH₃)—.

### Embodiment 298

The compound of any one of embodiments 1-152 and 288, wherein L is selected from a bond, —CH₂— and —CH(CH₃)—.

### Embodiment 299

The compound of any one of embodiments 1-152 and 288, wherein L is selected from a bond and —CH₂—.

### Embodiment 300

The compound of any one of embodiments 1-152 and 288, wherein L is a bond.

### Embodiment 301

The compound of any one of embodiments 1-152 and 288, wherein L is —CH₂—.

### Embodiment 302

The compound of any one of embodiments 1-152 and 288, wherein L is —CH(CH₃)—.

### Embodiment 303

The compound of any one of embodiments 1-152 and 288-302, wherein R^{f} is a 4-10 membered heterocycle containing a nitrogen atom and one or two additional heteroatoms independently selected from oxygen and sulfur, including sulfur dioxide, wherein the 4-10 membered heterocycle is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 304

The compound of embodiment 303, wherein R^{f} is a 4-10 membered heterocycle containing a nitrogen atom and one or two additional heteroatoms independently selected from oxygen and sulfur, including sulfur dioxide, selected from the group consisting of a 4-8 member monocyclic heterocycle, a 6-10 member fused bicyclic heterocycle, a 6-10 member bridged heterocycle and a 6-10 member spiro heterocycle, each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 305

The compound of embodiment 303, wherein R^{f} is a 4-8 member monocyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 306

The compound of embodiment 303, wherein R^{f} is a 6-10 member fused bicyclic heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 307

The compound of embodiment 303, wherein R^{f} is a 6-10 member bridged heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 308

The compound of embodiment 303, wherein R^{f} is a 6-10 member spiro heterocycle substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 309

The compound of embodiment 303, wherein R^{f} is selected from azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 310

The compound of embodiment 303, wherein R^{f} is morpholine substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 311

The compound of any one of embodiments 303-310, wherein the attachment point for R^{f} is the nitrogen atom of the heterocycle.

### Embodiment 312

The compound of embodiment 311, wherein R^{f} is selected from the group consisting of:

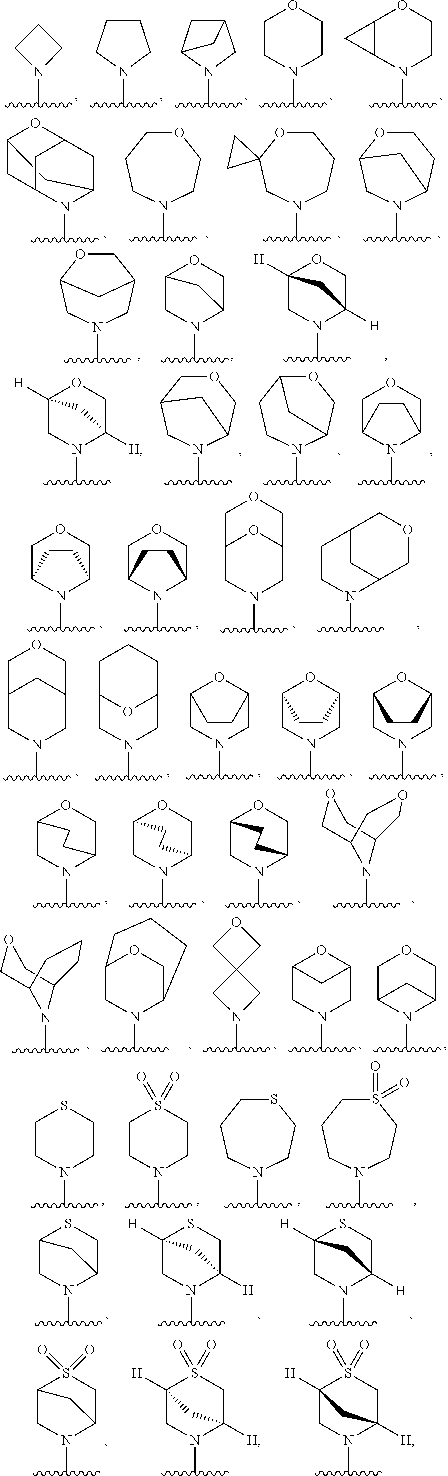

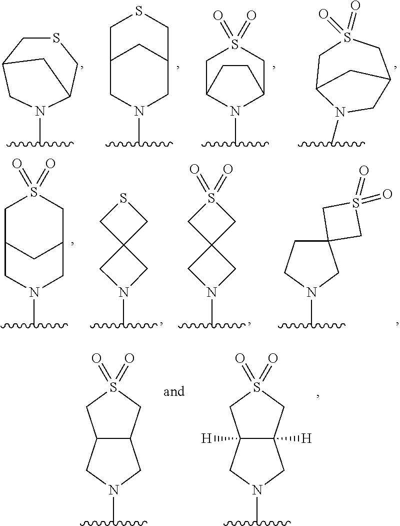

each substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

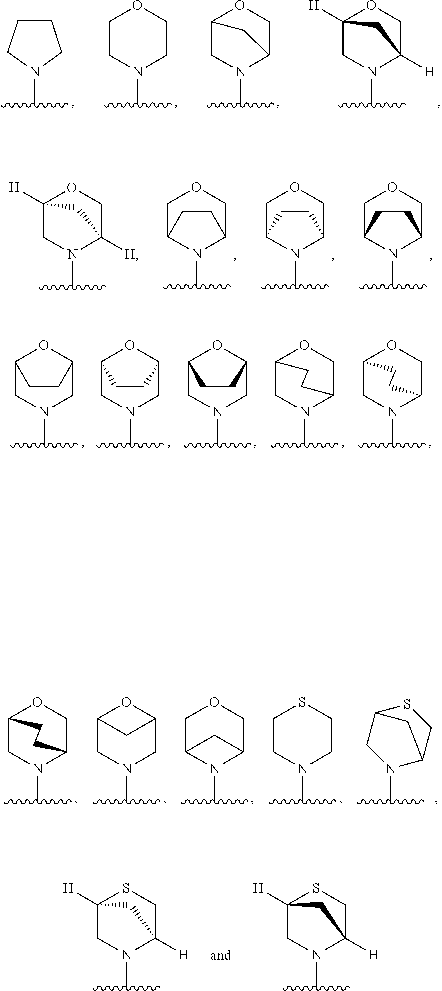

### Embodiment 313

The compound of embodiment 311, wherein R^{f} is selected from substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

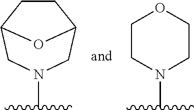 [SMILES: *N1CC2CCC(C1)O2 ; *N1CCOCC1]

### Embodiment 314

The compound of embodiment 311, wherein R^{f} is selected from substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

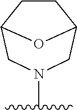 [SMILES: *N1CC2CCC(C1)O2]

### Embodiment 315

The compound of embodiment 311, wherein R^{f} is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

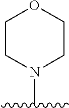 [SMILES: *N1CCOCC1]

### Embodiment 316

The compound of embodiment 311, wherein R^{f} is substituted with 0, 1, 2, 3 or 4 substituents independently selected from halo, hydroxy, C₁-C₄ alkyl, C₁-C₆ alkoxy, C₁-C₆ alkoxyalkoxy, C₁-C₄ haloalkyl and C₁-C₄ haloalkoxy.

### Embodiment 317

The compound of any one of embodiments 303-316, wherein the 4-10 membered heterocycle of R^{f} is substituted with 0, 1 or 2 substituents independently selected from —F, —OMe and -Me.

### Embodiment 318

The compound of any one of embodiments 303-316, wherein the 4-10 membered heterocycle of R^{f} is unsubstituted.

### Embodiment 319

The compound of embodiment 311, wherein R^{f} is selected from the group consisting of:

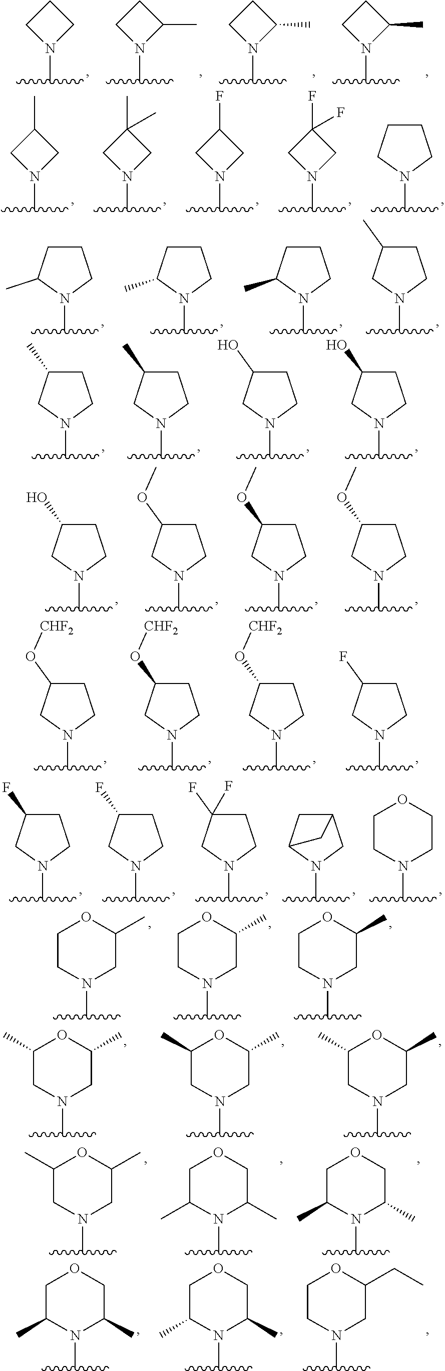

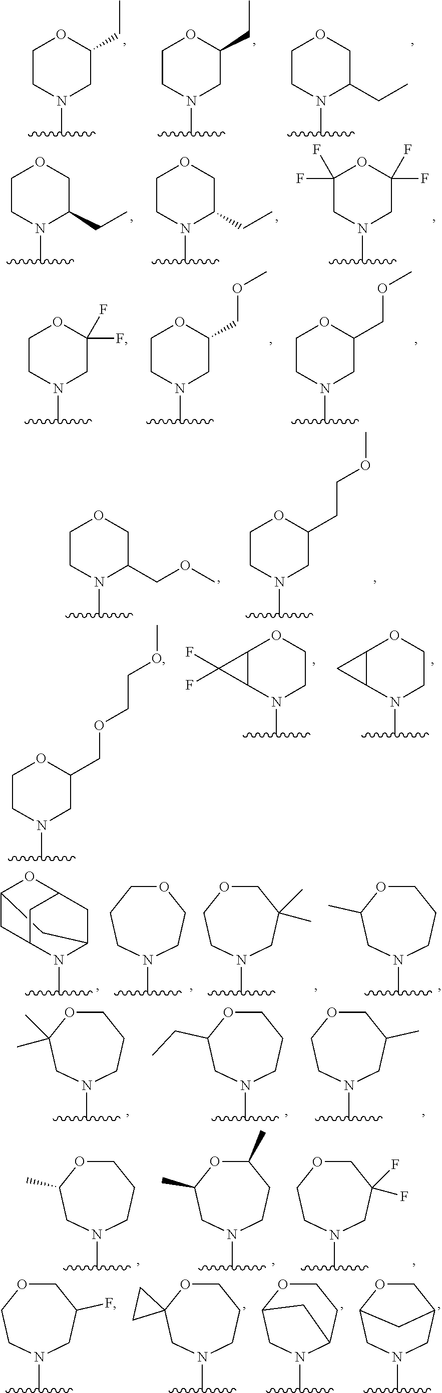

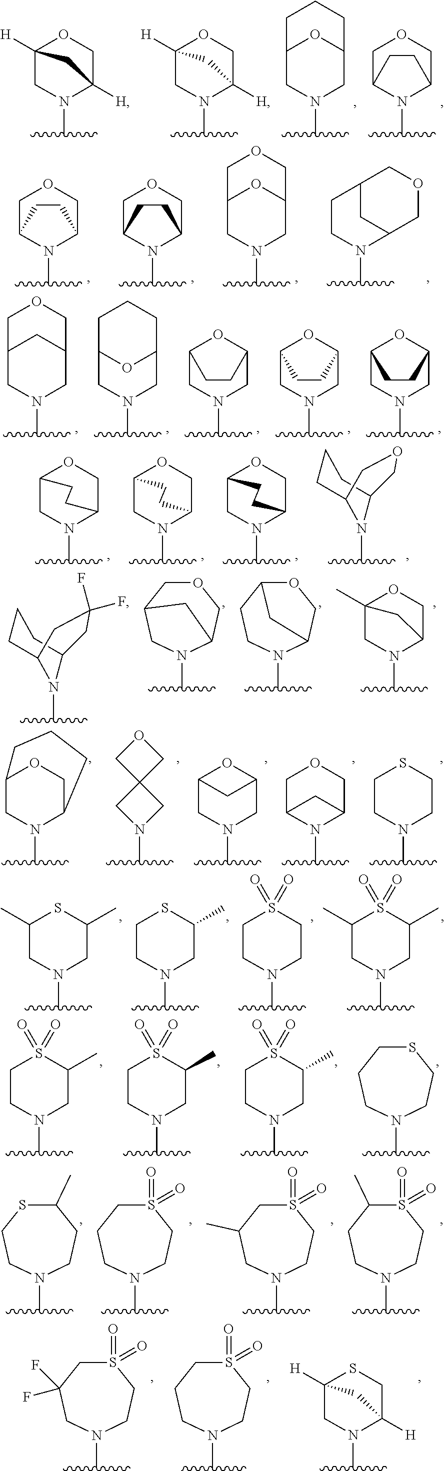

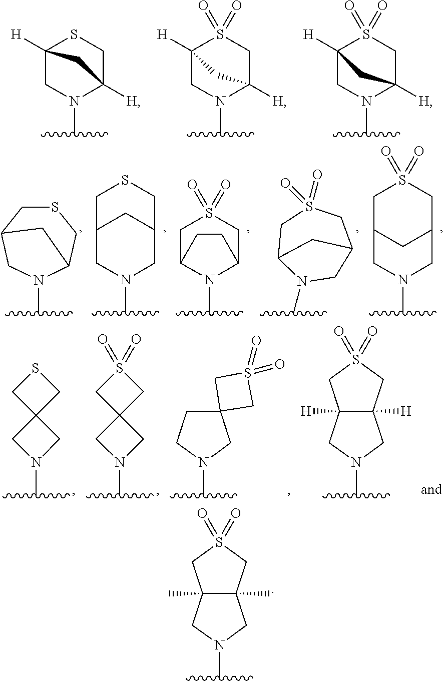

### Embodiment 320

The compound of embodiment 311, wherein R^{f} is unsubstituted

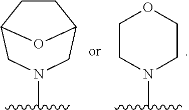

### Embodiment 321

The compound of embodiment 311, wherein R^{f} is unsubstituted

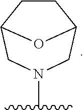 [SMILES: *N1CC2CCC(C1)O2]

### Embodiment 322

The compound of embodiment 311, wherein R^{f} is unsubstituted

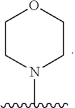 [SMILES: *N1CCOCC1]

### Embodiment 323

The compound of any one of embodiments 1-152 and 288, wherein L-R^{f} is selected from:

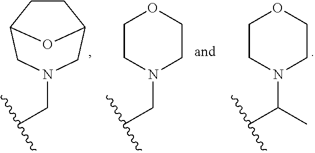

### Embodiment 324

The compound of any one of embodiments 1-152 and 288, wherein L-R^{f} is

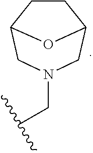 [SMILES: *CN1CC2CCC(C1)O2]

### Embodiment 325

The compound of any one of embodiments 1-152 and 288, wherein L-Rfis

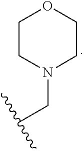 [SMILES: *CN1CCOCC1]

### Embodiment 326

The compound of any one of embodiments 1-152 and 288, wherein L-Rfis

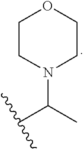 [SMILES: *C(C)N1CCOCC1]

### Embodiment 327

The compound of any one of embodiments 1-152, wherein R² is selected from the group consisting of:

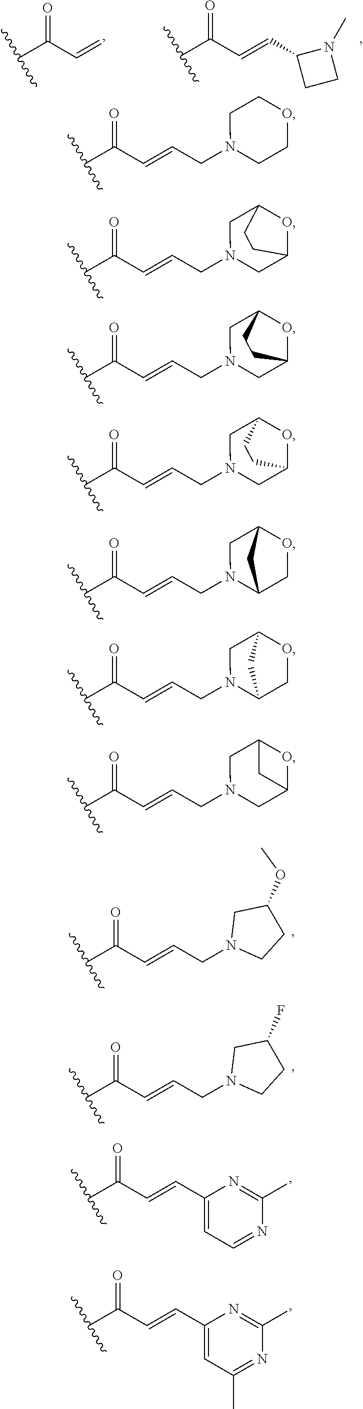

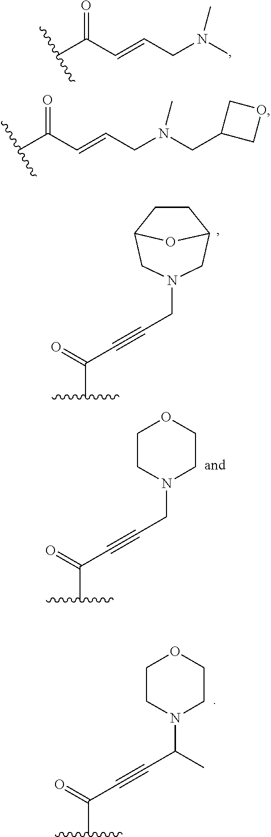

### Embodiment 328

The compound of any one of embodiments 1-152, wherein R² is

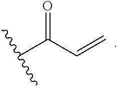 [SMILES: *C(=O)C=C]

### Embodiment 329

The compound of any one of embodiments 1-152, wherein R² is

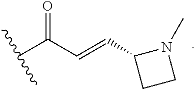 [SMILES: *C(=O)/C=C/[C@H]1CCN1C]

### Embodiment 330

The compound of any one of embodiments 1-152, wherein R² is

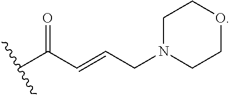 [SMILES: *C(=O)/C=C/CN1CCOCC1]

### Embodiment 331

The compound of any one of embodiments 1-152, wherein R² is

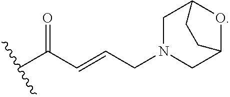 [SMILES: *C(=O)/C=C/CN1CC2CCC(C1)O2]

### Embodiment 332

The compound of any one of embodiments 1-152, wherein R² is

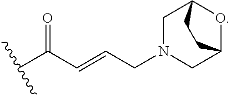 [SMILES: *C(=O)/C=C/CN1C[C@H]2CC[C@@H](C1)O2]

### Embodiment 333

The compound of any one of embodiments 1-152, wherein R² is

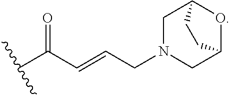 [SMILES: *C(=O)/C=C/CN1C[C@H]2CC[C@@H](C1)O2]

### Embodiment 334

The compound of any one of embodiments 1-152, wherein R² is

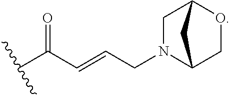 [SMILES: *C(=O)/C=C/CN1C[C@H]2C[C@@H]1CO2]

### Embodiment 335

The compound of any one of embodiments 1-152, wherein R² is

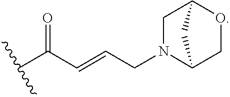 [SMILES: *C(=O)/C=C/CN1C[C@@H]2C[C@H]1CO2]

### Embodiment 336

The compound of any one of embodiments 1-152, wherein R² is

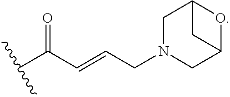 [SMILES: *C(=O)/C=C/CN1CC2CC(C1)O2]

### Embodiment 337

The compound of any one of embodiments 1-152, wherein R² is

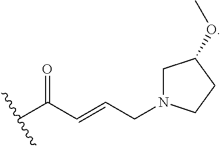 [SMILES: *C(=O)/C=C/CN1CC[C@@H](OC)C1]

### Embodiment 338

The compound of any one of embodiments 1-152, wherein R² is

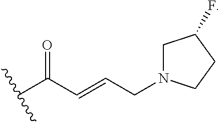 [SMILES: *C(=O)/C=C/CN1CC[C@@H](F)C1]

### Embodiment 339

The compound of any one of embodiments 1-152, wherein R² is

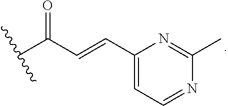 [SMILES: *C(=O)/C=C/c1ccnc(C)n1]

### Embodiment 340

The compound of any one of embodiments 1-152, wherein R² is

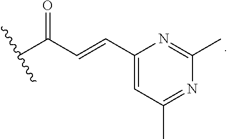 [SMILES: *C(=O)/C=C/c1cc(C)nc(C)n1]

### Embodiment 341

The compound of any one of embodiments 1-152, wherein R² is

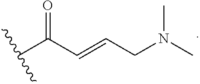 [SMILES: *C(=O)/C=C/CN(C)C]

### Embodiment 342

The compound of any one of embodiments 1-152, wherein R² is

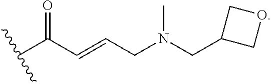 [SMILES: *C(=O)/C=C/CN(C)CC1COC1]

### Embodiment 343

The compound of any one of embodiments 1-152, wherein R² is

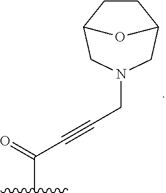 [SMILES: *C(=O)C#CCN1CC2CCC(C1)O2]

### Embodiment 344

The compound of any one of embodiments 1-152, wherein R² is

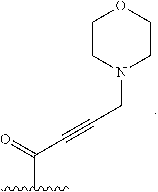 [SMILES: *C(=O)C#CCN1CCOCC1]

### Embodiment 345

The compound of any one of embodiments 1-152, wherein R² is

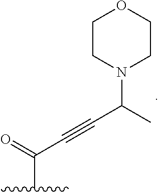 [SMILES: *C(=O)C#CC(C)N1CCOCC1]

### Embodiment 346

The compound of any one of embodiments 1-345, wherein R^{x} is selected from —H and -Me.

### Embodiment 347

The compound of any one of embodiments 1-345, wherein R^{x} is -Me.

### Embodiment 348

The compound of any one of embodiments 1-345, wherein R^{x} is —H.

### Embodiment 349

The compound of any one of embodiments 1, 4-6, 9-95, and 147-348, wherein R^{y} is selected from —Cl and —CF₃.

### Embodiment 350

The compound of any one of embodiments 1, 4-6, 9-95, and 147-349, wherein R^{y} is —Cl.

### Embodiment 351

The compound of any one of embodiments 1, 4-6, 9-95, and 147-349, wherein R^{y} is —CF₃.

### Embodiment 352A

The compound of any one of embodiments 1-351, wherein the compound is selected from the group consisting of:

[chemical structure]

[chemical structure]

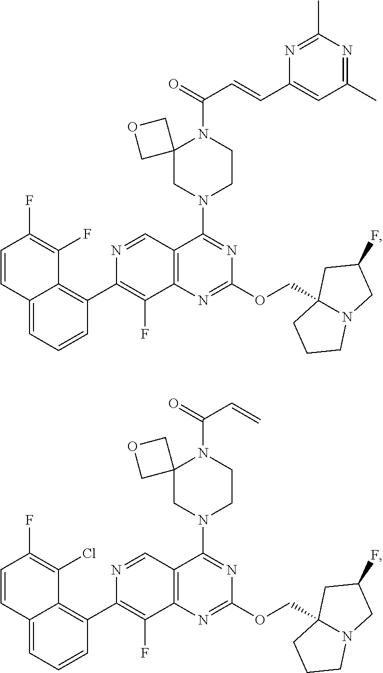 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)CC23COC3)nc(C)n1 ; C=CC(=O)N1CCN(c2nc(OC[C@@]34CCCN3C[C@H](F)C4)nc3c(F)c(-c4cccc5ccc(F)c(Cl)c45)ncc23)CC12COC2]

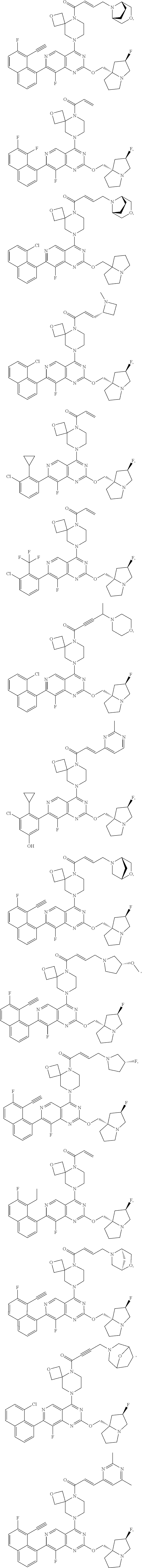

[chemical structure]

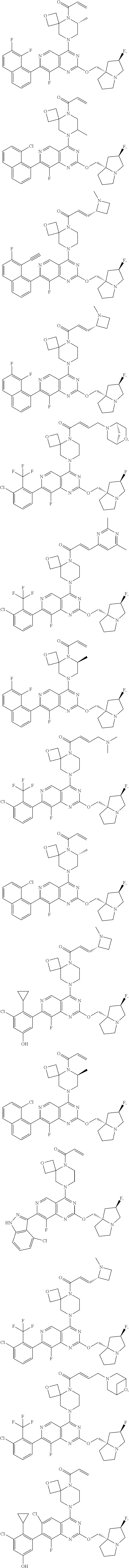

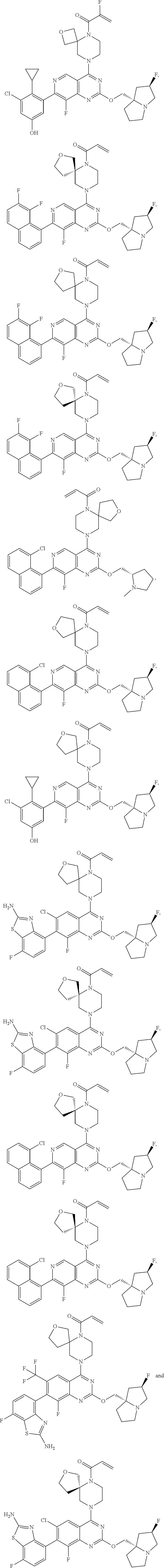

or a salt thereof; and/or an isotopologue thereof.

### Embodiment 352B

The compound of embodiment 1, or a salt thereof; and/or an isotopologue thereof, wherein the compound is

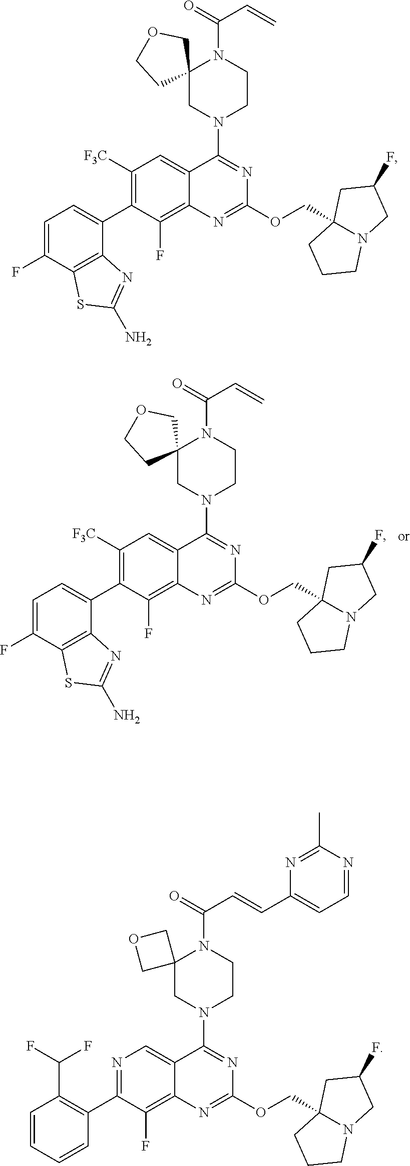 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@]12CCOC2 ; C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@@]12CCOC2 ; Cc1nccc(/C=C/C(=O)N2CCN(c3nc(OC[C@@]45CCCN4C[C@H](F)C5)nc4c(F)c(-c5ccccc5C(F)F)ncc34)CC23COC3)n1]

### Embodiment 353

The compound of any one of embodiments 1, 4-6, 9-95, and 147-351, wherein the compound is selected from the group consisting of:

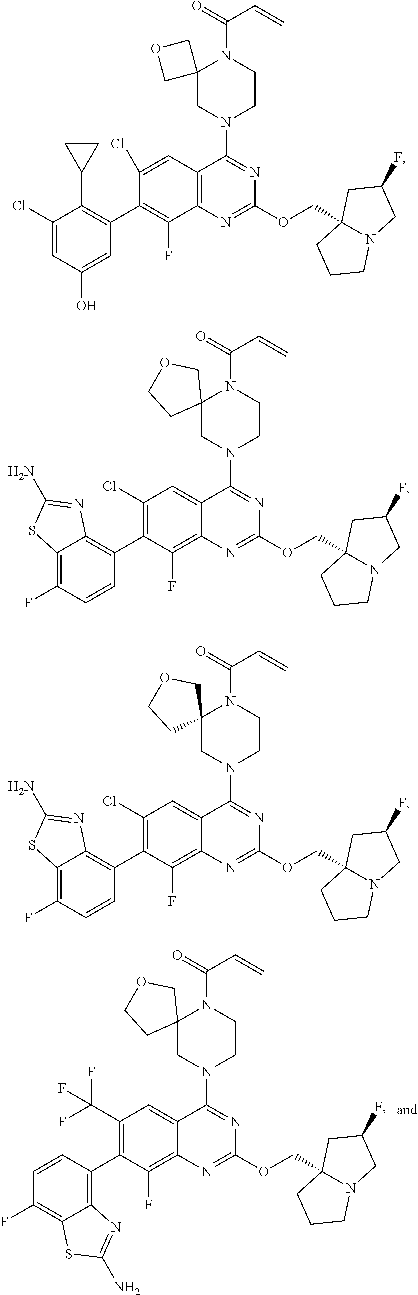 [SMILES: C=CC(=O)N1CCN(c2nc(OC[C@@]34CCCN3C[C@H](F)C4)nc3c(F)c(-c4cc(O)cc(Cl)c4C4CC4)c(Cl)cc23)CC12COC2 ; C=CC(=O)N1CCN(c2nc(OC[C@@]34CCCN3C[C@H](F)C4)nc3c(F)c(-c4ccc(F)c5sc(N)nc45)c(Cl)cc23)CC12CCOC2 ; C=CC(=O)N1CCN(c2nc(OC[C@@]34CCCN3C[C@H](F)C4)nc3c(F)c(-c4ccc(F)c5sc(N)nc45)c(Cl)cc23)C[C@]12CCOC2 ; C=CC(=O)N1CCN(c2nc(OC[C@@]34CCCN3C[C@H](F)C4)nc3c(F)c(-c4ccc(F)c5sc(N)nc45)c(C(F)(F)F)cc23)CC12CCOC2]

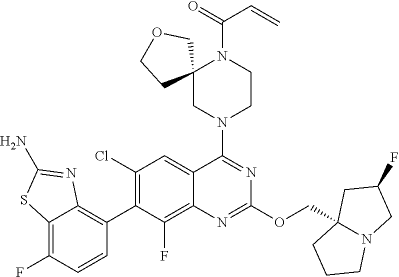 [SMILES: C=CC(=O)N1CCN(c2nc(OC[C@@]34CCCN3C[C@H](F)C4)nc3c(F)c(-c4ccc(F)c5sc(N)nc45)c(Cl)cc23)C[C@@]12CCOC2]

or a salt thereof; and/or an isotopologue thereof.

### Embodiment 354

The compound of any one of embodiments 1-353, wherein the compound is selected from the group consisting of:

[chemical structure]

[chemical structure]

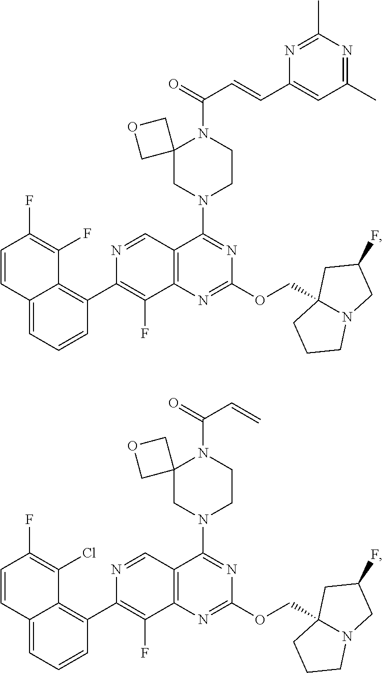 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)CC23COC3)nc(C)n1 ; C=CC(=O)N1CCN(c2nc(OC[C@@]34CCCN3C[C@H](F)C4)nc3c(F)c(-c4cccc5ccc(F)c(Cl)c45)ncc23)CC12COC2]

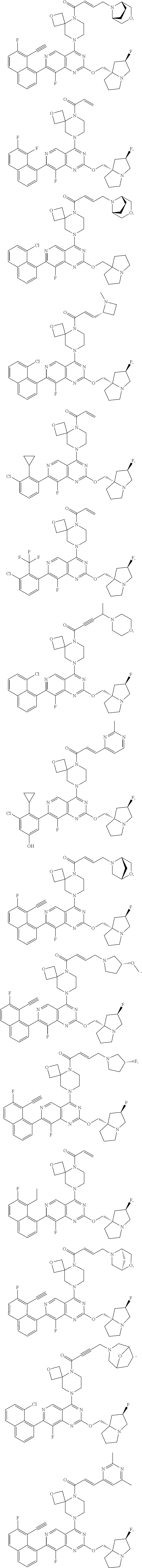

[chemical structure]

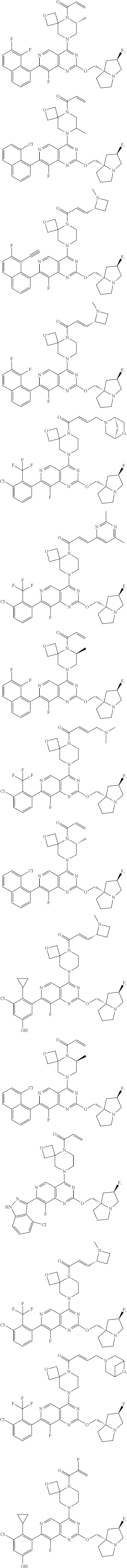

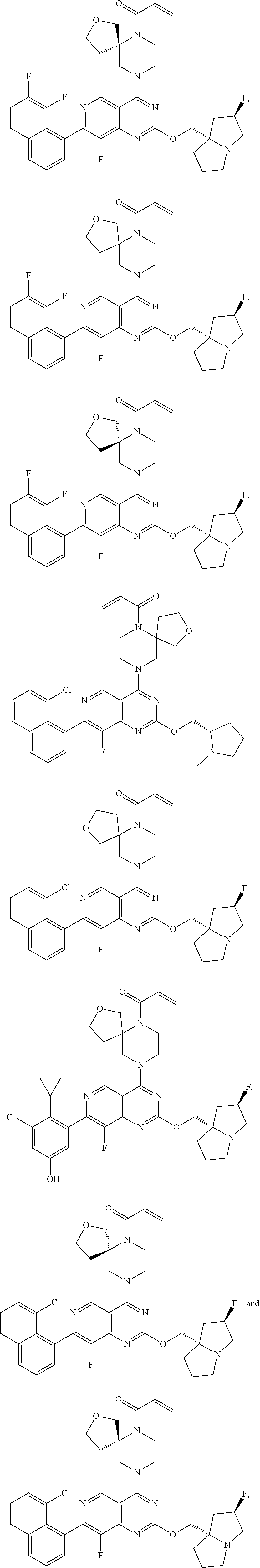

or a salt thereof; and/or an isotopologue thereof.

### Embodiment 355

The compound of any one of embodiments 1-354, wherein the compound is not a salt.

### Embodiment 356

The compound of any one of embodiments 1-354, wherein the compound is a salt.

### Embodiment 357

The compound of embodiment 356, wherein the salt is a formate salt.

### Embodiment 358

The compound of embodiment 356, wherein the salt is a trifluoroacetate salt.

### Embodiment 359

The compound of embodiment 356, wherein the salt is a pharmaceutically acceptable salt.

### Embodiment 360

A pharmaceutical formulation comprising the compound of any one of embodiments 1-359, wherein when the compound is a salt, the salt is a pharmaceutically acceptable salt, and a pharmaceutically acceptable carrier.

### Embodiment 361

A method of treating or suppressing cancer comprising: administering a therapeutically effective amount of a compound of any one of embodiments 1-359, wherein when the compound is a salt, the salt is a pharmaceutically acceptable salt, or a pharmaceutical formulation according to embodiment 360, to a subject in need thereof.

### Embodiment 362

The method of embodiment 361, wherein the cancer is selected from the group consisting of: lung, colorectal, pancreatic, bile duct, thyroid, gall bladder, uterine, mesothelioma, cervical, and bladder cancers.

### Embodiment 363

The method of embodiment 361, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cellmyeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cellmalignancies, pediatric neuroblastoma, and melanoma.

### Embodiment 364

The method of any one of embodiments 361 to 363, wherein the cancer is a KRAS G12C mediated cancer.

### Embodiment 365

The method of any one of embodiments 361 to 363, wherein the subject has been diagnosed as having a KRAS G12C mediated cancer.

### Embodiment 366

The method of any one of embodiments 361 to 363, wherein the method further comprises administering to the subject a therapeutically effective amount of an additional chemotherapeutic agent.

### General Synthetic Methods

Compounds 1-57 in Table 1 of the instant disclosure were prepared according to methods described in the Examples section or variations thereof that would be within the knowledge of one of skill in the art. The starting materials and reagents used in preparing these compounds are either available from commercial suppliers such as MilliporeSigma., Bachem., etc. or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

### EXAMPLES

The following preparations of compounds of Formula (I), Formula (II), Formula (III) and Formula (IV) and pharmaceutically acceptable salts thereof are given to enable those skilled in the art to more clearly understand and to practice the present disclosure. They should not be considered as limiting the scope of the disclosure, but merely as being illustrative and representative thereof.

The following abbreviations are used in this section:

| | |
|---|---|
| Boc | tert-butyloxycarbonyl |
| PMB | p-methoxybenzyl |
| ACN | acetonitrile |
| TFA | trifluoroacetic acid |
| FA | formic acid |
| mmol | millimoles |
| g | grams |
| mg | milligrams |
| h | hours |
| mL | milliliters |
| NMR | Nuclear magnetic resonance |
| MHz | megahertz |
| LCMS | Liquid chromatography-mass |
| | spectrometry |
| Rt | Retention time |
| m/z | Mass/charge |
| min | minutes |
| um | micrometers |
| mm | millimeters |
| umol | micromoles |

All reagents were obtained from commercial suppliers and used without further purification unless otherwise stated.

### Synthetic Examples

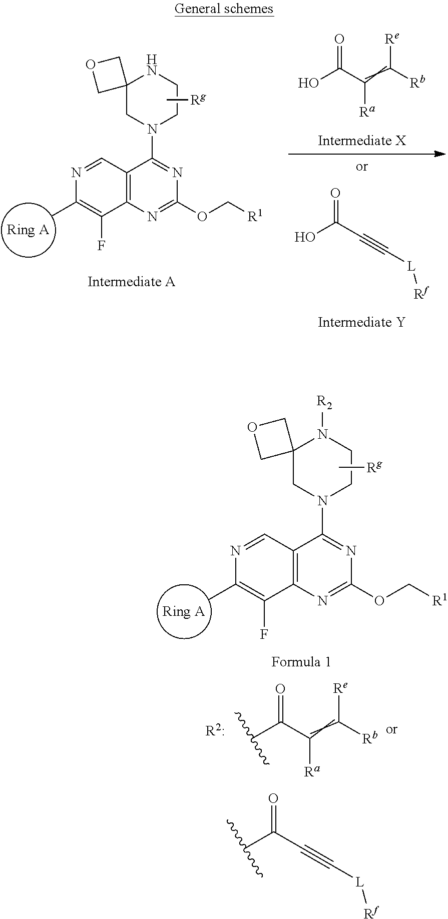

### General Scheme I General Procedure for the Synthesis of Compounds of Formula (I)

To a solution of NH piperazine intermediate A (1 eq) in dichloromethane was added substituted acrylic acid Intermediate X (2 eq) or substituted alkynyl acid Intermediate Y (2 eq), N,N-diisopropylethylamine (3 eq) and 2,4,6-tripropyl-1,3,5,2,4,6-trioxatriphosphinane 2,4,6-trioxide (1.5 eq, 50% purity in ethyl acetate) at 0° C. and the mixture was stirred at 25° C. for 1 h to provide crude product of Formula (I).

### Exemplary Compounds

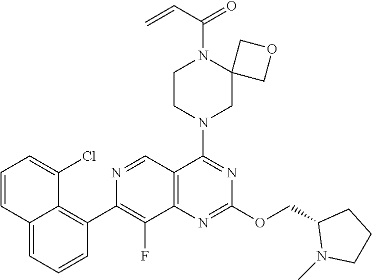 [SMILES: C=CC(=O)N1CCN(c2nc(OC[C@@H]3CCCN3C)nc3c(F)c(-c4cccc5cccc(Cl)c45)ncc23)CC12COC2]

### Example 1 (Method 1-Master): (S)-1-(8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonan-5-yl) prop-2-en-1-one

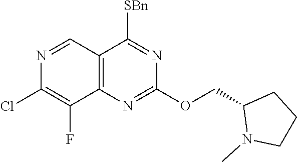

### Step 1: (S)-4-(benzylthio)-7-chloro-8-fluoro-2-((1-methylpyrrolidin-2-yl)methoxy)pyrido[4,3-d]pyrimidine

To a solution of 4-benzylsulfanyl-2,7-dichloro-8-fluoro-pyrido[4,3-d]pyrimidine (12 g, 35.27 mmol) in dioxane (50 mL) was added N,N-diisopropylethylamine (13.68 g, 105.82 mmol) and (S)-(1-methylpyrrolidin-2-yl) methanol (20.31 g, 176.37 mmol). The mixture was stirred at 80° C. for 1 h. The reaction mixture was diluted with water (100 mL) and extracted with ethyl acetate (2×150 mL). The combined organic layers were dried over sodium sulphate and concentrated in vacuo. The resulting residue was purified by column chromatography (silica gel, 100-200 mesh, 70% ethyl acetate in hexane) affording(S)-4-(benzylthio)-7-chloro-8-fluoro-2-((1-methylpyrrolidin-2-yl)methoxy)pyrido[4,3-d]pyrimidine (6 g, 40.61%) as a white solid. LCMS Rt=0.772 min, m/z=419.1 [M+H]⁺.

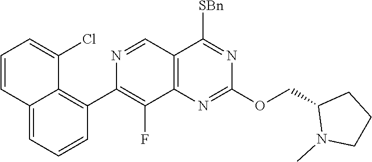

### Step 2: (S)-4-(benzylthio)-7-(8-chloronaphthalen-1-yl)-8-fluoro-2-((1-methylpyrrolidin-2-yl)methoxy)pyrido[4,3-d]pyrimidine

A mixture of(S)-4-(benzylthio)-7-chloro-8-fluoro-2-((1-methylpyrrolidin-2-yl)methoxy)pyrido[4,3-d]pyrimidine (4.7 g, 11.22 mmol), (8-chloro-1-naphthyl) boronic acid (4.63 g, 22.44 mmol), [1,1′-Bis(di-tert-butylphosphino) ferrocene]dichloropalladium (II)(731.22 mg, 1.12 mmol) and potassium phosphate (7.14 g, 33.66 mmol) in tetrahydrofuran (90 mL) and water (30 mL) was degassed and purged with nitrogen 3 times, and the mixture was stirred at 80° C. for 12 h under a nitrogen atmosphere. The reaction mixture was diluted with water (30 mL) and extracted with ethyl acetate (2×100 mL). The combined organic layers were dried over sodium sulphate and concentrated in vacuo. The resulting residue was purified by column chromatography (silica gel, 100-200 mesh, 80% ethyl acetate in hexane) affording(S)-4-(benzylthio)-7-(8-chloronaphthalen-1-yl)-8-fluoro-2-((1-methylpyrrolidin-2-yl)methoxy)pyrido[4,3-d]pyrimidine (3.3 g, 53.96%) as a brown oil. LCMS Rt=0.628 min, m/z=545.2 [M+H]⁺.

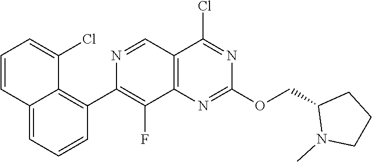 [SMILES: CN1CCC[C@H]1COc1nc(Cl)c2cnc(-c3cccc4cccc(Cl)c34)c(F)c2n1]

### Step 3: (S)-4-chloro-7-(8-chloronaphthalen-1-yl)-8-fluoro-2-((1-methylpyrrolidin-2-yl)methoxy)pyrido[4,3-d]pyrimidine

To a solution of(S)-4-(benzylthio)-7-(8-chloronaphthalen-1-yl)-8-fluoro-2-((1-methylpyrrolidin-2-yl)methoxy)pyrido[4,3-d]pyrimidine (400 mg, 733.85 μmol) in acetonitrile (5 mL) was added acetic acid (4.41 mg, 73.39 μmol), water (1.32 mg, 73.39 μmol) and 1,3-dichloro-5,5-dimethyl-imidazolidine-2,4-dione (361.46 mg, 1.83 mmol). The mixture was stirred at 0° C. for 1 h. The reaction mixture was concentrated in vacuo affording(S)-4-chloro-7-(8-chloronaphthalen-1-yl)-8-fluoro-2-((1-methylpyrrolidin-2-yl)methoxy)pyrido[4,3-d]pyrimidine (400 mg, crude) as a brown oil, which was used in the next step without any further purification. LCMS Rt=0.554 min, m/z=457.1 [M+H]⁺.

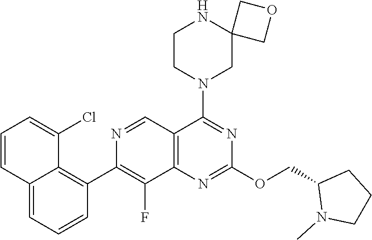 [SMILES: CN1CCC[C@H]1COc1nc(N2CCNC3(COC3)C2)c2cnc(-c3cccc4cccc(Cl)c34)c(F)c2n1]

### Step 4: (S)-8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonane

To a solution of 2-oxa-5,8-diazaspiro[3.5]nonane (164.89 mg, 819.98 μmol, 2 hydrochloric acid salt) and N,N-diisopropylethylamine (282.61 mg, 2.19 mmol) in tetrahydrofuran (3 mL) was added(S)-4-chloro-7-(8-chloronaphthalen-1-yl)-8-fluoro-2-((1-methylpyrrolidin-2-yl)methoxy)pyrido[4,3-d]pyrimidine (250 mg, 546.66 μmol) at 0° C., and the mixture was stirred at 25° C. for 1 h. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 25%-55%, 8 min) affording(S)-8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonane (60 mg, 19.99%) as a white solid. LCMS Rt=0.776 min, m/z=549.2 [M+H]⁺.

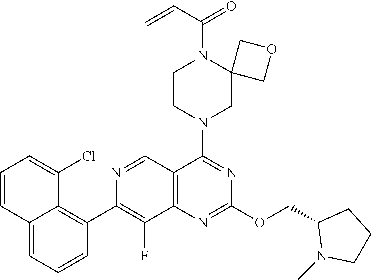 [SMILES: C=CC(=O)N1CCN(c2nc(OC[C@@H]3CCCN3C)nc3c(F)c(-c4cccc5cccc(Cl)c45)ncc23)CC12COC2]

### Step 5: (S)-1-(8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonan-5-yl) prop-2-en-1-one

To a solution of(S)-8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonane (50 mg, 91.07 μmol) in dichloromethane (1 mL) was added 2,6-dimethylpyridine (29.27 mg, 273.21 μmol) and prop-2-enoyl chloride (9.07 mg, 100.18 μmol) under a nitrogen atmosphere at −78° C. The mixture was stirred at −78° C. for 0.5 h. The reaction mixture was concentrated in vacuo and purified by reverse phase HPLC (column: Waters Xbridge BEH C18 100*30 mm*10 μm; mobile phase: [water (NH₄HCO₃)-acetonitrile]; B %: 30%-60%, 10 min) affording(S)-1-(8-(7-(8-chloronaphthalen-1-yl)-8-fluoro-2-((1-methylpyrrolidin-2-yl)methoxy)pyrido[4,3-d]pyrimidin-4-yl)-2-oxa-5,8-diazaspiro[3.5]nonan-5-yl) prop-2-en-1-one (8.17 mg, 14.88%) as a yellow solid: ¹H NMR (400 MHz, Acetonitrile-d3) δ 9.12 (s, 1H), 8.16 (dd, J=1.2, 8.1 Hz, 1H), 8.05 (dd, J=1.0, 8.2 Hz, 1H), 7.75-7.69 (m, 1H), 7.68-7.62 (m, 2H), 7.57-7.52 (m, 1H), 6.68 (dd, J=10.6, 16.8 Hz, 1H), 6.30 (dd, J=2.0, 16.7 Hz, 1H), 5.80 (dd, J=2.0, 10.5 Hz, 1H), 4.93-4.86 (m, 2H), 4.61-4.56 (m, 1H), 4.55-4.49 (m, 2H), 4.49-4.45 (m, 2H), 4.43 (d, J=12.7 Hz, 1H), 4.39-4.32 (m, 1H), 3.96 (t, J=5.1 Hz, 2H), 3.74-3.58 (m, 2H), 3.08-3.01 (m, 1H), 2.72-2.62 (m, 1H), 2.45 (d, J=1.1 Hz, 3H), 2.09-2.00 (m, 1H), 1.85-1.69 (m, 3H).

LCMS (5% to 95% acetonitrile in water+0.03% ammonium bicarbonate over 6 min); retention time 2.645 min, ESI+ found [M+H]=603.2.

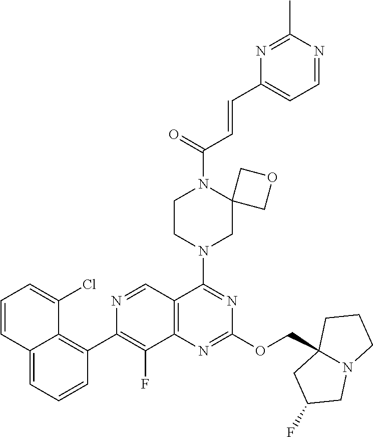 [SMILES: Cc1nccc(/C=C/C(=O)N2CCN(c3nc(OC[C@@]45CCCN4C[C@H](F)C5)nc4c(F)c(-c5cccc6cccc(Cl)c56)ncc34)CC23COC3)n1]

### Example 2 (Method 2-Master): (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

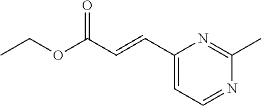

### Step 1: ethyl(E)-3-(2-methylpyrimidin-4-yl) acrylate

A mixture of 4-chloro-2-methyl-pyrimidine (4.9 g, 38.11 mmol), ethyl 3-(4,4,5,5-tetramethyl-1,3,2-dioxaborolan-2-yl) propanoate (13.04 g, 57.17 mmol), chloro(2-dicyclohexylphosphino-2′,4′,6′-tri-i-propyl-1,1′-biphenyl)(2′-amino-1,1′-biphenyl-2-yl) palladium (II)(3.00 g, 3.81 mmol) and potassium phosphate (24.27 g, 114.34 mmol) in dioxane (45 mL) and water (15 mL) was degassed and purged with nitrogen 3 times. The mixture was stirred at 100° C. for 2 h. Water (100 mL) was added, and the mixture was extracted with ethyl acetate (2×30 mL). The combined organic layers were dried over sodium sulphate and concentrated in vacuo. The residue was purified by flash column chromatography (silica gel, 100-200 mesh, 0-100% ethyl acetate in petroleum ether) affording ethyl(E)-3-(2-methylpyrimidin-4-yl) acrylate (11 g, crude) as yellow oil, which was used in the next step without further purification. LCMS Rt=0.522 min, m/z=193.1 [M+H]⁺.

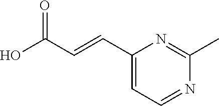

### Step 2: (E)-3-(2-methylpyrimidin-4-yl) acrylic acid

To a solution of ethyl(E)-3-(2-methylpyrimidin-4-yl) prop-2-enoate (10 g, 52.03 mmol) in THF (100 mL) was added lithium hydroxide monohydrate (2 M, 52.03 mL), and the mixture was stirred at 30° C. for 12 h. The mixture was extracted with ethyl acetate (2×80 mL), and the aqueous phase was adjusted to pH=2 with 1N hydrochloric acid at 0° C. to generate a precipitate. The precipitate was collected and dried in vacuo to afford (E)-3-(2-methylpyrimidin-4-yl) acrylic acid (3.76 g, 44.03%) as a white solid: ¹H NMR (400 MHz, Dimethyl sulfoxide-d6) δ 8.79-8.73 (m, 1H), 7.62-7.56 (m, 1H), 7.50-7.41 (m, 1H), 7.06-6.96 (m, 1H), 2.63 (s, 3H).

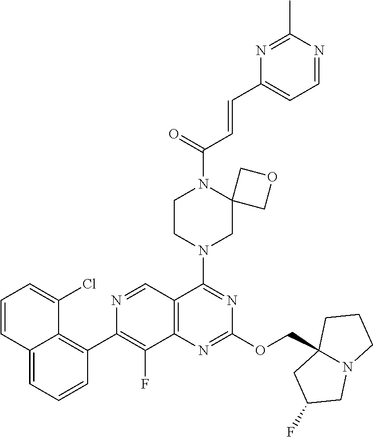 [SMILES: Cc1nccc(/C=C/C(=O)N2CCN(c3nc(OC[C@@]45CCCN4C[C@H](F)C5)nc4c(F)c(-c5cccc6cccc(Cl)c56)ncc34)CC23COC3)n1]

### Step 3: (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

To a solution of 8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (0.1 g, 135.28 μmol) and (E)-3-(2-methylpyrimidin-4-yl) prop-2-enoic acid (44.42 mg, 270.56 μmol) in N,N-dimethylformaldehyde (1 mL) was added diisopropylethylamine (87.42 mg, 676.40 μmol) and 2-chloro-1,3-dimethyl-4,5-dihydroimidazol-1-ium chloride (45.74 mg, 270.56 μmol) at 25° C. The mixture was stirred at 25° C. for 0.5 h. The reaction mixture was concentrated in vacuo and purified by reverse phase HPLC (column: Waters Xbridge Prep OBD C18 150*40 mm*10 μm; mobile phase: [water (NH₄HCO₃)-acetonitrile) affording (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (15.36 mg, 11.48%) as a yellow amorphous solid: ¹H NMR (400 MHz, Dimethylsulfoxide-d6) δ 9.17 (s, 1H), 8.79 (d, J=5.1 Hz, 1H), 8.21 (d, J=8.1 Hz, 1H), 8.11 (d, J=8.0 Hz, 1H), 7.75-7.62 (m, 5H), 7.60-7.54 (m, 1H), 7.46 (d, J=15.3 Hz, 1H), 5.40-5.16 (m, 1H), 4.90 (br t, J=5.7 Hz, 2H), 4.53-4.39 (m, 4H), 4.20-4.08 (m, 2H), 4.03-3.81 (m, 4H), 3.09 (br d, J=9.4 Hz, 2H), 3.02 (br s, 1H), 2.88-2.79 (m, 1H), 2.65 (s, 3H), 2.17-1.99 (m, 3H), 1.88-1.74 (m, 3H).

LCMS (5% to 95% acetonitrile in water+0.03% ammonium bicarbonate over 6 min); retention time 2.129 min, ESI+ found [M+H]=739.3.

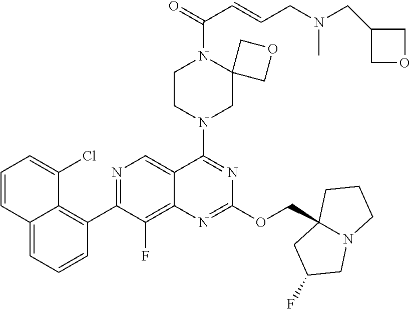 [SMILES: CN(C/C=C/C(=O)N1CCN(c2nc(OC[C@@]34CCCN3C[C@H](F)C4)nc3c(F)c(-c4cccc5cccc(Cl)c45)ncc23)CC12COC2)CC1COC1]

### Example 3 (Method 3-Master): (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(methyl(oxetan-3-ylmethyl)amino)but-2-en-1-one

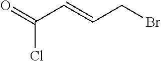

### Step 1: (E)-4-bromobut-2-enoyl chloride

To a solution of (E)-4-bromobut-2-enoic acid (100 mg, 606.12 μmol) in dichloromethane (50 mL) was added N,N-dimethylformamide (4.43 mg, 60.61 μmol) and oxalyl dichloride (92.32 mg, 727.34 μmol) at 0° C. The mixture was stirred at 20° C. for 1 h. The reaction mixture was concentrated in vacuo affording (E)-4-bromobut-2-enoyl chloride (230 mg, crude) as a yellow oil, which was used in the next step without further purification.

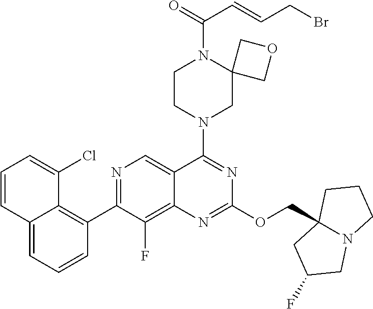 [SMILES: O=C(/C=C/CBr)N1CCN(c2nc(OC[C@@]34CCCN3C[C@H](F)C4)nc3c(F)c(-c4cccc5cccc(Cl)c45)ncc23)CC12COC2]

### Step 2: (E)-4-bromo-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

To a solution of (E)-4-bromobut-2-enoyl chloride (92.79 mg, 505.85 μmol) and sodium bicarbonate (56.66 mg, 674.46 μmol) in THF (1 mL) and water (1 mL) was added 8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (100 mg, 168.62 μmol) at 0° C. The mixture was stirred at 25° C. for 1 h. The reaction mixture was concentrated in vacuo affording (E)-4-bromo-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (130 mg, crude) as a yellow solution, which was used in the next step without further purification. LCMS Rt=1.477 min, m/z=741.2 [M+H]⁺.

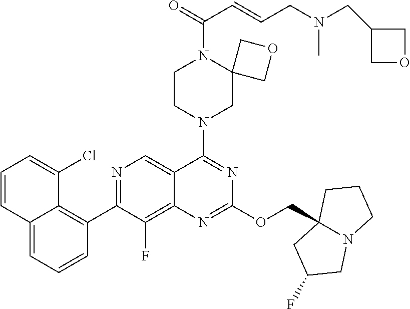 [SMILES: CN(C/C=C/C(=O)N1CCN(c2nc(OC[C@@]34CCCN3C[C@H](F)C4)nc3c(F)c(-c4cccc5cccc(Cl)c45)ncc23)CC12COC2)CC1COC1]

### Step 3: (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(methyl(oxetan-3-ylmethyl)amino)but-2-en-1-one

To a solution of oxetan-3-ylmethanamine (164.01 mg, 1.62 mmol) and diisopropylethylamine (251.48 mg, 1.95 mmol) in THF (1 mL) was added (E)-4-bromo-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (120 mg, 162.15 μmol) at 0° C. The mixture was stirred at 50° C. for 12 h. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 35%-65%, 8 min) affording (E)-1-(8-(7-(8-chloronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl)-4-(methyl(oxetan-3-ylmethyl)amino)but-2-en-1-one (14 mg, 10.86%) as a yellow solid: 1H NMR (400 MHz, Acetonitrile-d3) δ 9.09 (s, 1H), 8.12 (dd, J=1.1, 8.1 Hz, 1H), 8.02 (dd, J=0.9, 8.2 Hz, 1H), 7.72-7.66 (m, 1H), 7.64-7.59 (m, 2H), 7.55-7.48 (m, 1H), 6.77 (td, J=6.2, 15.2 Hz, 1H), 6.45 (br d, J=15.3 Hz, 1H), 5.35-5.16 (m, 1H), 4.84 (t, J=6.7 Hz, 2H), 4.70-4.65 (m, 2H), 4.52 (dd, J=6.6, 13.0 Hz, 1H), 4.43 (br t, J=5.6 Hz, 2H), 4.38 (dd, J=5.8, 13.1 Hz, 1H), 4.29 (t, J=6.0 Hz, 2H), 4.25-4.20 (m, 1H), 4.17-4.11 (m, 1H), 3.92 (t, J=5.0 Hz, 2H), 3.70-3.56 (m, 2H), 3.16-3.11 (m, 4H), 3.06 (s, 1H), 2.93-2.84 (m, 1H), 2.68 (d, J=7.5 Hz, 2H), 2.18 (br s, 3H), 2.10 (br d, J=2.6 Hz, 1H), 2.09-2.01 (m, 2H), 1.92-1.73 (m, 4H).

LCMS (5% to 95% acetonitrile in water+0.03% ammonium bicarbonate over 6 min); retention time 1.910 min, ESI+ found [M+H]=760.3.

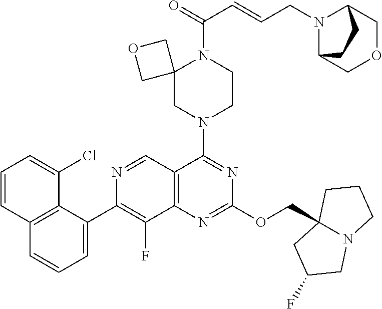

### Example 4 (Method 6-Master): (E)-4-((1R,5S)-3-oxa-8-azabicyclo[3.2.1]octan-8-yl)-1-(8-(7-(8-chloronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl) but-2-en-1-one

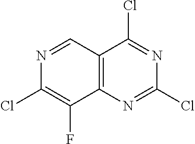 [SMILES: Fc1c(Cl)ncc2c(Cl)nc(Cl)nc12]

### Step 1: 2,4,7-trichloro-8-fluoropyrido[4,3-d]pyrimidine

To a solution of 7-chloro-8-fluoro-pyrido[4,3-d]pyrimidine-2,4-diol (5 g, 23.19 mmol) in toluene (150 mL) was added diisopropylethylamine (8.99 g, 69.58 mmol) and phosphorus oxychloride (17.78 g, 115.97 mmol) at 0° C. The mixture was stirred at 100° C. for 2 h. The reaction mixture was concentrated to dryness in vacuo affording 2,4,7-trichloro-8-fluoropyrido[4,3-d]pyrimidine (11 g, crude) as a yellow oil, which was used in the next step without further purification. LCMS Rt=0.444 min, m/z=251.9 [M+H]⁺.

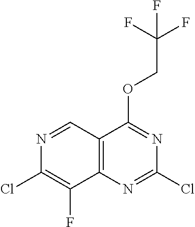 [SMILES: Fc1c(Cl)ncc2c(OCC(F)(F)F)nc(Cl)nc12]

### Step 2: 2,7-dichloro-8-fluoro-4-(2,2,2-trifluoroethoxy)pyrido[4,3-d]pyrimidine

To a solution of 2,2,2-trifluoroethanol (1.87 g, 18.72 mmol, 1.35 mL) in tetrahydrofuran (60 mL) was added sodium tert-butoxide (2.20 g, 22.87 mmol) at −78° C. The mixture was stirred at −78° C. for 0.5 h under a nitrogen atmosphere. The mixture was added to 2,4,7-trichloro-8-fluoropyrido[4,3-d]pyrimidine (5.25 g, 20.80 mmol) and stirred at −78° C. for 0.5 h under a nitrogen atmosphere. The reaction mixture was quenched with saturated ammonium chloride (50 mL) and extracted with ethyl acetate (3×20 mL). The combined organic layers were dried over sodium sulphate and concentrated in vacuo. The resulting residue was purified by column chromatography (silica gel, 100-200 mesh, 0-100% ethyl acetate in petroleum ether) affording 2,7-dichloro-8-fluoro-4-(2,2,2-trifluoroethoxy)pyrido[4,3-d]pyrimidine (2.6 g, 26.71%) as a yellow solid. LCMS Rt=0.500 min, m/z=317.0 [M+H]⁺.

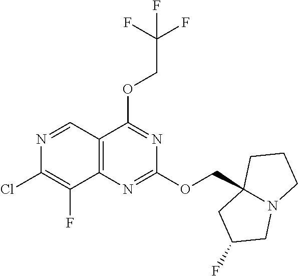 [SMILES: Fc1c(Cl)ncc2c(OCC(F)(F)F)nc(OC[C@@]34CCCN3C[C@H](F)C4)nc12]

### Step 3: 7-chloro-8-fluoro-2-(((2R,7aS)-2-fluorotetrahydro-1H-pyrrolizin-7a(5H)-yl) methoxy)-4-(2,2,2-trifluoroethoxy)pyrido[4,3-d]pyrimidine

A mixture of 2,7-dichloro-8-fluoro-4-(2,2,2-trifluoroethoxy)pyrido[4,3-d]pyrimidine (2 g, 6.33 mmol), ((2R,7aS)-2-fluorotetrahydro-1H-pyrrolizin-7a(5H)-yl) methanol (1.21 g, 7.59 mmol), diisopropylethylamine (2.45 g, 18.98 mmol) and 4A MS (1 g, 632.83 μmol) in dioxane (20 mL) was degassed and purged with nitrogen 3 times, and the mixture was stirred at 50° C. for 2 h under a nitrogen atmosphere. The reaction mixture was concentrated in vacuo and purified by column chromatography (silica gel, 100-200 mesh, 0-100% ethyl acetate in petroleum ether) affording 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (2 g, 72.03%) as an orange solid. LCMS Rt=0.588 min, m/z=439.1 [M+H]⁺.

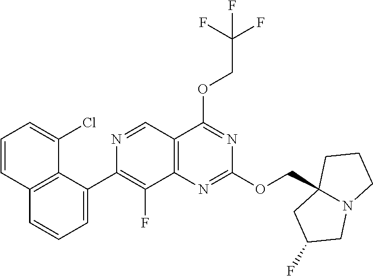 [SMILES: Fc1c(-c2cccc3cccc(Cl)c23)ncc2c(OCC(F)(F)F)nc(OC[C@@]34CCCN3C[C@H](F)C4)nc12]

### Step 4: 7-(8-chloronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

A mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (1 g, 2.28 mmol), (8-chloro-1-naphthyl) boronic acid (611.61 mg, 2.96 mmol), potassium phosphate (1.45 g, 6.84 mmol) and [2-(Amino-KN) [1,1-biphenyl]-2-yl-KC]chloro[dicyclohexyl[2,4,6-tris(1-methylethyl) [1,1-biphenyl]-2-yl]phosphine]palladium (179.32 mg, 227.91 μmol) in dioxane (20 mL) and water (7 mL) was degassed and purged with nitrogen 3 times, and the mixture was stirred at 80° C. for 2 h under a nitrogen atmosphere. The reaction mixture was diluted with water (10 mL) and extracted with dimethyl tetrahydrofuran (3×10 mL). The combined organic layers were dried over sodium sulphate and concentrated in vacuo. The resulting residue was purified by column chromatography (silica gel, 100-200 mesh, 0-100% ethyl acetate in petroleum ether) affording 7-(8-chloronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 38.83%) as a brown oil. LCMS Rt=2.428 min, m/z=565.1 [M+H]⁺.

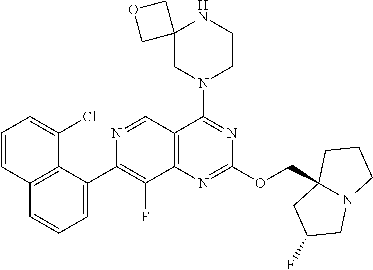 [SMILES: Fc1c(-c2cccc3cccc(Cl)c23)ncc2c(N3CCNC4(COC4)C3)nc(OC[C@@]34CCCN3C[C@H](F)C4)nc12]

### Step 5: 8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

A mixture of 7-(8-chloronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (400 mg, 708.05 μmol), 2-oxa-5,8-diazaspiro[3.5]nonane (427.15 mg, 708.05 μmol, 2 hydrochloride), diisopropylethylamine (274.53 mg, 2.12 mmol) and 4A molecule sieves (100 mg) in N,N-dimethylformaldehyde (5 mL) was degassed and purged with nitrogen 3 times, and the mixture was stirred at 60° C. for 12 h under a nitrogen atmosphere. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 25%-65%, 8 min) affording 8-(7-(8-chloronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonane (110 mg, 26.20%) as a white solid. LCMS Rt=1.532 min, m/z=593.2 [M+H]⁺.

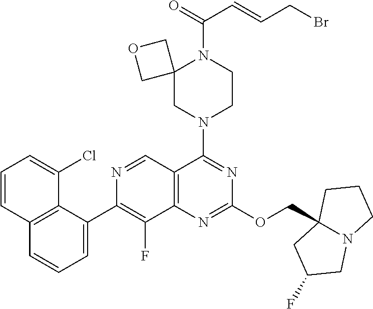 [SMILES: O=C(/C=C/CBr)N1CCN(c2nc(OC[C@@]34CCCN3C[C@H](F)C4)nc3c(F)c(-c4cccc5cccc(Cl)c45)ncc23)CC12COC2]

### Step 6: (E)-4-bromo-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The acylation reaction was prepared in a similar fashion to Method #3, Step 2. The reaction mixture was concentrated in vacuo affording (E)-4-bromo-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (130 mg, crude) as a brown oil, which was used in the next step without further purification. LCMS Rt=0.745 min, m/z=741.2 [M+H]⁺.

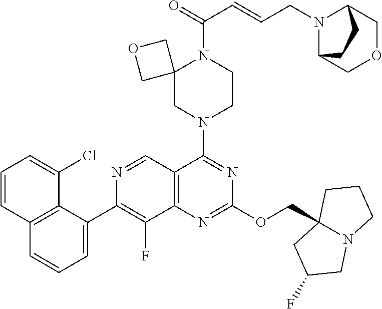

### Step 7: (E)-4-((1R,5S)-3-oxa-8-azabicyclo[3.2.1]octan-8-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 30%-70%, 8 min) affording (E)-4-((1R,5S)-3-oxa-8-azabicyclo[3.2.1]octan-8-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (71.34 mg, 97.23%) as a yellow solid: 1H NMR (400 MHz, Dimethylsulfoxide-d6) δ 9.16 (s, 1H), 8.24-8.17 (m, 1H), 8.10 (d, J=7.9 Hz, 1H), 7.76-7.70 (m, 1H), 7.64 (dd, J=7.1, 13.6 Hz, 2H), 7.60-7.54 (m, 1H), 6.78-6.69 (m, 1H), 6.65-6.56 (m, 1H), 5.37-5.18 (m, 1H), 4.82 (br t, J=5.8 Hz, 2H), 4.47-4.37 (m, 4H), 4.19-4.06 (m, 2H), 3.94 (br s, 2H), 3.69 (br d, J=3.3 Hz, 2H), 3.56 (br d, J=10.0 Hz, 2H), 3.42 (br d, J=9.5 Hz, 2H), 3.12-3.05 (m, 4H), 3.01 (br s, 3H), 2.86-2.78 (m, 1H), 2.14 (br d, J=4.5 Hz, 1H), 2.06 (br s, 1H), 2.01 (br s, 1H), 1.88-1.82 (m, 3H), 1.81-1.75 (m, 2H), 1.75-1.69 (m, 2H).

LCMS (5% to 95% acetonitrile in water+0.03% ammonium bicarbonate over 6 min); retention time 2.944 min, ESI+ found [M+H]=772.3.

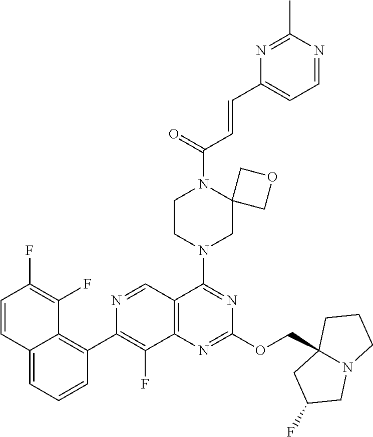 [SMILES: Cc1nccc(/C=C/C(=O)N2CCN(c3nc(OC[C@@]45CCCN4C[C@H](F)C5)nc4c(F)c(-c5cccc6ccc(F)c(F)c56)ncc34)CC23COC3)n1]

### Example 5 (Method 2): (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

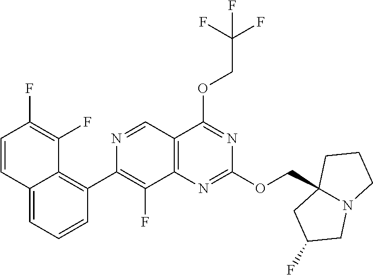 [SMILES: Fc1ccc2cccc(-c3ncc4c(OCC(F)(F)F)nc(OC[C@@]56CCCN5C[C@H](F)C6)nc4c3F)c2c1F]

### Step 1: 7-(7,8-difluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The crude product was purified by column chromatography (silica gel, 100-200 mesh, 0-100% methanol in dichloromethane) affording 7-(7,8-difluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (600 mg, 92.95%) as a yellow oil. LCMS Rt=0.654 min, m/z=567.2 [M+H]⁺.

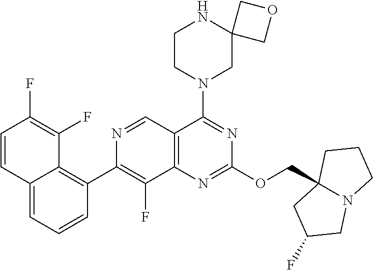 [SMILES: Fc1ccc2cccc(-c3ncc4c(N5CCNC6(COC6)C5)nc(OC[C@@]56CCCN5C[C@H](F)C6)nc4c3F)c2c1F]

### Step 2: 8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The crude product was purified by reverse phase HPLC (column: Waters Xbridge BEH C18 250*50 mm*10 μm; mobile phase: [water (NH₄HCO₃)-ACN]; B %: 30%-55%, 10 min) affording 8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (140 mg, 22.23%) as a yellow solid. LCMS Rt=1.467 min, m/z=595.2 [M+H]⁺.

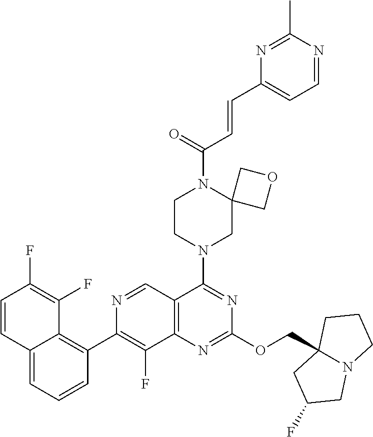 [SMILES: Cc1nccc(/C=C/C(=O)N2CCN(c3nc(OC[C@@]45CCCN4C[C@H](F)C5)nc4c(F)c(-c5cccc6ccc(F)c(F)c56)ncc34)CC23COC3)n1]

### Step 3: (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

The amide coupling reaction was prepared in a similar fashion to Method #2, Step 3. The crude product was purified by reverse phase HPLC (column: Waters Xbridge Prep OBD C18 150*40 mm*10 μm; mobile phase: [water (NH₄HCO₃)-ACN]; B %: 40%-70%, 8 min) affording (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (102 mg, 57.12%) as a gray solid: ¹H NMR (400 MHz, Dimethylsulfoxide-d6) δ 9.21 (s, 1H), 8.79 (d, J=5.1 Hz, 1H), 8.22 (td, J=2.0, 7.2 Hz, 1H), 8.03 (td, J=4.0, 5.1 Hz, 1H), 7.78-7.65 (m, 5H), 7.46 (d, J=15.4 Hz, 1H), 5.37-5.20 (m, 1H), 4.90 (br t, J=7.3 Hz, 2H), 4.57-4.50 (m, 1H), 4.46 (br d, J=6.4 Hz, 3H), 4.21-4.16 (m, 1H), 4.14-4.08 (m, 1H), 4.01 (br d, J=4.0 Hz, 2H), 3.90-3.78 (m, 2H), 3.15-3.05 (m, 2H), 3.02 (s, 1H), 2.87-2.80 (m, 1H), 2.66 (s, 3H), 2.17-2.12 (m, 1H), 2.09-1.98 (m, 2H), 1.88-1.74 (m, 3H). LCMS Rt=2.929 min, m/z=740.3 [M+H]⁺.

LCMS (5% to 95% acetonitrile in water+0.03% ammonium bicarbonate over 6 min); retention time 2.929 min, ESI+ found [M+H]=741.3.

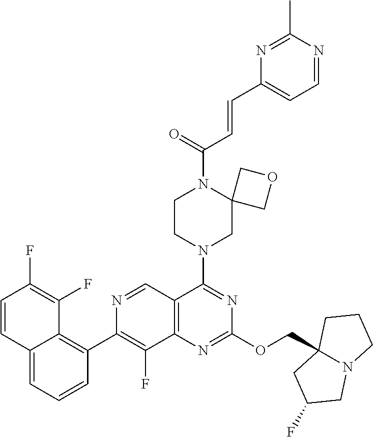 [SMILES: Cc1nccc(/C=C/C(=O)N2CCN(c3nc(OC[C@@]45CCCN4C[C@H](F)C5)nc4c(F)c(-c5cccc6ccc(F)c(F)c56)ncc34)CC23COC3)n1]

### Example 6 (Method 2): (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

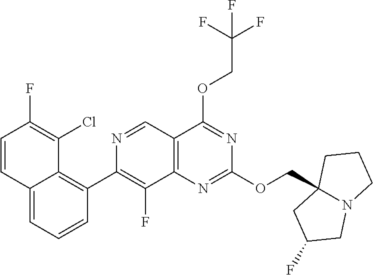 [SMILES: Fc1ccc2cccc(-c3ncc4c(OCC(F)(F)F)nc(OC[C@@]56CCCN5C[C@H](F)C6)nc4c3F)c2c1Cl]

### Step 1: 7-(8-chloro-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The crude product was purified by column chromatography (silica gel, 100-200 mesh, 0-100% methanol in dichloromethane) affording 7-(8-chloro-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (900 mg, 67.74%) as a brown oil. LCMS Rt=0.798 min, m/z=583.1 [M+H]⁺.

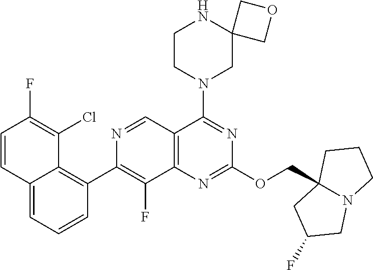 [SMILES: Fc1ccc2cccc(-c3ncc4c(N5CCNC6(COC6)C5)nc(OC[C@@]56CCCN5C[C@H](F)C6)nc4c3F)c2c1Cl]

### Step 2: 8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The crude product was purified by reverse phase HPLC (column: Waters Xbridge Prep OBD C18 150*40 mm*10 μm; mobile phase: [water (NH₄HCO₃)-ACN]; B %: 25%-65%, 8 min) affording 8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (300 mg, 35.77%) as a white solid. LCMS Rt=1.825 min, m/z=611.2 [M+H]⁺.

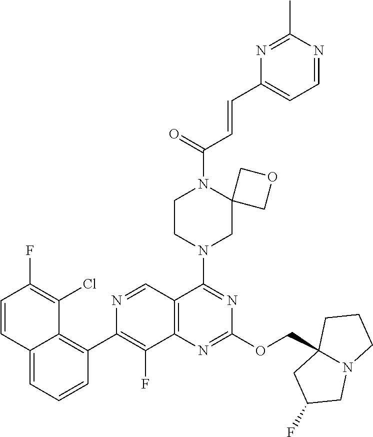 [SMILES: Cc1nccc(/C=C/C(=O)N2CCN(c3nc(OC[C@@]45CCCN4C[C@H](F)C5)nc4c(F)c(-c5cccc6ccc(F)c(Cl)c56)ncc34)CC23COC3)n1]

### Step 3: (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

The amide coupling reaction was prepared in a similar fashion to Method #2, Step 3. The crude product was purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (41.44 mg, 33.26%) as a pale yellow amorphous solid: ¹H NMR (400 MHz, Acetonitrile-d3) δ 9.09 (s, 1H), 8.69 (d, J=5.0 Hz, 1H), 8.15-8.09 (m, 1H), 8.05 (dd, J=5.6, 9.1 Hz, 1H), 7.70-7.65 (m, 2H), 7.62-7.56 (m, 1H), 7.54-7.44 (m, 2H), 7.34 (d, J=5.1 Hz, 1H), 5.34-5.16 (m, 1H), 4.91 (t, J=6.8 Hz, 2H), 4.57 (dd, J=6.6, 13.1 Hz, 1H), 4.51-4.38 (m, 3H), 4.25-4.19 (m, 1H), 4.17-4.11 (m, 1H), 3.96 (br t, J=4.9 Hz, 2H), 3.81-3.66 (m, 2H), 3.18-3.11 (m, 2H), 3.09-3.04 (m, 1H), 2.92-2.85 (m, 1H), 2.66 (s, 3H), 2.16-1.96 (m, 3H), 1.92-1.80 (m, 3H).

LCMS (5% to 95% acetonitrile in water+0.1% trifluoroacetic acid over 6 min); retention time 2.174 min, ESI+ found [M+H]=757.3.

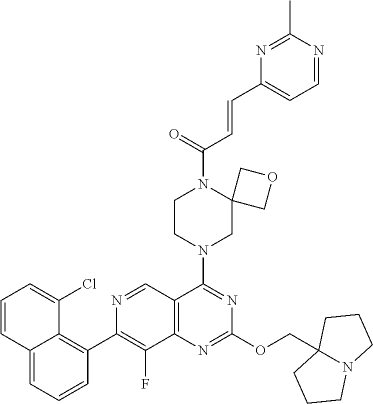 [SMILES: Cc1nccc(/C=C/C(=O)N2CCN(c3nc(OCC45CCCN4CCC5)nc4c(F)c(-c5cccc6cccc(Cl)c56)ncc34)CC23COC3)n1]

### Example 7 (Method 2): (E)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

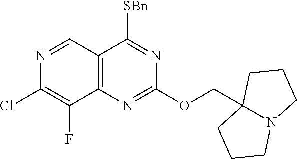

### Step 1: 4-(benzylthio)-7-chloro-8-fluoro-2-((tetrahydro-1H-pyrrolizin-7a(5H)-yl)methoxy)pyrido[4,3-d]pyrimidine

The substitution reaction was prepared in a similar fashion to Method #1, Step 1. The residue was purified by column chromatography (silica gel, 100-200 mesh, 0-100% ethyl acetate in petroleum ether) affording 4-(benzylthio)-7-chloro-8-fluoro-2-((hexahydro-1H-pyrrolizin-7a-yl)methoxy)pyrido[4,3-d]pyrimidine (10 g, 47.79%) as a yellow oil. LCMS Rt=0.694 min, m/z=445.1 [M+H]⁺.

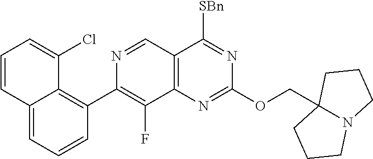

### Step 2: 4-(benzylthio)-7-(8-chloronaphthalen-1-yl)-8-fluoro-2-((tetrahydro-1H-pyrrolizin-7a(5H)-yl)methoxy)pyrido[4,3-d]pyrimidine

The Suzuki reaction was prepared in a similar fashion to Method #1, Step 2. The mixture was purified by column chromatography (silica gel, 100-200 mesh, 10-50% ethyl acetate in petroleum ether) affording 4-(benzylthio)-7-(8-chloronaphthalen-1-yl)-8-fluoro-2-((tetrahydro-1H-pyrrolizin-7a(5H)-yl)methoxy)pyrido[4,3-d]pyrimidine (4.5 g, 80.45%) as a yellow solid. LCMS Rt=0.818 min, m/z=571.2 [M+H]⁺.

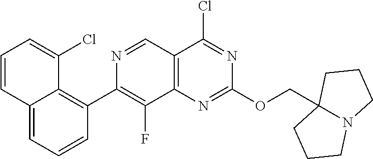 [SMILES: Fc1c(-c2cccc3cccc(Cl)c23)ncc2c(Cl)nc(OCC34CCCN3CCC4)nc12]

### Step 3: 4-chloro-7-(8-chloronaphthalen-1-yl)-8-fluoro-2-((hexahydro-1H-pyrrolizin-7a-yl)methoxy)pyrido[4,3-d]pyrimidine

The chlorination reaction was prepared in a similar fashion to Method #1, Step 3. The mixture was dried over sodium sulphate affording 4-chloro-7-(8-chloronaphthalen-1-yl)-8-fluoro-2-((hexahydro-1H-pyrrolizin-7a-yl)methoxy)pyrido[4,3-d]pyrimidine (400 mg, crude) as a yellow oil, used in next step without any further purification. LCMS Rt=0.837 min, m/z=483.1 [M+H]⁺.

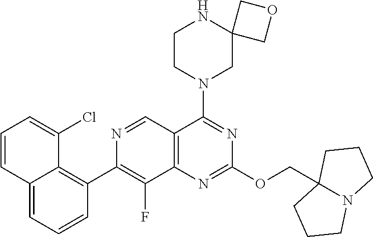 [SMILES: Fc1c(-c2cccc3cccc(Cl)c23)ncc2c(N3CCNC4(COC4)C3)nc(OCC34CCCN3CCC4)nc12]

### Step 4: 8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #1, Step 4. The reaction mixture was concentrated in vacuo and purified by reverse phase HPLC (column: Phenomenex C18 80*30 mm*3 μm; mobile phase: [water (TFA)-ACN]; B %: 1%-30%, 8 min) affording 8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonane (300 mg, 70.14%, trifluoroacetate salt) as a yellow solid. LCMS Rt=0.972 min, m/z=575.2 [M+H]⁺.

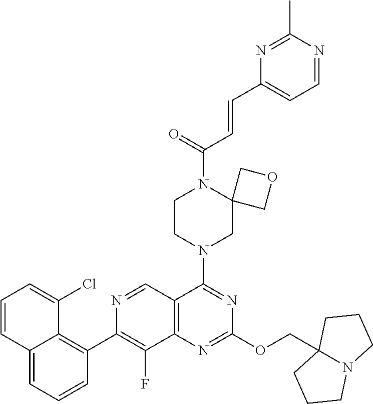 [SMILES: Cc1nccc(/C=C/C(=O)N2CCN(c3nc(OCC45CCCN4CCC5)nc4c(F)c(-c5cccc6cccc(Cl)c56)ncc34)CC23COC3)n1]

### Step 5: (E)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

The amide coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording (E)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (27.02 mg, 15.85%) as a yellow oil: ¹H NMR (400 MHz, Acetonitrile-d3) δ 9.14 (s, 1H), 8.74 (d, J=5.1 Hz, 1H), 8.17 (d, J=8.4 Hz, 1H), 8.06 (d, J=8.1 Hz, 1H), 7.76-7.71 (m, 1H), 7.69-7.61 (m, 3H), 7.58-7.48 (m, 2H), 7.40 (d, J=5.3 Hz, 1H), 4.96 (t, J=6.3 Hz, 2H), 4.61 (br d, J=12.7 Hz, 1H), 4.55-4.45 (m, 3H), 4.22 (s, 2H), 4.01 (br t, J=4.9 Hz, 2H), 3.86-3.72 (m, 2H), 3.06-2.99 (m, 2H), 2.71 (s, 3H), 2.68-2.62 (m, 2H), 2.02 (br s, 2H), 1.92-1.81 (m, 4H), 1.73-1.62 (m, 2H).

LCMS (5% to 95% acetonitrile in water+0.1% trifluoroacetic acid over 6 min); retention time 2.217 min, ESI+ found [M+H]=721.3.

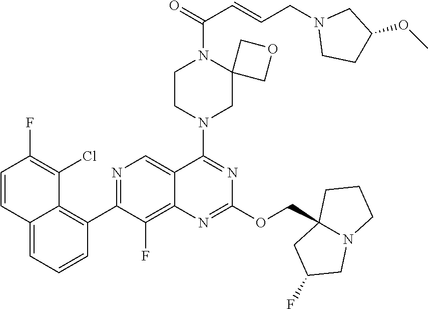 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)CC23COC3)C1]

### Example 8 (Method 3): (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one

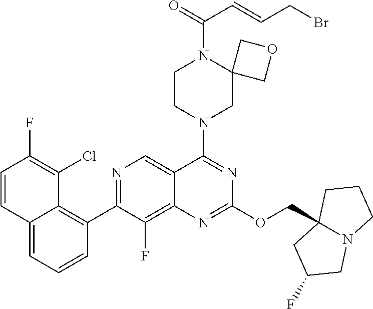 [SMILES: O=C(/C=C/CBr)N1CCN(c2nc(OC[C@@]34CCCN3C[C@H](F)C4)nc3c(F)c(-c4cccc5ccc(F)c(Cl)c45)ncc23)CC12COC2]

### Step 1: (E)-4-bromo-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The coupling reaction was prepared in a similar fashion to Method #3, Step 2. The reaction mixture was concentrated to dryness in vacuo affording (E)-4-bromo-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (200 mg, crude) as a brown oil. LCMS Rt=0.650 min, m/z=759.1 [M+H]+.

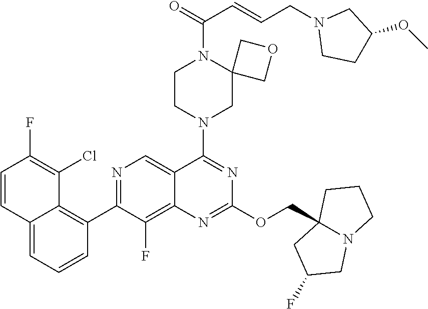 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)CC23COC3)C1]

### Step 2: (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one (16.35 mg, 13.38%) as a yellow amorphous solid: ¹H NMR (400 MHz, Dimethylsulfoxide-d6) δ 9.17 (s, 1H), 8.28-8.19 (m, 2H), 7.78-7.67 (m, 3H), 6.79-6.69 (m, 1H), 6.57 (br d, J=15.3 Hz, 1H), 5.41-5.18 (m, 1H), 4.82 (br t, J=5.9 Hz, 2H), 4.46-4.40 (m, 3H), 4.21-4.15 (m, 1H), 4.13-4.08 (m, 1H), 4.00-3.83 (m, 3H), 3.76-3.62 (m, 2H), 3.32 (br s, 1H), 3.22 (br d, J=5.6 Hz, 2H), 3.17 (s, 3H), 3.12-3.07 (m, 2H), 3.02 (s, 1H), 2.87-2.80 (m, 1H), 2.71 (dd, J=6.3, 10.1 Hz, 1H), 2.61-2.55 (m, 1H), 2.49-2.40 (m, 2H), 2.17-2.05 (m, 2H), 2.04-1.93 (m, 2H), 1.88-1.76 (m, 3H), 1.71-1.61 (m, 1H).

LCMS (5% to 95% acetonitrile in water+0.1% trifluoroacetic acid over 6 min); retention time 1.994 min, ESI+ found [M+H]=778.3.

### Example 9 (Method 3): (E)-4-((1R,4R)-2-oxa-5-azabicyclo[2.2.1]heptan-5-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

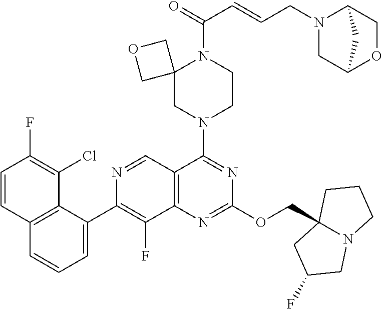 [SMILES: O=C(/C=C/CN1C[C@H]2C[C@@H]1CO2)N1CCN(c2nc(OC[C@@]34CCCN3C[C@H](F)C4)nc3c(F)c(-c4cccc5ccc(F)c(Cl)c45)ncc23)CC12COC2]

### Step 1: (E)-4-((1R,4R)-2-oxa-5-azabicyclo[2.2.1]heptan-5-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-4-((1R,4R)-2-oxa-5-azabicyclo[2.2.1]heptan-5-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (28.06 mg, 24.50%) as a yellow amorphous solid: ¹H NMR (400 MHz, Dimethylsulfoxide-d6) δ 9.21 (s, 1H), 8.32-8.23 (m, 2H), 7.81-7.72 (m, 3H), 6.83-6.73 (m, 1H), 6.61 (br d, J=15.0 Hz, 1H), 5.42-5.24 (m, 1H), 4.87 (br t, J=6.5 Hz, 2H), 4.54-4.34 (m, 6H), 4.24-4.20 (m, 1H), 4.17-4.12 (m, 1H), 3.99 (br s, 2H), 3.89 (d, J=7.5 Hz, 1H), 3.79-3.68 (m, 2H), 3.57 (dd, J=1.6, 7.6 Hz, 1H), 3.51 (s, 1H), 3.45-3.40 (m, 1H), 3.14 (br d, J=9.9 Hz, 2H), 3.09-3.05 (m, 1H), 2.91-2.79 (m, 2H), 2.50 (br d, J=10.4 Hz, 1H), 2.23-2.16 (m, 1H), 2.13-2.01 (m, 2H), 1.92-1.84 (m, 2H), 1.84-1.78 (m, 2H), 1.63 (br d, J=9.4 Hz, 1H).

LCMS (5% to 95% acetonitrile in water+0.1% trifluoroacetic acid over 6 min); retention time 1.967 min, ESI+ found [M+H]=776.3.

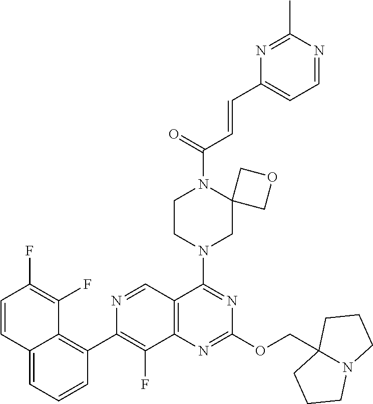 [SMILES: Cc1nccc(/C=C/C(=O)N2CCN(c3nc(OCC45CCCN4CCC5)nc4c(F)c(-c5cccc6ccc(F)c(F)c56)ncc34)CC23COC3)n1]

### Example 10 (Method 2): (E)-1-(8-(7-(7,8-difluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

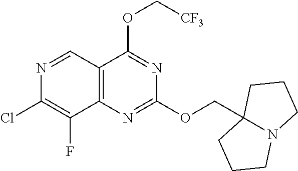 [SMILES: Fc1c(Cl)ncc2c(OCC(F)(F)F)nc(OCC34CCCN3CCC4)nc12]

### Step 1: 7-chloro-8-fluoro-2-((tetrahydro-1H-pyrrolizin-7a(5H)-yl)methoxy)-4-(2,2,2-trifluoroethoxy)pyrido[4,3-d]pyrimidine

The substitution reaction was prepared in a similar fashion to Method #6, Step 3. The reaction mixture was concentrated in vacuo and purified by column chromatography (silica gel, 100-200 mesh, 0-100% ethyl acetate in petroleum ether) affording 7-chloro-8-fluoro-2-((tetrahydro-1H-pyrrolizin-7a(5H)-yl)methoxy)-4-(2,2,2-trifluoroethoxy)pyrido[4,3-d]pyrimidine (350 mg, 26.29%) as a yellow oil. LCMS Rt=0.658 min, m/z=421.1 [M+H]⁺.

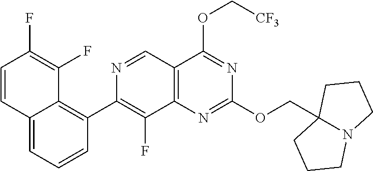 [SMILES: Fc1ccc2cccc(-c3ncc4c(OCC(F)(F)F)nc(OCC56CCCN5CCC6)nc4c3F)c2c1F]

### Step 2: 7-(7,8-difluoronaphthalen-1-yl)-8-fluoro-2-((tetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The crude product was purified by column chromatography (silica gel, 100-200 mesh, 0-100% methanol in dichloromethane) affording 7-(7,8-difluoronaphthalen-1-yl)-8-fluoro-2-((tetrahydro-1H-pyrrolizin-7a(5H)-yl)methoxy)-4-(2,2,2-trifluoroethoxy)pyrido[4,3-d]pyrimidine (240 mg, 92.06%) as a yellow oil. LCMS Rt=0.724 min, m/z=549.2 [M+H]⁺.

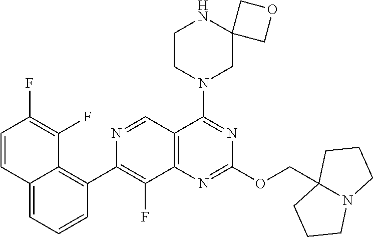 [SMILES: Fc1ccc2cccc(-c3ncc4c(N5CCNC6(COC6)C5)nc(OCC56CCCN5CCC6)nc4c3F)c2c1F]

### Step 3: 8-(7-(7,8-difluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 20%-50%, 8 min]; B %: 35%-70%, 8 min) affording 8-(7-(7,8-difluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonane (40 mg, 25.37%) as a white solid. LCMS Rt=1.161 min, m/z=577.3 [M+H]⁺.

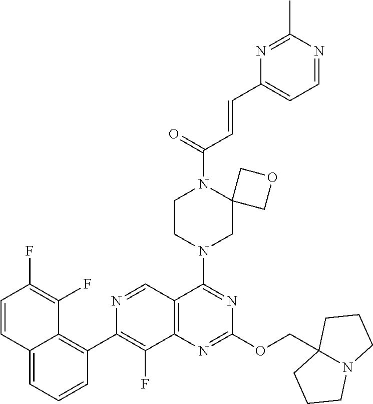 [SMILES: Cc1nccc(/C=C/C(=O)N2CCN(c3nc(OCC45CCCN4CCC5)nc4c(F)c(-c5cccc6ccc(F)c(F)c56)ncc34)CC23COC3)n1]

### Step 4: (E)-1-(8-(7-(7,8-difluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

The amide coupling reaction was prepared in a similar fashion to Method #2, Step 3. The reaction mixture was concentrated in vacuo and purified by reverse phase HPLC (column: Waters Xbridge BEH C18 100*30 mm*10 μm; mobile phase: [water (NH₄HCO₃)-ACN]; B %: 25%-55%, 8 min) affording (E)-1-(8-(7-(7,8-difluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (5.19 mg, 18.69%) as a yellow solid: ¹H NMR (400 MHz, Acetonitrile-d3) δ 9.16 (s, 1H), 8.73 (d, J=5.0 Hz, 1H), 8.17-8.10 (m, 1H), 7.92 (br dd, J=5.6, 8.6 Hz, 1H), 7.76-7.68 (m, 2H), 7.67-7.47 (m, 3H), 7.39 (d, J=4.9 Hz, 1H), 4.95 (br t, J=6.7 Hz, 2H), 6.62-4.66 (m, 1H), 4.64-4.57 (m, 2H), 4.49-4.43 (m, 1H), 4.20 (s, 2H), 4.03-3.97 (m, 2H), 3.81-3.76 (m, 1H), 3.68 (br t, J=6.4 Hz, 2H), 3.04-2.97 (m, 2H), 2.70 (s, 3H), 2.65-2.62 (m, 1H), 1.89-1.80 (m, 6H), 1.72-1.62 (m, 2H).

LCMS (5% to 95% acetonitrile in water+0.1% trifluoroacetic acid over 6 min); retention time 2.035 min, ESI+ found [M+H]=723.3.

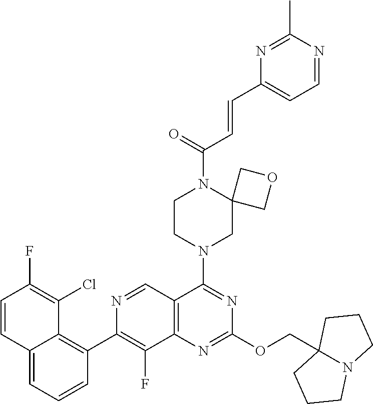 [SMILES: Cc1nccc(/C=C/C(=O)N2CCN(c3nc(OCC45CCCN4CCC5)nc4c(F)c(-c5cccc6ccc(F)c(Cl)c56)ncc34)CC23COC3)n1]

### Example 11 (Method 2): (E)-1-(8-(7-(8-chloro-7-fluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

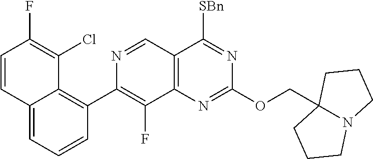

### Step 1: 4-(benzylthio)-7-(8-chloro-7-fluoronaphthalen-1-yl)-8-fluoro-2-((hexahydro-1H-pyrrolizin-7a-yl)methoxy)pyrido[4,3-d]pyrimidine

The Suzuki reaction was prepared in a similar fashion to Method #1, Step 2. The mixture was diluted with water (20 mL) and extracted with ethyl acetate (3×50 mL). The combined organic layers were dried over sodium sulphate, concentrated in vacuo and purified by column chromatography (silica gel, 100-200 mesh, 0-100% ethyl acetate in petroleum ether) affording 4-(benzylthio)-7-(8-chloro-7-fluoronaphthalen-1-yl)-8-fluoro-2-((hexahydro-1H-pyrrolizin-7a-yl)methoxy)pyrido[4,3-d]pyrimidine (1 g, crude) as a yellow oil, which was used in the next step without any further purification. LCMS Rt=0.759 min, m/z=590.2 [M+H]⁺.

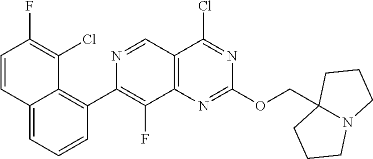 [SMILES: Fc1ccc2cccc(-c3ncc4c(Cl)nc(OCC56CCCN5CCC6)nc4c3F)c2c1Cl]

### Step 2: 4-chloro-7-(8-chloro-7-fluoronaphthalen-1-yl)-8-fluoro-2-((hexahydro-1H-pyrrolizin-7a-yl)methoxy)pyrido[4,3-d]pyrimidine

The chlorination reaction was prepared in a similar fashion to Method #1, Step 3. The mixture was concentrated to dryness in vacuo affording 4-chloro-7-(8-chloro-7-fluoronaphthalen-1-yl)-8-fluoro-2-((hexahydro-1H-pyrrolizin-7a-yl)methoxy)pyrido[4,3-d]pyrimidine (510 mg, crude) as a yellow oil, which was used in next step without any further purification. LCMS Rt=0.678 min, m/z=501.1 [M+H]⁺.

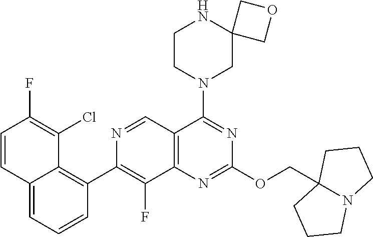 [SMILES: Fc1ccc2cccc(-c3ncc4c(N5CCNC6(COC6)C5)nc(OCC56CCCN5CCC6)nc4c3F)c2c1Cl]

### Step 3: 8-(7-(8-chloro-7-fluoronaphthalen-1-yl)-8-fluoro-2-((hexahydro-1H-pyrrolizin-7a-yl)methoxy)pyrido[4,3-d]pyrimidin-4-yl)-2-oxa-5,8-diazaspiro[3.5]nonane

The substitution reaction was prepared in a similar fashion to Method #1, Step 4. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording 8-(7-(8-chloro-7-fluoronaphthalen-1-yl)-8-fluoro-2-((hexahydro-1H-pyrrolizin-7a-yl)methoxy)pyrido[4,3-d]pyrimidin-4-yl)-2-oxa-5,8-diazaspiro[3.5]nonane (30.00 mg, 21.98%) as a white solid: LCMS Rt=1.555 min, m/z=593.2 [M+H]⁺.

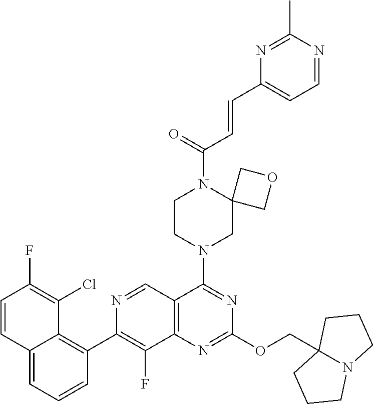 [SMILES: Cc1nccc(/C=C/C(=O)N2CCN(c3nc(OCC45CCCN4CCC5)nc4c(F)c(-c5cccc6ccc(F)c(Cl)c56)ncc34)CC23COC3)n1]

### Step 4: (E)-1-(8-(7-(8-chloro-7-fluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

The amide coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording (E)-1-(8-(7-(8-chloro-7-fluoronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (4.27 mg, 16.60%) as an amorphous yellow solid: ¹H NMR (400 MHz, Acetonitrile-d3) δ 9.14-9.09 (m, 1H), 8.72 (d, J=5.1 Hz, 1H), 8.20-8.05 (m, 2H), 7.74-7.68 (m, 2H), 7.66-7.59 (m, 1H), 7.57-7.46 (m, 2H), 7.38 (d, J=5.0 Hz, 1H), 4.95 (t, J=6.8 Hz, 2H), 4.63-4.57 (m, 1H), 4.56-4.49 (m, 2H), 4.48-4.43 (m, 1H), 4.23-4.16 (m, 2H), 4.00 (t, J=5.1 Hz, 2H), 3.86-3.70 (m, 2H), 3.03-2.96 (m, 2H), 2.69 (s, 3H), 2.62 (td, J=6.7, 10.1 Hz, 2H), 2.14 (br s, 2H), 1.91-1.78 (m, 4H), 1.70-1.61 (m, 2H). LCMS (5% to 95% acetonitrile in water+0.03% ammonium bicarbonate over 6 min); retention time 2.758 min, ESI+ found [M+H]=739.3.

### Example 12 (Method 3): (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

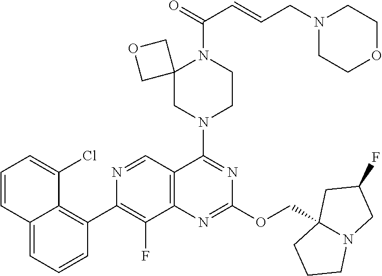 [SMILES: O=C(/C=C/CN1CCOCC1)N1CCN(c2nc(OC[C@@]34CCCN3C[C@H](F)C4)nc3c(F)c(-c4cccc5cccc(Cl)c45)ncc23)CC12COC2]

### Step 1: (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-acetonitrile]; B %: 40%-70%, 8 min) affording (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one (36 mg, 59.18%) as a yellow solid: ¹H NMR (400 MHz, Dimethylsulfoxide-d6) δ 9.17-9.14 (m, 1H), 8.24-8.18 (m, 1H), 8.12-8.08 (m, 1H), 7.76-7.70 (m, 1H), 7.64 (dd, J=7.0, 13.8 Hz, 2H), 7.60-7.54 (m, 1H), 6.75-6.67 (m, 1H), 6.63-6.55 (m, 1H), 5.38-5.20 (m, 1H), 4.81 (br t, J=5.7 Hz, 2H), 4.45-4.38 (m, 4H), 4.21-4.08 (m, 2H), 3.94 (br s, 2H), 3.69 (br d, J=3.0 Hz, 2H), 3.61-3.56 (m, 4H), 3.15-3.08 (m, 4H), 3.03 (br s, 1H), 2.87-2.80 (m, 1H), 2.38 (br s, 4H), 2.15 (br d, J=4.4 Hz, 1H), 2.07 (br s, 1H), 2.02 (br s, 1H), 1.88-1.76 (m, 3H). LCMS (5% to 95% acetonitrile in water+0.03% ammonium bicarbonate over 6 min); retention time 2.860 min, ESI+ found [M+H]=746.3.

### Example 13 (Method 3): (E)-4-((1R,4R)-2-oxa-5-azabicyclo[2.2.1]heptan-5-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

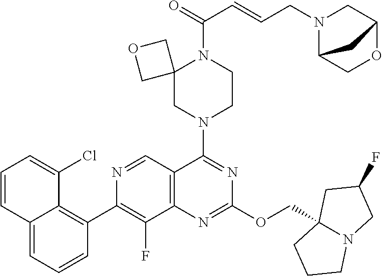 [SMILES: O=C(/C=C/CN1C[C@H]2C[C@@H]1CO2)N1CCN(c2nc(OC[C@@]34CCCN3C[C@H](F)C4)nc3c(F)c(-c4cccc5cccc(Cl)c45)ncc23)CC12COC2]

### Step 1: (E)-4-((1R,4R)-2-oxa-5-azabicyclo[2.2.1]heptan-5-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-acetonitrile]; B %: 40%-70%, 8 min) affording (E)-4-((1R,4R)-2-oxa-5-azabicyclo[2.2.1]heptan-5-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (30 mg, 48.49%) as a yellow solid: ¹H NMR (400 MHz, Dimethylsulfoxide-d6) δ 9.16 (s, 1H), 8.21 (d, J=7.6 Hz, 1H), 8.10 (d, J=7.5 Hz, 1H), 7.76-7.71 (m, 1H), 7.64 (dd, J=7.1, 14.6 Hz, 2H), 7.60-7.54 (m, 1H), 6.73 (td, J=5.4, 15.0 Hz, 1H), 6.56 (br d, J=15.0 Hz, 1H), 5.37-5.19 (m, 1H), 4.82 (br t, J=6.0 Hz, 2H), 4.47-4.38 (m, 4H), 4.34 (s, 1H), 4.20-4.07 (m, 2H), 3.94 (br s, 2H), 3.84 (d, J=7.5 Hz, 1H), 3.69 (br d, J=3.5 Hz, 2H), 3.52 (dd, J=1.5, 7.5 Hz, 1H), 3.46 (s, 1H), 3.34-3.28 (m, 2H), 3.09 (br d, J=9.5 Hz, 2H), 3.01 (s, 1H), 2.86-2.80 (m, 1H), 2.77 (br d, J=10.0 Hz, 1H), 2.45 (br d, J=10.0 Hz, 1H), 2.14 (br d, J=4.5 Hz, 1H), 2.06 (br s, 1H), 2.01 (br s, 1H), 1.88-1.72 (m, 4H), 1.58 (br d, J=9.5 Hz, 1H).

LCMS (5% to 95% acetonitrile in water+0.03% ammonium bicarbonate over 6 min); retention time 2.826 min, ESI+ found [M+H]=758.3.

### Example 14 (Method 3): (E)-4-(6-oxa-3-azabicyclo[3.1.1]heptan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

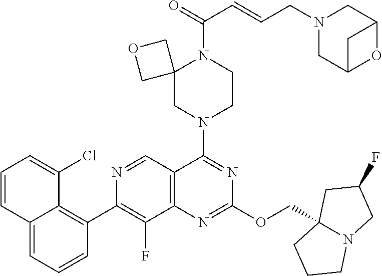 [SMILES: O=C(/C=C/CN1CC2CC(C1)O2)N1CCN(c2nc(OC[C@@]34CCCN3C[C@H](F)C4)nc3c(F)c(-c4cccc5cccc(Cl)c45)ncc23)CC12COC2]

### Step 1: (E)-4-(6-oxa-3-azabicyclo[3.1.1]heptan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-acetonitrile]; B %: 40%-70%, 8 min) affording (E)-4-(6-oxa-3-azabicyclo[3.1.1]heptan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (30 mg, 48.33%) as a yellow solid: ¹H NMR (400 MHz, Dimethylsulfoxide-d6) δ 9.16 (s, 1H), 8.20 (d, J=8.1 Hz, 1H), 8.10 (d, J=8.1 Hz, 1H), 7.73 (t, J=7.6 Hz, 1H), 7.64 (dd, J=7.1, 13.7 Hz, 2H), 7.60-7.54 (m, 1H), 6.79 (td, J=6.1, 15.0 Hz, 1H), 6.69-6.61 (m, 1H), 5.37-5.18 (m, 1H), 4.82 (br t, J=5.8 Hz, 2H), 4.43 (br d, J=5.9 Hz, 4H), 4.41-4.39 (m, 1H), 4.20-4.08 (m, 2H), 3.94 (br s, 2H), 3.71 (br s, 2H), 3.39 (br s, 1H), 3.13-3.00 (m, 6H), 2.85 (td, J=6.4, 13.3 Hz, 2H), 2.69 (br d, J=11.3 Hz, 2H), 2.23 (d, J=7.8 Hz, 1H), 2.14 (br d, J=4.1 Hz, 1H), 2.06 (br s, 1H), 2.01 (br s, 1H), 1.91-1.71 (m, 4H).

LCMS (5% to 95% acetonitrile in water+0.03% ammonium bicarbonate over 6 min); retention time 2.871 min, ESI+ found [M+H]=758.3.

### Example 15 (Method 3): (E)-4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

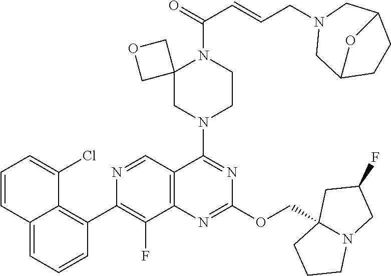 [SMILES: O=C(/C=C/CN1CC2CCC(C1)O2)N1CCN(c2nc(OC[C@@]34CCCN3C[C@H](F)C4)nc3c(F)c(-c4cccc5cccc(Cl)c45)ncc23)CC12COC2]

### Step 1: (E)-4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-acetonitrile]; B %: 40%-70%, 8 min) affording (E)-4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (30 mg, 47.91%) as a yellow solid: ¹H NMR (400 MHz, Dimethylsulfoxide-d6) δ 9.16 (s, 1H), 8.20 (br d, J=8.0 Hz, 1H), 8.10 (br d, J=8.0 Hz, 1H), 7.73 (br t, J=7.6 Hz, 1H), 7.64 (br dd, J=7.1, 13.4 Hz, 2H), 7.59-7.54 (m, 1H), 6.70 (td, J=5.9, 14.9 Hz, 1H), 6.62-6.52 (m, 1H), 5.37-5.17 (m, 1H), 4.82 (br t, J=5.3 Hz, 2H), 4.41 (br s, 4H), 4.26-4.00 (m, 5H), 3.94 (br s, 2H), 3.69 (br s, 2H), 3.09 (br d, J=5.5 Hz, 4H), 3.01 (br s, 1H), 2.83 (br d, J=6.0 Hz, 1H), 2.55 (br d, J=10.6 Hz, 2H), 2.20 (br d, J=9.9 Hz, 2H), 2.14 (br s, 1H), 2.06 (br s, 1H), 2.01 (br s, 1H), 1.86 (br d, J=6.1 Hz, 2H), 1.78 (br d, J=5.9 Hz, 2H), 1.75-1.69 (m, 2H).

LCMS (5% to 95% acetonitrile in water+0.03% ammonium bicarbonate over 6 min); retention time 3.023 min, ESI+ found [M+H]=772.3.

### Example 16 (Method 2): 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-yn-1-one

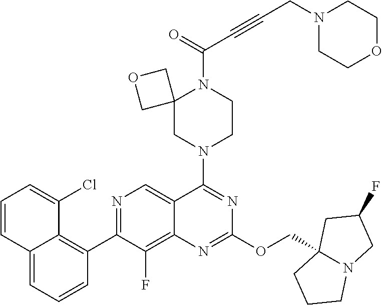 [SMILES: O=C(C#CCN1CCOCC1)N1CCN(c2nc(OC[C@@]34CCCN3C[C@H](F)C4)nc3c(F)c(-c4cccc5cccc(Cl)c45)ncc23)CC12COC2]

### Step 1: 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-yn-1-one

The amide coupling reaction was prepared in a similar fashion to Method #2, Step 3. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 30%-60%, 8 min) affording 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-yn-1-one (71.34 mg, 51.13%) as a yellow solid: ¹H NMR (400 MHz, Dimethylsulfoxide-d6) δ 9.24 (s, 1H), 8.24 (dd, J=1.1, 8.3 Hz, 1H), 8.14 (dd, J=1.0, 8.3 Hz, 1H), 7.79-7.74 (m, 1H), 7.68 (dd, J=6.9, 13.1 Hz, 2H), 7.63-7.58 (m, 1H), 5.41-5.22 (m, 1H), 5.13-5.02 (m, 2H), 4.56-4.47 (m, 2H), 4.46-4.38 (m, 2H), 4.24-4.11 (m, 4H), 4.05-3.93 (m, 2H), 3.68-3.62 (m, 4H), 3.61 (s, 2H), 3.18-3.10 (m, 2H), 3.06 (s, 1H), 2.91-2.82 (m, 1H), 2.55-2.52 (m, 4H), 2.23-2.14 (m, 1H), 2.13-2.02 (m, 2H), 1.91-1.78 (m, 3H).

LCMS (5% to 95% acetonitrile in water+0.1% trifluoroacetic acid over 6 min); retention time 1.873 min, ESI+ found [M+H]=744.3.

### Example 17 (Method 1): 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

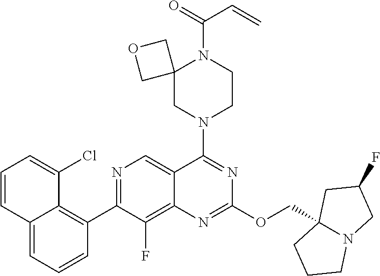 [SMILES: C=CC(=O)N1CCN(c2nc(OC[C@@]34CCCN3C[C@H](F)C4)nc3c(F)c(-c4cccc5cccc(Cl)c45)ncc23)CC12COC2]

### Step 1: 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The acylation reaction was prepared in a similar fashion to Method #1, Step 5. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 35%-65%, 8 min) affording 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (53 mg, 52.07%) as a white solid: ¹H NMR (400 MHz, Acetonitrile-d3) δ 9.10 (s, 1H), 8.13 (dd, J=1.0, 8.1 Hz, 1H), 8.02 (dd, J=0.8, 8.1 Hz, 1H), 7.73-7.67 (m, 1H), 7.65-7.59 (m, 2H), 7.55-7.49 (m, 1H), 6.65 (dd, J=10.6, 16.8 Hz, 1H), 6.28 (dd, J=1.9, 16.8 Hz, 1H), 5.77 (dd, J=1.9, 10.4 Hz, 1H), 5.36-5.19 (m, 1H), 4.87 (t, J=7.4 Hz, 2H), 4.54 (dd, J=6.8, 13.0 Hz, 1H), 4.45 (br t, J=4.9 Hz, 2H), 4.39 (dd, J=5.8, 13.1 Hz, 1H), 4.26-4.21 (m, 1H), 4.18-4.13 (m, 1H), 3.93 (t, J=5.1 Hz, 2H), 3.71-3.56 (m, 2H), 3.18-3.12 (m, 2H), 3.08 (s, 1H), 2.95-2.87 (m, 1H), 2.22-2.19 (m, 1H), 2.12 (d, J=2.8 Hz, 1H), 2.10-2.05 (m, 1H), 1.94-1.80 (m, 3H).

LCMS (5% to 95% acetonitrile in water+0.03% ammonium bicarbonate over 6 min); retention time 2.149 min, ESI+ found [M+H]=647.2.

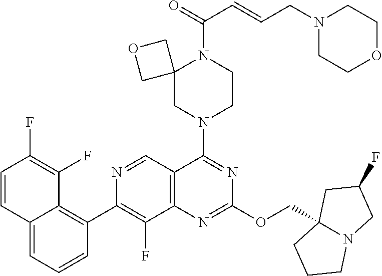 [SMILES: O=C(/C=C/CN1CCOCC1)N1CCN(c2nc(OC[C@@]34CCCN3C[C@H](F)C4)nc3c(F)c(-c4cccc5ccc(F)c(F)c45)ncc23)CC12COC2]

### Example 18 (Method 3): (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

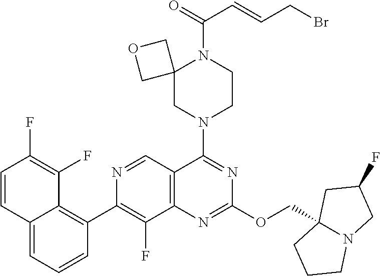 [SMILES: O=C(/C=C/CBr)N1CCN(c2nc(OC[C@@]34CCCN3C[C@H](F)C4)nc3c(F)c(-c4cccc5ccc(F)c(F)c45)ncc23)CC12COC2]

### Step 1: (E)-4-bromo-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The coupling reaction was prepared in a similar fashion to Method #3, Step 2. The crude product was concentrated to dryness in vacuo affording (E)-4-bromo-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (620 mg, crude) as a pale yellow gum. LCMS Rt=0.618 min, m/z=741.2 [M+H]⁺.

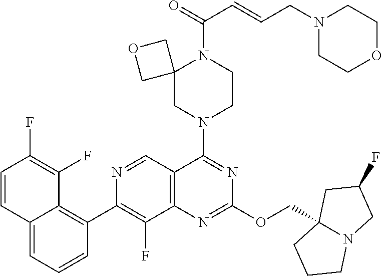 [SMILES: O=C(/C=C/CN1CCOCC1)N1CCN(c2nc(OC[C@@]34CCCN3C[C@H](F)C4)nc3c(F)c(-c4cccc5ccc(F)c(F)c45)ncc23)CC12COC2]

### Step 2: (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 25%-65%, 8 min) affording (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one (86.63 mg, 71.46%) as a white amorphous solid: ¹H NMR (400 MHz, Chloroform-d) δ 9.12-9.05 (m, 1H), 8.04-7.93 (m, 1H), 7.77-7.70 (m, 1H), 7.68-7.57 (m, 2H), 7.46-7.36 (m, 1H), 7.02-6.90 (m, 1H), 6.47-6.37 (m, 1H), 5.40-5.19 (m, 1H), 4.95-4.80 (m, 2H), 4.59-4.38 (m, 4H), 4.35-4.19 (m, 2H), 4.03-3.79 (m, 2H), 3.78-3.71 (m, 4H), 3.71-3.56 (m, 2H), 3.31-3.22 (m, 2H), 3.21-3.14 (m, 3H), 3.03-2.94 (m, 1H), 2.50 (br d, J=3.8 Hz, 4H), 2.30-2.09 (m, 3H), 2.00-1.84 (m, 3H).

LCMS (5% to 95% acetonitrile in water+0.03% ammonium bicarbonate over 6 min); retention time 2.903 min, ESI+ found [M+H]=748.3.

### Example 19 (Method 3): (E)-4-((1R,4R)-2-oxa-5-azabicyclo[2.2.1]heptan-5-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

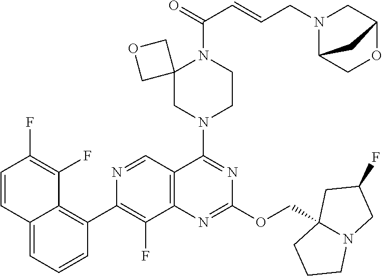 [SMILES: O=C(/C=C/CN1C[C@H]2C[C@@H]1CO2)N1CCN(c2nc(OC[C@@]34CCCN3C[C@H](F)C4)nc3c(F)c(-c4cccc5ccc(F)c(F)c45)ncc23)CC12COC2]

### Step 1: (E)-4-((1R,4R)-2-oxa-5-azabicyclo[2.2.1]heptan-5-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge BEH C18 100*30 mm*10 μm; mobile phase: [water (NH₄HCO₃)-ACN]; B %: 30%-60%, 8 min) affording (E)-4-((1R,4R)-2-oxa-5-azabicyclo[2.2.1]heptan-5-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (48.24 mg, 36.88%) as a yellow oil: ¹H NMR (400 MHz, Chloroform-d) δ 9.13-9.05 (m, 1H), 8.04-7.93 (m, 1H), 7.77-7.69 (m, 1H), 7.68-7.59 (m, 2H), 7.46-7.36 (m, 1H), 7.02-6.91 (m, 1H), 6.48-6.38 (m, 1H), 5.40-5.19 (m, 1H), 4.97-4.81 (m, 2H), 4.61-4.36 (m, 5H), 4.34-4.27 (m, 1H), 4.27-4.20 (m, 1H), 4.04 (br d, J=7.9 Hz, 1H), 4.01-3.91 (m, 1H), 3.90-3.78 (m, 1H), 3.74-3.59 (m, 3H), 3.52 (s, 1H), 3.38 (br s, 2H), 3.26 (br d, J=8.5 Hz, 2H), 3.21-3.14 (m, 1H), 3.03-2.94 (m, 1H), 2.94-2.88 (m, 1H), 2.67-2.57 (m, 1H), 2.33-2.24 (m, 1H), 2.24-2.10 (m, 2H), 2.01-1.84 (m, 4H), 1.79 (br d, J=10.3 Hz, 1H).

LCMS (5% to 95% acetonitrile in water+0.03% ammonium bicarbonate over 6 min); retention time 2.651 min, ESI+ found [M+H]=760.3.

### Example 20 (Method 3): (E)-4-(6-oxa-3-azabicyclo[3.1.1]heptan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

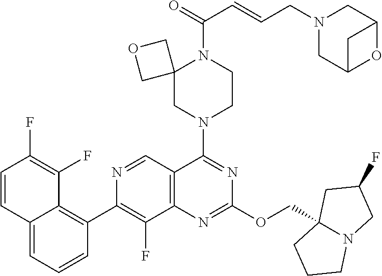 [SMILES: O=C(/C=C/CN1CC2CC(C1)O2)N1CCN(c2nc(OC[C@@]34CCCN3C[C@H](F)C4)nc3c(F)c(-c4cccc5ccc(F)c(F)c45)ncc23)CC12COC2]

### Step 1: (E)-4-(6-oxa-3-azabicyclo[3.1.1]heptan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-4-(6-oxa-3-azabicyclo[3.1.1]heptan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (52.21 mg, 42.47%) as a yellow amorphous solid: ¹H NMR (400 MHz, Chloroform-d) 8 9.12-9.06 (m, 1H), 7.98 (br d, J=8.0 Hz, 1H), 7.76-7.70 (m, 1H), 7.67-7.58 (m, 2H), 7.45-7.36 (m, 1H), 7.00 (td, J=5.9, 15.1 Hz, 1H), 6.49-6.40 (m, 1H), 5.42-5.13 (m, 1H), 4.95-4.82 (m, 2H), 4.57-4.51 (m, 3H), 4.50-4.41 (m, 2H), 4.34-4.20 (m, 2H), 4.02-3.81 (m, 2H), 3.74-3.56 (m, 2H), 3.47-3.43 (m, 2H), 3.31-3.22 (m, 2H), 3.19-3.11 (m, 3H), 3.09-2.95 (m, 2H), 2.90-2.82 (m, 2H), 2.37 (d, J=8.0 Hz, 1H), 2.30-2.12 (m, 3H), 2.11-1.74 (m, 4H).

LCMS (5% to 95% acetonitrile in water+0.03% ammonium bicarbonate over 6 min); retention time 2.880 min, ESI+ found [M+H]=760.3.

### Example 21 (Method 3): (E)-4-((1R,5S)-3-oxa-8-azabicyclo[3.2.1]octan-8-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

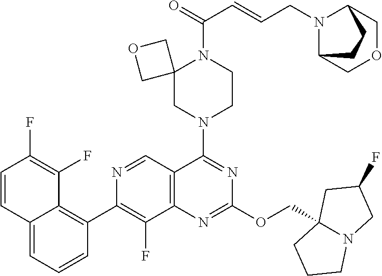

### Step 1: (E)-4-((1R,5S)-3-oxa-8-azabicyclo[3.2.1]octan-8-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge BEH C18 100*30 mm*10 μm; mobile phase: [water (NH₄HCO₃)-ACN]; B %: 35%-60%, 8 min) affording (E)-4-((1R,5S)-3-oxa-8-azabicyclo[3.2.1]octan-8-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (40.87 mg, 32.21%) as a yellow solid: ¹H NMR (400 MHz, Chloroform-d) δ 9.09 (s, 1H), 7.98 (d, J=8.0 Hz, 1H), 7.79-7.70 (m, 1H), 7.68-7.57 (m, 2H), 7.46-7.36 (m, 1H), 7.04-6.92 (m, 1H), 6.55-6.45 (m, 1H), 5.40-5.16 (m, 1H), 4.96-4.80 (m, 2H), 4.59-4.40 (m, 4H), 4.36-4.20 (m, 2H), 4.03-3.80 (m, 2H), 3.79-3.73 (m, 2H), 3.73-3.59 (m, 2H), 3.58-3.53 (m, 2H), 3.31-3.22 (m, 2H), 3.21-3.16 (m, 1H), 3.15-3.11 (m, 2H), 3.09-3.03 (m, 2H), 3.02-2.94 (m, 1H), 2.32-2.25 (m, 1H), 2.15 (br d, J=12.0 Hz, 2H), 1.93 (s, 7H).

LCMS (5% to 95% acetonitrile in water+0.03% ammonium bicarbonate over 6 min); retention time 2.768 min, ESI+ found [M+H]=774.3.

### Example 22 (Method 3): (E)-4-(8-oxa-3-azabicyclo[3.2.1]octan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

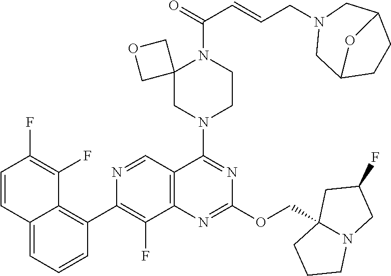 [SMILES: O=C(/C=C/CN1CC2CCC(C1)O2)N1CCN(c2nc(OC[C@@]34CCCN3C[C@H](F)C4)nc3c(F)c(-c4cccc5ccc(F)c(F)c45)ncc23)CC12COC2]

### Step 1: (E)-4-(8-oxa-3-azabicyclo[3.2.1]octan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-4-(8-oxa-3-azabicyclo[3.2.1]octan-3-yl)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (33.07 mg, 26.41%) as a yellow solid: ¹H NMR (400 MHz, Chloroform-d) δ 9.09 (s, 1H), 7.98 (br d, J=8.0 Hz, 1H), 7.76-7.70 (m, 1H), 7.67-7.59 (m, 2H), 7.41 (dt, J=7.4, 9.3 Hz, 1H), 6.92 (td, J=5.7, 15.2 Hz, 1H), 6.40 (br d, J=15.3 Hz, 1H), 5.38-5.20 (m, 1H), 4.88 (br dd, J=6.4, 19.1 Hz, 2H), 4.57-4.39 (m, 4H), 4.34-4.28 (m, 3H), 4.26-4.20 (m, 1H), 4.02-3.80 (m, 2H), 3.71-3.56 (m, 2H), 3.30-3.20 (m, 2H), 3.18-3.12 (m, 3H), 2.99 (dt, J=5.0, 9.2 Hz, 1H), 2.61 (d, J=10.8 Hz, 2H), 2.40-2.35 (m, 2H), 2.27 (br d, J=9.3 Hz, 1H), 2.23-2.11 (m, 2H), 2.03-1.94 (m, 4H), 1.89 (br s, 3H). LCMS (5% to 95% acetonitrile in water+0.03% ammonium bicarbonate over 6 min); retention time 2.851 min, ESI+ found [M+H]=774.3.

### Example 23 (Method 3): (E)-4-((1R,5S)-3-oxa-8-azabicyclo[3.2.1]octan-8-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

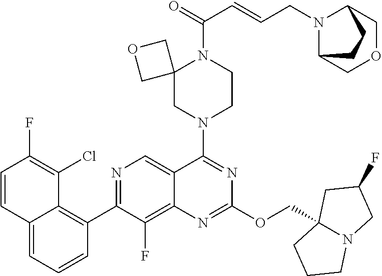

### Step 1: (E)-4-((1R,5S)-3-oxa-8-azabicyclo[3.2.1]octan-8-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The crude product was purified by reverse phase HPLC (column: Waters Xbridge Prep OBD C18 150*40 mm*10 μm; mobile phase: [water (NH₄HCO₃)-ACN]; B %: 40%-65%, 8 min) affording (E)-4-((1R,5S)-3-oxa-8-azabicyclo[3.2.1]octan-8-yl)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (17.32 mg, 14.46%) as a yellow amorphous solid: ¹H NMR (400 MHz, Dimethyl sulfoxide-d6) δ 9.22 (s, 1H), 8.32-8.24 (m, 2H), 7.81-7.73 (m, 3H), 6.83-6.75 (m, 1H), 6.71-6.60 (m, 1H), 5.43-5.24 (m, 1H), 4.88 (br t, J=6.1 Hz, 2H), 4.53-4.43 (m, 4H), 4.25-4.20 (m, 1H), 4.18-4.13 (m, 1H), 4.00 (br s, 2H), 3.75 (br d, J=5.4 Hz, 2H), 3.61 (d, J=10.1 Hz, 2H), 3.49 (s, 1H), 3.46 (br s, 1H), 3.18-3.10 (m, 4H), 3.07 (br s, 3H), 2.92-2.85 (m, 1H), 2.20 (br d, J=4.5 Hz, 1H), 2.14-2.11 (m, 1H), 2.09-2.02 (m, 1H), 1.94-1.88 (m, 3H), 1.87-1.82 (m, 2H), 1.81-1.75 (m, 2H).

LCMS (5% to 95% acetonitrile in water+0.1% trifluoroacetic acid over 6 min); retention time 1.894 min, ESI+ found [M+H]=790.3.

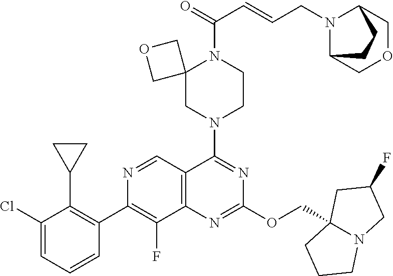

### Example 24 (Method 3): (E)-4-((1R,5S)-3-oxa-8-azabicyclo[3.2.1]octan-8-yl)-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

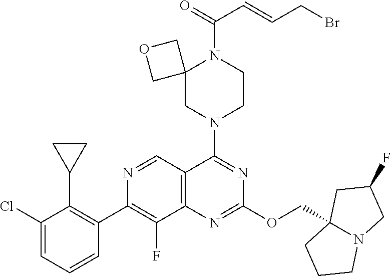 [SMILES: O=C(/C=C/CBr)N1CCN(c2nc(OC[C@@]34CCCN3C[C@H](F)C4)nc3c(F)c(-c4cccc(Cl)c4C4CC4)ncc23)CC12COC2]

### Step 1: (E)-4-bromo-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The amide coupling reaction was prepared in a similar fashion to Method #3, Step 2. The mixture was concentrated in vacuo affording (E)-4-bromo-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (81 mg, crude) as a yellow oil, which was used in the next step without further purification. LCMS Rt=0.623 min, m/z=731.2 [M+H]⁺.

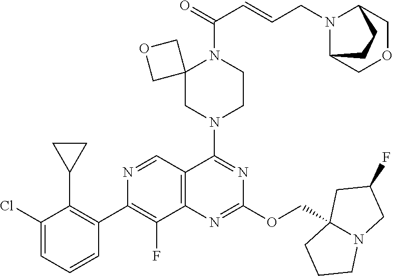

### Step 2: (E)-4-((1R,5S)-3-oxa-8-azabicyclo[3.2.1]octan-8-yl)-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by reverse phase HPLC (column: Waters Xbridge BEH C18 100*30 mm*10 μm; mobile phase: [water (NH₄HCO₃)-ACN]; B %: 35%-65%, 8 min) affording (E)-4-((1R,5S)-3-oxa-8-azabicyclo[3.2.1]octan-8-yl)-1-(8-(7-(3-chloro-2-cyclopropylphenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl) but-2-en-1-one (49.19 mg, 58.16%) as a yellow solid: ¹H NMR (400 MHz, Chloroform-d) δ 9.09 (s, 1H), 7.49 (dd, J=1.0, 7.9 Hz, 1H), 7.42-7.36 (m, 1H), 7.34-7.28 (m, 1H), 7.02-6.92 (m, 1H), 6.50 (br d, J=15.1 Hz, 1H), 5.40-5.20 (m, 1H), 4.88 (br d, J=6.5 Hz, 2H), 4.52-4.44 (m, 4H), 4.35-4.21 (m, 2H), 3.90 (br s, 2H), 3.75 (d, J=10.4 Hz, 2H), 3.66 (br d, J=4.4 Hz, 2H), 3.56 (br d, J=9.3 Hz, 2H), 3.34-3.22 (m, 2H), 3.21-3.11 (m, 3H), 3.09-2.95 (m, 3H), 2.34-2.21 (m, 2H), 2.21-2.02 (m, 3H), 2.01-1.95 (m, 2H), 1.93 (br s, 4H), 0.72 (br d, J=7.3 Hz, 2H), 0.24-0.10 (m, 2H).

LCMS (5% to 95% acetonitrile in water+0.03% ammonium bicarbonate over 6 min); retention time 3.101 min, ESI+ found [M+H]=762.3.

### Example 25 (Method 1): (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one

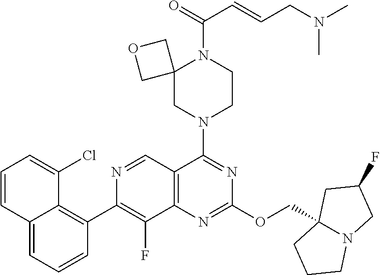 [SMILES: CN(C)C/C=C/C(=O)N1CCN(c2nc(OC[C@@]34CCCN3C[C@H](F)C4)nc3c(F)c(-c4cccc5cccc(Cl)c45)ncc23)CC12COC2]

### Step 1: (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one

The amide coupling reaction was prepared in a similar fashion to Method #1, Step 5. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 35%-65%, 8 min) affording (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one (5.42 mg, 17.00%) as a yellow solid: ¹H NMR (400 MHz, Acetonitrile-d3) δ 9.16-9.11 (m, 1H), 8.17 (d, J=8.1 Hz, 1H), 8.07 (d, J=7.8 Hz, 1H), 7.76-7.70 (m, 1H), 7.69-7.63 (m, 2H), 7.59-7.53 (m, 1H), 6.78 (s, 1H), 6.54-6.44 (m, 1H), 5.41-5.21 (m, 1H), 4.92-4.85 (m, 2H), 4.60-4.53 (m, 1H), 4.51-4.38 (m, 3H), 4.29-4.23 (m, 1H), 4.21-4.15 (m, 1H), 3.99-3.94 (m, 2H), 3.71-3.59 (m, 2H), 3.21-3.14 (m, 2H), 3.14-3.06 (m, 3H), 2.97-2.90 (m, 1H), 2.24 (s, 6H), 2.15 (br d, J=2.2 Hz, 2H), 2.12-2.07 (m, 1H), 1.93-1.80 (m, 3H).

LCMS (5% to 95% acetonitrile in water+0.03% ammonium bicarbonate over 6 min); retention time 2.963 min, ESI+ found [M+H]=704.3.

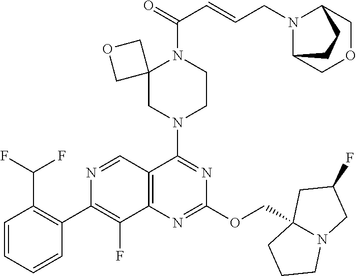

### Example 26 (Method 3): (E)-4-((1R,5S)-3-oxa-8-azabicyclo[3.2.1]octan-8-yl)-1-(8-(7-(2-(difluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

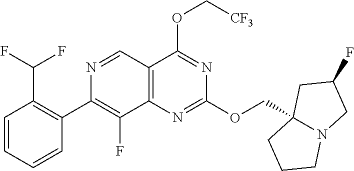 [SMILES: Fc1c(-c2ccccc2C(F)F)ncc2c(OCC(F)(F)F)nc(OC[C@@]34CCCN3C[C@H](F)C4)nc12]

### Step 1: 7-(2-(difluoromethyl)phenyl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine

The Suzuki reaction was prepared in a similar fashion to Method #6, Step 4. The residue was purified by column chromatography (silica gel, 100-200 mesh, 0-100% ethyl acetate in petroleum ether) affording 7-(2-(difluoromethyl)phenyl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (530.00 mg, 80.64%) as a yellow oil: ¹H NMR (400 MHz, Chloroform-d)_{8 9.27} (s, 1H), 7.90-7.82 (m, 1H), 7.64-7.58 (m, 3H), 7.16-6.87 (m, 1H), 5.43-5.22 (m, 1H), 5.13-5.00 (m, 2H), 4.45-4.28 (m, 2H), 3.38-3.19 (m, 3H), 3.09-2.94 (m, 1H), 2.30-2.13 (m, 3H), 2.02-1.91 (m, 3H) LCMS Rt=0.633 min, m/z=531.2 [M+H]⁺.

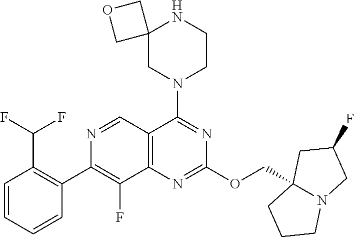 [SMILES: Fc1c(-c2ccccc2C(F)F)ncc2c(N3CCNC4(COC4)C3)nc(OC[C@@]34CCCN3C[C@H](F)C4)nc12]

### Step 2: 8-(7-(2-(difluoromethyl)phenyl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonane

The substitution reaction was prepared in a similar fashion to Method #6, Step 5. The residue was purified by column chromatography (silica gel, 100-200 mesh, 0-30% methanol in dichloromethane) affording 8-(7-(2-(difluoromethyl)phenyl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonane (380.00 mg, 63.74%) as a colorless oil. LCMS Rt=0.443 min, m/z=559.2 [M+H]⁺.

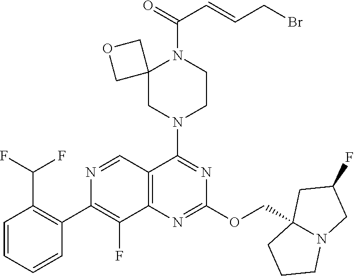 [SMILES: O=C(/C=C/CBr)N1CCN(c2nc(OC[C@@]34CCCN3C[C@H](F)C4)nc3c(F)c(-c4ccccc4C(F)F)ncc23)CC12COC2]

### Step 3: (E)-4-bromo-1-(8-(7-(2-(difluoromethyl)phenyl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonan-5-yl) but-2-en-1-one

The amide coupling reaction was prepared in a similar fashion to Method #3, Step 2. The reaction mixture was concentrated in vacuo affording (E)-4-bromo-1-(8-(7-(2-(difluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (126 mg, crude) as a yellow oil. LCMS Rt=0.589 min, m/z=705.2 [M+H]⁺.

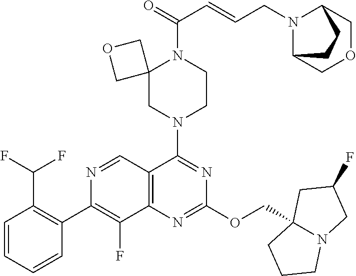

### Step 4: (E)-4-((1R,5S)-3-oxa-8-azabicyclo[3.2.1]octan-8-yl)-1-(8-(7-(2-(difluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording (E)-4-((1R,5S)-3-oxa-8-azabicyclo[3.2.1]octan-8-yl)-1-(8-(7-(2-(difluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (37.65 mg, 28.57%) as an amorphous solid: ¹H NMR (400 MHz, Chloroform-d)_{8 9.12}-9.04 (m, 1H), 7.90-7.81 (m, 1H), 7.66-7.57 (m, 3H), 7.19-6.84 (m, 2H), 6.55-6.43 (m, 1H), 5.39-5.21 (m, 1H), 4.87 (br d, J=5.6 Hz, 2H), 4.51-4.42 (m, 4H), 4.36-4.25 (m, 2H), 3.90 (br s, 2H), 3.76 (d, J=10.4 Hz, 2H), 3.65 (br t, J=4.6 Hz, 2H), 3.56 (dd, J=1.4, 10.4 Hz, 2H), 3.38-3.18 (m, 3H), 3.13 (dd, J=1.5, 5.1 Hz, 2H), 3.09-2.95 (m, 3H), 2.34-2.11 (m, 3H), 2.01-1.89 (m, 7H).

LCMS (5% to 95% acetonitrile in water+0.03% ammonium bicarbonate over 6 min); retention time 2.892 min, ESI+ found [M+H]=738.3.

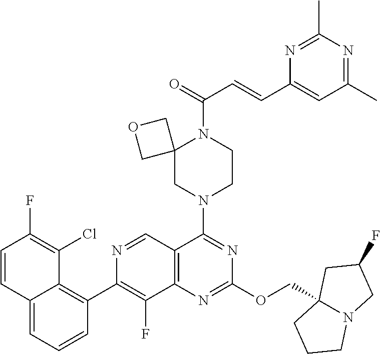 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)CC23COC3)nc(C)n1]

### Example 27 (Method 2): (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

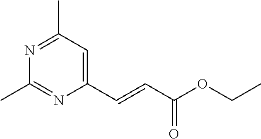

### Step 1: ethyl(E)-3-(2,6-dimethylpyrimidin-4-yl) acrylate

The Suzuki reaction was prepared in a similar fashion to Method #2, Step 1. The crude product was purified by column chromatography (silica gel, 100-200 mesh, 0-100% ethyl acetate in petroleum ether) affording ethyl(E)-3-(2,6-dimethylpyrimidin-4-yl) acrylate (7 g, 96.79%) as a yellow oil: ¹H NMR (400 MHz, Chloroform-d) δ 7.44 (d, J=15.6 Hz, 1H), 7.20 (s, 1H), 7.01 (d, J=15.8 Hz, 1H), 4.21 (q, J=7.1 Hz, 2H), 2.64 (s, 3H), 2.45 (s, 3H), 1.21-1.19 (m, 3H). LCMS Rt=0.546 min, m/z=207.1 [M+H]⁺.

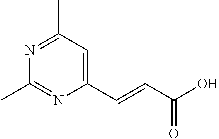

### Step 2: (E)-3-(2,6-dimethylpyrimidin-4-yl) acrylic acid

The hydrolysis reaction was prepared in a similar fashion to Method #2, Step 2. The crude product was purified by reverse phase HPLC (column: Phenomenex luna c18 250 mm*100 mm*10 μm; mobile phase: [water (TFA)-ACN]; B %: 0%-25%, 20 min) affording (E)-3-(2,6-dimethylpyrimidin-4-yl) acrylic acid (6 mg, 99.21%) as a yellow solid: ¹H NMR (400 MHz, Dimethylsuldoxide-d6) δ 7.52-7.41 (m, 2H), 6.99 (d, J=15.8 Hz, 1H), 2.60 (s, 3H), 2.45 (s, 3H). LCMS Rt=0.577 min, m/z=179.1 [M+H]⁺.

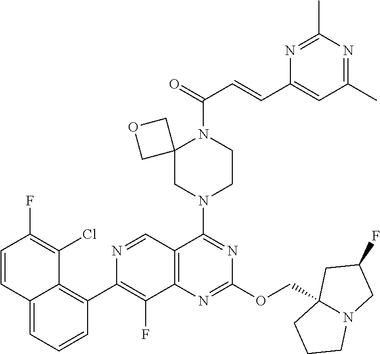 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)CC23COC3)nc(C)n1]

### Step 3: (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

The amide coupling reaction was prepared in a similar fashion to Method #2, Step 3. The crude product was purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one (6 mg, 11.76%) as a yellow oil: ¹H NMR (Acetonitrile-d3) δ 9.13 (s, 1H), 8.20-8.15 (m, 1H), 8.13-8.06 (m, 1H), 7.79-7.68 (m, 2H), 7.65-7.52 (m, 2H), 7.47 (br d, 1H), 5.49-5.14 (m, 1H), 4.95 (br m, 2H), 4.61 (br m, 1H), 4.54-4.41 (m, 3H), 4.31-4.11 (m, 2H), 4.01 (br s, 2H), 3.78 (m, 2H), 3.25-3.04 (m, 3H), 2.97-2.87 (m, 1H), 2.65 (s, 3H), 2.49 (s, 3H), 2.16-2.05 (m, 3H), 1.94-1.84 (m, 3H).

LCMS (5% to 95% acetonitrile in water+0.03% ammonium bicarbonate over 6 min); retention time 2.852 min, ESI+ found [M+H]=771.3.

### Example 28 (Method 2): (E)-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

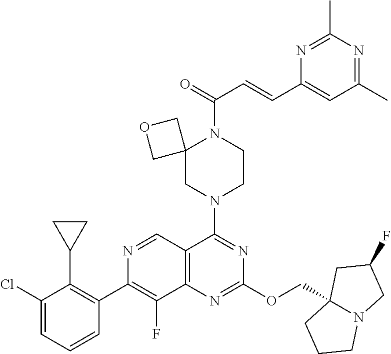 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)CC23COC3)nc(C)n1]

### Step 1: (E)-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

The amide coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one (28.53 mg, 55.95%) as a yellow amorphous solid: ¹H NMR (400 MHz, Chloroform-d) δ 9.10 (s, 1H), 7.68-7.61 (m, 1H), 7.55-7.47 (m, 2H), 7.41-7.37 (m, 1H), 7.34-7.28 (m, 1H), 7.01-6.98 (m, 1H), 5.43-5.21 (m, 1H), 4.96-4.89 (m, 2H), 4.55-4.47 (m, 4H), 4.33 (br s, 2H), 3.99-3.89 (m, 2H), 3.80 (br d, J=4.5 Hz, 2H), 3.43-3.17 (m, 3H), 3.07-2.96 (m, 1H), 2.73 (s, 3H), 2.56-2.49 (m, 3H), 2.31 (br s, 1H), 2.26-2.14 (m, 2H), 2.09-1.91 (m, 4H), 0.79-0.63 (m, 2H), 0.22-0.09 (m, 2H).

LCMS (5% to 95% acetonitrile in water+0.03% ammonium bicarbonate over 6 min); retention time 3.141 min. ESI+ found [M+H]=743.3.

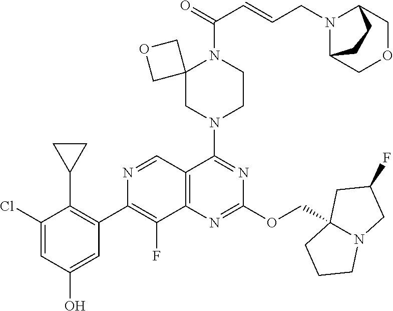

### Example 29 (Method 3): (E)-4-((1R,5S)-3-oxa-8-azabicyclo[3.2.1]octan-8-yl)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

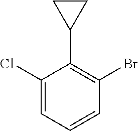 [SMILES: Clc1cccc(Br)c1C1CC1]

### Step 1: 1-bromo-3-chloro-2-cyclopropyl-benzene

To a solution of 1-bromo-3-chloro-2-iodo-benzene (25 g, 78.78 mmol) in dioxane (240 mL) and water (80 mL) was added cyclopropylboronic acid (8.80 g, 102.41 mmol), potassium phosphate (60.20 g, 283.60 mmol) and [1,1-Bis(diphenylphosphino) ferrocene]dichloropalladium (II)(5.76 g, 7.88 mmol). The mixture was degassed and purged with nitrogen three times, heated to 100° C., and stirred for 12 h. The mixture was diluted with water (80 mL) and extracted with ethyl acetate (3×100 mL). The combined organic layers were dried over sodium sulphate and concentrated in vacuo. The resulting residue was purified by reverse phase HPLC column: (column: Welch Xtimate C18 180*70 mm #10 μm; mobile phase: [water (NH₄HCO₃)-ACN]; B %: 65%-95%, 15 min) affording 1-bromo-3-chloro-2-cyclopropyl-benzene (9.4 g, 51.54%) as a colorless oil: ¹H NMR (400 MHz, Chloroform-d) & 7.52-7.43 (m, 1H), 7.31 (dd, J=1.1, 8.0 Hz, 1H), 7.00 (dt, J=0.8, 8.0 Hz, 1H), 1.84-1.71 (m, 1H), 1.23-1.15 (m, 2H), 0.84-0.72 (m, 2H).

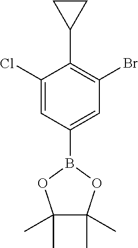 [SMILES: CC1(C)OB(c2cc(Cl)c(C3CC3)c(Br)c2)OC1(C)C]

### Step 2: 2-(3-bromo-5-chloro-4-cyclopropyl-phenyl)-4,4,5,5-tetramethyl-1,3,2-dioxaborolane

To a solution of 1-bromo-3-chloro-2-cyclopropyl-benzene (4.4 g, 1 eq) in hexane (40 mL) was added 4,4,5,5-tetramethyl-1,3,2-dioxaborolane (7.30 g, 57.02 mmol), (1,5-cyclooctadiene)(methoxy) iridium (I) dimer (629.89 mg, 950.26 μmol) and 4-tert-butyl-2-(4-tert-butyl-2-pyridyl)pyridine (306.05 mg, 1.14 mmol). The mixture was degassed and purged with nitrogen three times, heated to 60° C., and stirred for 4 h. The mixture was diluted with water (30 mL) and extracted with ethyl acetate (3×40 mL). The combined organic layers were dried over sodium sulphate and concentrated in vacuo. The residue was purified by column chromatography (silica gel, 100-200 mesh, 0-5% ethyl acetate in petroleum ether) affording 2-(3-bromo-5-chloro-4-cyclopropyl-phenyl)-4,4,5,5-tetramethyl-1,3,2-dioxaborolane (4.8 g, 63.59%) as a colorless oil: ¹H NMR (400 MHz, Chloroform-d) δ 7.87 (s, 1H), 7.71 (s, 1H), 1.84-1.74 (m, 1H), 1.34 (s, 12H), 1.22-1.15 (m, 2H), 0.79-0.76 (m, 2H).

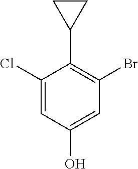 [SMILES: Oc1cc(Cl)c(C2CC2)c(Br)c1]

### Step 3: 3-bromo-5-chloro-4-cyclopropyl-phenol

To a solution of 2-(3-bromo-5-chloro-4-cyclopropyl-phenyl)-4,4,5,5-tetramethyl-1,3,2-dioxaborolane (3.7 g, 10.35 mmol) in tetrahydrofuran (20 mL) and water (20 mL) was added sodium 1,2,3-dioxaboriran-3-olate tetrahydrate (6.69 g, 43.47 mmol). The mixture was stirred at 25° C. for 4 h. The mixture was diluted with water (10 mL) and extracted with ethyl acetate (3×20 mL). The combined organic layers were dried over sodium sulphate and concentrated in vacuo. The residue was purified by column chromatography (silica gel, 100-200 mesh, 0-2% ethyl acetate in petroleum ether) affording 3-bromo-5-chloro-4-cyclopropyl-phenol (2.3 g, 80.80%) as a colorless oil: 1H NMR (400 MHz, Chloroform-d) δ 7.02-7.01 (d, J=2.4 Hz, 1H), 6.85 (d, J=2.4 Hz, 1H), 1.72-1.59 (m, 1H), 1.15-1.09 (m, 2H), 0.75-0.66 (m, 2H). LCMS Rt=0.795 min, m/z=248.9 [M+H]⁺.

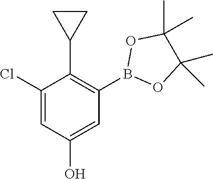 [SMILES: CC1(C)OB(c2cc(O)cc(Cl)c2C2CC2)OC1(C)C]

### Step 4: 3-chloro-4-cyclopropyl-5-(4,4,5,5-tetramethyl-1,3,2-dioxaborolan-2-yl) phenol

To a solution of 3-bromo-5-chloro-4-cyclopropyl-phenol (2.8 g, 11.31 mmol) in dioxane (50 mL) was added 4,4,5,5-tetramethyl-2-(4,4,5,5-tetramethyl-1,3,2-dioxaborolan-2-yl)-1,3,2-dioxaborolane (5.75 g, 22.62 mmol), potassium acetate (3.33 g, 33.94 mmol) and [1,1-Bis(diphenylphosphino) ferrocene]dichloropalladium (II)(827.74 mg, 1.13 mmol). The mixture was degassed with nitrogen three times, heated to 100° C. and stirred for 12 h. The mixture was diluted with water (40 mL) and extracted with ethyl acetate (3×50 mL). The combined organic layers were dried over sodium sulphate and concentrated in vacuo. The residue was purified by column chromatography (silica gel, 100-200 mesh, 0-30% ethyl acetate in petroleum ether) affording 3-chloro-4-cyclopropyl-5-(4,4,5,5-tetramethyl-1,3,2-dioxaborolan-2-yl) phenol (3.5 g, crude) as a colorless oil: ¹H NMR (400 MHz, Chloroform-d) δ 6.92 (d, J=2.8 Hz, 1H), 6.88 (d, J=2.8 Hz, 1H), 2.00-1.89 (m, 1H), 1.40-1.36 (m, 12H), 1.01-0.93 (m, 2H), 0.50 (q, J=5.5 Hz, 2H).

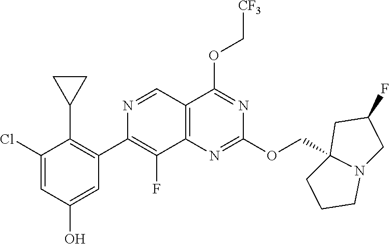 [SMILES: Oc1cc(Cl)c(C2CC2)c(-c2ncc3c(OCC(F)(F)F)nc(OC[C@@]45CCCN4C[C@H](F)C5)nc3c2F)c1]

### Step 5: 3-chloro-4-cyclopropyl-5-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl) phenol

The Suzuki reaction was prepared in a similar fashion to Method #3, Step 4. The reaction mixture was diluted with water (20 mL) and extracted with ethyl acetate (2×60 mL). The combined organic layers were dried over sodium sulphate and concentrated in vacuo. The resulting residue was purified by column chromatography (silica gel, 100-200 mesh, 10% ethyl acetate in petroleum ether) affording 3-chloro-4-cyclopropyl-5-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl) phenol (600 mg, 69.17%) as yellow gum. LCMS Rt=2.108 min, m/z=571.2 [M+H]⁺.

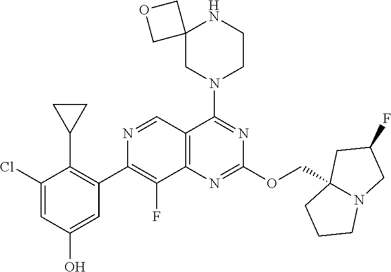 [SMILES: Oc1cc(Cl)c(C2CC2)c(-c2ncc3c(N4CCNC5(COC5)C4)nc(OC[C@@]45CCCN4C[C@H](F)C5)nc3c2F)c1]

### Step 6: 3-chloro-4-cyclopropyl-5-(8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl)pyrido[4,3-d]pyrimidin-7-yl) phenol

The substitution reaction was prepared in a similar fashion to Method #6, Step 5. The residue was purified by prep-HPLC (column: Waters Xbridge Prep OBD C18 150*40 mm*10 μm; mobile phase: [water (NH₄HCO₃)-ACN]; B %: 30%-60%, 8 min) affording 3-chloro-4-cyclopropyl-5-(8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl)pyrido[4,3-d]pyrimidin-7-yl) phenol (390 mg, 74.34%) as a white solid. LCMS Rt=0.509 min, m/z=599.2 [M+H]⁺.

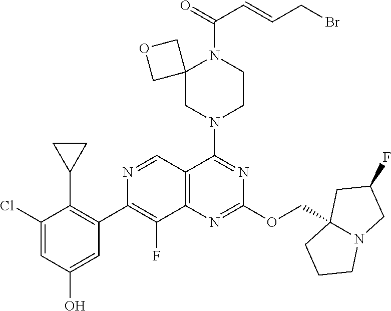 [SMILES: O=C(/C=C/CBr)N1CCN(c2nc(OC[C@@]34CCCN3C[C@H](F)C4)nc3c(F)c(-c4cc(O)cc(Cl)c4C4CC4)ncc23)CC12COC2]

### Step 7: (E)-4-bromo-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The amide coupling reaction was prepared in a similar fashion to Method #3, Step 2. The mixture was concentrated to dryness in vacuo affording (E)-4-bromo-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (100 mg, crude) as yellow oil, which was used in the next step without any further purification. LCMS Rt=0.594 min, m/z=747.2 [M+H]⁺.

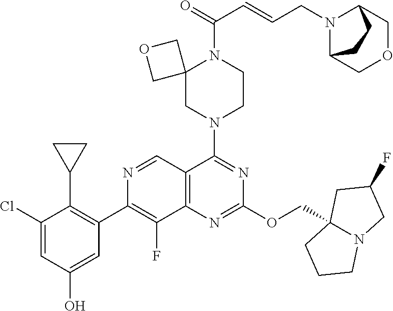

### Step 8: (E)-4-((1R,5S)-3-oxa-8-azabicyclo[3.2.1]octan-8-yl)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-ACN]; B %: 35%-65%, 8 min) affording (E)-4-((1R,5S)-3-oxa-8-azabicyclo[3.2.1]octan-8-yl)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (11.75 mg, 11.26%) as yellow amorphous solid: ¹H NMR (400 MHz, Chloroform-d) δ 8.97 (s, 1H), 7.00-6.90 (m, 2H), 6.79-6.65 (m, 1H), 6.57-6.41 (m, 1H), 5.48-5.24 (m, 1H), 4.97-4.75 (m, 2H), 4.36 (br s, 6H), 3.96-3.71 (m, 4H), 3.60-3.51 (m, 5H), 3.38-3.25 (m, 1H), 3.20-3.06 (m, 5H), 2.04 (br s, 4H), 1.94 (br s, 3H), 1.94-1.87 (m, 4H), 1.86-1.80 (m, 2H), 0.63-0.55 (m, 2H), 0.07-0.03 (m, 2H). LCMS (5% to 95% acetonitrile in water+0.03% ammonium bicarbonate over 6 min); retention time 2.875 min, ESI+ found [M+H]=778.3.

### Example 30 (Method 3): (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

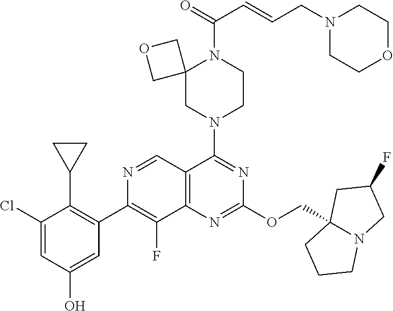 [SMILES: O=C(/C=C/CN1CCOCC1)N1CCN(c2nc(OC[C@@]34CCCN3C[C@H](F)C4)nc3c(F)c(-c4cc(O)cc(Cl)c4C4CC4)ncc23)CC12COC2]

### Step 1: (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one

The substitution reaction was prepared in a similar fashion to Method #3, Step 3. The residue was purified by prep-HPLC (column: Waters Xbridge Prep OBD C18 150*40 mm*10 μm; mobile phase: [water (NH₄HCO₃)-ACN]; B %: 35%-65%, 8 min) affording (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinobut-2-en-1-one (10.7 mg, 9.47%) as pale yellow solid: ¹H NMR (400 MHz, Chloroform-d) δ 8.96 (s, 1H), 7.02-6.91 (m, 2H), 6.68 (br s, 1H), 6.41 (br d, J=15.3 Hz, 1H), 5.45-5.25 (m, 1H), 4.86 (br d, J=6.6 Hz, 2H), 4.53-4.39 (m, 5H), 4.33 (br d, J=10.6 Hz, 1H), 3.84 (br s, 2H), 3.77-3.73 (m, 4H), 3.56 (br s, 2H), 3.50-3.42 (m, 1H), 3.33-3.26 (m, 1H), 3.20 (br d, J=5.6 Hz, 2H), 3.11-3.01 (m, 1H), 2.52 (br d, J=4.0 Hz, 4H), 2.38-2.26 (m, 2H), 2.22-2.16 (m, 1H), 2.10-1.97 (m, 4H), 1.85 (br d, J=6.0 Hz, 2H), 0.67-0.54 (m, 2H), 0.15-0.09 (m, 2H). LCMS (5% to 95% acetonitrile in water+0.03% ammonium bicarbonate over 6 min); retention time 2.804 min, ESI+ found [M+H]=752.3.

### Example 31 (Method 1): 1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

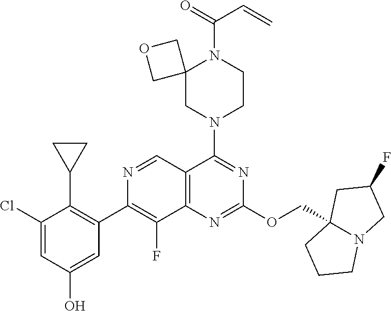 [SMILES: C=CC(=O)N1CCN(c2nc(OC[C@@]34CCCN3C[C@H](F)C4)nc3c(F)c(-c4cc(O)cc(Cl)c4C4CC4)ncc23)CC12COC2]

### Step 1: 1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The residue was purified by prep-HPLC (column: Waters Xbridge Prep OBD C18 150*40 mm*10 μm; mobile phase: [water (NH₄HCO₃)-ACN]; B %: 30%-60%, 8 min) affording 1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (80.32 mg, 24.56%) as a yellow solid: 1H NMR (400 MHz, Chloroform-d) δ 8.91 (s, 1H), 6.94-6.84 (m, 1H), 6.73-6.63 (m, 1H), 6.56-6.32 (m, 2H), 5.90-5.74 (m, 1H), 5.42-5.13 (m, 1H), 4.93-4.76 (m, 2H), 4.45-4.21 (m, 6H), 3.91-3.73 (m, 2H), 3.57-3.43 (m, 2H), 3.33 (br s, 3H), 3.05-2.96 (m, 1H), 2.39-2.29 (m, 1H), 2.27-2.20 (m, 1H), 2.15-2.07 (m, 1H), 2.01-1.89 (m, 3H), 1.83-1.75 (m, 1H), 0.66-0.47 (m, 2H), 0.11-0.17 (m, 2H).

LCMS (5% to 95% acetonitrile in water+0.03% ammonium bicarbonate over 6 min) retention time 2.880 min, ESI+ found [M+H]=653.2.

### Example 32 (Method 2): (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

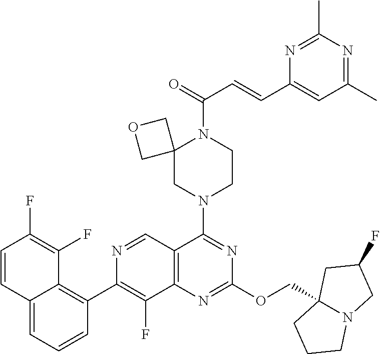 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)CC23COC3)nc(C)n1]

### Step 1: (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one

The amide coupling reaction was prepared in a similar fashion to Method #2, Step 3. The crude product was purified by reverse phase HPLC (column: Waters Xbridge BEH C18 100*30 mm*10 μm; mobile phase: [water (NH₄HCO₃)-ACN]; B %: 35%-65%, 8 min) affording (E)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl) prop-2-en-1-one (15.16 mg, 13.77%) as a pale yellow amorphous solid: ¹H NMR (400 MHz, Chloroform-d) δ 9.13-9.06 (m, 1H), 7.98 (d, J=8.0 Hz, 1H), 7.76-7.58 (m, 4H), 7.56-7.49 (m, 1H), 7.41 (dt, J=7.4, 9.3 Hz, 1H), 7.01-6.97 (m, 1H), 5.39-5.20 (m, 1H), 5.00-4.86 (m, 2H), 4.63-4.41 (m, 4H), 4.39-4.24 (m, 2H), 4.07-3.68 (m, 4H), 3.39-3.15 (m, 3H), 3.04-2.95 (m, 1H), 2.77-2.69 (m, 3H), 2.58-2.49 (m, 3H), 2.34-2.11 (m, 3H), 2.02-1.89 (m, 3H). LCMS (5% to 95% acetonitrile in water+0.03% ammonium bicarbonate over 6 min); retention time 2.986 min, ESI+ found [M+H]=755.3.

### Example 33 (Method 1): 1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

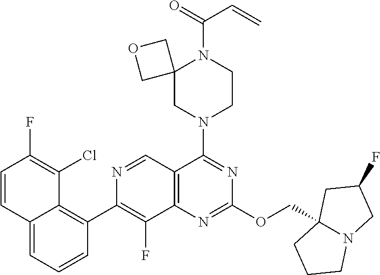 [SMILES: C=CC(=O)N1CCN(c2nc(OC[C@@]34CCCN3C[C@H](F)C4)nc3c(F)c(-c4cccc5ccc(F)c(Cl)c45)ncc23)CC12COC2]

### Step 1: 1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The crude product was purified by reverse phase HPLC (column: Waters Xbridge Prep OBD C18 150*40 mm*10 μm; mobile phase: [water (NH₄HCO₃)-ACN]; B %: 30%-60%, 8 min) affording 1-(8-(7-(8-chloro-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (15.16 mg, 13.77%) as a yellow solid: 1H NMR (400 MHz, Acetonitrile-d3) δ 9.13 (s, 1H), 8.17 (dd, J=3.4, 6.1 Hz, 1H), 8.11 (dd, J=5.6, 9.2 Hz, 1H), 7.73-7.69 (m, 2H), 7.56 (t, J=9.0 Hz, 1H), 6.69 (dd, J=10.5, 16.7 Hz, 1H), 6.31 (dd, J=2.0, 16.7 Hz, 1H), 5.80 (dd, J=2.0, 10.4 Hz, 1H), 5.39-5.21 (m, 1H), 4.90 (t, J=7.1 Hz, 2H), 4.58 (dd, J=7.0, 13.2 Hz, 1H), 4.51-4.46 (m, 2H), 4.42 (dd, J=5.8, 12.9 Hz, 1H), 4.28-4.24 (m, 1H), 4.20-4.15 (m, 1H), 3.97 (t, J=5.1 Hz, 2H), 3.76-3.59 (m, 2H), 3.22-3.13 (m, 2H), 3.10 (s, 1H), 2.98-2.88 (m, 1H), 2.23-2.21 (m, 1H), 2.14 (br d, J=2.6 Hz, 1H), 2.12-2.05 (m, 1H), 1.95-1.83 (m, 3H).

LCMS (5% to 95% acetonitrile in water+0.03% ammonium bicarbonate over 6 min); retention time 1.807 min, ESI+ found [M+H]=665.2.

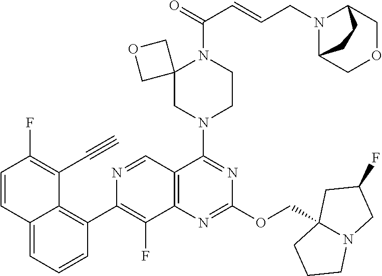

### Example 34 (Method 3): (E)-4-((1R,5S)-3-oxa-8-azabicyclo[3.2.1]octan-8-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

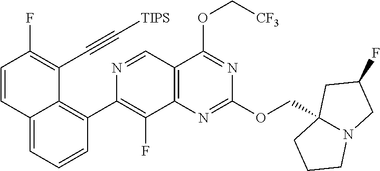

### Step 1: 8-fluoro-7-(7-fluoro-8-((triisopropylsilyl) ethynyl) naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The crude residue was purified by column chromatography (silica gel, 100-200 mesh, 20-50% ethyl acetate in petroleum ether) affording 8-fluoro-7-(7-fluoro-8-((triisopropylsilyl) ethynyl) naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (4.2 g, 84.28%) as a yellow solid. LCMS Rt=0.863 min, m/z=729.3 [M+H]⁺.

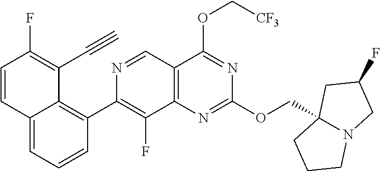 [SMILES: C#Cc1c(F)ccc2cccc(-c3ncc4c(OCC(F)(F)F)nc(OC[C@@]56CCCN5C[C@H](F)C6)nc4c3F)c12]

### Step 2: 7-(8-ethynyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

To a solution of 8-fluoro-7-(7-fluoro-8-((triisopropylsilyl) ethynyl) naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (3 g, 4.12 mmol) in diisopropylethylamine (45 mL) was added cesium fluoride (625.25 mg, 4.12 mmol). The mixture was stirred at 0° C. for 1 h. The mixture was diluted with water (40 mL) and extracted with ethyl acetate (3×40 mL). The combined organic layers were dried over sodium sulphate and concentrated in vacuo. The crude residue was purified by column chromatography (silica gel, 100-200 mesh, 20-50% ethyl acetate in petroleum ether) affording 7-(8-ethynyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (2.2 g, 93.36%) as a yellow solid. LCMS Rt=1.552 min, m/z=573.2 [M+H]⁺.

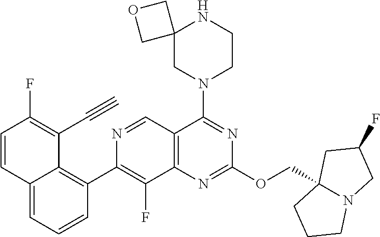 [SMILES: C#Cc1c(F)ccc2cccc(-c3ncc4c(N5CCNC6(COC6)C5)nc(OC[C@@]56CCCN5C[C@H](F)C6)nc4c3F)c12]

### Step 3: 8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The reaction mixture was concentrated in vacuo and purified by reverse phase HPLC (column: Waters Xbridge BEH C18 250*50 mm*10 μm; mobile phase: [water (NH₄HCO₃)-ACN]; B %: 30%-60%, 10 min) affording 8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (500 mg, 95.32%) as a yellow solid. LCMS Rt=1.830 min, m/z=601.3 [M+H]⁺.

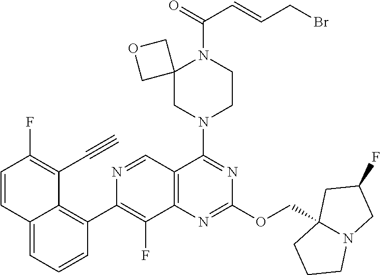 [SMILES: C#Cc1c(F)ccc2cccc(-c3ncc4c(N5CCN(C(=O)/C=C/CBr)C6(COC6)C5)nc(OC[C@@]56CCCN5C[C@H](F)C6)nc4c3F)c12]

### Step 4: (E)-4-bromo-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The amide coupling reaction was prepared in a similar fashion to Method #3, Step 2. The reaction mixture was concentrated in vacuo affording (E)-4-bromo-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (224 mg, crude) as a yellow solid, which was used in the next step without further purification. LCMS Rt=0.791 min, m/z=747.2 [M+H]⁺.

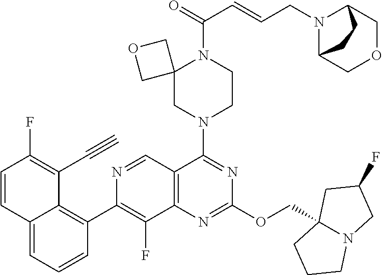

### Step 5: (E)-4-((1R,5S)-3-oxa-8-azabicyclo[3.2.1]octan-8-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-4-((1R,5S)-3-oxa-8-azabicyclo[3.2.1]octan-8-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (54.4 mg, 26.08%) as a white amorphous solid: ¹H NMR (400 MHz, Acetonitrile-d3) δ 9.09 (s, 1H), 8.19-8.11 (m, 2H), 7.73-7.64 (m, 2H), 7.48 (t, J=9.1 Hz, 1H), 6.80 (td, J=5.5, 15.2 Hz, 1H), 6.54 (br d, J=15.1 Hz, 1H), 5.38-5.19 (m, 1H), 4.86 (dd, J=6.6, 19.4 Hz, 2H), 4.58-4.48 (m, 2H), 4.48-4.41 (m, 1H), 4.38 (d, J=6.5 Hz, 1H), 4.28-4.22 (m, 1H), 4.19-4.14 (m, 1H), 4.11-4.01 (m, 1H), 3.86-3.77 (m, 1H), 3.72-3.61 (m, 4H), 3.49 (dd, J=1.4, 10.3 Hz, 2H), 3.33 (s, 1H), 3.23-3.14 (m, 2H), 3.11 (dd, J=1.5, 5.8 Hz, 3H), 3.04 (br s, 2H), 2.97-2.88 (m, 1H), 2.14 (br s, 4H), 1.94-1.78 (m, 6H).

LCMS (5% to 95% acetonitrile in water+0.03% ammonium bicarbonate over 6 min); retention time 2.691 min, ESI+ found [M+H]=780.3.

### Example 35 (Method 1): 1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

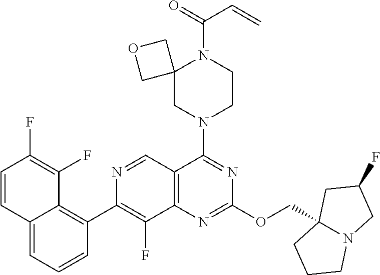 [SMILES: C=CC(=O)N1CCN(c2nc(OC[C@@]34CCCN3C[C@H](F)C4)nc3c(F)c(-c4cccc5ccc(F)c(F)c45)ncc23)CC12COC2]

### Step 1: 1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The crude product was purified by reverse phase HPLC (column: Waters Xbridge BEH C18 100*30 mm*10 μm; mobile phase: [water (NH₄HCO₃)-ACN]; B %: 30%-60%, 8 min) affording 1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (8.83 mg, 16.19%) as a yellow amorphous solid: ¹H NMR (400 MHz, Chloroform-d) δ 9.09 (s, 1H), 8.02-7.95 (m, 1H), 7.73 (dt, J=3.6, 4.5 Hz, 1H), 7.68-7.59 (m, 2H), 7.45-7.37 (m, 1H), 6.60-6.48 (m, 1H), 6.46-6.38 (m, 1H), 5.88-5.81 (m, 1H), 5.42-5.23 (m, 1H), 4.96-4.83 (m, 2H), 4.60-4.40 (m, 4H), 4.39-4.27 (m, 2H), 4.05-3.80 (m, 2H), 3.75-3.55 (m, 2H), 3.45-3.18 (m, 3H), 3.09-2.96 (m, 1H), 2.37-2.13 (m, 3H), 2.07-1.91 (m, 3H).

LCMS (5% to 95% acetonitrile in water+0.03% ammonium bicarbonate over 6 min); retention time 2.977 min, ESI+ found [M+H]=649.3.

### Example 36 (Method 3): (E)-4-((1R,5S)-3-oxa-8-azabicyclo[3.2.1]octan-8-yl)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

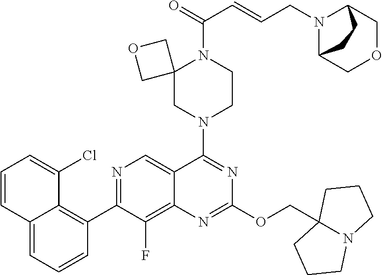

### Step 1: (E)-4-((1R,5S)-3-oxa-8-azabicyclo[3.2.1]octan-8-yl)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-4-((1R,5S)-3-oxa-8-azabicyclo[3.2.1]octan-8-yl)-1-(8-(7-(8-chloronaphthalen-1-yl)-8-fluoro-2-((tetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (10.04 mg, 22.78%) as a yellow solid: ¹H NMR (400 MHz, Acetonitrile-d3) δ 9.11 (s, 1H), 8.15 (dd, J=1.1, 8.1 Hz, 1H), 8.04 (d, J=7.4 Hz, 1H), 7.75-7.68 (m, 1H), 7.67-7.61 (m, 2H), 7.57-7.51 (m, 1H), 6.82 (td, J=5.5, 15.2 Hz, 1H), 6.55 (br d, J=15.3 Hz, 1H), 4.87 (t, J=6.9 Hz, 2H), 4.55 (d, J=13.0 Hz, 1H), 4.47 (t, J=5.8 Hz, 2H), 4.40 (d, J=13.0 Hz, 1H), 4.23-4.15 (m, 2H), 3.95 (t, J=5.1 Hz, 2H), 3.74-3.57 (m, 4H), 3.48 (dd, J=1.4, 10.3 Hz, 2H), 3.10 (dd, J=1.4, 5.5 Hz, 2H), 3.07-2.95 (m, 4H), 2.63 (td, J=6.8, 9.9 Hz, 2H), 2.03-1.98 (m, 2H), 1.93 (br d, J=4.8 Hz, 2H), 1.90-1.80 (m, 6H), 1.71-1.61 (m, 2H).

LCMS (5% to 95% acetonitrile in water+0.03% ammonium bicarbonate over 6 min); retention time 2.826 min, ESI+ found [M+H]=754.3.

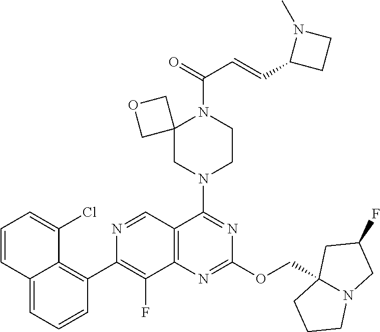 [SMILES: CN1CC[C@@H]1/C=C/C(=O)N1CCN(c2nc(OC[C@@]34CCCN3C[C@H](F)C4)nc3c(F)c(-c4cccc5cccc(Cl)c45)ncc23)CC12COC2]

### Example 37 (Method 5): (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one

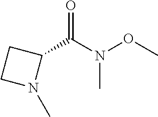

### Step 1: (R)—N-methoxy-N,1-dimethylazetidine-2-carboxamide

To a solution of N,O-dimethylhydroxylamine hydrochloride (169.45 mg, 1.74 mmol) in N,N-dimethylformaldehyde (3 mL) was added (2R)-1-methylazetidine-2-carboxylic acid (100 mg, 868.58 μmol), 1-benzotriazolol (234.73 mg, 1.74 mmol), 1-(3-Dimethylaminopropyl)-3-ethylcarbodiimide hydrochloride (333.01 mg, 1.74 mmol) and 4-methylmorpholine (263.57 mg, 2.61 mmol, 286.49 μL). The mixture was stirred at 25° C. for 12 h. The reaction mixture was concentrated in vacuo and purified by reverse phase HPLC (column: Phenomenex Luna C18 100*30 mm*5 μm; mobile phase: [water (TFA)-acetonitrile]; B %: 1%-20%, 10 min) affording (R)—N-methoxy-N,1-dimethylazetidine-2-carboxamide (200 mg, crude) as a white oil. LCMS Rt=0.640 min, m/z=159.1 [M+H]⁺.

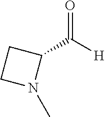

### Step 2: (R)-1-methylazetidine-2-carbaldehyde

A mixture of (R)—N-methoxy-N,1-dimethylazetidine-2-carboxamide (100 mg, 632.12 μmol) in tetrahydrofuran (2 mL) was degassed and purged with nitrogen 3 times. Lithium aluminum hydride (47.98 mg, 1.26 mmol) was added to the mixture at 0° C., and the mixture was stirred at 0° C. for 1 h under a nitrogen atmosphere. The reaction mixture was quenched with saturated sodium sulfite (2 mL) and extracted with ethyl acetate (2×3 mL). The combined organic layers were dried over sodium sulfate, filtered and concentrated in vacuo affording (R)-1-methylazetidine-2-carbaldehyde (60 mg, crude) as a yellow oil, which was used in the next step without further purification: ¹H NMR (400 MHz, Acetonitrile-d3) δ 3.70-3.81 (m, 1H), 3.14-3.39 (m, 2H), 2.19-2.29 (m, 3H), 1.64-1.76 (m, 2H).

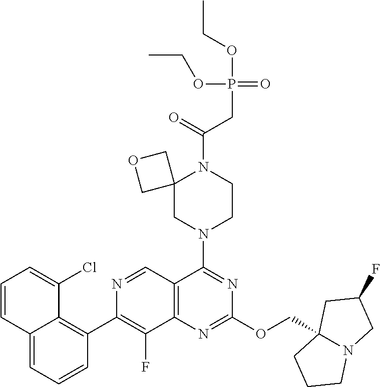 [SMILES: CCOP(=O)(CC(=O)N1CCN(c2nc(OC[C@@]34CCCN3C[C@H](F)C4)nc3c(F)c(-c4cccc5cccc(Cl)c45)ncc23)CC12COC2)OCC]

### Step 3: diethyl(2-(8-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonan-5-yl)-2-oxoethyl)phosphonate

To a solution of 8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (100 mg, 141.43 μmol, trifluoroacetate salt) and 2-diethoxyphosphorylacetic acid (55.48 mg, 282.85 μmol, 45.47 μL) in dichloromethane (2.0 mL) was added diisopropylethylamine (54.83 mg, 424.28 μmol) and O-(7-Azabenzotriazol-1-yl)-N,N,N′,N′-tetramethyluronium Hexafluorophosphate (80.66 mg, 212.14 μmol). The mixture was stirred at 0° C. for 1 h. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 40%-60%, 8 min) affording diethyl(2-(8-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonan-5-yl)-2-oxoethyl)phosphonate (50 mg, 45.84%) as a yellow solid. LCMS Rt=1.766 min, m/z=771.3 [M+H]⁺.

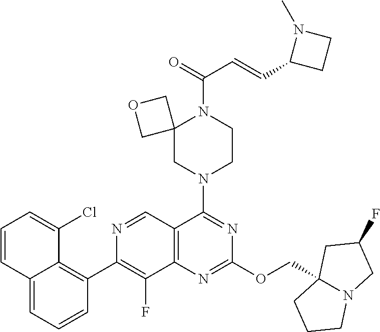 [SMILES: CN1CC[C@@H]1/C=C/C(=O)N1CCN(c2nc(OC[C@@]34CCCN3C[C@H](F)C4)nc3c(F)c(-c4cccc5cccc(Cl)c45)ncc23)CC12COC2]

### Step 4: (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one

To a solution of diethyl(2-(8-(7-(8-chloronaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-oxa-5,8-diazaspiro[3.5]nonan-5-yl)-2-oxoethyl)phosphonate (30 mg, 38.90 μmol) in acetonitrile (1 mL) was added diisopropylethylamine (15.08 mg, 116.70 μmol), lithium chloride (4.95 mg, 116.70 μmol) and (2R)-1-methylazetidine-2-carbaldehyde (7.71 mg, 77.80 μmol) at 0° C. The mixture was stirred at 25° C. for 1 h. The reaction mixture was concentrated in vacuo and purified by reverse phase HPLC (column: Waters Xbridge Prep OBD C18 150*40 mm*10 μm; mobile phase: [water (FA)-acetonitrile]; B %: 40%-60%, 8 min) affording (E)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one (1.89 mg, 6.37%, formate salt) as a yellow solid: ¹H NMR (Acetonitrile-d3) δ 9.05-9.15 (m, 1H), 8.21 (br d, 1H), 8.13 (d, 1H), 8.03 (d, 1H), 7.67-7.73 (m, 1H), 7.60-7.66 (m, 2H), 7.48-7.56 (m, 1H), 6.85 (m, 1H), 6.50 (br d, 1H), 5.16-5.41 (m, 1H), 4.86 (br m, 2H), 4.49-4.61 (m, 1H), 4.35-4.47 (m, 3H), 4.21-4.33 (m, 2H), 3.94 (br m, 2H), 3.80-3.90 (m, 1H), 3.57-3.76 (m, 2H), 3.46 (br m, 1H), 3.15-3.30 (m, 3H), 2.94-3.06 (m, 2H), 2.35 (s, 3H), 2.20-2.29 (m, 2H), 2.18 (br s, 1H), 2.03-2.14 (m, 2H), 2.01 (br s, 2H), 1.84-1.92 (m, 1H). LCMS (5% to 95% acetonitrile in water+0.03% ammonium bicarbonate over 6 min); retention time 1.943 min, ESI+ found [M+H]=716.3.

### Example 38 (Method 1): 1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

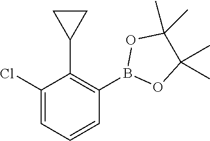 [SMILES: CC1(C)OB(c2cccc(Cl)c2C2CC2)OC1(C)C]

### Step 1: 2-(3-chloro-2-cyclopropylphenyl)-4,4,5,5-tetramethyl-1,3,2-dioxaborolane

To a solution of 1-bromo-3-chloro-2-cyclopropyl-benzene (4 g, 17.28 mmol) in dioxane (40 mL) were added 4,4,5,5-tetramethyl-2-(4,4,5,5-tetramethyl-1,3,2-dioxaborolan-2-yl)-1,3,2-dioxaborolane (8.77 g, 34.55 mmol), potassium acetate (5.09 g, 51.83 mmol) and [1,1-Bis(diphenylphosphino) ferrocene]dichloropalladium (II)(1.26 g, 1.73 mmol). The mixture was degassed and purged with nitrogen three times and subsequently stirred at 100° C. for 12 h. The mixture was diluted with water (30 mL) and extracted with ethyl acetate (3×40 mL). The combined organic layers were dried over sodium sulphate and concentrated in vacuo. The residue was purified by column chromatography (silica gel, 100-200 mesh, 0-5% ethyl acetate in petroleum ether) affording 2-(3-chloro-2-cyclopropylphenyl)-4,4,5,5-tetramethyl-1,3,2-dioxaborolane (7 g, 54.54%) as a pale yellow solid: ¹H NMR (400 MHz, Chloroform-d) δ 7.47-7.43 (m, 1H), 7.39-7.35 (m, 1H), 7.15-7.12 (m, 1H), 2.12-2.02 (m, 1H), 1.40-1.38 (m, 12H), 1.07-1.03 (m, 1H), 1.02-1.00 (m, 1H), 0.61-0.55 (m, 2H).

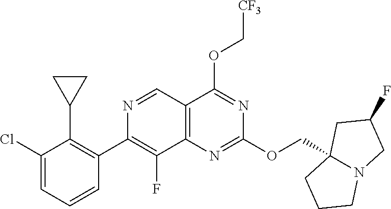 [SMILES: Fc1c(-c2cccc(Cl)c2C2CC2)ncc2c(OCC(F)(F)F)nc(OC[C@@]34CCCN3C[C@H](F)C4)nc12]

### Step 2: 7-(3-chloro-2-cyclopropylphenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The mixture were concentrated in vacuo. The resulting residue was purified by column chromatography (silica gel, 100-200 mesh, 0-50% ethyl acetate in petroleum ether) affording 7-(3-chloro-2-cyclopropylphenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (170 mg, 30.04%) as an orange oil. LCMS Rt=0.693 min, m/z=555.2 [M+H]⁺.

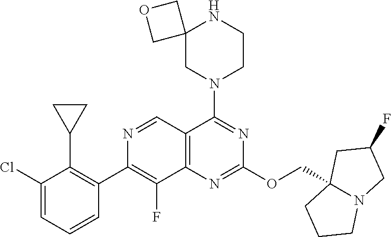 [SMILES: Fc1c(-c2cccc(Cl)c2C2CC2)ncc2c(N3CCNC4(COC4)C3)nc(OC[C@@]34CCCN3C[C@H](F)C4)nc12]

### Step 3: 8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The reaction mixture was concentrated in vacuo affording 8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (150 mg, crude) as a brown oil, which was used in the next step without further purification. LCMS Rt=0.509 min, m/z=583.2 [M+H]⁺.

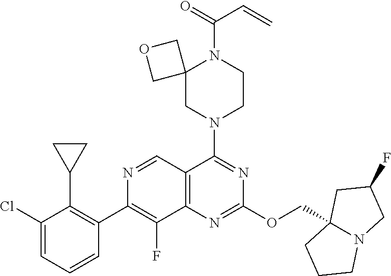 [SMILES: C=CC(=O)N1CCN(c2nc(OC[C@@]34CCCN3C[C@H](F)C4)nc3c(F)c(-c4cccc(Cl)c4C4CC4)ncc23)CC12COC2]

### Step 4: 1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The resulting residue was purified by reverse phase HPLC (column: Waters Xbridge Prep OBD C18 150*40 mm*10 μm; mobile phase: [water (NH₄HCO₃)-ACN]; B %: 35%-65%, 8 min) affording 1-(8-(7-(3-chloro-2-cyclopropylphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (5.51 mg, 5.04%) as a white solid: 1H

NMR (400 MHz, Chloroform-d) δ 9.12-9.07 (m, 1H), 7.54-7.47 (m, 1H), 7.42-7.36 (m, 1H), 7.34-7.29 (m, 1H), 6.60-6.50 (m, 1H), 6.46-6.37 (m, 1H), 5.88-5.81 (m, 1H), 5.45-5.20 (m, 1H), 4.93-4.85 (m, 2H), 4.53-4.43 (m, 4H), 4.43-4.27 (m, 2H), 3.97-3.86 (m, 2H), 3.71-3.60 (m, 2H), 3.58-3.18 (m, 3H), 3.10-2.97 (m, 1H), 2.43-2.14 (m, 3H), 2.11-1.91 (m, 4H), 0.78-0.66 (m, 2H), 0.21-0.11 (m, 2H).

LCMS (5% to 95% acetonitrile in water+0.03% ammonium bicarbonate over 6 min); retention time 3.133 min, ESI+ found [M+H]=637.2.

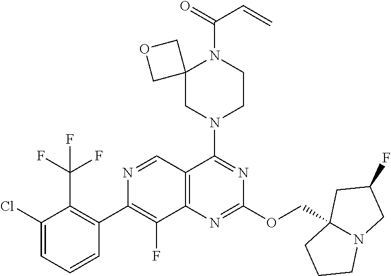 [SMILES: C=CC(=O)N1CCN(c2nc(OC[C@@]34CCCN3C[C@H](F)C4)nc3c(F)c(-c4cccc(Cl)c4C(F)(F)F)ncc23)CC12COC2]

### Example 39 (Method 1): 1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

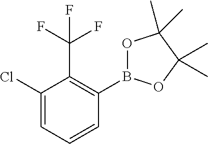 [SMILES: CC1(C)OB(c2cccc(Cl)c2C(F)(F)F)OC1(C)C]

### Step 1: 2-[3-chloro-2-(trifluoromethyl)phenyl]-4,4,5,5-tetramethyl-1,3,2-dioxaborolane

A solution of 1-bromo-3-chloro-2-(trifluoromethyl)benzene (5 g, 19.27 mmol), 4,4,5,5-tetramethyl-2-(4,4,5,5-tetramethyl-1,3,2-dioxaborolan-2-yl)-1,3,2-dioxaborolane (7.34 g, 28.91 mmol), potassium acetate (1.89 g, 19.27 mmol) and cis-dichloro-1,1′-bis(diphenylphosphino) ferrocene palladium (II)(1.41 g, 1.93 mmol) in dioxane (100 mL) was degassed and purged with nitrogen 3 times. The mixture was stirred at 100° C. for 12 h under a nitrogen atmosphere. The mixture was filtered, the filtrate was concentrated in vacuo and the residue was purified by column chromatography (silica gel, 100-200 mesh, 50% ethyl acetate in petroleum ether) affording 2-[3-chloro-2-(trifluoromethyl)phenyl]-4,4,5,5-tetramethyl-1,3,2-dioxaborolane (1.9 g, 32.17%) as a yellow oil.

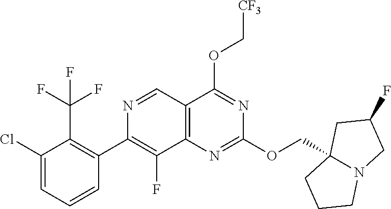 [SMILES: Fc1c(-c2cccc(Cl)c2C(F)(F)F)ncc2c(OCC(F)(F)F)nc(OC[C@@]34CCCN3C[C@H](F)C4)nc12]

### Step 2: 7-(3-chloro-2-(trifluoromethyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The reaction mixture was filtered, concentrated and purified by column chromatography (silica gel, 100-200 mesh, 2-30% ethyl acetate in petroleum ether) affording 7-(3-chloro-2-(trifluoromethyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (600 mg, 90.33%) as a yellow oil. LCMS Rt=1.009 min, m/z=583.1 [M+H]⁺.

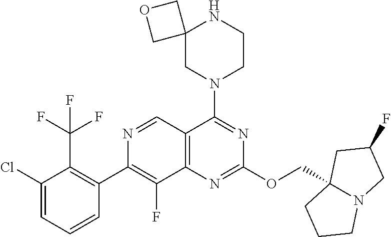 [SMILES: Fc1c(-c2cccc(Cl)c2C(F)(F)F)ncc2c(N3CCNC4(COC4)C3)nc(OC[C@@]34CCCN3C[C@H](F)C4)nc12]

### Step 3: 8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The reaction mixture was concentrated in vacuo and the crude product was purified by reverse phase HPLC (column: Waters Xbridge Prep OBD C18 150*40 mm*10 μm; mobile phase: [water (NH₄HCO₃)-ACN]; B %: 35%-65%, 8 min) affording 8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (150 mg, 28.62%) as a brown oil, which was used in the next step without further purification. LCMS Rt=1.889 min, m/z=611.2 [M+H]⁺

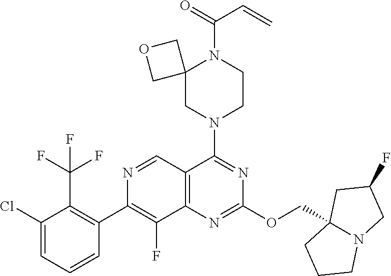 [SMILES: C=CC(=O)N1CCN(c2nc(OC[C@@]34CCCN3C[C@H](F)C4)nc3c(F)c(-c4cccc(Cl)c4C(F)(F)F)ncc23)CC12COC2]

### Step 4: 1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The resulting residue was purified by reverse phase HPLC (column: Waters Xbridge Prep OBD C18 150*40 mm*10 μm; mobile phase: [water (NH₄HCO₃)-ACN]; B %: 35%-65%, 8 min) affording 1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (29.5 mg, 54.21%) as a yellow solid: 1H NMR (400 MHz, Acetonitrile-d3) δ 9.09 (s, 1H), 7.87-7.64 (m, 2H), 7.48 (d, J=7.8 Hz, 1H), 6.67 (dd, J=10.5, 16.8 Hz, 1H), 6.29 (dd, J=2.0, 16.8 Hz, 1H), 5.79 (dd, J=2.0, 10.5 Hz, 1H), 5.40-5.15 (m, 1H), 4.88 (br d, J=5.5 Hz, 2H), 4.51 (br d, J=5.9 Hz, 1H), 4.45 (d, J=6.9 Hz, 3H), 4.27-4.23 (m, 1H), 4.20-4.14 (m, 1H), 3.93 (br d, J=4.5 Hz, 2H), 3.66 (br s, 2H), 3.22-3.11 (m, 2H), 3.09 (s, 1H), 2.97-2.88 (m, 1H), 2.23-2.19 (m, 1H), 2.13 (d, J=3.0 Hz, 1H), 2.11-2.03 (m, 1H), 1.93-1.82 (m, 3H).

LCMS (5% to 95% acetonitrile in water+0.03% ammonium bicarbonate over 6 min); retention time 3.111 min, ESI+ found [M+H]=665.2.

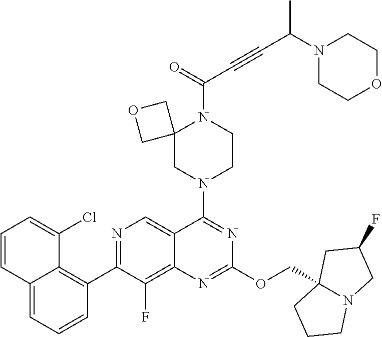 [SMILES: CC(C#CC(=O)N1CCN(c2nc(OC[C@@]34CCCN3C[C@H](F)C4)nc3c(F)c(-c4cccc5cccc(Cl)c45)ncc23)CC12COC2)N1CCOCC1]

### Example 40 (Method 2): 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinopent-2-yn-1-one

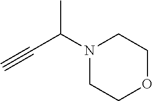

### Step 1: 4-(but-3-yn-2-yl) morpholine

To a solution of morpholine (3.50 g, 40.13 mmol) in methanol (40 mL) were added potassium carbonate (1.02 g, 7.36 mmol) and 1-methylprop-2-ynyl 4-methylbenzenesulfonate (3 g, 13.38 mmol) at 0° C. The mixture was warmed to 25° C. and stirred for 12 h. The reaction mixture was concentrated to dryness in vacuo. The crude product was purified by column chromatography (silica gel, 100-200 mesh, 0-100% ethyl acetate in petroleum ether) affording 4-(but-3-yn-2-yl) morpholine (1 g, 53.71%) as a colorless oil: ¹H NMR (400 MHz, Chloroform-d) δ 3.82-3.68 (m, 4H), 3.52-3.42 (m, 1H), 2.73-2.62 (m, 2H), 2.56-2.45 (m, 2H), 2.31 (d, J=2.1 Hz, 1H), 1.36 (d, J=7.1 Hz, 3H).

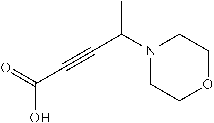

### Step 2: 4-morpholinopent-2-ynoic acid

To a solution of 4-(but-3-yn-2-yl) morpholine (1 g, 7.18 mmol) in tetrahydrofuran (10 mL) was added n-butyllithium (2.5 M, 3.16 mL) at −70° C. under nitrogen. The mixture was stirred at −70° C. for 0.5 h, and carbon dioxide (3.16 g, 71.84 mmol) was added at −70° C. The mixture was warmed to 25° C. and stirred for 2 h. The reaction mixture was quenched with saturated ammonium chloride (20 mL) and extracted with ethyl acetate (3×10 mL). The combined organic layers were dried over sodium sulfate, filtered and concentrated in vacuo affording 4-morpholinopent-2-ynoic acid (1 g, crude) as a white gum, which was used in the next step without further purification.

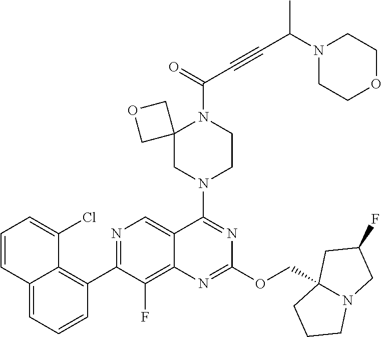 [SMILES: CC(C#CC(=O)N1CCN(c2nc(OC[C@@]34CCCN3C[C@H](F)C4)nc3c(F)c(-c4cccc5cccc(Cl)c45)ncc23)CC12COC2)N1CCOCC1]

### Step 3: 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinopent-2-yn-1-one

The coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-acetonitrile]; B %: 40%-70%, 8 min) affording 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-morpholinopent-2-yn-1-one (27.52 mg, 29.06%) as a white solid: ¹H NMR (400 MHz, Acetonitrile-d3) δ 9.16 (s, 1H), 8.16 (dd, J=1.1, 8.1 Hz, 1H), 8.05 (dd, J=0.9, 8.1 Hz, 1H), 7.75-7.69 (m, 1H), 7.67-7.62 (m, 2H), 7.58-7.49 (m, 1H), 5.38-5.19 (m, 1H), 5.08 (br d, J=4.8 Hz, 2H), 4.61-4.50 (m, 1H), 4.48-4.38 (m, 3H), 4.28-4.22 (m, 1H), 4.21-4.14 (m, 1H), 4.12-4.07 (m, 2H), 4.03-3.86 (m, 2H), 3.77-3.62 (m, 5H), 3.22-3.12 (m, 2H), 3.09 (s, 1H), 2.97-2.86 (m, 1H), 2.71-2.61 (m, 2H), 2.54-2.43 (m, 2H), 2.14 (br d, J=2.0 Hz, 3H), 1.94-1.80 (m, 3H), 1.39 (d, J=7.0 Hz, 3H).

LCMS (5% to 95% acetonitrile in water+0.03% ammonium bicarbonate over 6 min); retention time 1.996 min, ESI+ found [M+H]=758.3.

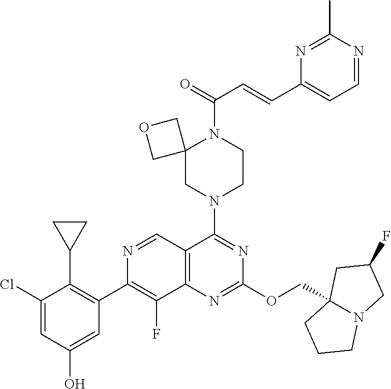 [SMILES: Cc1nccc(/C=C/C(=O)N2CCN(c3nc(OC[C@@]45CCCN4C[C@H](F)C5)nc4c(F)c(-c5cc(O)cc(Cl)c5C5CC5)ncc34)CC23COC3)n1]

### Example 41 (Method 2): (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

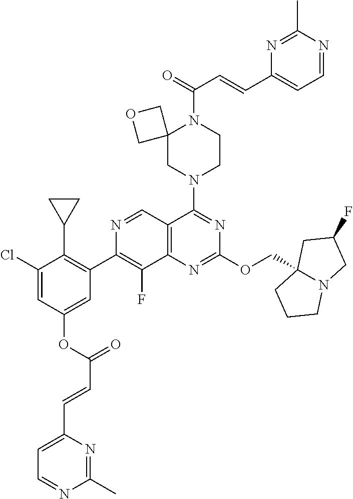 [SMILES: Cc1nccc(/C=C/C(=O)Oc2cc(Cl)c(C3CC3)c(-c3ncc4c(N5CCN(C(=O)/C=C/c6ccnc(C)n6)C6(COC6)C5)nc(OC[C@@]56CCCN5C[C@H](F)C6)nc4c3F)c2)n1]

### Step 1: 3-chloro-4-cyclopropyl-5-(8-fluoro-2-(((2R,7aS)-2-fluorotetrahydro-1H-pyrrolizin-7a(5H)-yl)methoxy)-4-(5-((E)-3-(2-methylpyrimidin-4-yl) acryloyl)-2-oxa-5,8-diazaspiro[3.5]nonan-8-yl)pyrido[4,3-d]pyrimidin-7-yl)phenyl(E)-3-(2-methylpyrimidin-4-yl) acrylate

The amide coupling reaction was prepared in a similar fashion to Method #2, Step

3. The mixture was extracted with ethyl acetate (3 mL*2). The combined organic layers were washed with brine (1 mL) and dried over sodium sulphate. The combined organic layers were concentrated in vacuo affording 3-chloro-4-cyclopropyl-5-(8-fluoro-2-(((2R,7aS)-2-fluorotetrahydro-1H-pyrrolizin-7a(5H)-yl)methoxy)-4-(5-((E)-3-(2-methylpyrimidin-4-yl) acryloyl)-2-oxa-5,8-diazaspiro[3.5]nonan-8-yl)pyrido[4,3-d]pyrimidin-7-yl)phenyl(E)-3-(2-methylpyrimidin-4-yl) acrylate (100 mg, crude) as a yellow oil, which was used in next step without any further purification.

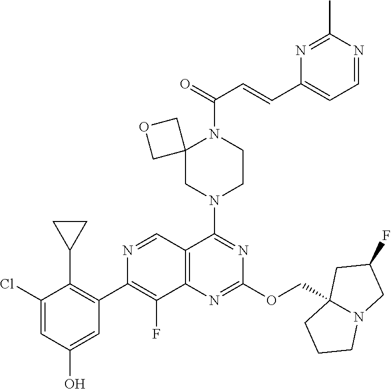 [SMILES: Cc1nccc(/C=C/C(=O)N2CCN(c3nc(OC[C@@]45CCCN4C[C@H](F)C5)nc4c(F)c(-c5cc(O)cc(Cl)c5C5CC5)ncc34)CC23COC3)n1]

### Step 2: (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one

To a solution of 3-chloro-4-cyclopropyl-5-(8-fluoro-2-(((2R,7aS)-2-fluorotetrahydro-1H-pyrrolizin-7a(5H)-yl)methoxy)-4-(5-((E)-3-(2-methylpyrimidin-4-yl) acryloyl)-2-oxa-5,8-diazaspiro[3.5]nonan-8-yl)pyrido[4,3-d]pyrimidin-7-yl)phenyl(E)-3-(2-methylpyrimidin-4-yl) acrylate (90 mg, 100.97 μmol) in tetrahydrofuran (1 mL) was added lithium hydroxide (2 M, 151.45 μL in water). The reaction mixture was stirred at 20° C. for 1 h, diluted with water (2 mL), and extracted with ethyl acetate (3×5 mL). The combined organic layers were dried over sodium sulphate and concentrated in vacuo. The resulting residue was purified by reverse phase HPLC (column: Waters Xbridge Prep OBD C18 150*40 mm*10 μm; mobile phase: [H₂O (10 mM NH₄HCO₃)-ACN]; gradient: 25%-55% B over 8 min) affording (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2-methylpyrimidin-4-yl) prop-2-en-1-one (5.98 mg, 7.95%) as a white solid: ¹H NMR (400 MHz, Chloroform-d) 8.87 (s, 1H), 8.65 (d, J=5.1 Hz, 1H), 7.59-7.55 (m, 1H), 7.49-7.45 (m, 1H), 7.08 (d, J=5.0 Hz, 1H), 6.82 (d, J=2.4 Hz, 1H), 6.55 (br s, 1H), 5.32-5.18 (d, J=52.8 Hz, 1H), 4.84 (br d, J=6.8 Hz, 2H), 4.43 (br s, 2H), 4.37 (br s, 2H), 4.21 (br d, J=10.0 Hz, 1H), 3.85-3.70 (m, 3H), 3.60 (br s, 2H), 3.34-3.30 (m, 1H), 3.23-3.14 (m, 2H), 3.02-2.98 (m, 1H), 2.70 (s, 3H), 2.40-2.31 (m, 1H), 2.34-2.21 (m, 2H), 2.12-2.06 (m, 1H), 1.87 (br d, J=8.3 Hz, 2H), 1.77-1.73 (m, 1H), 0.49 (br s, 2H), 0.08 (br d, J=3.0 Hz, 2H).

LCMS (5% to 95% acetonitrile in water+0.03% ammonium bicarbonate over 6 min); retention time 2.845 min. ESI+ found [M+H]=745.3.

### Example 42 (Method 3): (E)-4-((1S,4S)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

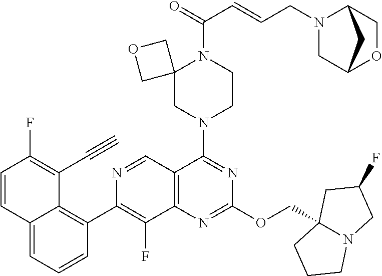 [SMILES: C#Cc1c(F)ccc2cccc(-c3ncc4c(N5CCN(C(=O)/C=C/CN6C[C@@H]7C[C@H]6CO7)C6(COC6)C5)nc(OC[C@@]56CCCN5C[C@H](F)C6)nc4c3F)c12]

### Step 1: (E)-4-((1S,4S)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-4-((1S,4S)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (12 mg, 11.25%) as a white solid: ¹H NMR (400 MHz, Dimethylsulfoxide-d6) δ 9.12 (s, 1H), 8.28-8.18 (m, 2H), 7.74-7.67 (m, 2H), 7.62 (t, J=9.0 Hz, 1H), 6.77-6.67 (m, 1H), 6.61-6.53 (m, 1H), 5.38-5.19 (m, 1H), 4.83 (dd, J=6.7, 10.4 Hz, 2H), 4.47-4.33 (m, 5H), 4.19-4.14 (m, 1H), 4.11-4.06 (m, 1H), 4.04-3.96 (m, 2H), 3.87-3.79 (m, 2H), 3.75-3.65 (m, 2H), 3.52 (dd, J=1.6, 7.5 Hz, 1H), 3.47 (s, 1H), 3.42-3.35 (m, 2H), 3.33-3.28 (m, 1H), 3.16-3.06 (m, 2H), 3.02 (s, 1H), 2.87-2.75 (m, 2H), 2.45 (dd, J=3.3, 9.9 Hz, 1H), 2.15 (br d, J=3.1 Hz, 1H), 2.08-2.05 (m, 1H), 2.02 (br t, J=5.3 Hz, 1H), 1.89-1.83 (m, 1H), 1.81 (br d, J=5.1 Hz, 1H), 1.76 (dd, J=1.7, 9.6 Hz, 1H), 1.59 (br d, J=9.5 Hz, 1H). LCMS (5% to 95% acetonitrile in water+0.03% ammonium bicarbonate over 6 min); retention time 2.818 min, ESI+ found [M+H]=766.3.

### Example 43 (Method 3): (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one

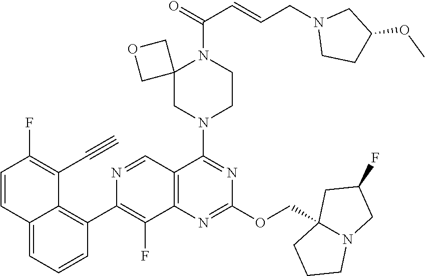 [SMILES: C#Cc1c(F)ccc2cccc(-c3ncc4c(N5CCN(C(=O)/C=C/CN6CC[C@@H](OC)C6)C6(COC6)C5)nc(OC[C@@]56CCCN5C[C@H](F)C6)nc4c3F)c12]

### Step 1: (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-methoxypyrrolidin-1-yl) but-2-en-1-one (5 mg, 4.81%) as a white solid: ¹H NMR (400 MHz, Dimethylsulfoxide-d6) δ 9.12 (s, 1H), 8.28-8.18 (m, 2H), 7.70 (q, J=7.0 Hz, 2H), 7.62 (br t, J=9.0 Hz, 1H), 6.73 (td, J=5.9, 15.0 Hz, 1H), 6.57 (br d, J=15.5 Hz, 1H), 5.38-5.19 (m, 1H), 4.83 (br dd, J=6.6, 11.0 Hz, 2H), 4.48-4.36 (m, 4H), 4.19-4.14 (m, 1H), 4.08 (br dd, J=3.8, 10.4 Hz, 1H), 4.05-3.96 (m, 2H), 3.91-3.79 (m, 2H), 3.72 (br s, 2H), 3.22 (br d, J=5.4 Hz, 2H), 3.17 (s, 3H), 3.10 (br d, J=10.0 Hz, 2H), 3.02 (br s, 1H), 2.88-2.80 (m, 1H), 2.71 (br dd, J=6.2, 9.9 Hz, 1H), 2.62-2.52 (m, 2H), 2.47-2.43 (m, 1H), 2.14 (br s, 1H), 2.07 (br s, 1H), 2.03-1.92 (m, 2H), 1.91-1.74 (m, 3H), 1.72-1.62 (m, 1H).

LCMS (5% to 95% acetonitrile in water+0.03% ammonium bicarbonate over 6 min); retention time 2.911 min, ESI+ found [M+H]=768.3.

### Example 44 (Method 3): (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-fluoropyrrolidin-1-yl) but-2-en-1-one

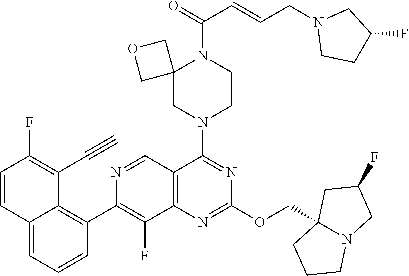 [SMILES: C#Cc1c(F)ccc2cccc(-c3ncc4c(N5CCN(C(=O)/C=C/CN6CC[C@@H](F)C6)C6(COC6)C5)nc(OC[C@@]56CCCN5C[C@H](F)C6)nc4c3F)c12]

### Step 1: (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-fluoropyrrolidin-1-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-((R)-3-fluoropyrrolidin-1-yl) but-2-en-1-one (8.1 mg, 7.78%) as a yellow solid: ¹H NMR (400 MHz, Dimethylsulfoxide-d6) δ 9.12 (s, 1H), 8.27-8.19 (m, 2H), 7.70 (q, J=7.0 Hz, 2H), 7.62 (t, J=9.0 Hz, 1H), 6.78-6.70 (m, 1H), 6.63-6.54 (m, 1H), 5.36-5.26 (m, 1H), 5.24-5.11 (m, 1H), 4.83 (br dd, J=6.7, 10.6 Hz, 2H), 4.45 (br d, J=6.4 Hz, 1H), 4.42 (br d, J=6.8 Hz, 1H), 4.40-4.36 (m, 1H), 4.19-4.14 (m, 1H), 4.09 (dd, J=3.6, 10.4 Hz, 1H), 4.04-3.98 (m, 2H), 3.83 (br d, J=13.0 Hz, 1H), 3.73 (br s, 2H), 3.27 (br d, J=6.3 Hz, 2H), 3.10 (br d, J=10.5 Hz, 2H), 3.05-3.01 (m, 1H), 2.87-2.81 (m, 2H), 2.81-2.76 (m, 1H), 2.69 (dd, J=4.9, 11.6 Hz, 1H), 2.62 (dd, J=4.9, 11.6 Hz, 1H), 2.40-2.33 (m, 1H), 2.20-2.10 (m, 2H), 2.08-2.00 (m, 2H), 1.97-1.84 (m, 2H), 1.81 (br dd, J=7.3, 12.3 Hz, 2H).

LCMS (5% to 95% acetonitrile in water+0.1% trifluoroacetic acid over 6 min); retention time 1.969 min, ESI+ found [M+H]=756.3.

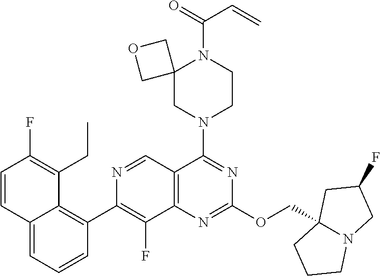 [SMILES: C=CC(=O)N1CCN(c2nc(OC[C@@]34CCCN3C[C@H](F)C4)nc3c(F)c(-c4cccc5ccc(F)c(CC)c45)ncc23)CC12COC2]

### Example 45 (Method 1): 1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido|4,3-d]pyrimidin-4-yl)-2-oxa-5,8-diazaspiro[3.5]nonan-5-yl) prop-2-en-1-one

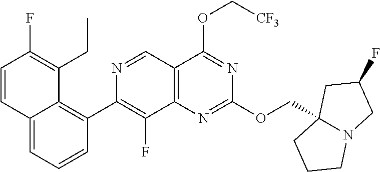 [SMILES: CCc1c(F)ccc2cccc(-c3ncc4c(OCC(F)(F)F)nc(OC[C@@]56CCCN5C[C@H](F)C6)nc4c3F)c12]

### Step 1: 7-(8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The reaction mixture was filtered and concentrated in vacuo, and the crude residue was purified by column chromatography (silica gel, 100-200 mesh, 0-100% ethyl acetate in petroleum ether) affording 7-(8-ethyl-7-fluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (650 mg, 98.94%) as a yellow oil. LCMS Rt=0.709 min, m/z=577.2 [M+H]⁺.

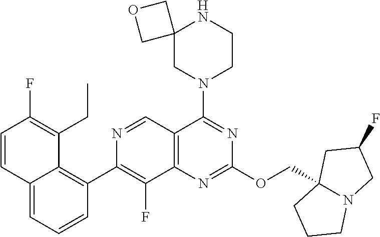 [SMILES: CCc1c(F)ccc2cccc(-c3ncc4c(N5CCNC6(COC6)C5)nc(OC[C@@]56CCCN5C[C@H](F)C6)nc4c3F)c12]

### Step 2: 8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The reaction mixture was concentrated in vacuo, and the crude product was purified by reverse phase HPLC (column: Waters Xbridge Prep OBD C18 150*40 mm*10 μm; mobile phase: [water (NH₄HCO₃)-ACN]; B %: 30%-60%, 8 min) affording 8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (450 mg, 66.01%) as a white solid. LCMS Rt=0.761 min, m/z=605.3 [M+H]⁺.

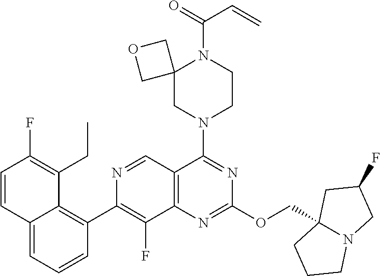 [SMILES: C=CC(=O)N1CCN(c2nc(OC[C@@]34CCCN3C[C@H](F)C4)nc3c(F)c(-c4cccc5ccc(F)c(CC)c45)ncc23)CC12COC2]

### Step 3: 1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The resulting residue was purified by reverse phase HPLC (column: Waters Xbridge Prep OBD C18 150*40 mm*10 μm; mobile phase: [water (NH₄HCO₃)-ACN]; B %: 30%-60%, 8 min) affording 1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (46.29 mg, 28.16%) as a yellow amorphous solid: ¹H NMR (400 MHz, Chloroform-d) δ 9.11 (s, 1H), 8.05 (dd, J=1.1, 8.1 Hz, 1H), 7.94 (dd, J=6.1, 8.9 Hz, 1H), 7.58-7.53 (m, 1H), 7.51-7.47 (m, 1H), 7.39 (t, J=9.4 Hz, 1H), 6.65 (dd, J=10.5, 16.8 Hz, 1H), 6.28 (dd, J=1.9, 16.8 Hz, 1H), 5.77 (dd, J=1.9, 10.5 Hz, 1H), 5.36-5.19 (m, 1H), 4.88 (br d, J=6.4 Hz, 2H), 4.56-4.49 (m, 1H), 4.47-4.39 (m, 3H), 4.27-4.23 (m, 1H), 4.19-4.15 (m, 1H), 3.93 (br t, J=5.0 Hz, 2H), 3.68-3.59 (m, 2H), 3.20-3.12 (m, 2H), 3.11-3.06 (m, 1H), 2.94-2.87 (m, 1H), 2.52-2.45 (m, 1H), 2.31-2.25 (m, 1H), 2.22-2.18 (m, 1H), 2.12 (br d, J=2.9 Hz, 1H), 2.10-2.04 (m, 1H), 2.00-1.93 (m, 1H), 1.92-1.89 (m, 1H), 1.88-1.84 (m, 1H), 0.82 (t, J=7.4 Hz, 3H).

LCMS (5% to 95% acetonitrile in water+0.1% trifluoroacetic acid over 6 min); retention time 2.259 min, ESI+ found [M+H]=659.3.

### Example 46 (Method 3): (E)-4-((1R,4R)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

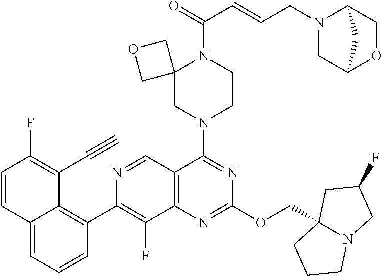 [SMILES: C#Cc1c(F)ccc2cccc(-c3ncc4c(N5CCN(C(=O)/C=C/CN6C[C@H]7C[C@@H]6CO7)C6(COC6)C5)nc(OC[C@@]56CCCN5C[C@H](F)C6)nc4c3F)c12]

### Step 1: (E)-4-((1R,4R)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 30%-60%, 8 min) affording (E)-4-((1R,4R)-2-oxa-5-azabicyclo[2.2.1]heptan-5-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (7.06 mg, 6.51%) as a yellow solid: ¹H NMR (400 MHz, Dimethylsulfoxide-d6) δ 9.12 (s, 1H), 8.28-8.18 (m, 2H), 7.75-7.65 (m, 2H), 7.62 (t, J=9.0 Hz, 1H), 6.79-6.67 (m, 1H), 6.62-6.50 (m, 1H), 5.41-5.19 (m, 1H), 4.83 (dd, J=6.6, 10.5 Hz, 2H), 4.47-4.33 (m, 4H), 4.20-4.13 (m, 1H), 4.12-4.05 (m, 1H), 4.04-3.93 (m, 2H), 3.88-3.79 (m, 2H), 3.76-3.64 (m, 2H), 3.53 (dd, J=1.6, 7.5 Hz, 1H), 3.47 (s, 1H), 3.42-3.33 (m, 3H), 3.17-3.06 (m, 2H), 3.06-2.99 (m, 1H), 2.88-2.80 (m, 1H), 2.77 (ddd, J=1.5, 3.4, 9.8 Hz, 1H), 2.45 (br dd, J=3.3, 9.8 Hz, 1H), 2.15 (d, J=3.0 Hz, 1H), 2.09-1.98 (m, 2H), 1.92-1.72 (m, 4H), 1.59 (br d, J=9.9 Hz, 1H).

LCMS (5% to 95% acetonitrile in water+0.1% trifluoroacetic acid over 6 mins); retention time 1.946 min, ESI+ found [M+H]=766.3.

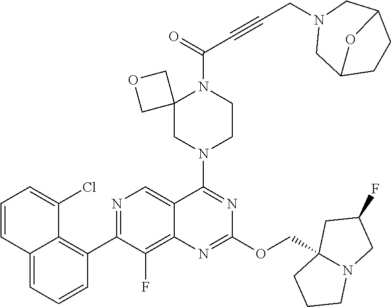 [SMILES: O=C(C#CCN1CC2CCC(C1)O2)N1CCN(c2nc(OC[C@@]34CCCN3C[C@H](F)C4)nc3c(F)c(-c4cccc5cccc(Cl)c45)ncc23)CC12COC2]

### Example 47 (Method 4): 4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one

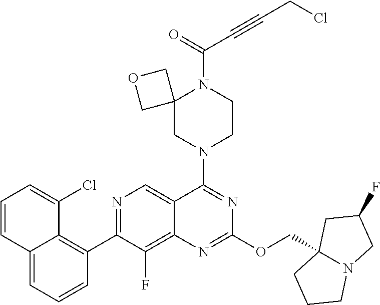 [SMILES: O=C(C#CCCl)N1CCN(c2nc(OC[C@@]34CCCN3C[C@H](F)C4)nc3c(F)c(-c4cccc5cccc(Cl)c45)ncc23)CC12COC2]

### Step 1: 4-chloro-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one

The amide coupling reaction was prepared in a similar fashion to Method #2, Step 3. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 40%-70%, 8 min) affording 4-chloro-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one (45 mg, 38.48%) as a white solid. LCMS Rt=2.150 min, m/z=693.2 [M+H]⁺.

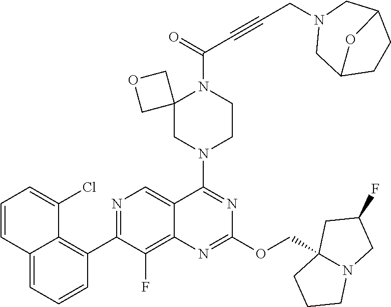 [SMILES: O=C(C#CCN1CC2CCC(C1)O2)N1CCN(c2nc(OC[C@@]34CCCN3C[C@H](F)C4)nc3c(F)c(-c4cccc5cccc(Cl)c45)ncc23)CC12COC2]

### Step 2: 4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one

To a solution of (1R,5S)-8-oxa-3-azabicyclo[3.2.1]octane (14.68 mg, 129.76 μmol) and diisopropylethylamine (22.36 mg, 173.02 μmol) in N,N-dimethylformaldehyde (1 mL) was added 4-chloro-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one (30 mg, 43.25 μmol). The mixture was stirred at 50° C. for 1 h. The reaction mixture was concentrated in vacuo and purified by reverse phase HPLC (column: Waters Xbridge BEH C18 100*30 mm*10 μm; mobile phase: [water (NH₄HCO₃)-acetonitrile]; B %: 40%-70%, 8 min) affording 4-(8-oxa-3-azabicyclo[3.2.1]octan-3-yl)-1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-yn-1-one (4 mg, 11.65%) as a yellow solid: ¹H NMR (400 MHz, Dimethylsulfoxide-d6) δ 9.18-9.12 (m, 1H), 8.17 (d, J=8.1 Hz, 1H), 8.07 (br d, J=8.3 Hz, 1H), 7.72-7.67 (m, 1H), 7.64-7.61 (m, 1H), 7.58 (br d, J=7.0 Hz, 1H), 7.56-7.52 (m, 1H), 5.33-5.16 (m, 1H), 5.00-4.94 (m, 2H), 4.41 (br s, 2H), 4.35-4.31 (m, 2H), 4.22 (br s, 2H), 4.16-4.11 (m, 1H), 4.07 (br s, 3H), 3.91-3.86 (m, 2H), 3.50 (s, 2H), 3.05 (br dd, J=1.1, 7.0 Hz, 2H), 2.98 (br s, 1H), 2.82-2.76 (m, 1H), 2.49 (br s, 1H), 2.43 (br s, 2H), 2.40 (br s, 1H), 2.10 (br s, 1H), 2.03 (br d, J=0.9 Hz, 1H), 1.97 (br d, J=5.9 Hz, 1H), 1.84-1.81 (m, 1H), 1.78 (br d, J=7.0 Hz, 2H), 1.74 (br d, J=2.2 Hz, 2H), 1.69 (br dd, J=2.6, 4.6 Hz, 2H).

LCMS (5% to 95% acetonitrile in water+0.03% ammonium bicarbonate over 6 min); retention time 3.066 min, ESI+ found [M+H]=770.3.

### Example 48 (Method 2): (E)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

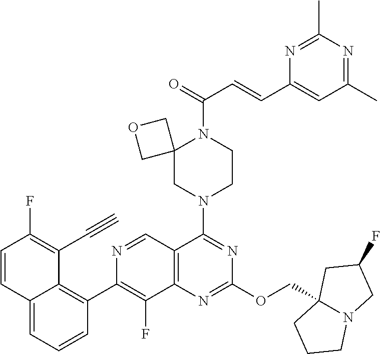 [SMILES: C#Cc1c(F)ccc2cccc(-c3ncc4c(N5CCN(C(=O)/C=C/c6cc(C)nc(C)n6)C6(COC6)C5)nc(OC[C@@]56CCCN5C[C@H](F)C6)nc4c3F)c12]

### Step 1: (E)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-ACN]; B %: 40%-70%, 8 min) affording (E)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (60.97 mg, 47.03%) as a yellow solid: ¹H NMR (400 MHz, Acetonitrile-d3) δ 9.10 (s, 1H), 8.18-8.11 (m, 2H), 7.72-7.68 (m, 2H), 7.59 (br d, J=15.3 Hz, 1H), 7.51-7.40 (m, 2H), 7.27 (s, 1H), 5.37-5.18 (m, 1H), 4.98-4.88 (m, 2H), 4.62-4.47 (m, 3H), 4.42 (br d, J=6.6 Hz, 1H), 4.26-4.07 (m, 3H), 3.90-3.74 (m, 3H), 3.35 (s, 1H), 3.22-3.06 (m, 3H), 2.96-2.87 (m, 1H), 2.64 (s, 3H), 2.49 (s, 3H), 2.15-2.00 (m, 3H), 1.94-1.82 (m, 3H).

LCMS (5% to 95% acetonitrile in water+0.03% ammonium bicarbonate over 6 min); retention time 2.952 min, ESI+ found [M+H]=761.3.

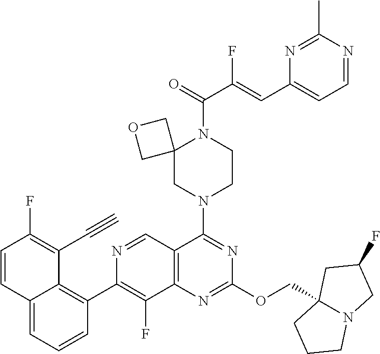 [SMILES: C#Cc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12]

### Example 49 (Method 2): (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

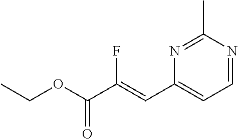

### Step 1: ethyl(Z)-2-fluoro-3-(2-methylpyrimidin-4-yl) acrylate

To a solution of 2-methylpyrimidine-4-carbaldehyde (4.20 g, 34.41 mmol) in acetonitrile (130 mL) was added lithium chloride (4.38 g, 103.23 mmol), diisopropylethylamine (13.34 g, 103.23 mmol) and ethyl 2-diethoxyphosphoryl-2-fluoro-acetate (10 g, 41.29 mmol). The mixture was stirred at 25° C. for 12 h. The reaction mixture was concentrated to dryness in vacuo. The residue was purified by column chromatography (silica gel, 100-200 mesh, 0-100% ethyl acetate in petroleum ether) affording ethyl ethyl(Z)-2-fluoro-3-(2-methylpyrimidin-4-yl) acrylate (4.2 g, 58.07%) as a yellow oil. LCMS Rt=0.498 min, m/z=211.1 [M+H]⁺.

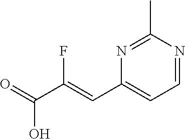

### Step 2: (Z)-2-fluoro-3-(2-methylpyrimidin-4-yl) acrylic acid

To a solution of ethyl(Z)-2-fluoro-3-(2-methylpyrimidin-4-yl) prop-2-enoate (4.2 g, 19.98 mmol) in tetrahydrofuran (15 mL) and water (15 mL) was added lithium hydroxide monohydrate (1.68 g, 39.96 mmol) at 0° C. The mixture was stirred at 25° C. for 2 h. The reaction mixture was quenched with saturated potassium hydrogen sulfate (20 mL) at 0° C. until pH=2, concentrated under reduced pressure to remove tetrahydrofuran, and filtered. The filter cake was dried in vacuo. The residue was purified by reverse phase HPLC (column: Phenomenex luna C18 (250*70 mm, 15 μm); mobile phase: [water (TFA)-ACN]; B %: 1%-25%, 23 min) affording (Z)-2-fluoro-3-(2-methylpyrimidin-4-yl) acrylic acid (400 mg, 10.99%) as a red solid. LCMS Rt=0.571 min, m/z=183.1 [M+H]⁺.

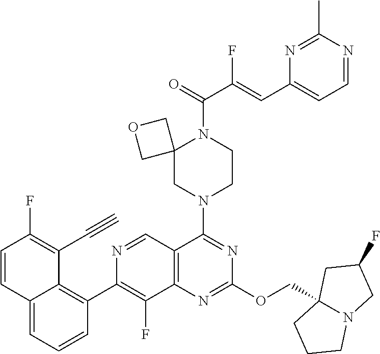 [SMILES: C#Cc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12]

### Step 3: (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

The amide coupling reaction was prepared in a similar fashion to Method #2, Step 3. The residue was purified by reverse phase HPLC (column: Waters Xbridge Prep OBD C18 150*40 mm*10 μm; mobile phase: [water (NH₄HCO₃)-ACN]; B %: 35%-65%, 8 min) affording (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one (16.41 mg, 27.58%) as a yellow oil: ¹H NMR (400 MHz, Acetonitrile-d3) δ 9.10 (s, 1H), 8.74 (d, J=5.3 Hz, 1H), 8.20-8.11 (m, 2H), 7.70 (br d, J=4.0 Hz, 2H), 7.63-7.56 (m, 1H), 7.47 (br t, J=9.0 Hz, 1H), 6.68-6.55 (m, 1H), 5.39-5.17 (m, 1H), 4.97-4.88 (m, 2H), 4.61-4.48 (m, 3H), 4.43 (br d, J=6.6 Hz, 1H), 4.28-4.20 (m, 1H), 4.18-4.07 (m, 2H), 3.94-3.82 (m, 3H), 3.32 (s, 1H), 3.19-3.06 (m, 3H), 2.98-2.86 (m, 1H), 2.68 (s, 3H), 2.14-2.00 (m, 3H), 1.88 (br dd, J=7.4, 10.5 Hz, 3H).

LCMS (5% to 95% acetonitrile in water+0.03% ammonium bicarbonate over 6 min); retention time 2.998 min, ESI+ found [M+H]=765.3.

### Example 50 (Method 1): 1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

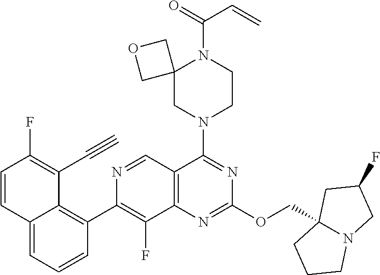 [SMILES: C#Cc1c(F)ccc2cccc(-c3ncc4c(N5CCN(C(=O)C=C)C6(COC6)C5)nc(OC[C@@]56CCCN5C[C@H](F)C6)nc4c3F)c12]

### Step 1: 1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The residue was purified by reverse phase HPLC (column: Waters Xbridge BEH C18 100*30 mm*10 μm; mobile phase: [water (NH₄HCO₃)-ACN]; B %: 40%-70%, 8 min) affording 1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (75.53 mg, 46.20%) as a yellow solid: 1H NMR (400 MHz, Acetonitrile-d3) δ 9.03 (s, 1H), 8.13-8.04 (m, 2H), 7.70-7.60 (m, 2H), 7.42 (t, J=9.1 Hz, 1H), 6.62 (dd, J=10.5, 16.8 Hz, 1H), 6.23 (dd, J=1.9, 16.8 Hz, 1H), 5.73 (dd, J=1.9, 10.4 Hz, 1H), 5.33-5.13 (m, 1H), 4.85 (d, J=6.4 Hz, 1H), 4.80 (d, J=6.6 Hz, 1H), 4.53-4.43 (m, 2H), 4.43-4.36 (m, 1H), 4.33 (d, J=6.5 Hz, 1H), 4.23-4.16 (m, 1H), 4.14-4.07 (m, 1H), 4.06-3.95 (m, 1H), 3.81-3.72 (m, 1H), 3.64 (br t, J=4.9 Hz, 2H), 3.28 (d, J=0.6 Hz, 1H), 3.17-3.06 (m, 2H), 3.04 (s, 1H), 2.92-2.81 (m, 1H), 2.18-2.15 (m, 1H), 2.08 (d, J=2.9 Hz, 1H), 2.06-1.98 (m, 1H), 1.90-1.74 (m, 3H). LCMS (5% to 95% acetonitrile in water+0.03% ammonium bicarbonate over 6 min); retention time 2.952 min. ESI+ found [M+H]=655.3.

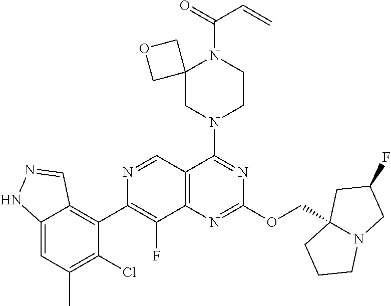 [SMILES: C=CC(=O)N1CCN(c2nc(OC[C@@]34CCCN3C[C@H](F)C4)nc3c(F)c(-c4c(Cl)c(C)cc5[nH]ncc45)ncc23)CC12COC2]

### Example 51 (Method 1): 1-(8-(7-(5-chloro-6-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

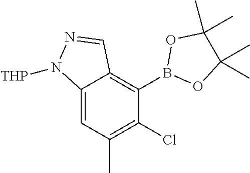

### Step 1: 5-chloro-6-methyl-1-(tetrahydro-2H-pyran-2-yl)-4-(4,4,5,5-tetramethyl-1,3,2-dioxaborolan-2-yl)-1H-indazole

To a solution of 4-bromo-5-chloro-6-methyl-1-tetrahydropyran-2-yl-indazole (650 mg, 1.97 mmol) and 4,4,5,5-tetramethyl-2-(4,4,5,5-tetramethyl-1,3,2-dioxaborolan-2-yl)-1,3,2-dioxaborolane (1.00 g, 3.94 mmol) in methoxycyclopentane (100 mL) was added potassium acetate (774.13 mg, 7.89 mmol) and Dichloro[1,1-bis(diphenylphosphino) ferrocene]palladium (II) dichloromethane adduct (161.04 mg, 197.20 μmol). The mixture was stirred at 80° C. for 1 h under a nitrogen atmosphere. The mixture was concentrated in vacuo and purified by column chromatography (silica gel, 100-200 mesh, 0-100% ethyl acetate in petroleum ether) affording 5-chloro-6-methyl-1-(tetrahydro-2H-pyran-2-yl)-4-(4,4,5,5-tetramethyl-1,3,2-dioxaborolan-2-yl)-1H-indazole (600 mg, 80.77%) as a yellow oil. LCMS Rt=2.544 min, m/z=377.2 [M+H]⁺.

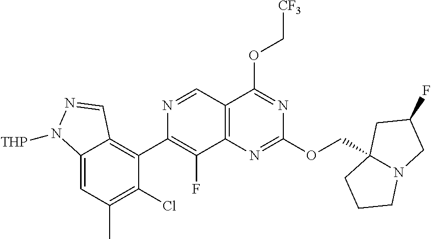

### Step 2: 7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The residue was purified by column chromatography (silica gel, 100-200 mesh, 0-30% ethyl acetate in petroleum ether) affording 7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (150 mg, 43.82%) as a yellow oil. LCMS Rt=2.534 min, m/z=653.2 [M+H]⁺.

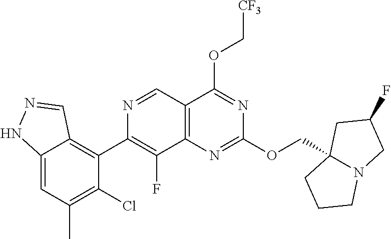 [SMILES: Cc1cc2[nH]ncc2c(-c2ncc3c(OCC(F)(F)F)nc(OC[C@@]45CCCN4C[C@H](F)C5)nc3c2F)c1Cl]

### Step 3: 7-(5-chloro-6-methyl-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine

A solution of 7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 153.13 μmol) in dichloromethane (2 mL) and trifluoroacetic acid (0.1 mL) was stirred at 25° C. for 0.5 h. The reaction mixture was concentrated in vacuo affording 7-(5-chloro-6-methyl-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, crude, trifluoroacetate salt) as a yellow oil, which was used in the next step without further purification. LCMS Rt=0.635 min, m/z=569.1 [M+H]⁺.

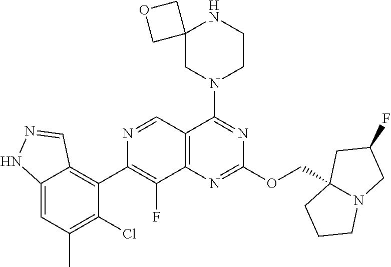 [SMILES: Cc1cc2[nH]ncc2c(-c2ncc3c(N4CCNC5(COC5)C4)nc(OC[C@@]45CCCN4C[C@H](F)C5)nc3c2F)c1Cl]

### Step 4: 8-(7-(5-chloro-6-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The residue was purified by reverse phase HPLC (column: Waters Xbridge BEH C18 100*30 mm*10 μm; mobile phase: [water (NH₄HCO₃)-ACN]; B %: 40%-60%, 8 min) affording 8-(7-(5-chloro-6-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (25 mg, 28.60%) as a yellow solid. LCMS Rt=1.605 min, m/z=597.2 [M+H]⁺.

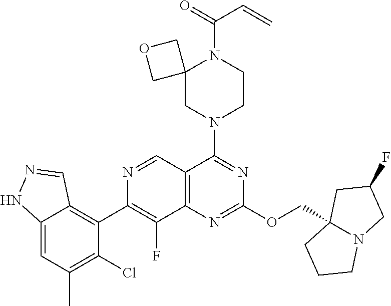 [SMILES: C=CC(=O)N1CCN(c2nc(OC[C@@]34CCCN3C[C@H](F)C4)nc3c(F)c(-c4c(Cl)c(C)cc5[nH]ncc45)ncc23)CC12COC2]

### Step 5: 1-(8-(7-(5-chloro-6-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The amide coupling reaction was prepared in a similar fashion to Method #1, Step 5. The residue was purified by reverse phase HPLC (column: Waters Xbridge Prep OBD C18 150*40 mm*10 μm; mobile phase: [water (NH₄HCO₃)-ACN]; B %: 30%-60%, 8 min) affording 1-(8-(7-(5-chloro-6-methyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (1.1 mg, 5.04%) as yellow oil: ¹H NMR (400 MHz, Acetonitrile-d3) δ 9.20 (s, 1H), 7.69 (d, J=3.1 Hz, 2H), 6.68 (dd, J=10.6, 16.8 Hz, 1H), 6.30 (dd, J=1.9, 16.8 Hz, 1H), 5.79 (dd, J=1.9, 10.5 Hz, 1H), 5.42-5.18 (m, 1H), 4.89 (br t, J=5.5 Hz, 2H), 4.66-4.52 (m, 1H), 4.50-4.39 (m, 3H), 4.31-4.17 (m, 2H), 4.01-3.92 (m, 2H), 3.70-3.62 (m, 2H), 3.23-3.15 (m, 2H), 3.14-3.08 (m, 1H), 2.96-2.90 (m, 1H), 2.61 (s, 3H), 2.15 (br d, J=3.0 Hz, 2H), 2.12-2.08 (m, 2H), 1.93-1.87 (m, 2H).

LCMS (5% to 95% acetonitrile in water+0.03% ammonium bicarbonate over 6 min); retention time 2.716 min. ESI+ found [M+H]=651.2.

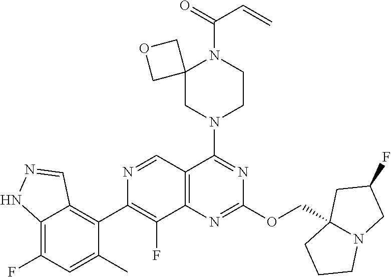 [SMILES: C=CC(=O)N1CCN(c2nc(OC[C@@]34CCCN3C[C@H](F)C4)nc3c(F)c(-c4c(C)cc(F)c5[nH]ncc45)ncc23)CC12COC2]

### Example 52 (Method 1): 1-(8-(8-fluoro-7-(7-fluoro-5-methyl-1H-indazol-4-yl)-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

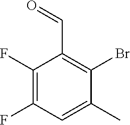

### Step 1: 2-bromo-5,6-difluoro-3-methylbenzaldehyde

To a solution of 1-bromo-4,5-difluoro-2-methyl-benzene (9.01 g, 43.52 mmol) in tetrahydrofuran (100 mL) was added lithium diisopropylamide (2 M, 23.94 mL) at −78° C. under a nitrogen atmosphere. The mixture was stirred at −78° C. for 20 min. N,N-dimethylformamide (3.82 g, 52.23 mmol) was added to the mixture, and the mixture was stirred at −78° C. for 15 min. The mixture was diluted with water (40 mL) and acetic acid (8 mL) and extracted with ethyl acetate (3×30 mL). The combined organic layers were dried over sodium sulphate and concentrated to dryness in vacuo and purified by column chromatography (silica gel, 100-200 mesh, 2-20% ethyl acetate in petroleum ether) affording 2-bromo-5,6-difluoro-3-methylbenzaldehyde (9.5 g, 92.87%) as a brown solid: ¹H NMR (400 MHz, Chloroform-d) δ 10.25 (dd, J=0.9, 1.6 Hz, 1H), 7.24 (dd, J=8.0, 10.4 Hz, 1H), 2.35 (s, 3H).

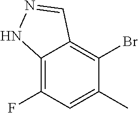

### Step 2: 4-bromo-7-fluoro-5-methyl-1H-indazole

To a solution of 2-bromo-5,6-difluoro-3-methylbenzaldehyde (9.5 g, 40.42 mmol) in dioxane (8 mL) was added hydrazine hydrate (185.77 g, 3.60 mol). The mixture was stirred at 100° C. for 12 h. The mixture was diluted with water (50 mL) and extracted with ethyl acetate (3×50 mL). The combined organic layers were dried over sodium sulphate, concentrated to dryness in vacuo, and purified by column chromatography (silica gel, 100-200 mesh, 2-20% ethyl acetate in petroleum ether) affording 4-bromo-7-fluoro-5-methyl-1H-indazole (6.5 g, 70.21%) as a brown solid. LCMS Rt=0.683 min, m/z=229.0 [M+H]⁺.

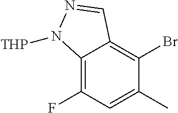

### Step 3: 4-bromo-7-fluoro-5-methyl-1-(tetrahydro-2H-pyran-2-yl)-1H-indazole

To a solution of 4-bromo-7-fluoro-5-methyl-1H-indazole (6.5 g, 28.38 mmol) in dichloromethane (100 mL) was added pyridinium p-toluenesulfonate (713.15 mg, 2.84 mmol) and 3,4-dihydro-2H-pyran (16.71 g, 198.65 mmol). The mixture was stirred at 30° C. for 12 h. The mixture was diluted with water (50 mL) and extracted with ethyl acetate (3×50 mL). The combined organic layers were dried over sodium sulphate, concentrated to dryness in vacuo, and purified by column chromatography (silica gel, 100-200 mesh, 2-20% ethyl acetate in petroleum ether) affording 4-bromo-7-fluoro-5-methyl-1-(tetrahydro-2H-pyran-2-yl)-1H-indazole (8.8 g, 99.02%) as a white solid. LCMS Rt=0.924 min, m/z=313.0 [M+H]⁺.

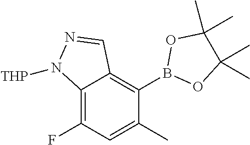

### Step 4: 7-fluoro-5-methyl-1-(tetrahydro-2H-pyran-2-yl)-4-(4,4,5,5-tetramethyl-1,3,2-dioxaborolan-2-yl)-1H-indazole

A mixture of 4-bromo-7-fluoro-5-methyl-1-(tetrahydro-2H-pyran-2-yl)-1H-indazole (8.8 g, 28.10 mmol), 4,4,5,5-tetramethyl-2-(4,4,5,5-tetramethyl-1,3,2-dioxaborolan-2-yl)-1,3,2-dioxaborolane (10.70 g, 42.15 mmol), potassium acetate (8.27 g, 84.30 mmol) and [1,1′-bis(diphenylphosphino) ferrocene]dichloropalladium (II)(2.06 g, 2.81 mmol) in dioxane (100 mL) was degassed and purged with nitrogen 3 times. The mixture was stirred at 100° C. for 12 h under a nitrogen atmosphere. The reaction mixture was concentrated in vacuo. The residue was purified by reverse phase HPLC (column: Agela DuraShell C18 250*70 mm*10 μm; mobile phase: [water (NH₄HCO₃)-ACN]; B %: 95%-95%, 20 min) affording 7-fluoro-5-methyl-1-(tetrahydro-2H-pyran-2-yl)-4-(4,4,5,5-tetramethyl-1,3,2-dioxaborolan-2-yl)-1H-indazole (5 g, 42.81%) as a brown solid. LCMS Rt=1.048 min, m/z=361.2 [M+H]⁺.

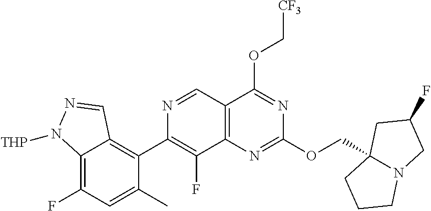

### Step 5: 8-fluoro-7-(7-fluoro-5-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine

The Suzuki reaction was prepared in a similar fashion to Method #6, Step 4. The mixture was diluted with water (50 mL) and extracted with ethyl acetate (3×50 mL). The combined organic layers were dried over sodium sulphate, concentrated to dryness in vacuo, and purified by column chromatography (silica gel, 100-200 mesh, 2%-50% ethyl acetate in petroleum ether) affording 8-fluoro-7-(7-fluoro-5-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (700 mg, 96.46%) as a brown oil. LCMS Rt=0.679 min, m/z=637.2 [M+H]⁺.

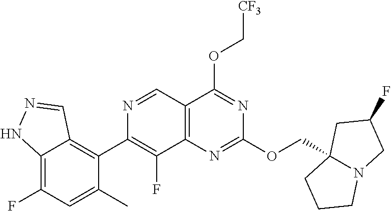 [SMILES: Cc1cc(F)c2[nH]ncc2c1-c1ncc2c(OCC(F)(F)F)nc(OC[C@@]34CCCN3C[C@H](F)C4)nc2c1F]

### Step 6: 8-fluoro-7-(7-fluoro-5-methyl-1H-indazol-4-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine

To a solution of 8-fluoro-7-(7-fluoro-5-methyl-1-(tetrahydro-2H-pyran-2-yl)-1H-indazol-4-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (300 mg, 471.26 μmol) in dichloromethane (4 mL) was added trifluoroacetic acid (770.00 mg, 6.75 mmol). The mixture was stirred at 25° C. for 0.5 h and concentrated in vacuo affording 8-fluoro-7-(7-fluoro-5-methyl-1H-indazol-4-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (300 mg, crude, trifluoroacetate salt) as a brown oil, which was used in the next step without further purification. LCMS Rt=0.557 min, m/z=553.2 [M+H]⁺.

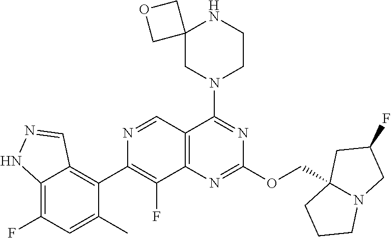 [SMILES: Cc1cc(F)c2[nH]ncc2c1-c1ncc2c(N3CCNC4(COC4)C3)nc(OC[C@@]34CCCN3C[C@H](F)C4)nc2c1F]

### Step 7: 8-(8-fluoro-7-(7-fluoro-5-methyl-1H-indazol-4-yl)-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

The substitution reaction was prepared in a similar fashion to Method #6, Step 5. The mixture was concentrated in vacuo, and the residue was purified by reverse phase HPLC (column: Waters Xbridge Prep OBD C18 150*40 mm*10 μm; mobile phase: [water (NH₄HCO₃)-ACN]; B %: 20%-50%, 8 min) affording 8-(8-fluoro-7-(7-fluoro-5-methyl-1H-indazol-4-yl)-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (50 mg, 18.65%) as a white solid. LCMS Rt=0.744 min, m/z=581.3 [M+H]⁺.

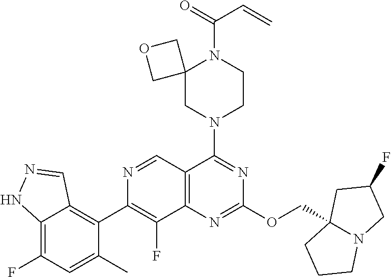 [SMILES: C=CC(=O)N1CCN(c2nc(OC[C@@]34CCCN3C[C@H](F)C4)nc3c(F)c(-c4c(C)cc(F)c5[nH]ncc45)ncc23)CC12COC2]

### Step 8: 1-(8-(8-fluoro-7-(7-fluoro-5-methyl-1H-indazol-4-yl)-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The acylation reaction was prepared in a similar fashion to Method #1, Step 5. The residue was purified by reverse phase HPLC (column: Waters Xbridge Prep OBD C18 150*40 mm*10 μm; mobile phase: [water (NH₄HCO₃)-ACN]; B %: 20%-50%, 8 min) affording 1-(8-(8-fluoro-7-(7-fluoro-5-methyl-1H-indazol-4-yl)-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (12.38 mg, 26.65%) as a white solid: 1H NMR (400 MHz, Acetonitrile-d3) δ 12.53-11.22 (m, 1H), 9.17 (s, 1H), 7.75 (d, J=3.4 Hz, 1H), 7.16 (d, J=12.0 Hz, 1H), 6.67 (dd, J=10.5, 16.8 Hz, 1H), 6.29 (dd, J=1.9, 16.8 Hz, 1H), 5.79 (dd, J=1.9, 10.5 Hz, 1H), 5.43-5.17 (m, 1H), 4.88 (d, J=6.5 Hz, 2H), 4.52-4.39 (m, 4H), 4.35-4.12 (m, 2H), 3.93 (br d, J=4.8 Hz, 2H), 3.66 (t, J=5.0 Hz, 2H), 3.24-3.07 (m, 3H), 3.02-2.87 (m, 1H), 2.34 (s, 3H), 2.14 (br d, J=2.8 Hz, 4H), 1.93-1.87 (m, 2H).

LCMS (5% to 95% acetonitrile in water+0.1% trifluoroacetic acid over 6 min); retention time 1.878 min, ESI+ found [M+H]=635.3.

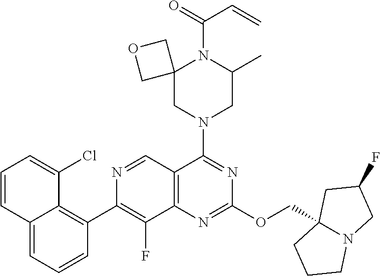 [SMILES: C=CC(=O)N1C(C)CN(c2nc(OC[C@@]34CCCN3C[C@H](F)C4)nc3c(F)c(-c4cccc5cccc(Cl)c45)ncc23)CC12COC2]

### Example 53 (Method 1): 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one

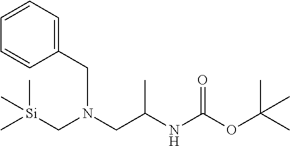 [SMILES: CC(CN(Cc1ccccc1)C[Si](C)(C)C)NC(=O)OC(C)(C)C]

### Step 1: tert-butyl(1-(benzyl((trimethylsilyl)methyl) amino) propan-2-yl) carbamate

To a solution of N-(trimethylsilylmethyl)benzylamine (5 g, 25.86 mmol) in acetonitrile (150 mL) was added triethylamine (5.23 g, 51.72 mmol) and (2-Iodo-1-methyl-ethyl)-carbamic acid tert-butyl ester (7.37 g, 25.86 mmol) at 0° C. The mixture was stirred at 30° C. for 1 h. The reaction mixture was concentrated in vacuo and purified by reverse phase HPLC (column: Phenomenex luna C18 250 mm*100 mm*10 μm; mobile phase: [water (HCl)-acetonitrile]; B %: 25%-55%, 20 min) affording tert-butyl(1-(benzyl((trimethylsilyl)methyl) amino) propan-2-yl) carbamate (1.5 g, 16.55%, hydrochloric acid salt) as a yellow oil. LCMS Rt=1.839 min, m/z=351.2 [M+H]⁺.

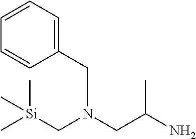 [SMILES: CC(N)CN(Cc1ccccc1)C[Si](C)(C)C]

### Step 2: N1-benzyl-N1-((trimethylsilyl)methyl) propane-1,2-diamine

A mixture of tert-butyl(1-(benzyl((trimethylsilyl)methyl) amino) propan-2-yl) carbamate (600 mg, 1.71 mmol) in dichloromethane (6 mL) and trifluoroacetic acid (2 mL) was stirred at 25° C. for 1 h. The reaction mixture was quenched with saturated sodium bicarbonate (10 mL) and extracted with ethyl acetate (3×5 mL). The combined organic layers were dried over sodium sulfate, filtered and concentrated in vacuo affording N1-benzyl-N1-((trimethylsilyl)methyl) propane-1,2-diamine (650 mg, crude) as a yellow oil, which was used in the next step without further purification. LCMS Rt=0.390 min, m/z=251.2 [M+H]⁺.

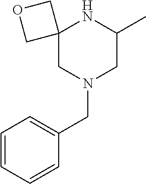 [SMILES: CC1CN(Cc2ccccc2)CC2(COC2)N1]

### Step 3: 8-benzyl-6-methyl-2-oxa-5,8-diazaspiro[3.5]nonane

A mixture of N1-benzyl-N1-((trimethylsilyl)methyl) propane-1,2-diamine (300 mg, 1.20 mmol), oxetan-3-one (86.32 mg, 1.20 mmol), (4,4′-di-tert-butyl-2,2′-bipyridine)bis[(2-pyridinyl)phenyl]iridium (III) hexafluorophosphate (109.48 mg, 119.78 μmol) and 4A molecule sieves (200 mg, 211.48 μmol) in acetonitrile (3 mL) and trifluoroethanol (1 mL) was degassed and purged with nitrogen 3 times. The mixture was stirred at 25° C. for 12 h under a nitrogen atmosphere and under irradiation by blue light. The reaction mixture was concentrated in vacuo and purified by reverse phase HPLC (column: Waters Xbridge BEH C18 250*50 mm*10 μm; mobile phase: [water (NH₄HCO₃)-acetonitrile]; B %: 15%-45%, 10 min) affording 8-benzyl-6-methyl-2-oxa-5,8-diazaspiro[3.5]nonane (200 mg, 17.97%) as a colorless oil. LCMS Rt=2.363 min, m/z=233.2 [M+H]⁺.

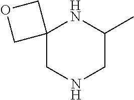

### Step 4: 6-methyl-2-oxa-5,8-diazaspiro[3.5]nonane

To a solution of Pd/C (200 mg, 1.72 mmol, 10% purity) in ethyl acetate (5 mL) was added 8-benzyl-6-methyl-2-oxa-5,8-diazaspiro[3.5]nonane (120 mg, 516.53 μmol). The mixture was stirred at 30° C. for 3 h under hydrogen (50 Psi). The reaction mixture was concentrated to dryness in vacuo affording 6-methyl-2-oxa-5,8-diazaspiro[3.5]nonane (60 mg, crude) as a yellow oil, which was used in the next step without further purification. LCMS Rt=0.239 min, m/z=143.1 [M+H]⁺.

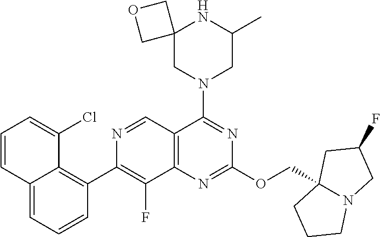 [SMILES: CC1CN(c2nc(OC[C@@]34CCCN3C[C@H](F)C4)nc3c(F)c(-c4cccc5cccc(Cl)c45)ncc23)CC2(COC2)N1]

pyrrolizin-7a(5H)-yl)methoxy)pyrido[4,3-d]pyrimidin-4-yl)-6-methyl-2-oxa-5,8-diazaspiro[3.5]nonane

The substitution reaction was prepared in a similar fashion to Method #6, Step 5. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 35%-70%, 8 min) affording 8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonane (20 mg, 46.53%) as a white solid. LCMS Rt=1.929 min, m/z=607.2 [M+H]⁺.

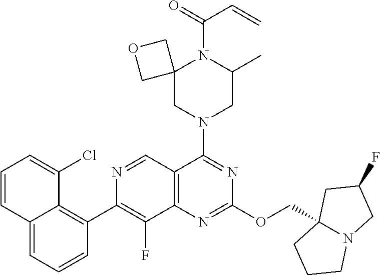 [SMILES: C=CC(=O)N1C(C)CN(c2nc(OC[C@@]34CCCN3C[C@H](F)C4)nc3c(F)c(-c4cccc5cccc(Cl)c45)ncc23)CC12COC2]

### Step 6: 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one

The acylation reaction was prepared in a similar fashion to Method #1, Step 5. The reaction mixture was concentrated in vacuo and purified by reverse phase HPLC (column: Waters Xbridge Prep OBD C18 150*40 mm*10 μm; mobile phase: [water (NH₄HCO₃)-acetonitrile]; B %: 30%-60%, 8 min) affording 1-(8-(7-(8-chloronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one (13.04 mg, 58.40%) as a yellow solid. ¹H NMR (400 MHz, Acetonitrile-d3) δ 9.20 (d, J=3.5 Hz, 1H), 8.14 (d, J=8.0 Hz, 1H), 8.04 (d, J=8.1 Hz, 1H), 7.74-7.68 (m, 1H), 7.68-7.61 (m, 2H), 7.57-7.50 (m, 1H), 6.62 (ddd, J=3.3, 10.5, 16.7 Hz, 1H), 6.30-6.19 (m, 1H), 5.76 (br d, J=10.4 Hz, 1H), 5.37-5.20 (m, 1H), 5.15-5.05 (m, 2H), 4.88 (br d, J=12.6 Hz, 1H), 4.53-4.44 (m, 2H), 4.39-4.31 (m, 1H), 4.28-4.22 (m, 1H), 4.21-4.13 (m, 2H), 4.06-3.90 (m, 2H), 3.22-3.14 (m, 2H), 3.13-3.07 (m, 1H), 2.96-2.88 (m, 1H), 2.21 (br s, 1H), 2.14 (br s, 1H), 2.11-2.04 (m, 1H), 1.94-1.82 (m, 3H), 1.37 (t, J=6.0 Hz, 3H).

LCMS (5% to 95% acetonitrile in water+0.03% ammonium bicarbonate over 6 min); retention time 2.206 min, ESI+ found [M+H]=661.2.

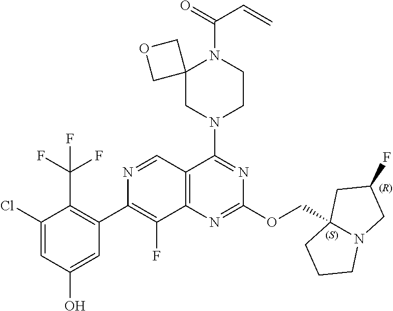

### Example 54 (Method 1): 1-(8-(7-(3-chloro-5-hydroxy-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

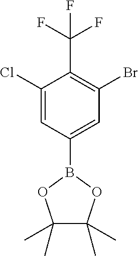 [SMILES: CC1(C)OB(c2cc(Cl)c(C(F)(F)F)c(Br)c2)OC1(C)C]

### Step 1: 2-(3-bromo-5-chloro-4-(trifluoromethyl)phenyl)-4,4,5,5-tetramethyl-1,3,2-dioxaborolane

To a solution of 1-bromo-3-chloro-2-(trifluoromethyl)benzene (10 g, 38.54 mmol) and 4,4,5,5-tetramethyl-1,3,2-dioxaborolane (9.87 g, 77.09 mmol) in tetrahydrofuran (20 mL) was added 4-tert-butyl-2-(4-tert-butyl-2-pyridyl)pyridine (1.24 g, 4.63 mmol) and (1,5-cyclooctadiene)(methoxy) iridium (I) dimer (2.55 g, 3.85 mmol). The mixture was stirred at 60° C. for 2 h under a nitrogen atmosphere. The mixture was concentrated in vacuo affording 2-(3-bromo-5-chloro-4-(trifluoromethyl)phenyl)-4,4,5,5-tetramethyl-1,3,2-dioxaborolane (15 g, crude) as a black oil, which was used in the next step without any further purification.

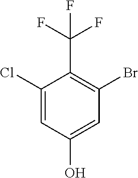 [SMILES: Oc1cc(Cl)c(C(F)(F)F)c(Br)c1]

### Step 2: 3-bromo-5-chloro-4-(trifluoromethyl) phenol

To a solution of 2-(3-bromo-5-chloro-4-(trifluoromethyl)phenyl)-4,4,5,5-tetramethyl-1,3,2-dioxaborolane (15 g, 38.92 mmol) in tetrahydrofuran (300 mL) and water (150 mL) was added acetic acid (93.49 g, 1.56 mol) and hydrogen peroxide (88.26 g, 778.39 mmol, 30% purity) at 0° C. The mixture was stirred at 25° C. for 1 h. The mixture was quenched with saturated sodium sulfite (500 mL) and extracted with ethyl acetate (3×200 mL). The combined organic layers were dried over sodium sulphate and concentrated in vacuo. The residue was purified by column chromatography (silica gel, 100-200 mesh, 0-100% ethyl acetate in petroleum ether) affording 3-bromo-5-chloro-4-(trifluoromethyl) phenol (9.5 g, 88.62%) as a yellow oil. LCMS Rt=0.741 min, m/z=276.9 [M+H]⁺.

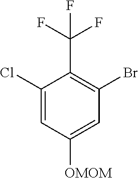

### Step 3: 1-bromo-3-chloro-5-(methoxymethoxy)-2-(trifluoromethyl)benzene

To a solution of 3-bromo-5-chloro-4-(trifluoromethyl) phenol (4.5 g, 16.34 mmol) in dichloromethane (10 mL) was added N,N-diisopropylethylamine (6.33 g, 49.01 mmol) and chloromethyl methyl ether (2.95 g, 36.64 mmol) at 0° C. The mixture was stirred at 25° C. for 1 h. The mixture was diluted with water (10 mL) and extracted with ethyl acetate (3×20 mL). The combined organic layers were dried over sodium sulphate and concentrated in vacuo. The residue was purified by column chromatography (silica gel, 100-200 mesh, 0-100% ethyl acetate in petroleum ether) affording 1-bromo-3-chloro-5-(methoxymethoxy)-2-(trifluoromethyl)benzene (4.7 g, 90.04%) as a yellow oil: ¹H NMR (400 MHz, Chloroform-d) δ 7.36 (d, J=2.0 Hz, 1H), 7.18 (d, J=1.8 Hz, 1H), 5.22 (s, 2H), 3.51 (s, 3H).

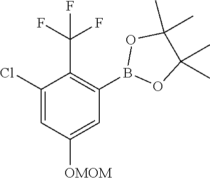

### Step 4: 2-(3-chloro-5-(methoxymethoxy)-2-(trifluoromethyl)phenyl)-4,4,5,5-tetramethyl-1,3,2-dioxaborolane

A mixture of 1-bromo-3-chloro-5-(methoxymethoxy)-2-(trifluoromethyl)benzene (3.8 g, 11.89 mmol), 4,4,4′,4′,5,5,5′,5′-octamethyl-2,2′-bi (1,3,2-dioxaborolane)(3.32 g, 13.08 mmol), [1,1-Bis(diphenylphosphino) ferrocene]dichloropalladium (II)(870.25 mg, 1.19 mmol) and potassium acetate (5.84 g, 59.47 mmol) in dioxane (100 mL) was degassed and purged with nitrogen 3 times. The mixture was stirred at 100° C. for 12 h under a nitrogen atmosphere. The mixture was concentrated to dryness in vacuo, and the resulting residue was purified by reverse phase HPLC (column: Welch Xtimate C_{18 250*100} mm #10 μm; mobile phase: [water (NH₄HCO₃)-ACN]; gradient: 55%-85% B over 20 min) affording 2-(3-chloro-5-(methoxymethoxy)-2-(trifluoromethyl)phenyl)-4,4,5,5-tetramethyl-1,3,2-dioxaborolane (0.9 g, 20.64%) as a yellow oil: ¹H NMR (400 MHz, Chloroform-d) δ 7.11 (s, 1H), 6.90 (s, 1H), 5.11 (s, 2H), 3.38 (s, 3H), 1.29 (s, 12H).

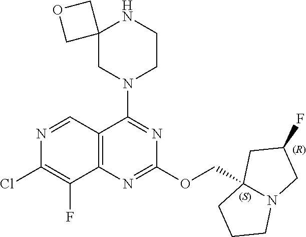

### Step 5: 8-(7-chloro-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

A mixture of 2-oxa-5,8-diazaspiro[3.5]nonane (2.75 g, 13.67 mmol, 2 hydrochloric acid salt), 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (3 g, 6.84 mmol), N,N-diisopropylethylamine (4.42 g, 34.19 mmol) and 4A molecular sieve (50 mg) in N,N-dimethylformaldehyde (3 mL) was degassed and purged with nitrogen 3 times. The mixture was stirred at 60° C. for 12 h under a nitrogen atmosphere. The mixture was concentrated to dryness in vacuo, and the resulting residue was purified by reverse phase HPLC (column: Waters Xbridge BEH C18 250*70 mm*10 μm; mobile phase: [water (NH₄HCO₃)-ACN]; B %: 15%-50%, 18 min) affording 8-(7-chloro-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (2 g, 62.65%) as a light yellow solid. LCMS Rt=0.256 min, m/z=467.2 [M+H]⁺.

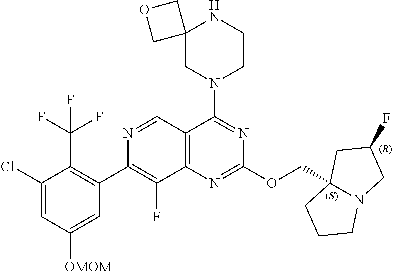

### Step 6: 8-(7-(3-chloro-5-(methoxymethoxy)-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane

A mixture of 8-(7-chloro-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (200 mg, 428.35 μmol), 2-[3-chloro-5-(methoxymethoxy)-2-(trifluoromethyl)phenyl]-4,4,5,5-tetramethyl-1,3,2-dioxaborolane (235.53 mg, 642.52 μmol), tetrakis(triphenylphosphine) palladium (0)(49.50 mg, 42.83 μmol) and sodium carbonate (136.20 mg, 1.29 mmol) in dioxane (3 mL) and water (1 mL) was degassed and purged with nitrogen 3 times. The mixture was stirred at 80° C. for 2 h under a nitrogen atmosphere. The mixture was diluted with water (3 mL) and extracted with ethyl acetate (3×3 mL). The combined organic layers were dried over sodium sulphate and concentrated in vacuo. The resulting residue was purified by reverse phase HPLC (column: Waters Xbridge BEH C18 250*50 mm*10 μm; mobile phase: [water (NH₄HCO₃)-ACN]; gradient: 35%-75% B over 10 min) affording 8-(7-(3-chloro-5-(methoxymethoxy)-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (0.4 g, 62.65%) as a brown solid. LCMS Rt=0.511 min, m/z=671.2 [M+H]⁺.

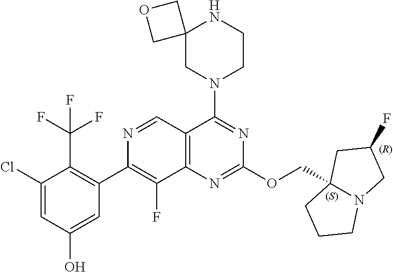

### Step 7: 3-chloro-5-(8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl)pyrido[4,3-d]pyrimidin-7-yl)-4-(trifluoromethyl) phenol

A solution of 8-(7-(3-chloro-5-(methoxymethoxy)-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (100 mg, 149.02 μmol) in dichloromethane (1.6 mL) and trifluoroacetic acid (0.4 mL) was stirred at 25° C. for 1 h. The mixture was concentrated in vacuo affording 3-chloro-5-(8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl)pyrido[4,3-d]pyrimidin-7-yl)-4-(trifluoromethyl) phenol (100 mg, crude, trifluoroacetate salt) as a yellow oil, which was used in the next step without any further purification. LCMS Rt=0.433 min, m/z=626.2 [M+H]⁺.

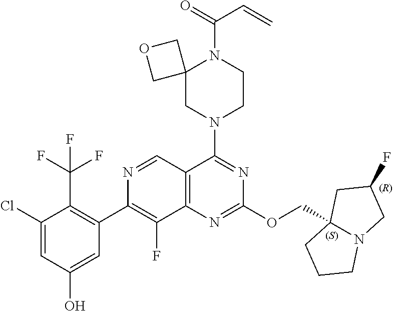

### Step 8: 1-(8-(7-(3-chloro-5-hydroxy-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one

The acylation reaction was prepared in a similar fashion to Method #1, Step 5. The resulting residue was purified by reverse phase HPLC (column: Waters Xbridge Prep OBD

C18 150*40 mm*10 μm; mobile phase: [H₂O (10 mM NH₄HCO₃)-ACN]; gradient: 25%-55% B over 8 min) affording 1-(8-(7-(3-chloro-5-hydroxy-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) prop-2-en-1-one (22.16 mg, 22.79%) as a yellow amorphous solid: ¹H NMR (400 MHz, Acetonitrile-d3) δ 9.07 (s, 1H), 7.20 (d, J=1.8 Hz, 1H), 6.83 (s, 1H), 6.67 (dd, J=10.4, 16.7 Hz, 1H), 6.29 (dd, J=1.9, 16.8 Hz, 1H), 5.79 (dd, J=2.0, 10.5 Hz, 1H), 5.40-5.18 (m, 1H), 4.88 (br d, J=5.8 Hz, 2H), 4.51 (br s, 1H), 4.44 (d, J=7.1 Hz, 3H), 4.31-4.18 (m, 2H), 3.93 (br d, J=2.6 Hz, 2H), 3.66 (br s, 2H), 3.25-3.09 (m, 3H), 3.02-2.91 (m, 1H), 2.24 (br d, J=4.1 Hz, 2H), 1.94-1.81 (m, 4H). LCMS (5% to 95% acetonitrile in water+0.03% ammonium bicarbonate over 6 min); retention time 2.192 min. ESI+ found [M+H]=681.2.

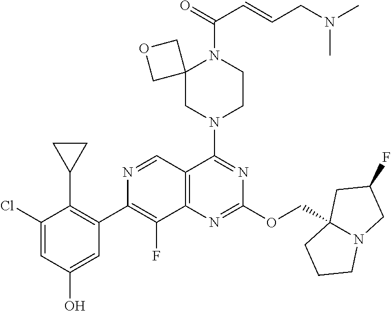 [SMILES: CN(C)C/C=C/C(=O)N1CCN(c2nc(OC[C@@]34CCCN3C[C@H](F)C4)nc3c(F)c(-c4cc(O)cc(Cl)c4C4CC4)ncc23)CC12COC2]

### Example 55 (Method 3): (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one

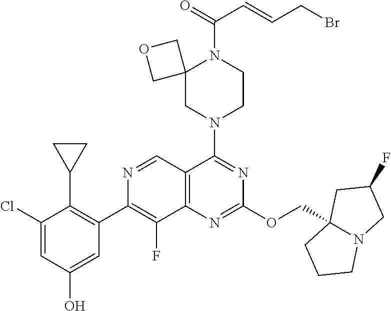 [SMILES: O=C(/C=C/CBr)N1CCN(c2nc(OC[C@@]34CCCN3C[C@H](F)C4)nc3c(F)c(-c4cc(O)cc(Cl)c4C4CC4)ncc23)CC12COC2]

### Step 1: (E)-4-bromo-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The amide coupling reaction was prepared in a similar fashion to Method #3, Step 2. The reaction mixture was concentrated in vacuo affording (E)-4-bromo-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (74 mg, crude) as a pale yellow gum. LCMS Rt=0.597 min, m/z=747.2 [M+H]⁺.

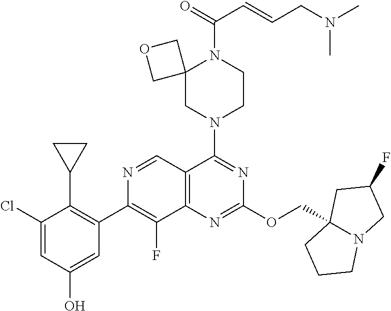 [SMILES: CN(C)C/C=C/C(=O)N1CCN(c2nc(OC[C@@]34CCCN3C[C@H](F)C4)nc3c(F)c(-c4cc(O)cc(Cl)c4C4CC4)ncc23)CC12COC2]

### Step 2: (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one

The substitution reaction was prepared in a similar fashion to Method #3, Step 3. The resulting residue was purified by reverse phase HPLC (column: Waters Xbridge Prep OBD C18 150*40 mm*10 μm; mobile phase: [water (NH₄HCO₃)-ACN]; B %: 20%-50%, 8 min) affording (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-4-(dimethylamino)but-2-en-1-one (21.54 mg, 30.43%) as a pale yellow amorphous solid: ¹H NMR (400 MHz, Chloroform-d) δ 8.95-8.86 (m, 1H), 7.00-6.89 (m, 1H), 6.85-6.78 (m, 1H), 6.56 (d, J=1.9 Hz, 1H), 6.46-6.34 (m, 1H), 5.43-5.20 (m, 1H), 4.90-4.79 (m, 2H), 4.45 (br t, J=6.0 Hz, 2H), 4.39-4.29 (m, 3H), 4.28-4.21 (m, 1H), 3.81-3.60 (m, 2H), 3.53-3.39 (m, 2H), 3.35-3.24 (m, 2H), 3.23-3.19 (m, 1H), 3.18-3.15 (m, 2H), 3.06-2.96 (m, 1H), 2.33 (s, 6H), 2.29-2.09 (m, 3H), 2.04-1.89 (m, 3H), 1.86-1.77 (m, 1H), 0.72-0.40 (m, 2H), 0.10-0.31 (m, 2H). LCMS (5% to 95% acetonitrile in water+0.1% trifluoroacetic acid over 6 min); retention time 2.801 min, ESI+ found [M+H]=710.3.

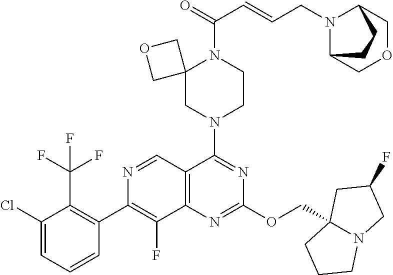

### Example 56 (Method 3): (E)-4-((1R,5S)-3-oxa-8-azabicyclo[3.2.1]octan-8-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

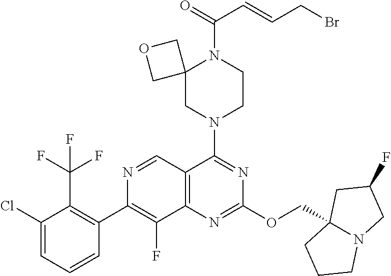 [SMILES: O=C(/C=C/CBr)N1CCN(c2nc(OC[C@@]34CCCN3C[C@H](F)C4)nc3c(F)c(-c4cccc(Cl)c4C(F)(F)F)ncc23)CC12COC2]

### Step 1: (E)-4-bromo-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The acylation reaction was prepared in a similar fashion to Method #3, Step 2. The mixture was concentrated in vacuo affording (E)-4-bromo-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (110 mg, crude) as a yellow oil, which was used in the next step without further purification. LCMS Rt=0.698 min, m/z=759.1 [M+H]⁺.

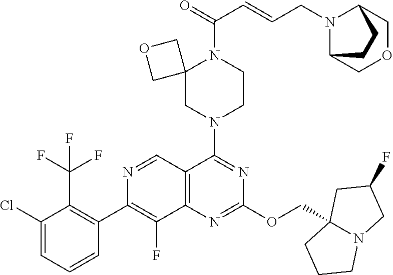

### Step 2: (E)-4-((1R,5S)-3-oxa-8-azabicyclo[3.2.1]octan-8-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording (E)-4-((1R,5S)-3-oxa-8-azabicyclo[3.2.1]octan-8-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (25.25 mg, 22.02%) as a yellow solid: ¹H NMR (400 MHz, Chloroform-d) δ 9.05-9.02 (m, 1H), 7.70-7.65 (m, 1H), 7.61-7.54 (m, 1H), 7.39-7.34 (m, 1H), 7.02-6.92 (m, 1H), 6.56-6.47 (m, 1H), 5.47-5.19 (m, 1H), 4.93-4.81 (m, 2H), 4.59-4.20 (m, 7H), 4.02-3.82 (m, 2H), 3.80-3.74 (m, 2H), 3.71-3.62 (m, 2H), 3.58-3.56 (m, 1H), 3.55 (d, J=1.0 Hz, 1H), 3.36-3.18 (m, 2H), 3.17-3.12 (m, 2H), 3.10-3.01 (m, 3H), 2.38-2.17 (m, 3H), 2.05-1.98 (m, 2H), 1.97-1.91 (m, 5H).

LCMS (5% to 95% acetonitrile in water+0.03% ammonium bicarbonate over 6 min); retention time 3.067 min, ESI+ found [M+H]=790.3.

### Example 57 (Method 3): (E)-4-((1S,4S)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

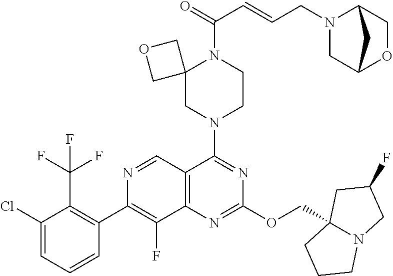 [SMILES: O=C(/C=C/CN1C[C@@H]2C[C@H]1CO2)N1CCN(c2nc(OC[C@@]34CCCN3C[C@H](F)C4)nc3c(F)c(-c4cccc(Cl)c4C(F)(F)F)ncc23)CC12COC2]

### Step 1: (E)-4-((1S,4S)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

The substitution reaction was prepared in a similar fashion to Method #3, Step 3. The residue was purified by reverse phase HPLC (column: Waters Xbridge Prep OBD C18 150*40 mm*10 μm; mobile phase: [water (NH₄HCO₃)-ACN]; B %: 25%-55%, 8 min) affording (E)-4-((1S,4S)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl) but-2-en-1-one (14.06 mg, 12.48%) as a yellow oil: ¹H NMR (400 MHz, Chloroform-d) δ 8.97-8.93 (m, 1H), 7.62-7.57 (m, 1H), 7.52-7.47 (m, 1H), 7.30-7.26 (m, 1H), 6.94-6.83 (m, 1H), 6.42-6.30 (m, 1H), 5.33-5.12 (m, 1H), 4.85-4.73 (m, 2H), 4.45-4.29 (m, 5H), 4.28-4.17 (m, 2H), 3.98-3.93 (m, 1H), 3.91-3.69 (m, 2H), 3.64-3.57 (m, 2H), 3.57-3.49 (m, 1H), 3.46-3.43 (m, 1H), 3.42-3.35 (m, 1H), 3.35-3.29 (m, 1H), 3.28-3.16 (m, 2H), 3.16-3.07 (m, 1H), 2.97-2.89 (m, 1H), 2.86-2.82 (m, 1H), 2.56-2.50 (m, 1H), 2.24-2.06 (m, 3H), 1.93-1.86 (m, 2H), 1.81 (br d, J=9.5 Hz, 3H).

LCMS (5% to 95% acetonitrile in water+0.03% ammonium bicarbonate over 6 min); retention time 2.958 min, ESI+ found [M+H]=776.3.

### Example 58 (Method 1): (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

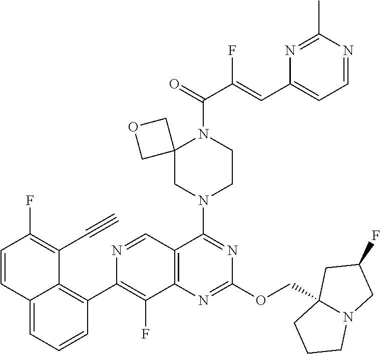 [SMILES: C#Cc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12]

### Step 1: (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

To a solution of 8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonane (45 mg, 74.92 μmol), (Z)-2-fluoro-3-(2-methylpyrimidin-4-yl) acrylic acid (27.29 mg, 149.84 μmol) and N,N-diisopropylethylamine (19.37 mg, 149.84 μmol) in N,N-dimethylformamide (1 mL) was added 2-chloro-1,3-dimethyl-4,5-dihydroimidazol-1-ium; chloride (19.00 mg, 112.38 μmol) at 0° C., the mixture was stirred at 0° C. for 1 h. The crude product was purified by reverse phase prep-HPLC (column: Waters Xbridge Prep OBD C18 150*40 mm*10 μm; mobile phase: [water (NH₄HCO₃)-ACN]; gradient: 35%-65% B over 8 min) affording (Z)-1-(8-(7-(8-ethynyl-7-fluoronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one (16.41 mg, 27.58%) as a white solid: ¹H NMR (400 MHz, Acetonitrile-d3) δ 9.10 (s, 1H), 8.74 (d, J=5.3 Hz, 1H), 8.20-8.11 (m, 2H), 7.70 (br d, J=4.0 Hz, 2H), 7.63-7.56 (m, 1H), 7.47 (br t, J=9.0 Hz, 1H), 6.68-6.55 (m, 1H), 5.39-5.17 (m, 1H), 4.97-4.88 (m, 2H), 4.61-4.48 (m, 3H), 4.43 (br d, J=6.6 Hz, 1H), 4.28-4.20 (m, 1H), 4.18-4.07 (m, 2H), 3.94-3.82 (m, 3H), 3.32 (s, 1H), 3.19-3.06 (m, 3H), 2.98-2.86 (m, 1H), 2.68 (s, 3H), 2.14-2.00 (m, 3H), 1.88 (br dd, J=7.4, 10.5 Hz, 3H). LCMS Rt=2.998 min, m/z=765.3 [M+H]⁺.

LCMS (5 to 95% acetonitrile in water+0.03% ammonium bicarbonate over 6 mins) retention time 2.998 min, ESI+ found [M+H]=765.3.

### Example 59 (Method 1): (Z)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl)-2-fluoroprop-2-en-1-one

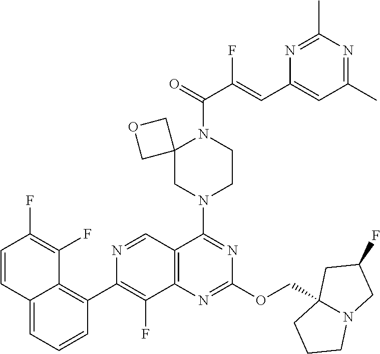 [SMILES: Cc1cc(/C=C(\F)C(=O)N2CCN(c3nc(OC[C@@]45CCCN4C[C@H](F)C5)nc4c(F)c(-c5cccc6ccc(F)c(F)c56)ncc34)CC23COC3)nc(C)n1]

### Step 1: (Z)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-(2,6-dimethylpyrimidin-4-yl)-2-fluoroprop-2-en-1-one

The amide coupling reaction was prepared in a similar fashion to Method #1, Step 1. The crude product was purified by reverse phase prep-HPLC (column: Waters Xbridge Prep OBD C18 150*40 mm*10 μm; mobile phase: [H₂O (10 mM NH₄HCO₃)-ACN]; gradient: 40%-70% B over 8.0 min) affording (Z)-1-(8-(7-(7,8-difluoronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl)-3-(2,6-dimethylpyrimidin-4-yl)-2-fluoroprop-2-en-1-one (201.39 mg, 30.99%) as a white solid: ¹H NMR (400 MHz, Acetonitrile-d3) δ 9.15 (s, 1H), 8.19-8.10 (m, 1H), 7.92 (ddd, J=1.4, 5.1, 9.1 Hz, 1H), 7.76-7.66 (m, 2H), 7.62-7.45 (m, 2H), 6.75-6.51 (m, 1H), 5.38-5.16 (m, 1H), 4.94 (br d, J=6.2 Hz, 2H), 4.69-4.42 (m, 4H), 4.29-4.12 (m, 2H), 4.05-3.93 (m, 2H), 3.86-3.75 (m, 2H), 3.26-3.01 (m, 3H), 2.92 (q, J=7.5 Hz, 1H), 2.62 (s, 3H), 2.50 (s, 3H), 2.20 (br s, 1H), 2.13 (br s, 2H), 1.93-1.79 (m, 3H). LCMS Rt=2.246 min, m/z=773.3 [M+H]⁺.

LCMS (5 to 95% acetonitrile in water+0.1% trifluoroacetic acid over 6 mins) retention time 2.246 min, ESI+ found [M+H]=773.3.

### Example 60 (Method 1): (Z)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

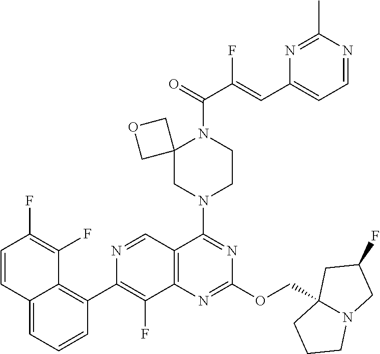 [SMILES: Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)CC23COC3)n1]

### Step 1: (Z)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

The amide coupling reaction was prepared in a similar fashion to Method #1, Step 1. The crude product was purified by reverse phase prep-HPLC (column: Waters Xbridge BEH C18 100*30 mm*10 μm; mobile phase: [water (NH₄HCO₃)-ACN]; gradient: 40%-70% B over 8 min) affording (Z)-1-(8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one (10.09 mg, 15.81%) as a white solid: ¹H NMR (400 MHz, Acetonitrile-d3) δ 9.22-9.11 (m, 1H), 8.80-8.61 (m, 1H), 8.21-8.09 (m, 1H), 7.92 (ddd, J=1.6, 5.1, 9.1 Hz, 1H), 7.78-7.67 (m, 2H), 7.64-7.46 (m, 2H), 6.72-6.57 (m, 1H), 5.37-5.21 (m, 1H), 4.94 (br d, J=3.1 Hz, 2H), 4.64-4.55 (m, 1H), 4.54-4.44 (m, 3H), 4.34-4.15 (m, 2H), 4.13-3.97 (m, 2H), 3.93-3.72 (m, 2H), 3.25-3.08 (m, 3H), 3.01-2.86 (m, 1H), 2.75-2.58 (m, 3H), 2.23 (br d, J=4.3 Hz, 2H), 2.16-2.12 (m, 2H), 1.94-1.88 (m, 2H). LCMS Rt=3.034 min, m/z=759.3 [M+H]⁺.

LCMS (5 to 95% acetonitrile in water+0.03% ammonium bicarbonate over 6 mins) retention time 3.034 min, ESI+ found [M+H]=759.3.

### Example 62 (Method 1): (Z)-1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

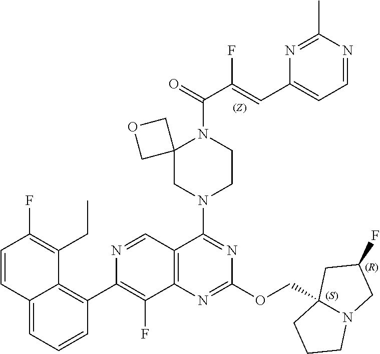

### Step 1: (Z)-1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one

The amide coupling reaction was prepared in a similar fashion to Method #1, Step 1. The crude product was purified by prep-HPLC (column: 2_Phenomenex Gemini C_{18 75*40} mm*3 μm; mobile phase: [H₂O (10 mM NH₄HCO₃)-ACN]; gradient: 30%-55% B over 8.0 min) affording (Z)-1-(8-(7-(8-ethyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoro-3-(2-methylpyrimidin-4-yl) prop-2-en-1-one (12 mg, 6.02%) as a yellow solid: ¹H NMR (400 MHz, Acetonitrile-d3) δ 9.13 (s, 1H), 8.74 (d, J=5.3 Hz, 1H), 8.08 (dd, J=1.3, 8.1 Hz, 1H), 7.96 (dd, J=6.1, 8.9 Hz, 1H), 7.62-7.48 (m, 3H), 7.41 (t, J=9.4 Hz, 1H), 6.70-6.57 (m, 1H), 5.38-5.19 (m, 1H), 4.94 (d, J=6.9 Hz, 2H), 4.63-4.46 (m, 4H), 4.29-4.24 (m, 1H), 4.21-4.16 (m, 1H), 4.05-3.98 (m, 2H), 3.83 (br d, J=5.0 Hz, 2H), 3.19-3.08 (m, 3H), 2.97-2.87 (m, 1H), 2.67 (s, 3H), 2.30 (dt, J=3.0, 7.2 Hz, 2H), 2.15-2.03 (m, 3H), 1.93-1.84 (m, 3H), 0.86-0.79 (m, 3H). LCMS Rt=3.185 min, m/z=769.3 [M+H]⁺.

LCMS (5% to 95% acetonitrile in water+0.03% ammonium bicarbonate over 6 min); retention time 3.185 min, ESI+ found [M+H]=769.3.

### Example 63 (Method 1): (Z)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl)-2-fluoroprop-2-en-1-one

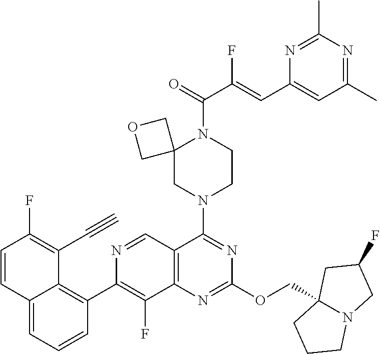 [SMILES: C#Cc1c(F)ccc2cccc(-c3ncc4c(N5CCN(C(=O)/C(F)=C/c6cc(C)nc(C)n6)C6(COC6)C5)nc(OC[C@@]56CCCN5C[C@H](F)C6)nc4c3F)c12]

### Step 1: (Z)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoroprop-2-en-1-one

The amide coupling reaction was prepared in a similar fashion to Method #1, Step 1. The crude product was purified by prep-HPLC (column: Waters Xbridge BEH C18 100*30 mm*10 μm; mobile phase: [water (NH₄HCO₃)-ACN]; B %: 30%-70%, 8 min) affording (Z)-3-(2,6-dimethylpyrimidin-4-yl)-1-(8-(7-(8-ethynyl-7-fluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-fluoroprop-2-en-1-one (21.57 mg, 20.79%) as a yellow amorphous solid: ¹H NMR (400 MHz, Chloroform-d) δ 9.03 (br d, J=2.8 Hz, 1H), 8.03-7.93 (m, 2H), 7.69-7.58 (m, 2H), 7.44-7.39 (m, 1H), 7.35 (t, J=8.8 Hz, 1H), 6.83-6.66 (m, 1H), 5.47-5.23 (m, 1H), 4.93-4.85 (m, 1H), 4.82-4.76 (m, 1H), 4.69-4.62 (m, 1H), 4.53-4.45 (m, 2H), 4.44-4.35 (m, 1H), 4.31 (br d, J=7.1 Hz, 1H), 4.24-4.12 (m, 1H), 3.92-3.73 (m, 3H), 3.50-3.20 (m, 2H), 3.18-3.01 (m, 2H), 2.73 (s, 3H), 2.57 (s, 3H), 2.44-2.16 (m, 3H), 2.14-1.94 (m, 3H), 1.85-1.64 (m, 2H). LCMS Rt=3.018 min, m/z=779.3 [M+H]⁺.

LCMS (5% to 95% acetonitrile in water+0.03% ammonium bicarbonate over 6 min); retention time 3.018 min, ESI+ found [M+H]=779.3.

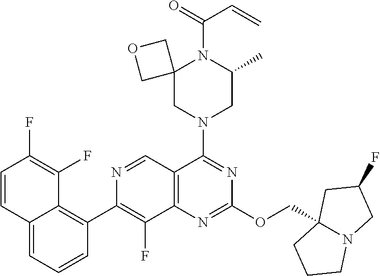 [SMILES: C=CC(=O)N1[C@H](C)CN(c2nc(OC[C@@]34CCCN3C[C@H](F)C4)nc3c(F)c(-c4cccc5ccc(F)c(F)c45)ncc23)CC12COC2]

### Example 64 (Method 2): 1-((R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one

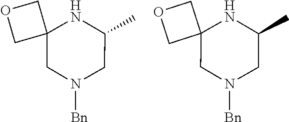

### Step 1: (R)-8-benzyl-6-methyl-2-oxa-5,8-diazaspiro[3.5]nonane and (S)-8-benzyl-6-methyl-2-oxa-5,8-diazaspiro[3.5]nonane

The racemic material (150 mg) was further purified by SFC to give arbitrarily assigned:

(R)-8-benzyl-6-methyl-2-oxa-5,8-diazaspiro[3.5]nonane (Peak 2, retention time=2.068 min)(70 mg, 46.67%) as a white solid. LCMS Rt=2.254 min, m/z=233.2 [M+H]⁺.

(S)-8-benzyl-6-methyl-2-oxa-5,8-diazaspiro[3.5]nonane (Peak 1, retention time=1.819 min)(70 mg, 46.67%) as a white solid. LCMS Rt=2.254 min, m/z=233.2 [M+H]⁺.

SFC (column: DAICEL CHIRALPAK AD (250 mm*30 mm, 10 μm); mobile phase: [CO₂-IPA (0.1% NH₃H₂O)]; B %: 10%, isocratic elution mode).

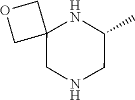

### Step 2: (R)-6-methyl-2-oxa-5,8-diazaspiro[3.5]nonane

To a solution of (R)-8-benzyl-6-methyl-2-oxa-5,8-diazaspiro[3.5]nonane (70 mg, 301.31 μmol) in ethyl acetate (5 mL) was added Pd/C (200 mg, 1.72 mmol, 10% purity). The mixture was stirred at 30° C. for 3 h under hydrogen (50 Psi). The reaction mixture was concentrated to dryness in vacuo affording (R)-6-methyl-2-oxa-5,8-diazaspiro[3.5]nonane (50 mg, crude) as a yellow oil, used in next step without further purification. LCMS Rt=0.157 min, m/z=143.3 [M+H]⁺.

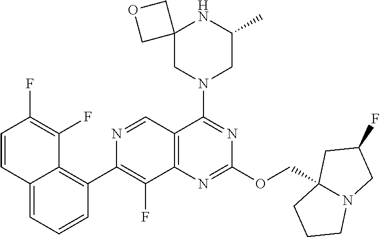 [SMILES: C[C@@H]1CN(c2nc(OC[C@@]34CCCN3C[C@H](F)C4)nc3c(F)c(-c4cccc5ccc(F)c(F)c45)ncc23)CC2(COC2)N1]

### Step 3: (R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonane

A mixture of 7-(7,8-difluoronaphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (60 mg, 105.92 μmol), (R)-6-methyl-2-oxa-5,8-diazaspiro[3.5]nonane (45.18 mg, 317.76 μmol), N,N-diisopropylethylamine (82.14 mg, 635.52 μmol) and 4A molecule sieve (20 mg) in N,N-dimethylformamide (0.1 mL) was degassed and purged with nitrogen 3 times, and the mixture was stirred at 60° C. for 12 h under nitrogen atmosphere. The reaction mixture was concentrated in vacuo and purified by reverse phase HPLC (column: Waters Xbridge Prep OBD C18 150*40 mm*10 μm; mobile phase: [H₂O (10 mM NH₄HCO₃)-ACN]; gradient: 20%-50% B over 8.0 min) affording (R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonane (50 mg, 77.56%) as a white solid. LCMS Rt=1.873 min, m/z=609.3 [M+H]⁺.

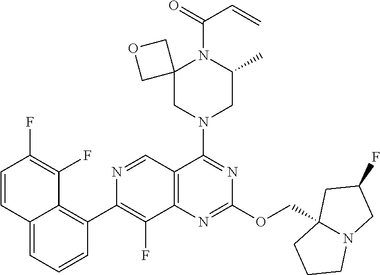 [SMILES: C=CC(=O)N1[C@H](C)CN(c2nc(OC[C@@]34CCCN3C[C@H](F)C4)nc3c(F)c(-c4cccc5ccc(F)c(F)c45)ncc23)CC12COC2]

### Step 4: 1-((R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one

To a solution of (R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonane (50 mg, 82.15 μmol) in dichloromethane (3 mL) was added 2,6-dimethylpyridine (17.61 mg, 164.30 μmol) and prop-2-enoyl chloride (8.92 mg, 98.58 μmol) under nitrogen atmosphere at −78° C. The mixture was stirred at −78° C. for 0.5 h. The reaction mixture was concentrated in vacuo and purified by reverse phase HPLC (column: Waters Xbridge Prep OBD C18 150*40 mm*10 μm; mobile phase: [H₂O (10 mM NH₄HCO₃)-ACN]; gradient: 35%-65% B over 8.0 min) affording 1-((R)-8-(7-(7,8-difluoronaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-methyl-2-oxa-5,8-diazaspiro[3.5]nonan-5-yl) prop-2-en-1-one (21.33 mg, 35.27%) as a white solid: ¹H NMR (400 MHz, Acetonitrile-d3) δ 9.23 (s, 1H), 8.19-8.10 (m, 1H), 7.92 (ddd, J=1.7, 5.1, 9.1 Hz, 1H), 7.78-7.67 (m, 2H), 7.56 (dt, J=7.5, 9.6 Hz, 1H), 6.63 (dd, J=10.5, 16.8 Hz, 1H), 6.26 (br dd, J=2.3, 16.8 Hz, 1H), 5.76 (br d, J=10.5 Hz, 1H), 5.38-5.18 (m, 1H), 5.12 (td, J=6.6, 10.7 Hz, 2H), 4.88 (d, J=12.8 Hz, 1H), 4.49 (br dd, J=6.2, 11.1 Hz, 2H), 4.41-4.34 (m, 1H), 4.29-4.12 (m, 3H), 4.09-3.99 (m, 1H), 3.98-3.90 (m, 1H), 3.20-3.12 (m, 2H), 3.09 (s, 1H), 2.96-2.87 (m, 1H), 2.24-2.20 (m, 1H), 2.14-2.04 (m, 2H), 1.93-1.78 (m, 3H), 1.39 (d, J=6.9 Hz, 3H). LCMS Rt=2.102 min, m/z=663.3 [M+H]+.

LCMS (5 to 95% acetonitrile in water+0.1% trifluoroacetic acid over 6 mins) retention time 2.102 min, ESI+ found [M+H]=663.3.

### Example 68 (Method 3): (E)-4-((1R,4R)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one

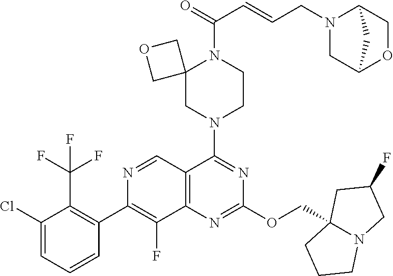 [SMILES: O=C(/C=C/CN1C[C@H]2C[C@@H]1CO2)N1CCN(c2nc(OC[C@@]34CCCN3C[C@H](F)C4)nc3c(F)c(-c4cccc(Cl)c4C(F)(F)F)ncc23)CC12COC2]

### Step 1: (E)-4-((1R,4R)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl) but-2-en-1-one

To a solution of (1R,4R)-2-oxa-5-azabicyclo[2.2.1]heptane (70.84 mg, 522.44 μmol) in tetrahydrofuran (1 mL) was added N,N-diisopropylethylamine (168.80 mg, 1.31 mmol), the mixture was stirred at 25° C. for 10 min. Then (E)-4-bromo-1-(8-(7-(3-chloro-2-(trifluoromethyl)phenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl) but-2-en-1-one (99 mg, 130.61 μmol) was added to the above solution at 0° C., the mixture was stirred at 25° C. for 2 h. The reaction mixture was concentrated in vacuo and purified by reverse phase HPLC (column: Waters Xbridge Prep OBD C18 150*40 mm*10 μm; mobile phase: [water (NH₄HCO₃)-ACN]; B %: 35%-65%, 8 min) affording (E)-4-((1R,4R)-2-oxa-5-azabicyclo[2.2.1]heptan-5-yl)-1-(8-(7-(3-chloro-2-(trifluoromethyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl) but-2-en-1-one (24.39 mg, 24.06%) as a yellow amorphous solid: ¹H NMR (400 MHz, Chloroform-d) δ 9.03 (s, 1H), 7.70-7.64 (m, 1H), 7.61-7.54 (m, 1H), 7.39-7.33 (m, 1H), 7.01-6.91 (m, 1H), 6.47-6.38 (m, 1H), 5.38-5.19 (m, 1H), 4.95-4.79 (m, 2H), 4.58-4.35 (m, 5H), 4.34-4.17 (m, 2H), 4.07-4.00 (m, 1H), 3.99-3.76 (m, 2H), 3.72-3.57 (m, 3H), 3.54-3.36 (m, 3H), 3.30-3.14 (m, 3H), 3.03-2.87 (m, 2H), 2.60 (d, J=10.1 Hz, 1H), 2.29-2.10 (m, 3H), 2.01-1.84 (m, 4H), 1.78 (br d, J=9.8 Hz, 1H). LCMS Rt=2.961 min, m/z=776.3/777.3 [M+H]⁺. LCMS (5% to 95% acetonitrile in water+0.03% ammonium bicarbonate over 6 min); retention time 2.961 min, ESI+ found [M+H]=776.3/777.3.

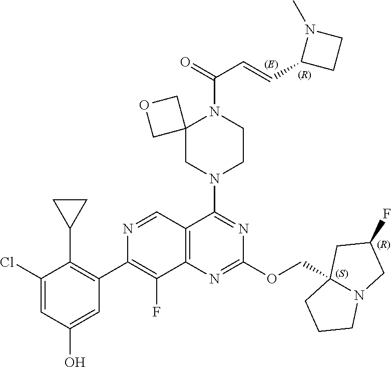

### Example 73 (Method 4): (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one

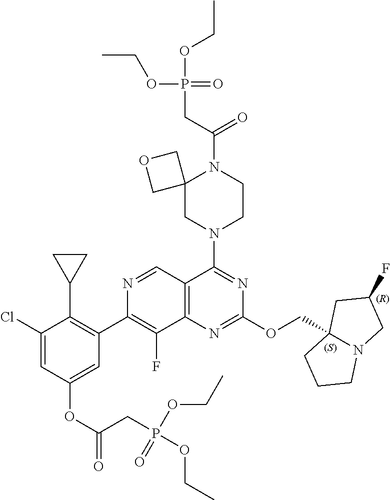

### Step 1: 3-chloro-4-cyclopropyl-5-(4-(5-(2-(diethoxyphosphoryl) acetyl)-2-oxa-5,8-diazaspiro[3.5]nonan-8-yl)-8-fluoro-2-(((2R,7aS)-2-fluorotetrahydro-1H-pyrrolizin-7a(5H)-yl) methoxy)pyrido[4,3-d]pyrimidin-7-yl)phenyl 2-(diethoxyphosphoryl)acetate

The amide coupling reaction was prepared in a similar fashion to Method #1, Step 1. The reaction mixture was concentrated to dryness in vacuo affording 3-chloro-4-cyclopropyl-5-(4-(5-(2-(diethoxyphosphoryl) acetyl)-2-oxa-5,8-diazaspiro[3.5]nonan-8-yl)-8-fluoro-2-(((2R,7aS)-2-fluorotetrahydro-1H-pyrrolizin-7a(5H)-yl)methoxy)pyrido[4,3-d]pyrimidin-7-yl)phenyl 2-(diethoxyphosphoryl)acetate (160 mg, crude) as an orange gum, used in next step without any further purification. LCMS Rt=0.415 min, m/z=955.4/956.4 [M+H]⁺.

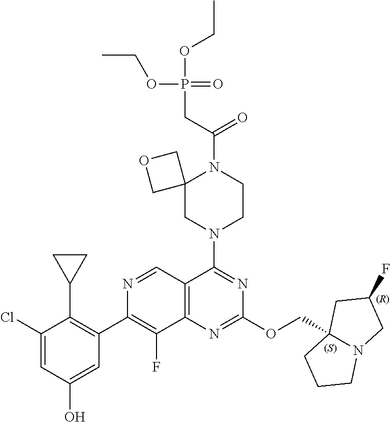

### Step 2: diethyl(2-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-oxoethyl)phosphonate

To a solution of 3-chloro-4-cyclopropyl-5-(4-(5-(2-(diethoxyphosphoryl) acetyl)-2-oxa-5,8-diazaspiro[3.5]nonan-8-yl)-8-fluoro-2-(((2R,7aS)-2-fluorotetrahydro-1H-pyrrolizin-7a(5H)-yl)methoxy)pyrido[4,3-d]pyrimidin-7-yl)phenyl 2-(diethoxyphosphoryl)acetate (160 mg, 167.48 μmol) in tetrahydrofuran (1.5 mL) was added lithium hydroxide monohydrate (21.08 mg, 502.45 μmol) at 0° C., the mixture was stirred at 20° C. for 1 h. The mixture was diluted with water (3 mL) and extracted with dichloromethane (3×2 mL). The combined organic layers were dried over sodium sulphate and concentrated in vacuo. The residue was purified by column chromatography (silica gel, 100-200 mesh, 0-100% tetrahydrofuran in petroleum ether) affording diethyl(2-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-oxoethyl)phosphonate (90 mg, 53.65%) as a yellow solid: ¹H NMR (400 MHz, dimethyl sulfoxide-d6) δ 10.11-9.91 (m, 1H), 9.31-9.15 (m, 1H), 6.97 (d, J=2.5 Hz, 1H), 6.79 (d, J=2.5 Hz, 1H), 5.68-5.39 (m, 1H), 4.85 (br d, J=6.4 Hz, 2H), 4.61-4.48 (m, 2H), 4.44-4.40 (m, 2H), 4.34 (br d, J=6.0 Hz, 2H), 4.12-4.01 (m, 8H), 3.72 (br s, 3H), 2.47-2.22 (m, 4H), 2.16-1.92 (m, 4H), 1.86-1.66 (m, 1H), 1.22 (br s, 6H), 1.14-1.03 (m, 1H), 0.66-0.53 (m, 2H), 0.05-0.07 (m, 2H). LCMS Rt=0.371 min, m/z=777.4/779.4 [M+H]⁺.

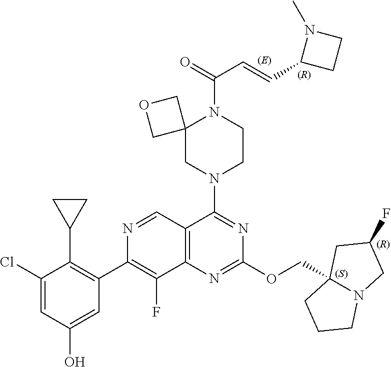

### Step 3: (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one

To a solution of diethyl(2-(8-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-5,8-diazaspiro[3.5]nonan-5-yl)-2-oxoethyl)phosphonate (20 mg, 25.73 μmol) in acetonitrile (0.5 mL) and N,N-dimethylformamide (0.1 mL) was added lithium chloride (5.45 mg, 128.67 μmol) and N,N-diisopropylethylamine (9.98 mg, 77.20 μmol), the mixture was stirred at 20° C. for 10 min, then (2R)-1-methylazetidine-2-carbaldehyde (51.02 mg, 514.67 μmol) was added at 0° C. and stirred at 20° C. for 1 h. The mixture was concentrated in vacuo and purified by reverse phase HPLC (column: Waters Xbridge Prep OBD C18 150*40 mm*10 μm; mobile phase: [H₂O (10 mM NH₄HCO₃)-ACN]; gradient: 30%-60% B over 8.0 min) affording (E)-1-(8-(7-(3-chloro-2-cyclopropyl-5-hydroxyphenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-5,8-diazaspiro[3.5]nonan-5-yl)-3-((R)-1-methylazetidin-2-yl) prop-2-en-1-one (2.84 mg, 15.17%) as a yellow oil: ¹H NMR (400 MHz, Chloroform-d) δ 9.04-8.89 (m, 1H), 7.03-6.85 (m, 2H), 6.60 (br s, 1H), 6.51-6.37 (m, 1H), 5.44-5.20 (m, 1H), 4.94-4.78 (m, 2H), 4.52-4.44 (m, 2H), 4.40 (br s, 1H), 4.32 (br d, J=5.9 Hz, 1H), 4.28 (s, 1H), 4.26-4.17 (m, 2H), 4.14-3.99 (m, 1H), 3.87-3.77 (m, 1H), 3.73-3.67 (m, 1H), 3.58-3.46 (m, 2H), 3.28 (br d, J=14.9 Hz, 2H), 3.21 (br s, 1H), 3.14 (s, 3H), 2.42-2.31 (m, 3H), 2.30-2.21 (m, 2H), 2.20-2.05 (m, 2H), 2.00-1.94 (m, 2H), 1.90-1.78 (m, 2H), 0.66-0.49 (m, 2H), 0.04-0.19 (m, 2H). LCMS Rt=2.859 min, m/z=722.3/723.3 [M+H]⁺.

LCMS (5 to 95% acetonitrile in water+0.03% ammonium bicarbonate over 6 mins) retention time 2.859 min, ESI+ found [M+H]=722.3/723.3.

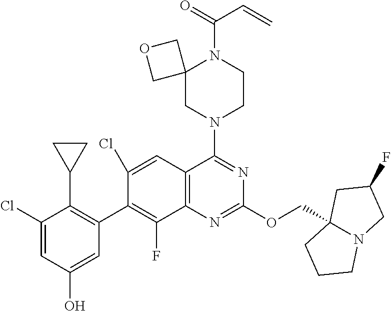 [SMILES: C=CC(=O)N1CCN(c2nc(OC[C@@]34CCCN3C[C@H](F)C4)nc3c(F)c(-c4cc(O)cc(Cl)c4C4CC4)c(Cl)cc23)CC12COC2]

### Example 78 (Method 2): 1-(8-(6-chloro-7-(3-chloro-2-cyclopropyl-5-hydroxyphenyl)-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonan-5-yl) prop-2-en-1-one

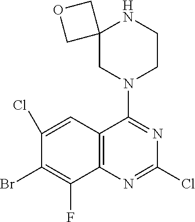 [SMILES: Fc1c(Br)c(Cl)cc2c(N3CCNC4(COC4)C3)nc(Cl)nc12]

### Step 1: 8-(7-bromo-2,6-dichloro-8-fluoroquinazolin-4-yl)-2-oxa-5,8-diazaspiro[3.5]nonane

To a solution of 7-bromo-2,4,6-trichloro-8-fluoroquinazoline (500 mg, 1.51 mmol) in dioxane (5 mL) was added N,N-diisopropylethylamine (586.80 mg, 4.54 mmol) and 2-oxa-5,8-diazaspiro[3.5]nonane (304.35 mg, 1.51 mmol, hydrochloride salt) at 0° C., the mixture was stirred at 0° C. for 1 h. The mixture was filtered and the filter cake was collected and concentrated in vacuo affording 8-(7-bromo-2,6-dichloro-8-fluoroquinazolin-4-yl)-2-oxa-5,8-diazaspiro[3.5]nonane (630 mg, crude) used in next step without any further purification. LCMS Rt-0.455 min, m/z=421.0/423.1 [M+H]⁺.

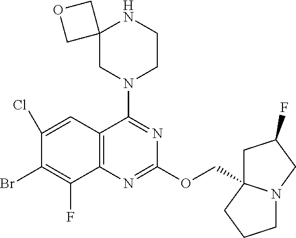 [SMILES: Fc1c(Br)c(Cl)cc2c(N3CCNC4(COC4)C3)nc(OC[C@@]34CCCN3C[C@H](F)C4)nc12]

### Step 2: 8-(7-bromo-6-chloro-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonane

To a solution of 8-(7-bromo-2,6-dichloro-8-fluoroquinazolin-4-yl)-2-oxa-5,8-diazaspiro[3.5]nonane (630 mg, 1.49 mmol) in dimethyl sulfoxide (15 mL) was added ((2R,7aS)-2-fluorohexahydro-1H-pyrrolizin-7a-yl) methanol (285.15 mg, 1.79 mmol), potassium fluoride (1.99 g, 34.33 mmol) and 1,4,7,10,13,16-hexaoxacyclooctadecane (394.52 mg, 1.49 mmol), the mixture was bubbled with nitrogen for 1 min and stirred at 120° C. for 4 h under nitrogen atmosphere. The mixture was diluted with water (12 mL) and extracted with ethyl acetate (3×15 mL). The combined organic layers were dried over sodium sulphate and concentrated in vacuo. The residue was purified by column chromatography (silica gel, 100-200 mesh, 0-100% methanol in dichloromethane) affording 8-(7-bromo-6-chloro-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonane (200 mg, 15.99%) as a yellow oil. LCMS Rt-0.475 min, m/z=544.2/546.2 [M+H]⁺.

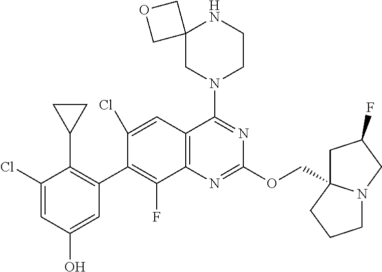 [SMILES: Oc1cc(Cl)c(C2CC2)c(-c2c(Cl)cc3c(N4CCNC5(COC5)C4)nc(OC[C@@]45CCCN4C[C@H](F)C5)nc3c2F)c1]

### Step 3: 3-chloro-5-(6-chloro-8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl) quinazolin-7-yl)-4-cyclopropylphenol

To a solution of 8-(7-bromo-6-chloro-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonane (200 mg, 367.09 μmol) in dioxane (1.8 mL) and water (0.6 mL) was added 3-chloro-4-cyclopropyl-5-(4,4,5,5-tetramethyl-1,3,2-dioxaborolan-2-yl) phenol (216.28 mg, 734.19 μmol), potassium phosphate (233.77 mg, 1.10 mmol) and Chloro(2-dicyclohexylphosphino-2′,4′,6′-triisopropyl-1,1′-biphenyl) [2-(2′-amino-1,1′-biphenyl)]pallaDIUM(II) (28.88 mg, 36.71 μmol), the mixture was degassed with nitrogen for three times and stirred at 80° C. for 2 h under nitrogen atmosphere. The mixture was diluted with water (5 mL) and extracted with ethyl acetate (3×8 mL). The combined organic layers were dried over sodium sulphate and concentrated in vacuo. The crude product was purified by reverse phase HPLC (column: Waters Xbridge Prep OBD C18 150*40 mm*10 μm; mobile phase: [H₂O (10 mM NH₄HCO₃)-ACN]; gradient: 30%-60% B over 8.0 min) affording 3-chloro-5-(6-chloro-8-fluoro-2-(((2R,7aS)-2-fluorotetrahydro-1H-pyrrolizin-7a(5H)-yl)methoxy)-4-(2-oxa-5,8-diazaspiro[3.5]nonan-8-yl) quinazolin-7-yl)-4-cyclopropylphenol (70 mg, 27.43%) as a white solid. LCMS Rt=0.501 min, m/z=632.3/634.3 [M+H]⁺.

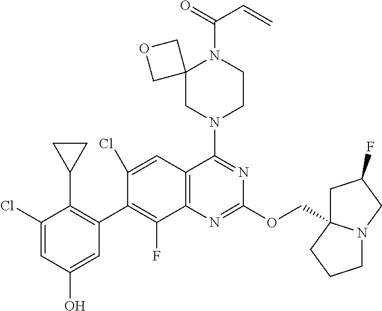 [SMILES: C=CC(=O)N1CCN(c2nc(OC[C@@]34CCCN3C[C@H](F)C4)nc3c(F)c(-c4cc(O)cc(Cl)c4C4CC4)c(Cl)cc23)CC12COC2]

### Step 4: 1-(8-(6-chloro-7-(3-chloro-2-cyclopropyl-5-hydroxyphenyl)-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonan-5-yl) prop-2-en-1-one

The acylation reaction was prepared in a similar fashion to Method #2, Step 4. The crude product was purified by reverse phase HPLC (column: Waters Xbridge Prep OBD C18 150*40 mm*10 μm; mobile phase: [H₂O (10 mM NH₄HCO₃)-ACN]; gradient: 30%-60% B over 8.0 min) affording 1-(8-(6-chloro-7-(3-chloro-2-cyclopropyl-5-hydroxyphenyl)-8-fluoro-2-(((2R,7aS)-2-fluorotetrahydro-1H-pyrrolizin-7a(5H)-yl)methoxy) quinazolin-4-yl)-2-oxa-5,8-diazaspiro[3.5]nonan-5-yl) prop-2-en-1-one (5.92 mg, 8.90%) as a yellow amorphous solid: ¹H NMR (400 MHz, Chloroform-d) δ 7.72-7.65 (m, 1H), 6.99-6.93 (m, 1H), 6.63-6.46 (m, 2H), 6.43-6.36 (m, 1H), 5.86-5.78 (m, 1H), 5.43-5.16 (m, 1H), 4.92-4.81 (m, 2H), 4.53-4.47 (m, 1H), 4.46-4.41 (m, 1H), 4.20 (br s, 4H), 3.87-3.74 (m, 1H), 3.69-3.55 (m, 3H), 3.39-3.11 (m, 3H), 3.05-2.95 (m, 1H), 2.10 (br dd, J=2.9, 5.3 Hz, 3H), 1.91 (br s, 3H), 1.73 (br s, 1H), 0.51 (br s, 2H), 0.31-0.17 (m, 2H). LCMS Rt=3.150 min, m/z=686.2/688.2 [M+H]⁺.

LCMS (5 to 95% acetonitrile in water+0.03% ammonium bicarbonate over 6 mins) retention time 3.150 min, ESI+ found [M+H]=686.2/688.2.

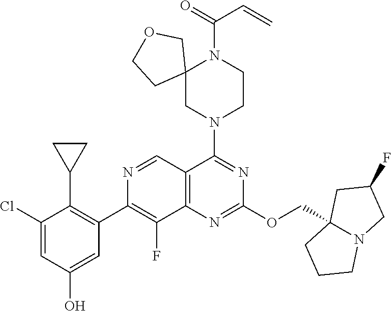 [SMILES: C=CC(=O)N1CCN(c2nc(OC[C@@]34CCCN3C[C@H](F)C4)nc3c(F)c(-c4cc(O)cc(Cl)c4C4CC4)ncc23)CC12CCOC2]

### Example 85 (Method 2): 1-(9-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-6,9-diazaspiro[4.5]decan-6-yl) prop-2-en-1-one

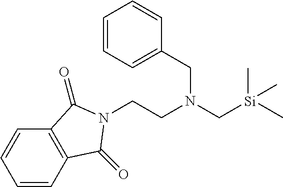 [SMILES: C[Si](C)(C)CN(CCN1C(=O)c2ccccc2C1=O)Cc1ccccc1]

### Step 1: 2-[2-[benzyl(trimethylsilylmethyl)amino]ethyl]isoindoline-1,3-dione

To a solution of 2-(2-bromoethyl) isoindoline-1,3-dione (10 g, 39.36 mmol) and 1-phenyl-N-(trimethylsilylmethyl) methanamine (8.37 g, 43.29 mmol) in acetonitrile (200 mL) was added potassium carbonate (10.88 g, 78.72 mmol), the mixture was stirred at 80° C. for 24 h. The reaction mixture was diluted with water (50 mL) and extracted with ethyl acetate (3×100 mL). The combined organic layers were dried over sodium sulphate and concentrated in vacuo. The resulting residue was purified by column chromatography (silica gel, 100-200 mesh, 0-10% ethyl acetate in petroleum ether) affording 2-[2-[benzyl(trimethylsilylmethyl)amino]ethyl]isoindoline-1,3-dione (3 g, 20.79%) as a yellow oil: ¹H NMR (400 MHz, Chloroform-d) δ 7.85-7.80 (m, 2H), 7.75-7.70 (m, 2H), 7.19-7.08 (m, 5H), 3.85-3.69 (m, 2H), 3.60-3.50 (m, 2H), 2.67-2.56 (m, 2H), 2.14-2.04 (m, 2H), 0.08-0.01 (m, 9H). LCMS Rt=2.543 min, m/z=367.2 [M+H]⁺.

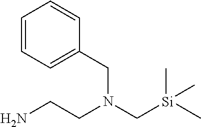 [SMILES: C[Si](C)(C)CN(CCN)Cc1ccccc1]

### Step 2: N1-benzyl-N1-((trimethylsilyl)methyl) ethane-1,2-diamine

A mixture of 2-[2-[benzyl(trimethylsilylmethyl)amino]ethyl]isoindoline-1,3-dione (20 g, 54.57 mmol) and hydrazine; hydrate (34.14 g, 545.66 mmol) in ethanol (200 mL) was stirred at 80° C. for 1 h. The reaction mixture was quenched with sodium hydroxide (30 mL, 5% in water) at 0° C. and extracted with dichloromethane (3×100 mL). The combined organic layers were dried over sodium sulphate and concentrated in vacuo affording N1-benzyl-N1-((trimethylsilyl)methyl) ethane-1,2-diamine (10 g, crude) as a yellow oil, which was used in the next step without further purification: ¹H NMR (400 MHz, Chloroform-d) 8 7.23 (br d, J=5.5 Hz, 4H), 7.21-7.15 (m, 1H), 3.46 (s, 2H), 2.71-2.62 (m, 2H), 2.32 (t, J=6.0 Hz, 2H), 1.93 (s, 2H), 0.03 (s, 9H). LCMS Rt=0.385 min, m/z=237.2 [M+H]⁺.

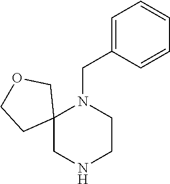 [SMILES: c1ccc(CN2CCNCC23CCOC3)cc1]

### Step 3: 6-benzyl-2-oxa-6,9-diazaspiro[4.5]decane

To a solution of N′-benzyl-N′-(trimethylsilylmethyl) ethane-1,2-diamine (1 g, 4.23 mmol) in acetonitrile (10 mL) and 2,2,2-trifluoroethanol (2.5 mL) was added tetrahydrofuran-3-one (364.12 mg, 4.23 mmol) and bis[2-(2-pyridyl)phenyl]iridium (1+); 4-tert-butyl-2-(4-tert-butyl-2-pyridyl)pyridine; hexafluorophosphate (386.57 mg, 422.96 μmol), the suspension was degassed under vacuum and purged with argon several times, the reaction was irradiated with external blue LED (λmax=455 nm, 34 W) at 25° C. for 6 h. The reaction mixture was diluted with water (20 mL) and extracted with ethyl acetate (3×50 mL). The combined organic layers were dried over sodium sulphate and concentrated in vacuo. The resulting residue was purified by reverse phase HPLC (column: Waters Xbridge BEH C18 250*50 mm*10 μm; mobile phase: [H₂O (10 mM NH₄HCO₃)-ACN]; gradient: 10%-40% B over 10.0 min) affording 6-benzyl-2-oxa-6,9-diazaspiro[4.5]decane (850 mg, 28.64%) as a yellow oil: ¹H NMR (400 MHz, Chloroform-d) δ 7.34-7.30 (m, 4H), 7.29-7.25 (m, 1H), 3.96-3.86 (m, 1H), 3.82-3.65 (m, 3H), 3.49 (s, 2H), 2.93 (br t, J=4.5 Hz, 2H), 2.58-2.26 (m, 4H), 1.98-1.91 (m, 1H), 1.85-1.78 (m, 1H). LCMS Rt=1.148 min, m/z=233.1 [M+H]⁺.

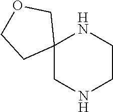

### Step 4: 2-oxa-6,9-diazaspiro[4.5]decane

To a solution of Pd/C (700.00 mg, 657.77 μmol) in ethyl acetate (80 mL) was added 9-benzyl-2-oxa-6,9-diazaspiro[4.5]decane (700 mg, 3.01 mmol) under argon atmosphere, the suspension was degassed under vacuum and purged with hydrogen for three times. The mixture was stirred under hydrogen (50 psi) at 30° C. for 8 h. The reaction mixture was concentrated to dryness in vacuo affording 2-oxa-6,9-diazaspiro[4.5]decane (60 mg, crude) as a pale yellow oil, which was used in the next step without further purification: ¹H NMR (400 MHz, Chloroform-d) δ 3.95-3.77 (m, 2H), 3.72-3.64 (m, 2H), 2.91-2.75 (m, 6H), 1.97-1.87 (m, 1H), 1.82-1.77 (m, 1H).

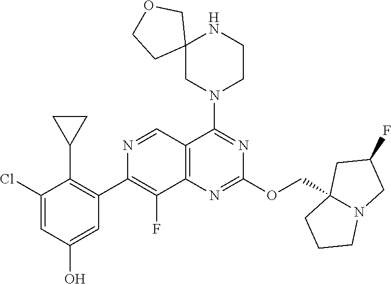 [SMILES: Oc1cc(Cl)c(C2CC2)c(-c2ncc3c(N4CCNC5(CCOC5)C4)nc(OC[C@@]45CCCN4C[C@H](F)C5)nc3c2F)c1]

### Step 5: 3-chloro-4-cyclopropyl-5-(8-fluoro-2-(((2R,7aS)-2-fluorotetrahydro-1H-pyrrolizin-7a(5H)-yl)methoxy)-4-(2-oxa-6,9-diazaspiro[4.5]decan-9-yl)pyrido[4,3-d]pyrimidin-7-yl) phenol

The substitution reaction was prepared in a similar fashion to Method #2, Step 3. The reaction mixture was concentrated in vacuo affording 3-chloro-4-cyclopropyl-5-(8-fluoro-2-(((2R,7aS)-2-fluorotetrahydro-1H-pyrrolizin-7a(5H)-yl)methoxy)-4-(2-oxa-6,9-diazaspiro[4.5]decan-9-yl)pyrido[4,3-d]pyrimidin-7-yl) phenol (569 mg, crude) as a pale yellow oil, which was used in the next step without further purification. LCMS Rt=0.304 min, m/z=613.3/614.3 [M+H]⁺.

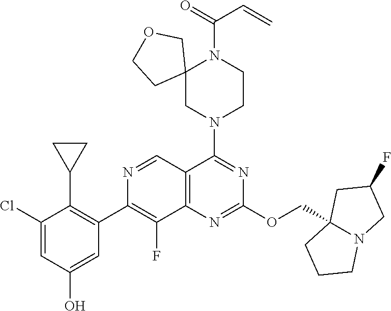 [SMILES: C=CC(=O)N1CCN(c2nc(OC[C@@]34CCCN3C[C@H](F)C4)nc3c(F)c(-c4cc(O)cc(Cl)c4C4CC4)ncc23)CC12CCOC2]

### Step 6: 1-(9-(7-(3-chloro-2-cyclopropyl-5-hydroxyphenyl)-8-fluoro-2-(((2R,7aS)-2-fluorotetrahydro-1H-pyrrolizin-7a(5H)-yl)methoxy)pyrido[4,3-d]pyrimidin-4-yl)-2-oxa-6,9-diazaspiro[4.5]decan-6-yl) prop-2-en-1-one

The acylation reaction was prepared in a similar fashion to Method #2, Step 4. The reaction mixture was concentrated in vacuo and purified by reverse phase HPLC (column: Waters Xbridge BEH C18 250*50 mm*10 μm; mobile phase: [H₂O (10 mM NH₄HCO₃)-ACN]; gradient: 25%-55% B over 10.0 min.) affording 1-(9-(7-(3-chloro-2-cyclopropyl-5-hydroxyphenyl)-8-fluoro-2-(((2R,7aS)-2-fluorotetrahydro-1H-pyrrolizin-7a(5H)-yl) methoxy)pyrido[4,3-d]pyrimidin-4-yl)-2-oxa-6,9-diazaspiro[4.5]decan-6-yl) prop-2-en-1-one (198.24 mg, 30.19%) as a yellow solid: ¹H NMR (400 MHz, Chloroform-d) δ 9.03-8.96 (m, 1H), 6.94-6.89 (m, 1H), 6.77-6.71 (m, 1H), 6.60-6.50 (m, 1H), 6.38-6.31 (m, 1H), 5.80 (d, J=10.6 Hz, 1H), 5.38-5.20 (m, 1H), 4.34-4.19 (m, 3H), 4.17-4.04 (m, 4H), 3.98 (dt, J=4.3, 8.1 Hz, 1H), 3.91 (br d, J=9.4 Hz, 2H), 3.75-3.66 (m, 1H), 3.37-3.18 (m, 3H), 3.07-2.97 (m, 1H), 2.67-2.51 (m, 1H), 2.35-2.18 (m, 2H), 2.16-2.08 (m, 1H), 2.03-1.72 (m, 6H), 0.68-0.49 (m, 2H), 0.13-0.09 (m, 2H). LCMS Rt=2.869 min, m/z=667.3/669.3 [M+H]⁺.

LCMS (5% to 95% acetonitrile in water+0.03% ammonium bicarbonate over 6 min), retention time 2.869 min, ESI+ found [M+H]=667.3/669.3

### Additional examples

The compounds in the table below were prepared by methods similar to those described in the synthetic examples disclosed herein or other methods within the knowledge of a person of skill in the art.

| | | | LCMS |
|---|---|---|---|
| | | | (m/z) |
| Example | Structure | ¹H NMR | rt |
| 61 | 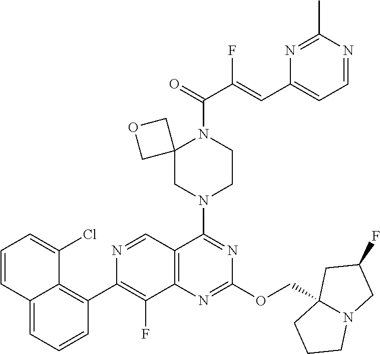 [SMILES: Cc1nccc(/C=C(\F)C(=O)N2CCN(c3nc(OC[C@@]45CCCN4C[C@H](F)C5)nc4c(F)c(-c5cccc6cccc(Cl)c56)ncc34)CC23COC3)n1] | ¹H NMR (400 MHz, Acetonitrile-d3) δ 9.01 (s, 1H), 8.62 (d, J = 5.3 Hz, 1H), 8.09-8.00 (m, 1H), 7.93 (d, J = 8.1 Hz, 1H), 7.64-7.57 (m, 1H), 7.55-7.47 (m, 3H), 7.46-7.38 (m, 1H), 6.62-6.39 (m, 1H), 5.28-5.03 (m, 1H), 4.89- 4.76 (m, 2H), 4.52-4.43 (m, 1H), 4.42-4.32 (m, 3H), 4.20-4.10 (m, 1H), 4.08-4.00 (m, 1H), 3.94-3.85 (m, 2H), 3.79-3.63 (m, 2H), 3.10-2.94 (m, 3H), 2.86-2.74 (m, 1H), 2.56 (s, 3H), 2.03-1.93 (m, 3H), 1.78 (br dd, J = 6.7, 11.1 Hz, 3H) | 757.3/758.3 3.038 min |
| 65 | 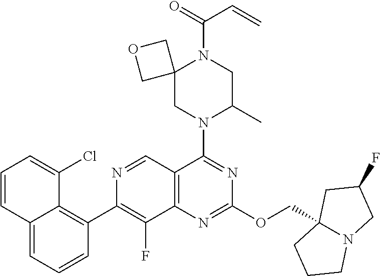 [SMILES: C=CC(=O)N1CC(C)N(c2nc(OC[C@@]34CCCN3C[C@H](F)C4)nc3c(F)c(-c4cccc5cccc(Cl)c45)ncc23)CC12COC2] | ¹H NMR (400 MHz, Acetonitrile-d3) δ 9.13 (d, J = 13.4 Hz, 1H), 8.16 (d, J = 6.9 Hz, 1H), 8.05 (dd, J = 1.1, 8.2 Hz, 1H), 7.81-7.68 (m, 1H), 7.67-7.61 (m, 2H), 7.59-7.50 (m, 1H), 6.69- 6.57 (m, 1H), 6.41-6.27 (m, 1H), 5.80 (dd, J = 1.9, 10.4 Hz, 1H), 5.39-5.20 (m, 1H), 5.13- 4.86 (m, 3H), 4.61 (br d, J = 5.1 Hz, 1H), 4.50 (br d, J = 7.5 Hz, 1H), 4.45-4.34 (m, 1H), 4.30- 4.13 (m, 2H), 4.10-3.77 (m, 2H), 3.52-3.38 (m, 1H), 3.21-3.05 (m, 3H), 2.99-2.86 (m, 1H), 2.12-2.03 (m, 2H), 1.94-1.77 (m, 4H), 1.36-1.27 (m, 3H) | 661.3/662.3 3.052 min |
| 66 | 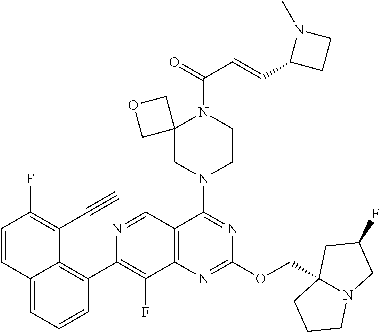 [SMILES: C#Cc1c(F)ccc2cccc(-c3ncc4c(N5CCN(C(=O)/C=C/[C@H]6CCN6C)C6(COC6)C5)nc(OC[C@@]56CCCN5C[C@H](F)C6)nc4c3F)c12] | ¹H NMR (400 MHz, Chloroform-d) δ 9.06 (s, 1H), 8.04-7.92 (m, 2H), 7.69-7.56 (m, 2H), 7.35 (t, J = 8.8 Hz, 1H), 6.98 (dd, J = 4.3, 15.4 Hz, 1H), 6.46 (br s, 1H), 5.39-5.19 (m, 1H), 4.94-4.73 (m, 2H), 4.67-4.53 (m, 1H), 4.46-4.37 (m, 2H), 4.33-4.28 (m, 1H), 4.24 (br s, 2H), 4.16-4.06 (m, 1H), 3.81-3.67 (m, 2H), 3.59 (br d, J = 4.9 Hz, 1H), 3.40 (s, 1H), 3.31-3.23 (m, 2H), 3.19-3.16 (m, 1H), 3.07 (d, J = 5.4 Hz, 1H), 3.00 (br s, 1H), 2.88 (br d, J = 9.4 Hz, 1H), 2.34 (s, 3H), 2.29-2.11 (m, 5H), 2.03-1.89 (m, 4H) | 724.4 1.806 min |
| 67 | 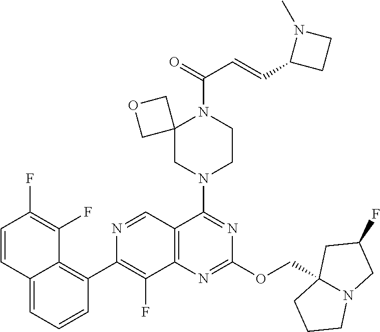 [SMILES: CN1CC[C@@H]1/C=C/C(=O)N1CCN(c2nc(OC[C@@]34CCCN3C[C@H](F)C4)nc3c(F)c(-c4cccc5ccc(F)c(F)c45)ncc23)CC12COC2] | ¹H NMR (400 MHz, Acetonitrile-d3) δ 9.24- 9.14 (m, 1H), 8.17-8.11 (m, 1H), 7.93 (dt, J = 5.47, 3.58 Hz, 1H), 7.776-7.68 (m, 2H), 7.62-7.52 (m, 1H), 6.87 (dd, J = 15.13, 5.75 Hz, 1H), 6.53 (br d, J = 15.26 Hz, 1H), 5.43-5.19 (m, 1H), 4.91-4.85 (m, 2H), 4.61-4.51 (m, 1H), 4.49-4.38 (m, 3H), 4.35-4.21 (m, 2H), 4.01-3.94 (m, 2H), 3.84-3.75 (m, 1H), 3.73- 3.62 (m, 2H), 3.48-3.39 (m, 1H), 3.32-3.16 (m, 3H), 3.05-2.94 (m, 2H), 2.36 (s, 3H), 2.09- 2.00 (m, 4H), 2.09-2.00 (m, 2H), 1.94-1.85 (m, 2H) | 718.3 1.979 min |
| 69 | 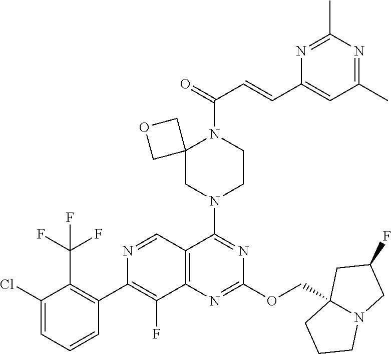 [SMILES: Cc1cc(/C=C/C(=O)N2CCN(c3nc(OC[C@@]45CCCN4C[C@H](F)C5)nc4c(F)c(-c5cccc(Cl)c5C(F)(F)F)ncc34)CC23COC3)nc(C)n1] | ¹H NMR (400 MHz, Acetonitrile-d3) δ 9.10 (s, 1H), 7.83-7.78 (m, 1H), 7.76-7.70 (m, 1H), 7.64-7.55 (m, 1H), 7.51-7.43 (m, 2H), 7.27 (s, 1H), 5.39-5.18 (m, 1H), 4.94 (br d, J = 5.5 Hz, 2H), 4.55 (br s, 1H), 4.48 (br d, J = 6.9 Hz, 3H), 4.28-4.22 (m, 1H), 4.20-4.14 (m, 1H), 3.97 (br d, J = 3.8 Hz, 2H), 3.76 (br d, J = 4.4 Hz, 2H), 3.21-3.13 (m, 2H), 3.09 (s, 1H), 2.96-2.88 (m, 1H), 2.64 (s, 3H), 2.49 (s, 3H), 2.13 (br d, J = 2.4 Hz, 3H), 1.94-1.83 (m, 3H) | 771.1/772.1 2.236 min |
| 70 | 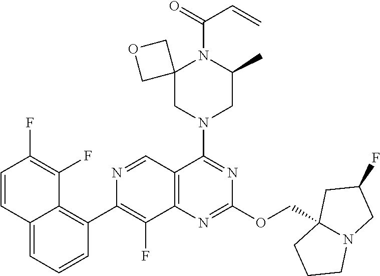 [SMILES: C=CC(=O)N1[C@@H](C)CN(c2nc(OC[C@@]34CCCN3C[C@H](F)C4)nc3c(F)c(-c4cccc5ccc(F)c(F)c45)ncc23)CC12COC2] | ¹H NMR (400 MHz, Acetonitrile-d3) δ 9.22 (s, 1H), 8.16-8.09 (m, 1H), 7.91 (ddd, J = 1.5, 5.0, 9.1 Hz, 1H), 7.75-7.66 (m, 2H), 7.61-7.51 (m, 1H), 6.62 (dd, J = 10.4, 16.6 Hz, 1H), 6.25 (br dd, J = 1.8, 16.8 Hz, 1H), 5.76 (br d, J = 10.5 Hz, 1H), 5.37-5.19 (m, 1H), 5.15-5.07 (m, 2H), 4.87 (d, J = 12.8 Hz, 1H), 4.53-4.45 (m, 2H), 4.40-4.31 (m, 1H), 4.26-4.22 (m, 1H), 4.21-4.14 (m, 2H), 4.06-3.91 (m, 2H), 3.21-3.13 (m, 2H), 3.09 (s, 1H), 2.95-2.88 (m, 1H), 2.21 (br s, 1H), 2.13 (d, J = 2.6 Hz, 1H), 2.10-2.04 (m, 1H), 1.95-1.82 (m, 3H), 1.38 (d, J = 6.9 Hz, 3H). | 663.3 2.099 min |
| 71 | 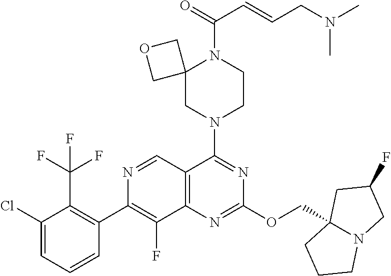 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)CC12COC2] | ¹H NMR (400 MHz, Acetonitrile-d3) δ 9.06 (s, 1H), 7.80-7.76 (m, 1H), 7.73-7.68 (m, 1H), 7.46 (d, J = 7.6 Hz, 1H), 6.78 (td, J = 6.1, 15.2 Hz, 1H), 6.44 (br d, J = 15.3 Hz, 1H), 5.37-5.14 (m, 1H), 4.83 (br d, J = 5.3 Hz, 2H), 4.48 (br s, 1H), 4.41 (d, J = 6.9 Hz, 3H), 4.24-4.20 (m, 1H), 4.16-4.12 (m, 1H), 3.90 (br d, J = 3.8 Hz, 2H), 3.62 (br s, 2H), 3.18- 3.10 (m, 2H), 3.06 (br d, J = 4.5 Hz, 3H), 2.94- 2.84 (m, 1H), 2.26-2.21 (m, 1H), 2.20-2.18 (m, 6H), 2.10 (d, J = 3.0 Hz, 1H), 2.08-2.01 (m, 1H), 1.92-1.79 (m, 3H). | 722.3/723.3 4.021 min |
| 72 | 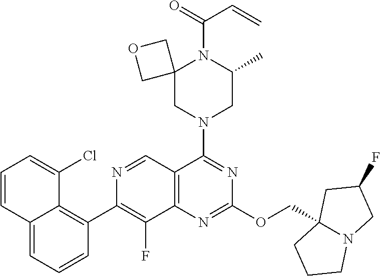 [SMILES: C=CC(=O)N1[C@H](C)CN(c2nc(OC[C@@]34CCCN3C[C@H](F)C4)nc3c(F)c(-c4cccc5cccc(Cl)c45)ncc23)CC12COC2] | ¹H NMR (400 MHz, Acetonitrile-d3) δ 9.21 (d, J = 2.4 Hz, 1H), 8.15 (dd, J = 1.2, 8.2 Hz, 1H), 8.09-8.02 (m, 1H), 7.75-7.62 (m, 3H), 7.60- 7.51 (m, 1H), 6.63 (ddd, J = 3.6, 10.5, 16.8 Hz, 1H), 6.25 (ddd, J = 2.0, 5.1, 16.8 Hz, 1H), 5.76 (td, J = 2.2, 10.4 Hz, 1H), 5.38-5.18 (m, 1H), 5.16-5.05 (m, 2H), 4.88 (dd, J = 2.7, 12.7 Hz, 1H), 4.55-4.44 (m, 2H), 4.41-4.31 (m, 1H), 4.26-4.12 (m, 3H), 4.08-3.91 (m, 2H), 3.22- 3.06 (m, 3H), 2.96-2.86 (m, 1H), 2.31-2.18 (m, 1H), 2.13 (d, J = 3.1 Hz, 1H), 2.11-2.04 (m, 1H), 1.93-1.80 (m, 3H), 1.38 (dd, J = 5.1, 6.8 Hz, 3H) | 661.2/662.2 3.026 min |
| 74 | 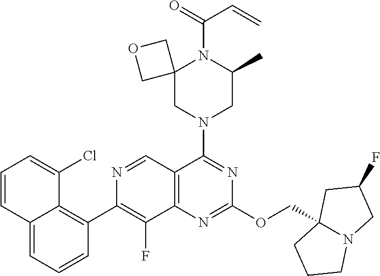 [SMILES: C=CC(=O)N1[C@@H](C)CN(c2nc(OC[C@@]34CCCN3C[C@H](F)C4)nc3c(F)c(-c4cccc5cccc(Cl)c45)ncc23)CC12COC2] | ¹H NMR (400 MHz, Acetonitrile-d3) δ 9.21 (d, J = 2.5 Hz, 1H), 8.15 (dd, J = 1.0, 8.1 Hz, 1H), 8.05 (d, J = 8.1 Hz, 1H), 7.74-7.69 (m, 1H), 7.68-7.62 (m, 2H), 7.57-7.51 (m, 1H), 6.63 (ddd, J = 3.5, 10.4, 16.7 Hz, 1H), 6.25 (ddd, J = 2.0, 5.0, 16.8 Hz, 1H), 5.76 (td, J = 2.2, 10.4 Hz, 1H), 5.37-5.20 (m, 1H), 5.15-5.06 (m, 2H), 4.88 (dd, J = 2.0, 12.8 Hz, 1H), 4.52- 4.45 (m, 2H), 4.36 (dt, J = 4.3, 7.1 Hz, 1H), 4.25-4.14 (m, 3H), 4.05-3.93 (m, 2H), 3.19- 3.13 (m, 2H), 3.09 (s, 1H), 2.96-2.88 (m, 1H), 2.23-2.17 (m, 2H), 2.13-2.06 (m, 2H), 1.92- 1.86 (m, 2H), 1.38 (dd, J = 5.6, 6.6 Hz, 3H) | 661.2/662.2 3.027 min |
| 75 | 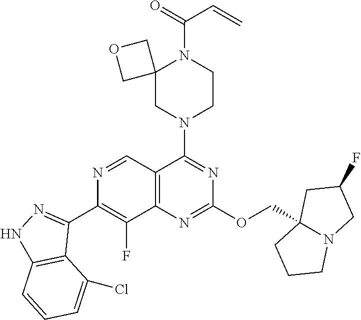 [SMILES: C=CC(=O)N1CCN(c2nc(OC[C@@]34CCCN3C[C@H](F)C4)nc3c(F)c(-c4n[nH]c5cccc(Cl)c45)ncc23)CC12COC2] | ¹H NMR (400 MHz, Acetonitrile-d3) δ 12.19- 11.57 (m, 1H), 9.31-8.99 (m, 1H), 7.66 (d, J = 8.4 Hz, 1H), 7.54-7.31 (m, 1H), 7.24-6.99 (m, 1H), 6.67 (dd, J = 10.5, 16.8 Hz, 1H), 6.29 (dd, J = 1.9, 16.9 Hz, 1H), 5.79 (dd, J = 1.9, 10.4 Hz, 1H), 5.45-5.17 (m, 1H), 4.92-4.84 (m, 2H), 4.51-4.40 (m, 4H), 4.34-4.17 (m, 2H), 4.00-3.88 (m, 2H), 3.65 (br t, J = 4.6 Hz, 2H), 3.28-3.08 (m, 3H), 3.01-2.86 (m, 1H), 2.16 (br s, 3H), 1.95-1.86 (m, 3H) | 637.3/638.3 2.686 min |
| 76 | 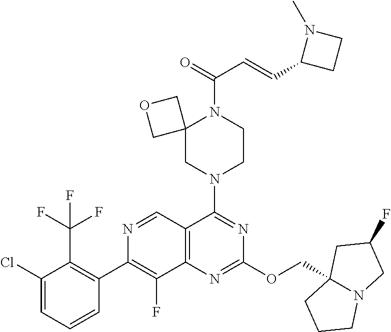 [SMILES: CN1CC[C@@H]1/C=C/C(=O)N1CCN(c2nc(OC[C@@]34CCCN3C[C@H](F)C4)nc3c(F)c(-c4cccc(Cl)c4C(F)(F)F)ncc23)CC12COC2] | ¹H NMR (400 MHz, Acetonitrile-d3) δ 9.10 (s, 1H), 8.11 (s, 1H), 7.84-7.78 (m, 1H), 7.77- 7.69 (m, 1H), 7.49 (d, J = 7.5 Hz, 1H), 6.85 (dd, J = 5.6, 15.1 Hz, 1H), 6.49 (br d, J = 15.5 Hz, 1H), 5.44-5.14 (m, 1H), 4.87 (br d, J = 4.4 Hz, 2H), 4.59-4.48 (m, 1H), 4.47-4.33 (m, 3H), 4.31-4.15 (m, 2H), 4.01-3.88 (m, 2H), 3.77- 3.56 (m, 3H), 3.36 (br t, J = 6.8 Hz, 1H), 3.27- 3.09 (m, 3H), 3.02-2.84 (m, 2H), 2.31 (s, 3H), 2.16-2.03 (m, 6H), 1.93-1.78 (m, 2H) | 734.3/735.3 1.959 min |
| 77 | 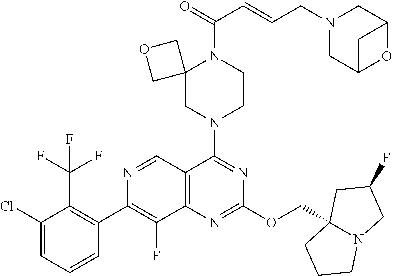 [SMILES: O=C(/C=C/CN1CC2CC(C1)O2)N1CCN(c2nc(OC[C@@]34CCCN3C[C@H](F)C4)nc3c(F)c(-c4cccc(Cl)c4C(F)(F)F)ncc23)CC12COC2] | ¹H NMR (400 MHz, Chloroform-d) δ 9.03 (s, 1H), 7.71-7.63 (m, 1H), 7.58 (t, J = 7.9 Hz, 1H), 7.36 (d, J = 7.8 Hz, 1H), 7.00 (td, J = 5.9, 15.2 Hz, 1H), 6.44 (br d, J = 15.1 Hz, 1H), 5.37- 5.19 (m, 1H), 4.93-4.81 (m, 2H), 4.53 (d, J = 6.1 Hz, 2H), 4.45 (br d, J = 1.3 Hz, 4H), 4.33- 4.27 (m, 1H), 4.25-4.19 (m, 1H), 4.02-3.75 (m, 2H), 3.64 (br d, J = 13.3 Hz, 2H), 3.45 (dd, J = 1.2, 5.8 Hz, 2H), 3.30-3.22 (m, 2H), 3.18- 3.11 (m, 3H), 3.09-2.95 (m, 2H), 2.86 (d, J = 11.3 Hz, 2H), 2.37 (d, J = 8.0 Hz, 1H), 2.29- 2.11 (m, 3H), 2.00-1.84 (m, 3H) | 776.3/777.3 3.003 min |
| 79 | 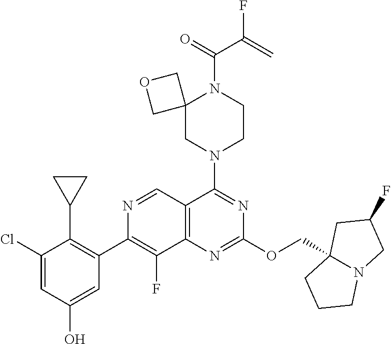 [SMILES: C=C(F)C(=O)N1CCN(c2nc(OC[C@@]34CCCN3C[C@H](F)C4)nc3c(F)c(-c4cc(O)cc(Cl)c4C4CC4)ncc23)CC12COC2] | ¹H NMR (400 MHz, Chloroform-d) δ 9.06- 8.85 (m, 1H), 6.96-6.91 (m, 1H), 6.69-6.56 (m, 1H), 5.64-5.45 (m, 1H), 5.29 (dd, J = 3.5, 15.8 Hz, 2H), 4.86 (d, J = 6.1 Hz, 2H), 4.52- 4.40 (m, 5H), 4.39-4.28 (m, 1H), 3.96-3.85 (m, 2H), 3.67 (br d, J = 4.4 Hz, 3H), 3.43-3.19 (m, 2H), 3.15-2.99 (m, 1H), 2.36-2.17 (m, 2H), 2.11-1.98 (m, 3H), 1.91-1.78 (m, 2H), 0.64-0.55 (m, 2H), 0.08-0.02 (m, 2H) | 671.0/672.0 2.962 min |
| 80 | 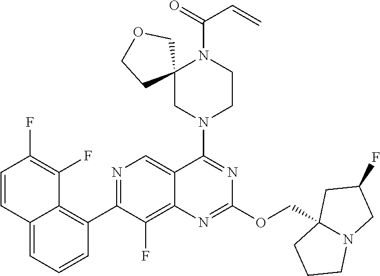 [SMILES: C=CC(=O)N1CCN(c2nc(OC[C@@]34CCCN3C[C@H](F)C4)nc3c(F)c(-c4cccc5ccc(F)c(F)c45)ncc23)C[C@@]12CCOC2] | ¹H NMR (400 MHz, Chloroform-d) δ 9.13 (s, 1H), 7.98 (d, J = 7.6 Hz, 1H), 7.76-7.70 (m, 1H), 7.64-7.55 (m, 2H), 7.45-7.36 (m, 1H), 6.65-6.55 (m, 1H), 6.38 (d, J = 16.4 Hz, 1H), 5.81 (d, J = 10.4 Hz, 1H), 5.36-5.23 (d, J = 54 Hz, 1H), 4.33-4.30 (m, 3H), 4.30-4.10 (m, 4H), 4.10-3.86 (m, 4H), 3.80-3.65 (m, 1H), 3.40-3.15 (m, 3H), 3.06-2.93 (m, 1H), 2.70- 2.61 (m, 1H), 2.32-2.10 (m, 3H), 2.01-1.80 (m, 4H) | 663.3 2.905 min |
| 81 | 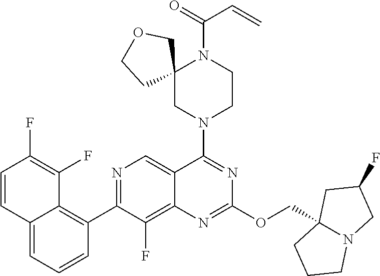 [SMILES: C=CC(=O)N1CCN(c2nc(OC[C@@]34CCCN3C[C@H](F)C4)nc3c(F)c(-c4cccc5ccc(F)c(F)c45)ncc23)C[C@]12CCOC2] | ¹H NMR (400 MHz, Chloroform-d) δ 9.13 (s, 1H), 7.98 (d, J = 7.6 Hz, 1H), 7.73-7.59 (m, 3H), 7.45-7.36 (m, 1H), 6.65-6.55 (m, 1H), 6.38 (d, J = 16.4 Hz, 1H), 5.81 (d, J = 10.4 Hz, 1H), 5.36-5.23 (d, J = 54 Hz, 1H), 4.33-4.30 (m, 3H), 4.30-4.10 (m, 4H), 4.10- 3.86 (m, 4H), 3.80-3.65 (m, 1H), 3.40-3.15 (m, 3H), 3.06-2.93 (m, 1H), 2.70-2.61 (m, 1H), 2.32-2.10 (m, 3H), 1.95-1.80 (m, 4H) | 663.3 2.905 min |
| 82 | 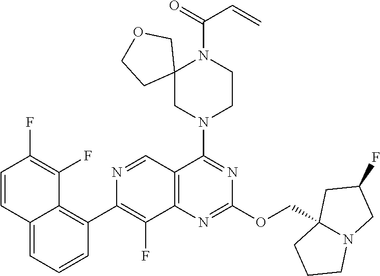 [SMILES: C=CC(=O)N1CCN(c2nc(OC[C@@]34CCCN3C[C@H](F)C4)nc3c(F)c(-c4cccc5ccc(F)c(F)c45)ncc23)CC12CCOC2] | ¹H NMR (400 MHz, Chloroform-d) δ 9.18- 9.09 (m, 1H), 8.02-7.93 (m, 1H), 7.68-7.58 (m, 1H), 7.45-7.36 (m, 1H), 7.34 (s, 2H), 6.62- 6.43 (m, 1H), 6.39-6.20 (m, 1H), 5.83-5.63 (m, 1H), 5.44-5.20 (m, 1H), 4.39-4.12 (m, 5H), 4.09-3.93 (m, 3H), 3.92-3.66 (m, 2H), 3.58-3.46 (m, 2H), 3.44-3.15 (m, 2H), 3.09- 2.94 (m, 1H), 2.74-2.61 (m, 1H), 2.61-2.42 (m, 2H), 2.37-2.13 (m, 3H), 2.06-1.79 (m, 3H) | 663.1 2.942 min |
| 83 | 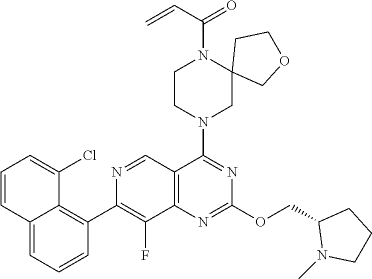 [SMILES: C=CC(=O)N1CCN(c2nc(OC[C@@H]3CCCN3C)nc3c(F)c(-c4cccc5cccc(Cl)c45)ncc23)CC12CCOC2] | ¹H NMR (400 MHz, Acetonitrile-d3) δ 9.24 (d, J = 2.8 Hz, 1H), 8.17 (br d, J = 8.2 Hz, 1H), 8.07 (br d, J = 8.2 Hz, 1H), 7.77-7.71 (m, 1H), 7.70-7.63 (m, 2H), 7.59-7.53 (m, 1H), 6.71 (br dd, J = 10.4, 16.5 Hz, 1H), 6.23 (br d, J = 16.7 Hz, 1H), 5.74 (br d, J = 10.5 Hz, 1H), 4.57-4.46 (m, 1H), 4.34 (br dd, J = 5.9, 10.0 Hz, 1H), 4.28-4.22 (m, 1H), 4.22-4.13 (m, 3H), 4.13-4.06 (m, 2H), 4.04 (br d, J = 3.8 Hz, 2H), 3.99 (br d, J = 8.1 Hz, 1H), 3.72 (br dd, J = 8.7, 15.6 Hz, 1H), 3.05 (br t, J = 7.0 Hz, 1H), 2.72-2.58 (m, 2H), 2.45 (s, 3H), 2.29 (br d, J = 8.2 Hz, 1H), 2.10-2.02 (m, 1H), 1.95-1.86 (m, 1H), 1.84-1.71 (m, 3H) | 617.2/618.2 2.622 min |
| 84 | 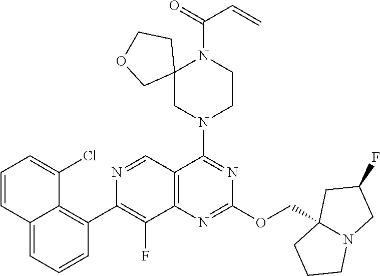 [SMILES: C=CC(=O)N1CCN(c2nc(OC[C@@]34CCCN3C[C@H](F)C4)nc3c(F)c(-c4cccc5cccc(Cl)c45)ncc23)CC12CCOC2] | ¹H NMR (400 MHz, Chloroform-d) δ 9.11 (d, J = 2.5 Hz, 1H), 8.01 (dd, J = 1.5, 7.5 Hz, 1H), 7.95-7.84 (m, 1H), 7.67-7.52 (m, 3H), 7.49- 7.38 (m, 1H), 6.67-6.48 (m, 1H), 6.43-6.27 (m, 1H), 5.88-5.72 (m, 1H), 5.44-5.16 (m, 1H), 4.42-4.23 (m, 3H), 4.23-4.10 (m, 4H), 4.07-3.93 (m, 4H), 3.78-3.67 (m, 1H), 3.41- 3.31 (m, 1H), 3.29-3.21 (m, 1H), 3.08-2.96 (m, 1H), 2.73-2.61 (m, 1H), 2.37-2.28 (m, 1H), 2.27-2.14 (m, 2H), 1.82 (br d, J = 7.5 Hz, 5H) | 661.2/662.2 2.981 min |
| 86 | 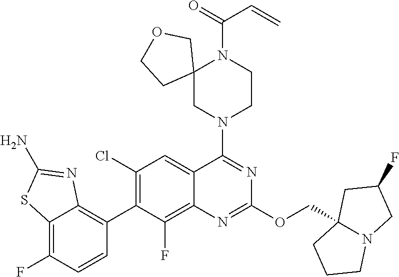 [SMILES: C=CC(=O)N1CCN(c2nc(OC[C@@]34CCCN3C[C@H](F)C4)nc3c(F)c(-c4ccc(F)c5sc(N)nc45)c(Cl)cc23)CC12CCOC2] | ¹H NMR (400 MHz, Chloroform-d) δ 7.83- 7.76 (m, 1H), 7.26-7.21 (m, 1H), 7.02-6.96 (m, 1H), 6.62-6.51 (m, 1H), 6.39-6.30 (m, 1H), 5.81-5.70 (m, 1H), 5.65-5.49 (m, 2H), 5.43-5.16 (m, 1H), 4.33-4.26 (m, 1H), 4.26- 4.16 (m, 2H), 4.13 (br d, J = 8.9 Hz, 1H), 4.08-3.99 (m, 4H), 3.98-3.92 (m, 3H), 3.91- 3.85 (m, 1H), 3.83-3.73 (m, 1H), 3.33-3.16 (m, 2H), 3.04-2.92 (m, 1H), 2.68-2.52 (m, 1H), 2.49-2.26 (m, 1H), 2.25-2.12 (m, 2H), 2.01-1.83 (m, 4H) | 700.2/702.2 3.039 min |
| 87 | 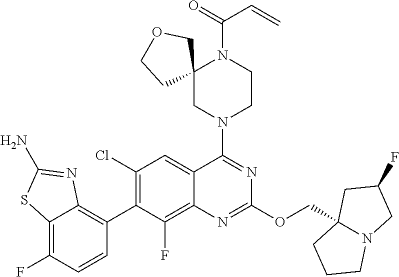 [SMILES: C=CC(=O)N1CCN(c2nc(OC[C@@]34CCCN3C[C@H](F)C4)nc3c(F)c(-c4ccc(F)c5sc(N)nc45)c(Cl)cc23)C[C@]12CCOC2] | ¹H NMR (400 MHz, Chloroform-d) δ 7.89- 7.69 (m, 1H), 7.26-7.20 (m, 1H), 7.01-6.93 (m, 1H), 6.61-6.50 (m, 1H), 6.38-6.30 (m, 1H), 6.18-5.69 (m, 3H), 5.42-5.22 (m, 1H), 4.37-4.19 (m, 3H), 4.16-4.09 (m, 1H), 4.08 (br s, 4H), 3.97-3.85 (m, 3H), 3.83-3.70 (m, 1H), 3.47-3.17 (m, 3H), 3.08-2.96 (m, 1H), 2.61 (dt, J = 3.9, 8.3 Hz, 1H), 2.38-2.32 (m, 1H), 2.30-2.15 (m, 2H), 2.04-1.87 (m, 4H) | 700.2/702.2 3.014 min |
| 88 | 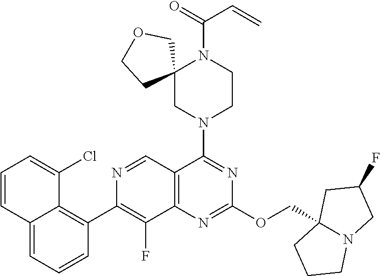 [SMILES: C=CC(=O)N1CCN(c2nc(OC[C@@]34CCCN3C[C@H](F)C4)nc3c(F)c(-c4cccc5cccc(Cl)c45)ncc23)C[C@@]12CCOC2] | ¹H NMR (400 MHz, Chloroform-d) δ 9.07- 9.00 (m, 1H), 7.97-7.92 (m, 1H), 7.85-7.78 (m, 1H), 7.59-7.45 (m, 3H), 7.39-7.31 (m, 1H), 6.55-6.42 (m, 1H), 6.32-6.24 (m, 1H), 5.75-5.68 (m, 1H), 5.33-5.13 (m, 1H), 4.29- 4.22 (m, 1H), 4.21-4.02 (m, 5H), 3.99-3.86 (m, 4H), 3.69-3.58 (m, 1H), 3.33-3.21 (m, 1H), 3.21-3.02 (m, 2H), 2.93 (br s, 1H), 2.64- 2.53 (m, 1H), 2.27-2.20 (m, 1H), 2.18-2.04 (m, 2H), 1.87 (br d, J = 12.0 Hz, 3H), 1.84- 1.70 (m, 2H) | 661.3/663.3 2.044 min |
| 89 | 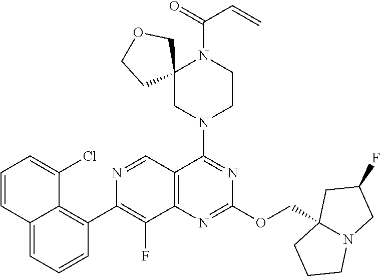 [SMILES: C=CC(=O)N1CCN(c2nc(OC[C@@]34CCCN3C[C@H](F)C4)nc3c(F)c(-c4cccc5cccc(Cl)c45)ncc23)C[C@]12CCOC2] | ¹H NMR (400 MHz, Chloroform-d) δ 9.04 (d, J = 2.6 Hz, 1H), 7.94 (d, J = 7.9 Hz, 1H), 7.85-7.78 (m, 1H), 7.58-7.45 (m, 3H), 7.38- 7.32 (m, 1H), 6.55-6.45 (m, 1H), 6.32-6.24 (m, 1H), 5.72 (dd, J = 1.3, 10.4 Hz, 1H), 5.37-5.16 (m, 1H), 4.46-4.25 (m, 2H), 4.24- 4.02 (m, 6H), 4.01-3.88 (m, 4H), 3.64 (dd, J = 8.8, 18.5 Hz, 1H), 3.28-3.14 (m, 1H), 3.05-2.88 (m, 1H), 2.65-2.54 (m, 1H), 2.38- 2.12 (m, 3H), 2.04-1.89 (m, 3H), 1.87-1.72 (m, 2H) | 661.3/663.3 2.043 min |
| 90 | 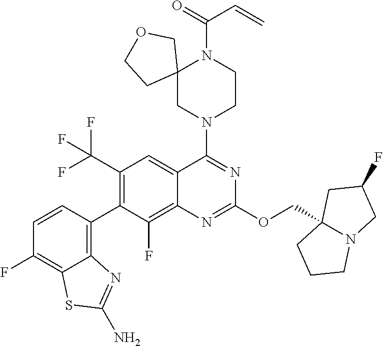 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)CC12CCOC2] | ¹H NMR (400 MHz, Chloroform-d) δ 8.08 (s, 1H), 7.22-7.15 (m, 1H), 6.99-6.91 (m, 1H), 6.61-6.51 (m, 1H), 6.35 (dd, J = 1.5, 16.8 Hz, 1H), 5.82-5.76 (m, 1H), 5.63 (br d, J = 13.5 Hz, 1H), 5.40-5.20 (m, 1H), 4.36-4.20 (m, 3H), 4.16-4.00 (m, 5H), 3.99-3.88 (m, 3H), 3.82-3.68 (m, 1H), 3.43-3.16 (m, 3H), 3.01 (br s, 1H), 2.63 (br d, J = 5.0 Hz, 1H), 2.36- 2.14 (m, 3H), 2.04-1.86 (m, 5H) | 734.2 3.081 min |
| 91 | 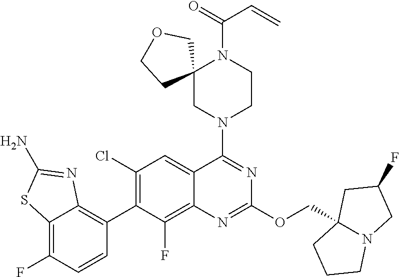 [SMILES: C=CC(=O)N1CCN(c2nc(OC[C@@]34CCCN3C[C@H](F)C4)nc3c(F)c(-c4ccc(F)c5sc(N)nc45)c(Cl)cc23)C[C@@]12CCOC2] | ¹H NMR (400 MHz, Chloroform-d) δ 7.86- 7.72 (m, 1H), 7.26-7.20 (m, 1H), 7.05-6.94 (m, 1H), 6.63-6.51 (m, 1H), 6.38-6.9 (m, 1H), 5.88-5.68 (m, 3H), 5.39-5.17 (m, 1H), 4.31-4.25 (m, 1H), 4.25-4.15 (m, 2H), 4.14- 4.10 (m, 1H), 4.08-3.98 (m, 4H), 3.97-3.85 (m, 3H), 3.83-3.71 (m, 1H), 3.35-3.22 (m, 2H), 3.21-3.14 (m, 1H), 3.03-2.94 (m, 1H), 2.68-2.53 (m, 1H), 2.34-2.24 (m, 1H), 2.24- 2.11 (m, 2H), 2.00-1.84 (m, 4H) | 700.2/702.2 3.011 min |
| 92 | 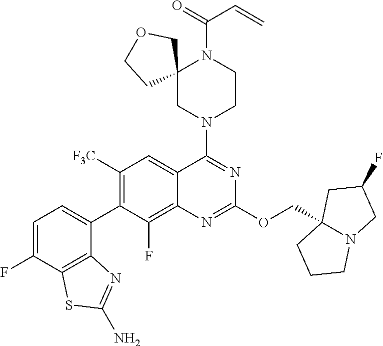 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@]12CCOC2] | ¹H NMR (400 MHz, Chloroform-d) δ 8.09 (s, 1H), 7.23-7.16 (m, 1H), 7.03-6.92 (m, 1H), 6.63-6.51 (m, 1H), 6.42-6.32 (m, 1H), 5.84- 5.75 (m, 1H), 5.43-5.15 (m, 3H), 4.34-4.14 (m, 3H), 4.01 (br s, 5H), 4.00-3.91 (m, 3H), 3.81-3.72 (m, 1H), 3.34-3.06 (m, 3H), 3.05- 2.90 (m, 1H), 2.67-2.58 (m, 1H), 2.31-2.07 (m, 3H), 1.98-1.82 (m, 4H) | 734.2 3.065 min |
| 93 | 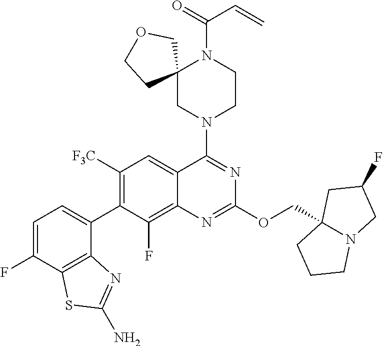 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@@]12CCOC2] | ¹H NMR (400 MHz, Chloroform-d) δ 8.09 (s, 1H), 7.23-7.15 (m, 1H), 7.02-6.94 (m, 1H), 6.63-6.52 (m, 1H), 6.40-6.31 (m, 1H), 5.83- 5.76 (m, 1H), 5.40-5.14 (m, 3H), 4.34-4.16 (m, 3H), 4.15-4.02 (m, 5H), 3.99-3.90 (m, 3H), 3.80-3.71 (m, 1H), 3.31-3.11 (m, 3H), 3.02-2.93 (m, 1H), 2.69-2.56 (m, 1H), 2.30- 2.09 (m, 3H), 1.97-1.83 (m, 4H) | 734.2 3.060 min |
| 94 | 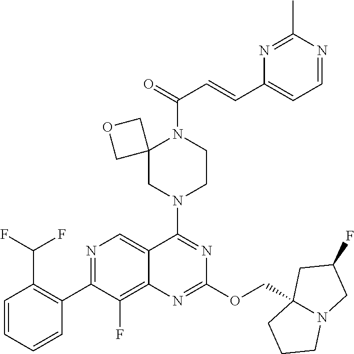 [SMILES: Cc1nccc(/C=C/C(=O)N2CCN(c3nc(OC[C@@]45CCCN4C[C@H](F)C5)nc4c(F)c(-c5ccccc5C(F)F)ncc34)CC23COC3)n1] | ¹H NMR (400 MHz, Chloroform-d) δ 9.09 (s, 1H), 8.72 (d, J = 5.0 Hz, 1H), 7.90-7.82 (m, 1H), 7.70-7.60 (m, 4H), 7.58-7.52 (m, 1H), 7.16 (m, 2H), 5.31 (br d, J = 53.8 Hz, 1H), 4.92 (br d, J = 5.8 Hz, 2H), 4.55-4.45 (m, 4H), 4.33 (br s, 2H), 4.02-3.89 (m, 2H), 3.85-3.73 (m, 2H), 3.45-3.17 (m, 3H), 3.10-2.95 (m, 1H), 2.78 (s, 3H), 2.33-2.11 (m, 3H), 2.00 (br d, J = 13.9 Hz, 3H) | 705.4 2.888 min |

### Biological Examples

### Example B-1: Inhibition of KRASG12C and cRAF Binding

The AlphaScreen technology was used to determine IC₅₀s for compound inhibition of KRAS G12C (present as the Cys-light (C51S, C80L and C₁₁₈S), truncated version comprising amino acids 1-169) and cRAF interaction. Compounds were diluted in 100% DMSO and each compound concentration was spotted at 200 nl/well onto low volume, white 384 well plates. The KRAS G12C contained a biotin-AviTag and the cRaf, as Ras-binding domain (amino acids 50-131, RBD), was GST-tagged. KRAS G12C was preloaded with the GTP analogue Guanosine 5′-[β,γ-imido]triphosphate (GMPPNP). The KRAS G12C was diluted in 25 mM Hepes, pH 7.4, 150 mM NaCl, 5 mM MgCl₂, 0.01% TritonX-100 and 10 μM GMPPNP and added at 10 μl/well to compound-spotted plates resulting in a DMSO concentration of 2%. Plates were incubated for 2 hours. A mixture of RBD and the AlphaScreen streptavidin donor and glutathione acceptor beads diluted in 25 mM Hepes, pH 7.4, 150 mM NaCl, 5 mM MgCl₂, 0.01% TritonX-100 and 2% DMSO was then added at 10 μl/well and incubated for 60-90 minutes before the samples were read for emission at 570 nm after excitation of the donor beads at 680 nm. All incubations were performed at room temperature. The final top compound concentration was 50 μM with 1:3 titrations for 10-point dose response curves. Final assay conditions were 0.5 nM KRAS G12C, 0.75 nM RBD and 5 μg/ml each of AlphaScreen donor and acceptor beads. IC₅₀S were determined using nonlinear regression fit of [inhibitor] vs. response (4 parameters).

A counter assay was also set up to rule out inhibitors of the AlphaScreen technology itself. Compound plates were incubated for 2 hours as above with buffer only.

The AlphaScreen beads were added as above except biotin-AviTag-GST was substituted for the RBD. Samples were read and analyzed as above.

Results for compounds are shown in Table 1.

TABLE 1
Inhibition of KRAS G12C and cRAF Binding (IC₅₀)

| Synthetic | | |
|---|---|---|
| Example | Structure | IC₅₀ |
| 1 | 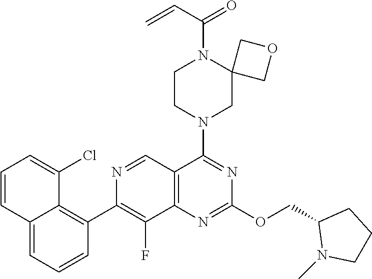 [SMILES: C=CC(=O)N1CCN(c2nc(OC[C@@H]3CCCN3C)nc3c(F)c(-c4cccc5cccc(Cl)c45)ncc23)CC12COC2] | ++ |
| 2 | 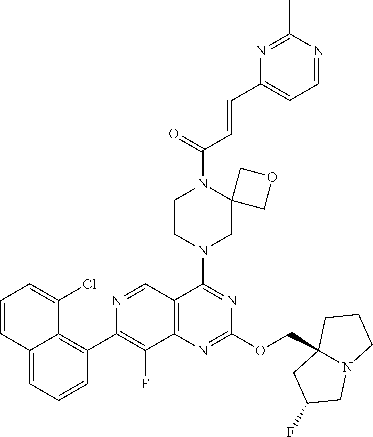 [SMILES: Cc1nccc(/C=C/C(=O)N2CCN(c3nc(OC[C@@]45CCCN4C[C@H](F)C5)nc4c(F)c(-c5cccc6cccc(Cl)c56)ncc34)CC23COC3)n1] | ++ |
| 3 | 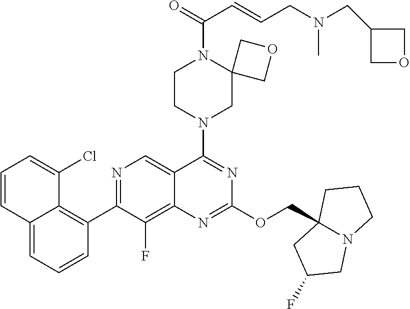 [SMILES: CN(C/C=C/C(=O)N1CCN(c2nc(OC[C@@]34CCCN3C[C@H](F)C4)nc3c(F)c(-c4cccc5cccc(Cl)c45)ncc23)CC12COC2)CC1COC1] | +++ |
| 4 | 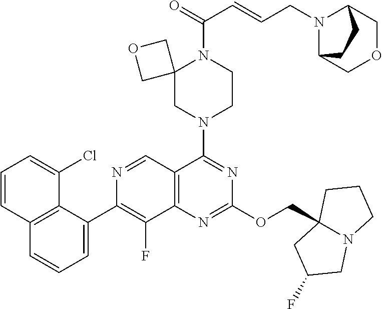 | ++++ |
| 5 | 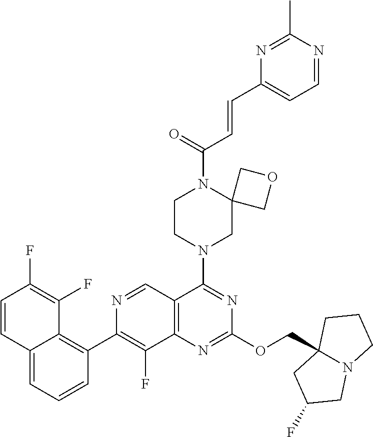 [SMILES: Cc1nccc(/C=C/C(=O)N2CCN(c3nc(OC[C@@]45CCCN4C[C@H](F)C5)nc4c(F)c(-c5cccc6ccc(F)c(F)c56)ncc34)CC23COC3)n1] | +++ |
| 6 | 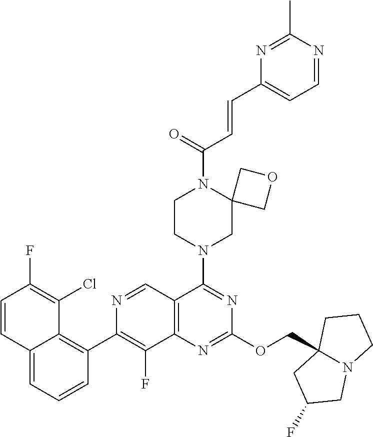 [SMILES: Cc1nccc(/C=C/C(=O)N2CCN(c3nc(OC[C@@]45CCCN4C[C@H](F)C5)nc4c(F)c(-c5cccc6ccc(F)c(Cl)c56)ncc34)CC23COC3)n1] | +++ |
| 7 | 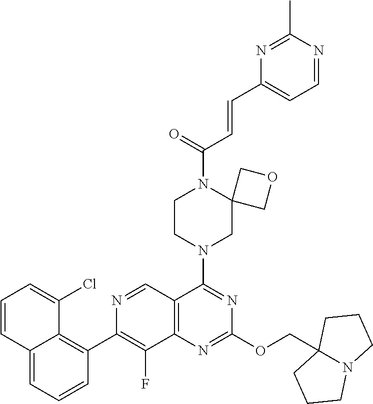 [SMILES: Cc1nccc(/C=C/C(=O)N2CCN(c3nc(OCC45CCCN4CCC5)nc4c(F)c(-c5cccc6cccc(Cl)c56)ncc34)CC23COC3)n1] | ++ |
| 8 | 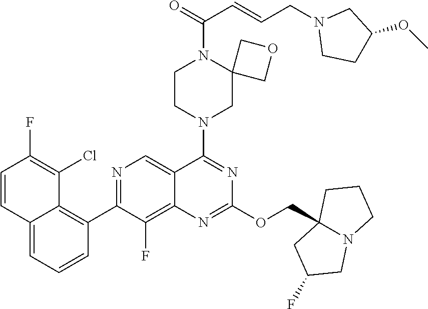 [SMILES: CO[C@@H]1CCN(C/C=C/C(=O)N2CCN(c3nc(OC[C@@]45CCCN4C[C@H](F)C5)nc4c(F)c(-c5cccc6ccc(F)c(Cl)c56)ncc34)CC23COC3)C1] | +++ |
| 9 | 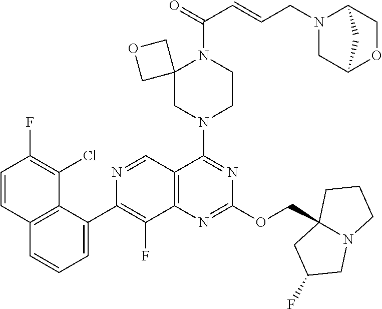 [SMILES: O=C(/C=C/CN1C[C@H]2C[C@@H]1CO2)N1CCN(c2nc(OC[C@@]34CCCN3C[C@H](F)C4)nc3c(F)c(-c4cccc5ccc(F)c(Cl)c45)ncc23)CC12COC2] | ++++ |
| 10 | 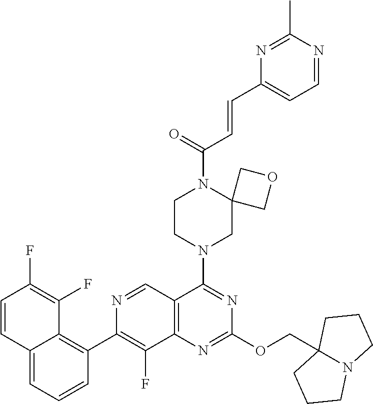 [SMILES: Cc1nccc(/C=C/C(=O)N2CCN(c3nc(OCC45CCCN4CCC5)nc4c(F)c(-c5cccc6ccc(F)c(F)c56)ncc34)CC23COC3)n1] | ++ |
| 11 | 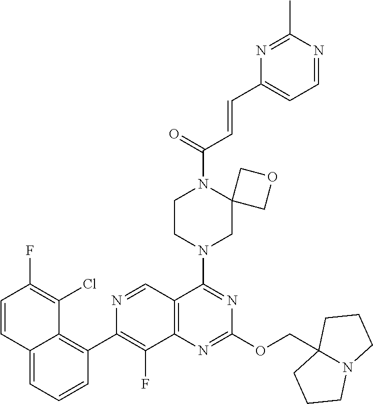 [SMILES: Cc1nccc(/C=C/C(=O)N2CCN(c3nc(OCC45CCCN4CCC5)nc4c(F)c(-c5cccc6ccc(F)c(Cl)c56)ncc34)CC23COC3)n1] | ++ |
| 12 | 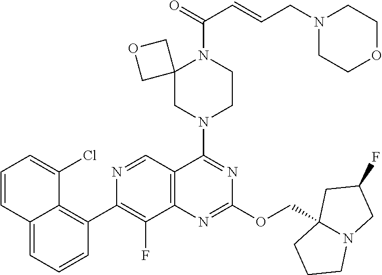 [SMILES: O=C(/C=C/CN1CCOCC1)N1CCN(c2nc(OC[C@@]34CCCN3C[C@H](F)C4)nc3c(F)c(-c4cccc5cccc(Cl)c45)ncc23)CC12COC2] | + |
| 13 | 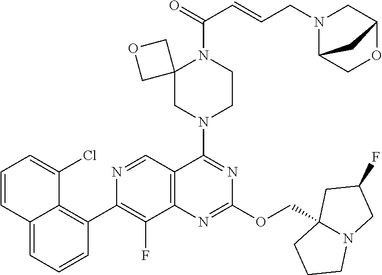 [SMILES: O=C(/C=C/CN1C[C@H]2C[C@@H]1CO2)N1CCN(c2nc(OC[C@@]34CCCN3C[C@H](F)C4)nc3c(F)c(-c4cccc5cccc(Cl)c45)ncc23)CC12COC2] | +++ |
| 14 | 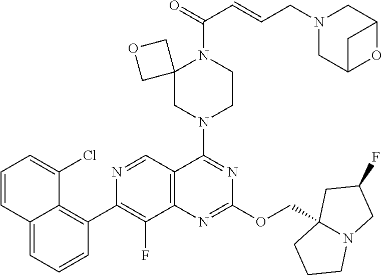 [SMILES: O=C(/C=C/CN1CC2CC(C1)O2)N1CCN(c2nc(OC[C@@]34CCCN3C[C@H](F)C4)nc3c(F)c(-c4cccc5cccc(Cl)c45)ncc23)CC12COC2] | +++ |
| 15 | 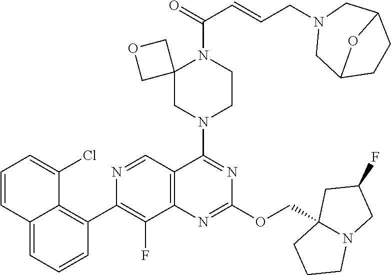 [SMILES: O=C(/C=C/CN1CC2CCC(C1)O2)N1CCN(c2nc(OC[C@@]34CCCN3C[C@H](F)C4)nc3c(F)c(-c4cccc5cccc(Cl)c45)ncc23)CC12COC2] | +++ |
| 16 | 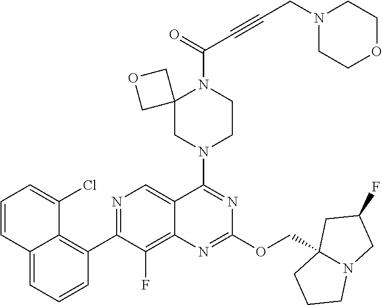 [SMILES: O=C(C#CCN1CCOCC1)N1CCN(c2nc(OC[C@@]34CCCN3C[C@H](F)C4)nc3c(F)c(-c4cccc5cccc(Cl)c45)ncc23)CC12COC2] | ++ |
| 17 | 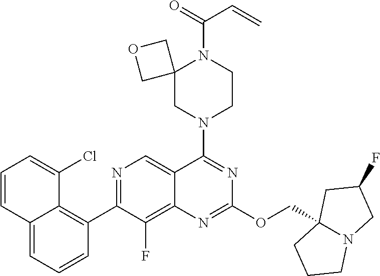 [SMILES: C=CC(=O)N1CCN(c2nc(OC[C@@]34CCCN3C[C@H](F)C4)nc3c(F)c(-c4cccc5cccc(Cl)c45)ncc23)CC12COC2] | +++ |
| 18 | 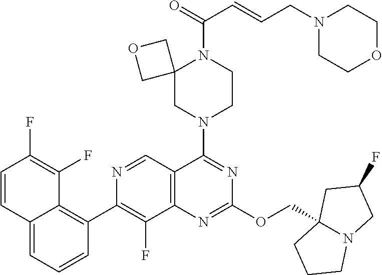 [SMILES: O=C(/C=C/CN1CCOCC1)N1CCN(c2nc(OC[C@@]34CCCN3C[C@H](F)C4)nc3c(F)c(-c4cccc5ccc(F)c(F)c45)ncc23)CC12COC2] | +++ |
| 19 | 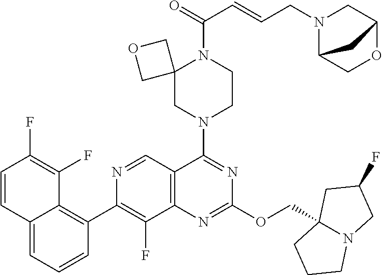 [SMILES: O=C(/C=C/CN1C[C@H]2C[C@@H]1CO2)N1CCN(c2nc(OC[C@@]34CCCN3C[C@H](F)C4)nc3c(F)c(-c4cccc5ccc(F)c(F)c45)ncc23)CC12COC2] | ++++ |
| 20 | 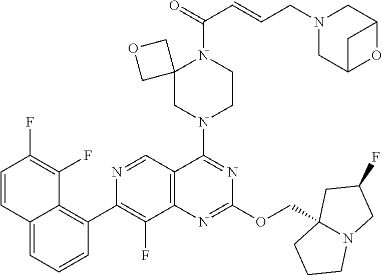 [SMILES: O=C(/C=C/CN1CC2CC(C1)O2)N1CCN(c2nc(OC[C@@]34CCCN3C[C@H](F)C4)nc3c(F)c(-c4cccc5ccc(F)c(F)c45)ncc23)CC12COC2] | ++++ |
| 21 | 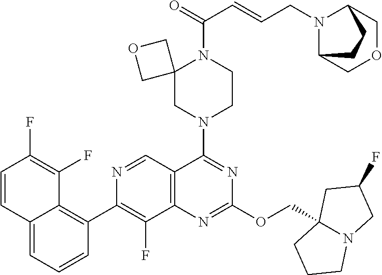 | ++++ |
| 22 | 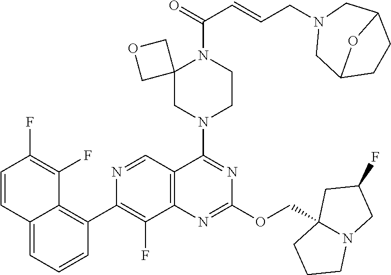 [SMILES: O=C(/C=C/CN1CC2CCC(C1)O2)N1CCN(c2nc(OC[C@@]34CCCN3C[C@H](F)C4)nc3c(F)c(-c4cccc5ccc(F)c(F)c45)ncc23)CC12COC2] | ++++ |
| 23 | 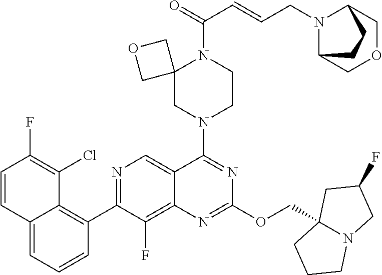 | ++++ |
| 24 | 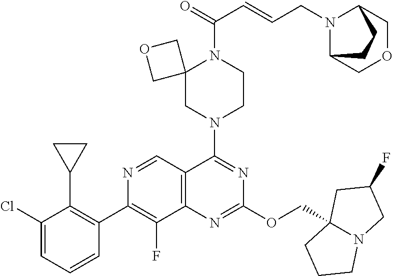 | ++++ |
| 25 | 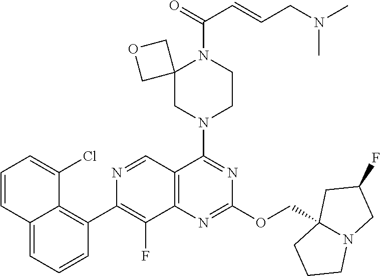 [SMILES: CN(C)C/C=C/C(=O)N1CCN(c2nc(OC[C@@]34CCCN3C[C@H](F)C4)nc3c(F)c(-c4cccc5cccc(Cl)c45)ncc23)CC12COC2] | +++ |
| 26 | 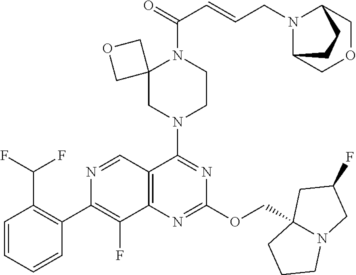 | ++ |
| 27 | 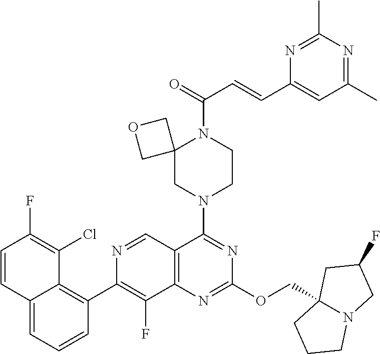 [SMILES: Cc1cc(/C=C/C(=O)N2CCN(c3nc(OC[C@@]45CCCN4C[C@H](F)C5)nc4c(F)c(-c5cccc6ccc(F)c(Cl)c56)ncc34)CC23COC3)nc(C)n1] | +++ |
| 28 | 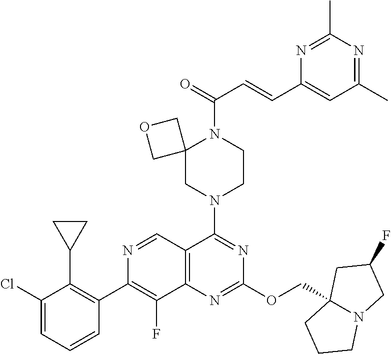 [SMILES: Cc1cc(/C=C/C(=O)N2CCN(c3nc(OC[C@@]45CCCN4C[C@H](F)C5)nc4c(F)c(-c5cccc(Cl)c5C5CC5)ncc34)CC23COC3)nc(C)n1] | + |
| 29 | 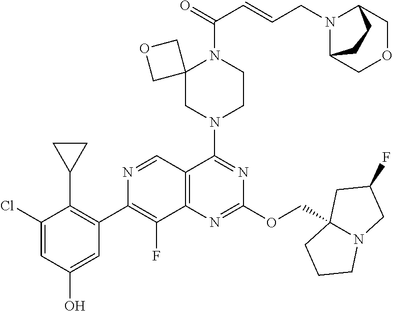 | ++++ |
| 30 | 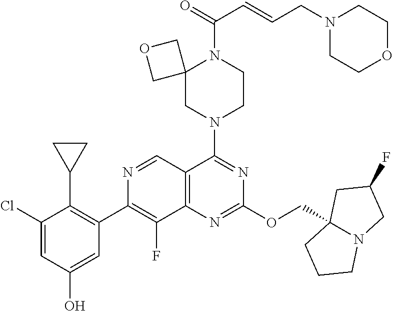 [SMILES: O=C(/C=C/CN1CCOCC1)N1CCN(c2nc(OC[C@@]34CCCN3C[C@H](F)C4)nc3c(F)c(-c4cc(O)cc(Cl)c4C4CC4)ncc23)CC12COC2] | ++++ |
| 31 | 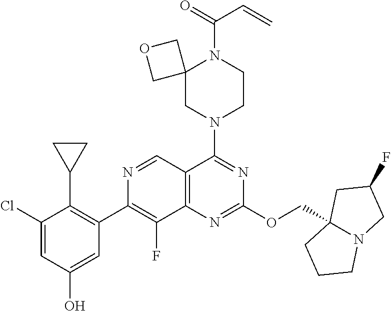 [SMILES: C=CC(=O)N1CCN(c2nc(OC[C@@]34CCCN3C[C@H](F)C4)nc3c(F)c(-c4cc(O)cc(Cl)c4C4CC4)ncc23)CC12COC2] | ++++ |
| 32 | 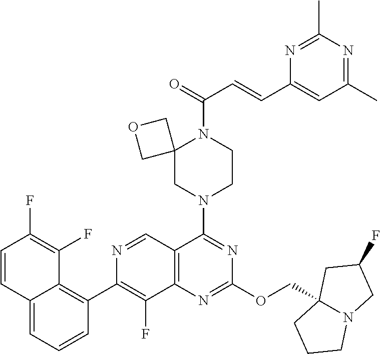 [SMILES: Cc1cc(/C=C/C(=O)N2CCN(c3nc(OC[C@@]45CCCN4C[C@H](F)C5)nc4c(F)c(-c5cccc6ccc(F)c(F)c56)ncc34)CC23COC3)nc(C)n1] | +++ |
| 33 | 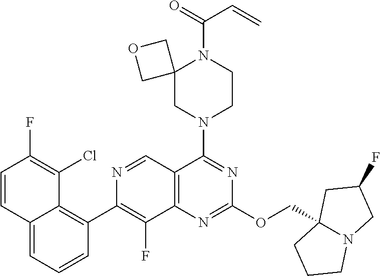 [SMILES: C=CC(=O)N1CCN(c2nc(OC[C@@]34CCCN3C[C@H](F)C4)nc3c(F)c(-c4cccc5ccc(F)c(Cl)c45)ncc23)CC12COC2] | +++ |
| 34 | 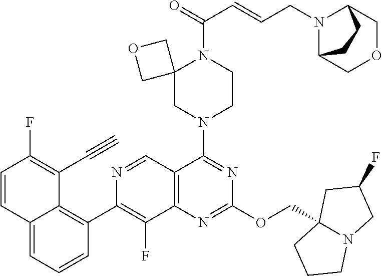 | ++++ |
| 35 | 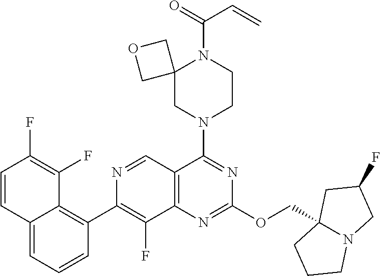 [SMILES: C=CC(=O)N1CCN(c2nc(OC[C@@]34CCCN3C[C@H](F)C4)nc3c(F)c(-c4cccc5ccc(F)c(F)c45)ncc23)CC12COC2] | ++++ |
| 36 | 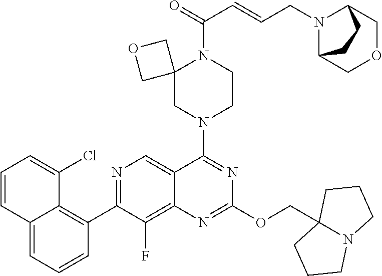 | ++++ |
| 37 | 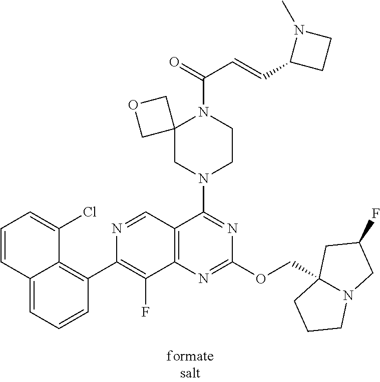 [SMILES: CN1CC[C@@H]1/C=C/C(=O)N1CCN(c2nc(OC[C@@]34CCCN3C[C@H](F)C4)nc3c(F)c(-c4cccc5cccc(Cl)c45)ncc23)CC12COC2] | +++ |
| 38 | 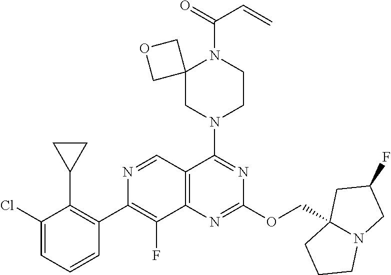 [SMILES: C=CC(=O)N1CCN(c2nc(OC[C@@]34CCCN3C[C@H](F)C4)nc3c(F)c(-c4cccc(Cl)c4C4CC4)ncc23)CC12COC2] | +++ |
| 39 | 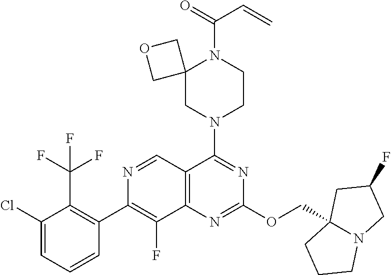 [SMILES: C=CC(=O)N1CCN(c2nc(OC[C@@]34CCCN3C[C@H](F)C4)nc3c(F)c(-c4cccc(Cl)c4C(F)(F)F)ncc23)CC12COC2] | +++ |
| 40 | 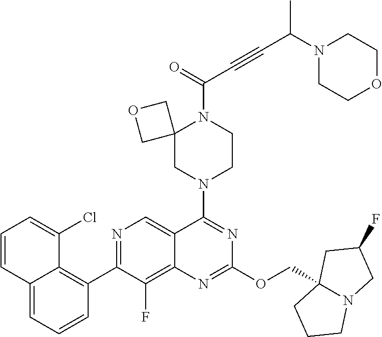 [SMILES: CC(C#CC(=O)N1CCN(c2nc(OC[C@@]34CCCN3C[C@H](F)C4)nc3c(F)c(-c4cccc5cccc(Cl)c45)ncc23)CC12COC2)N1CCOCC1] | ++ |
| 41 | 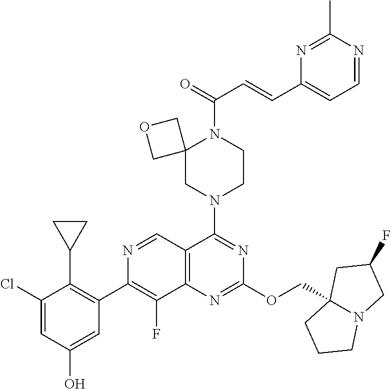 [SMILES: Cc1nccc(/C=C/C(=O)N2CCN(c3nc(OC[C@@]45CCCN4C[C@H](F)C5)nc4c(F)c(-c5cc(O)cc(Cl)c5C5CC5)ncc34)CC23COC3)n1] | ++++ |
| 42 | 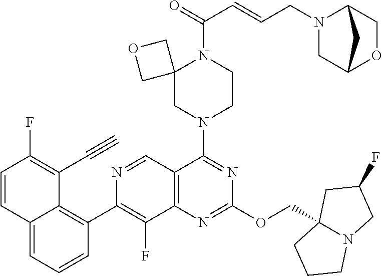 [SMILES: C#Cc1c(F)ccc2cccc(-c3ncc4c(N5CCN(C(=O)/C=C/CN6C[C@@H]7C[C@H]6CO7)C6(COC6)C5)nc(OC[C@@]56CCCN5C[C@H](F)C6)nc4c3F)c12] | ++++ |
| 43 | 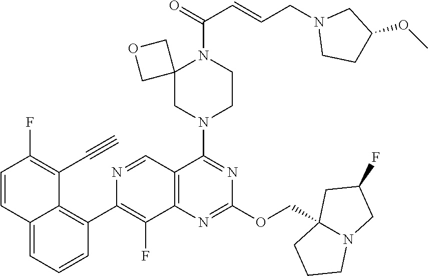 [SMILES: C#Cc1c(F)ccc2cccc(-c3ncc4c(N5CCN(C(=O)/C=C/CN6CC[C@@H](OC)C6)C6(COC6)C5)nc(OC[C@@]56CCCN5C[C@H](F)C6)nc4c3F)c12] | +++ |
| 44 | 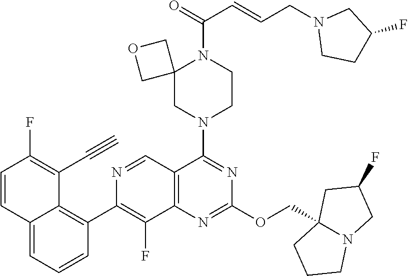 [SMILES: C#Cc1c(F)ccc2cccc(-c3ncc4c(N5CCN(C(=O)/C=C/CN6CC[C@@H](F)C6)C6(COC6)C5)nc(OC[C@@]56CCCN5C[C@H](F)C6)nc4c3F)c12] | ++++ |
| 45 | 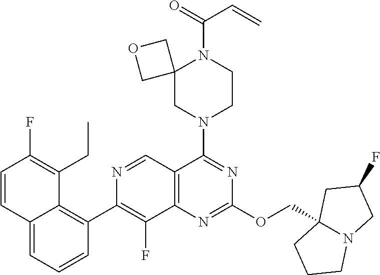 [SMILES: C=CC(=O)N1CCN(c2nc(OC[C@@]34CCCN3C[C@H](F)C4)nc3c(F)c(-c4cccc5ccc(F)c(CC)c45)ncc23)CC12COC2] | ++++ |
| 46 | 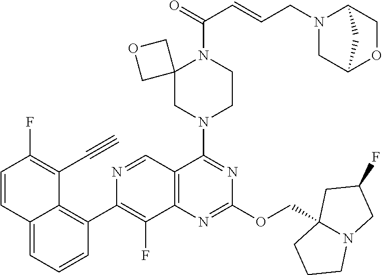 [SMILES: C#Cc1c(F)ccc2cccc(-c3ncc4c(N5CCN(C(=O)/C=C/CN6C[C@H]7C[C@@H]6CO7)C6(COC6)C5)nc(OC[C@@]56CCCN5C[C@H](F)C6)nc4c3F)c12] | ++++ |
| 47 | 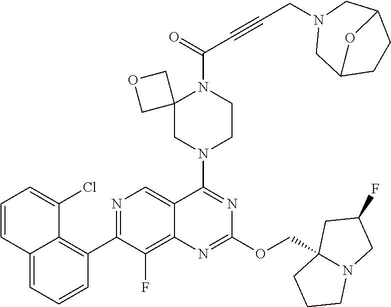 [SMILES: O=C(C#CCN1CC2CCC(C1)O2)N1CCN(c2nc(OC[C@@]34CCCN3C[C@H](F)C4)nc3c(F)c(-c4cccc5cccc(Cl)c45)ncc23)CC12COC2] | ++ |
| 48 | 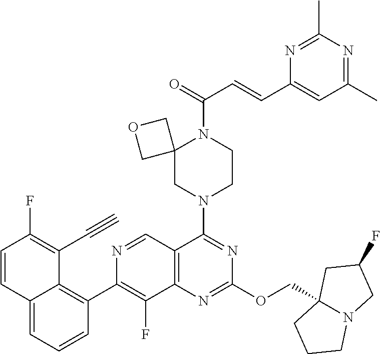 [SMILES: C#Cc1c(F)ccc2cccc(-c3ncc4c(N5CCN(C(=O)/C=C/c6cc(C)nc(C)n6)C6(COC6)C5)nc(OC[C@@]56CCCN5C[C@H](F)C6)nc4c3F)c12] | +++ |
| 49 | 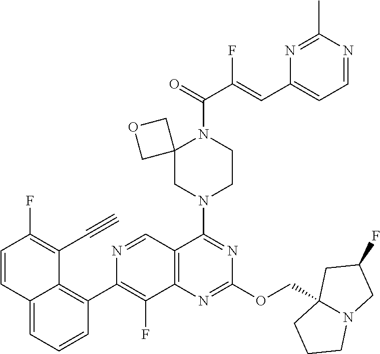 [SMILES: C#Cc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12] | ++++ |
| 50 | 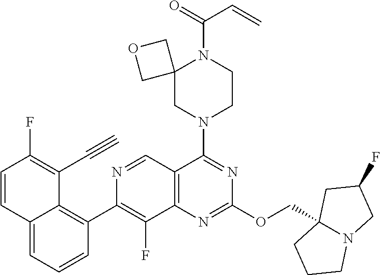 [SMILES: C#Cc1c(F)ccc2cccc(-c3ncc4c(N5CCN(C(=O)C=C)C6(COC6)C5)nc(OC[C@@]56CCCN5C[C@H](F)C6)nc4c3F)c12] | ++++ |
| 51 | 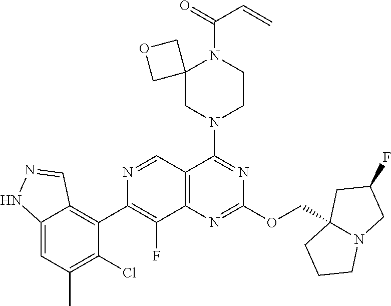 [SMILES: C=CC(=O)N1CCN(c2nc(OC[C@@]34CCCN3C[C@H](F)C4)nc3c(F)c(-c4c(Cl)c(C)cc5[nH]ncc45)ncc23)CC12COC2] | +++ |
| 52 | 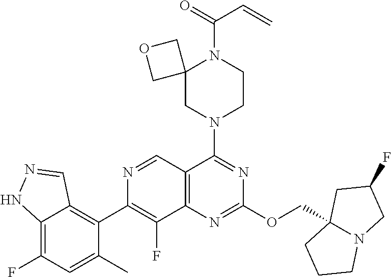 [SMILES: C=CC(=O)N1CCN(c2nc(OC[C@@]34CCCN3C[C@H](F)C4)nc3c(F)c(-c4c(C)cc(F)c5[nH]ncc45)ncc23)CC12COC2] | + |
| 53 | 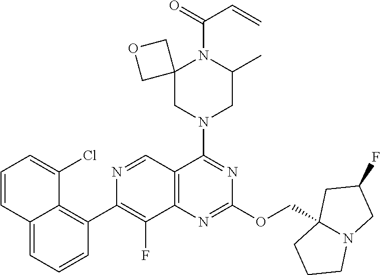 [SMILES: C=CC(=O)N1C(C)CN(c2nc(OC[C@@]34CCCN3C[C@H](F)C4)nc3c(F)c(-c4cccc5cccc(Cl)c45)ncc23)CC12COC2] | +++ |
| 54 | 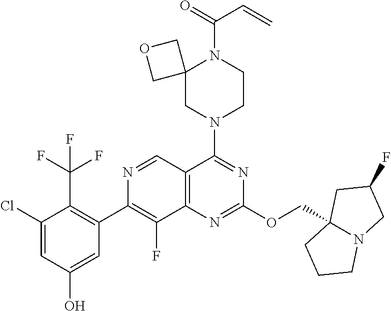 [SMILES: C=CC(=O)N1CCN(c2nc(OC[C@@]34CCCN3C[C@H](F)C4)nc3c(F)c(-c4cc(O)cc(Cl)c4C(F)(F)F)ncc23)CC12COC2] | ++++ |
| 55 | 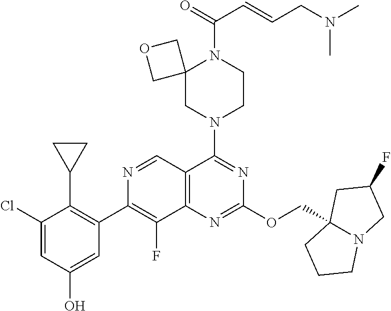 [SMILES: CN(C)C/C=C/C(=O)N1CCN(c2nc(OC[C@@]34CCCN3C[C@H](F)C4)nc3c(F)c(-c4cc(O)cc(Cl)c4C4CC4)ncc23)CC12COC2] | ++++ |
| 56 | 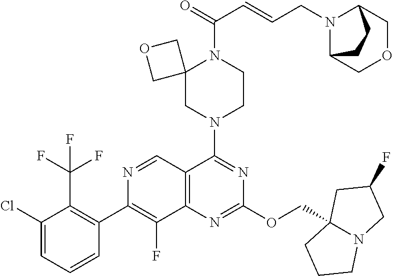 | ++++ |
| 57 | 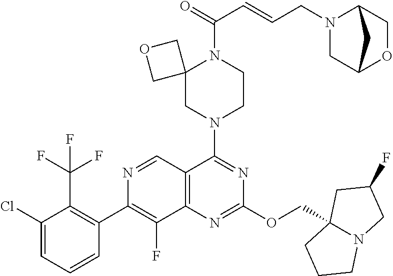 [SMILES: O=C(/C=C/CN1C[C@@H]2C[C@H]1CO2)N1CCN(c2nc(OC[C@@]34CCCN3C[C@H](F)C4)nc3c(F)c(-c4cccc(Cl)c4C(F)(F)F)ncc23)CC12COC2] | +++ |
| 58 | 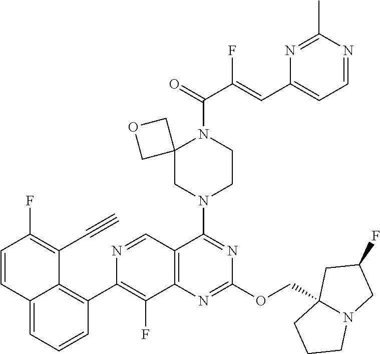 [SMILES: C#Cc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12] | ++++ |
| 59 | 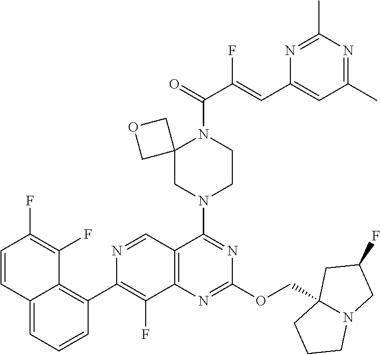 [SMILES: Cc1cc(/C=C(\F)C(=O)N2CCN(c3nc(OC[C@@]45CCCN4C[C@H](F)C5)nc4c(F)c(-c5cccc6ccc(F)c(F)c56)ncc34)CC23COC3)nc(C)n1] | ++++ |
| 60 | 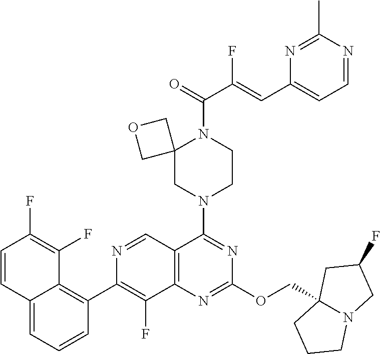 [SMILES: Cc1nccc(/C=C(\F)C(=O)N2CCN(c3nc(OC[C@@]45CCCN4C[C@H](F)C5)nc4c(F)c(-c5cccc6ccc(F)c(F)c56)ncc34)CC23COC3)n1] | ++++ |
| 61 | 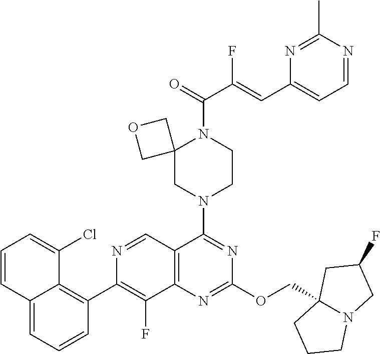 [SMILES: Cc1nccc(/C=C(\F)C(=O)N2CCN(c3nc(OC[C@@]45CCCN4C[C@H](F)C5)nc4c(F)c(-c5cccc6cccc(Cl)c56)ncc34)CC23COC3)n1] | + |
| 62 | 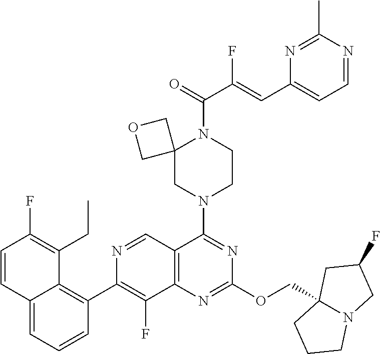 [SMILES: CCc1c(F)ccc2cccc(-c3ncc4c(N5CCN(C(=O)/C(F)=C/c6ccnc(C)n6)C6(COC6)C5)nc(OC[C@@]56CCCN5C[C@H](F)C6)nc4c3F)c12] | ++++ |
| 63 | 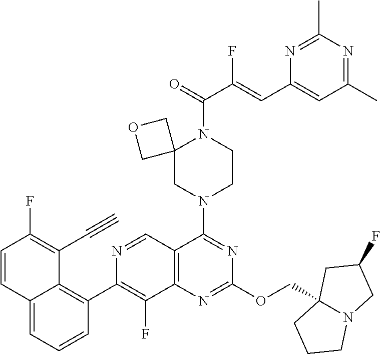 [SMILES: C#Cc1c(F)ccc2cccc(-c3ncc4c(N5CCN(C(=O)/C(F)=C/c6cc(C)nc(C)n6)C6(COC6)C5)nc(OC[C@@]56CCCN5C[C@H](F)C6)nc4c3F)c12] | ++++ |
| 64 | 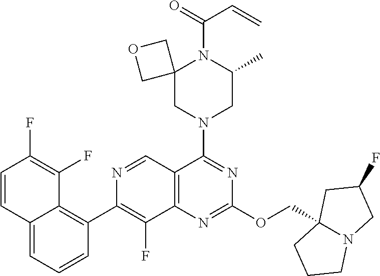 [SMILES: C=CC(=O)N1[C@H](C)CN(c2nc(OC[C@@]34CCCN3C[C@H](F)C4)nc3c(F)c(-c4cccc5ccc(F)c(F)c45)ncc23)CC12COC2] | ++++ |
| 65 | 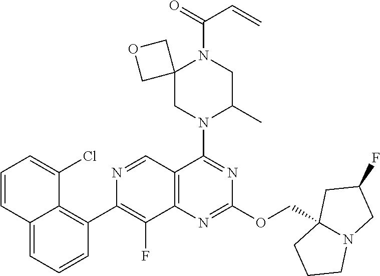 [SMILES: C=CC(=O)N1CC(C)N(c2nc(OC[C@@]34CCCN3C[C@H](F)C4)nc3c(F)c(-c4cccc5cccc(Cl)c45)ncc23)CC12COC2] | +++ |
| 66 | 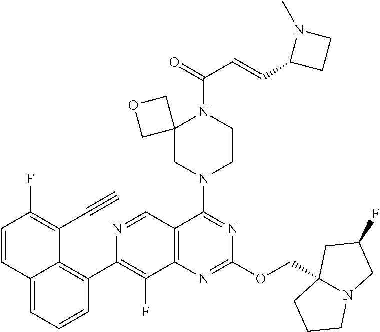 [SMILES: C#Cc1c(F)ccc2cccc(-c3ncc4c(N5CCN(C(=O)/C=C/[C@H]6CCN6C)C6(COC6)C5)nc(OC[C@@]56CCCN5C[C@H](F)C6)nc4c3F)c12] | +++ |
| 67 | 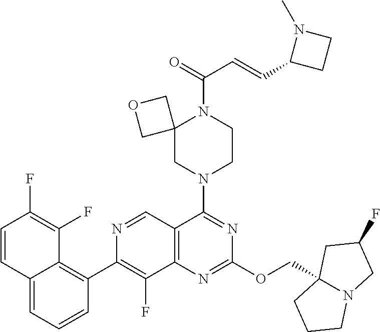 [SMILES: CN1CC[C@@H]1/C=C/C(=O)N1CCN(c2nc(OC[C@@]34CCCN3C[C@H](F)C4)nc3c(F)c(-c4cccc5ccc(F)c(F)c45)ncc23)CC12COC2] | +++ |
| 68 | 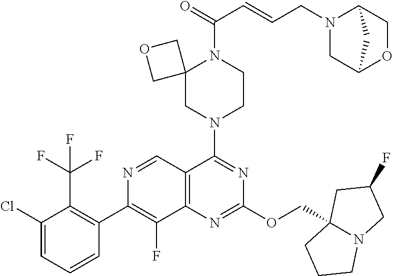 [SMILES: O=C(/C=C/CN1C[C@H]2C[C@@H]1CO2)N1CCN(c2nc(OC[C@@]34CCCN3C[C@H](F)C4)nc3c(F)c(-c4cccc(Cl)c4C(F)(F)F)ncc23)CC12COC2] | ++++ |
| 69 | 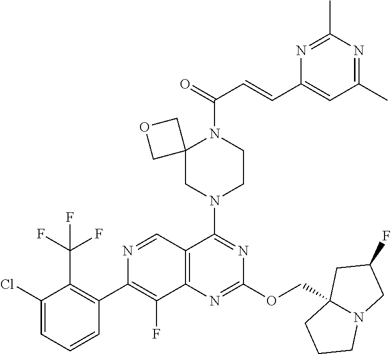 [SMILES: Cc1cc(/C=C/C(=O)N2CCN(c3nc(OC[C@@]45CCCN4C[C@H](F)C5)nc4c(F)c(-c5cccc(Cl)c5C(F)(F)F)ncc34)CC23COC3)nc(C)n1] | ++ |
| 70 | 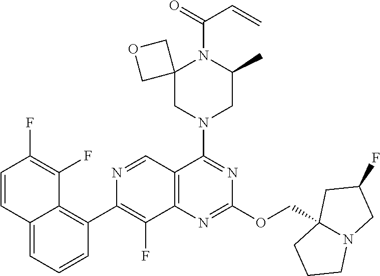 [SMILES: C=CC(=O)N1[C@@H](C)CN(c2nc(OC[C@@]34CCCN3C[C@H](F)C4)nc3c(F)c(-c4cccc5ccc(F)c(F)c45)ncc23)CC12COC2] | + |
| 71 | 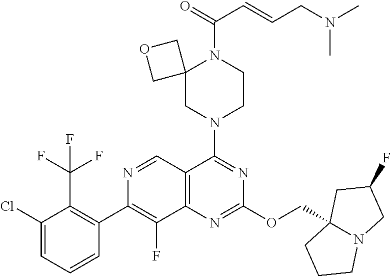 [SMILES: CN(C)C/C=C/C(=O)N1CCN(c2nc(OC[C@@]34CCCN3C[C@H](F)C4)nc3c(F)c(-c4cccc(Cl)c4C(F)(F)F)ncc23)CC12COC2] | ++ |
| 72 | 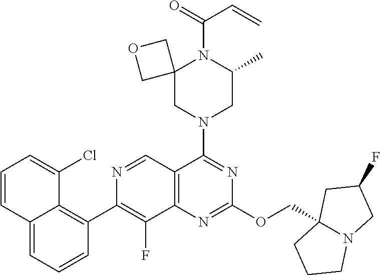 [SMILES: C=CC(=O)N1[C@H](C)CN(c2nc(OC[C@@]34CCCN3C[C@H](F)C4)nc3c(F)c(-c4cccc5cccc(Cl)c45)ncc23)CC12COC2] | ++ |
| 73 | 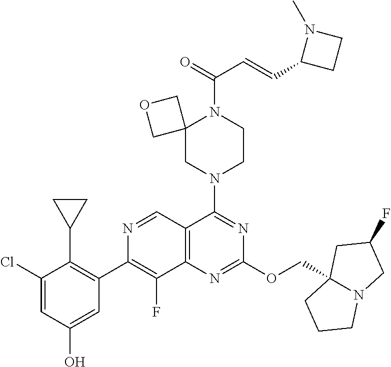 [SMILES: CN1CC[C@@H]1/C=C/C(=O)N1CCN(c2nc(OC[C@@]34CCCN3C[C@H](F)C4)nc3c(F)c(-c4cc(O)cc(Cl)c4C4CC4)ncc23)CC12COC2] | ++++ |
| 74 | 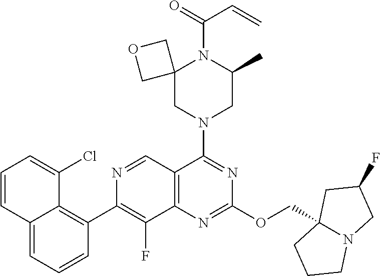 [SMILES: C=CC(=O)N1[C@@H](C)CN(c2nc(OC[C@@]34CCCN3C[C@H](F)C4)nc3c(F)c(-c4cccc5cccc(Cl)c45)ncc23)CC12COC2] | + |
| 75 | 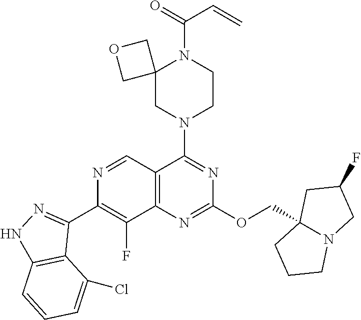 [SMILES: C=CC(=O)N1CCN(c2nc(OC[C@@]34CCCN3C[C@H](F)C4)nc3c(F)c(-c4n[nH]c5cccc(Cl)c45)ncc23)CC12COC2] | + |
| 76 | 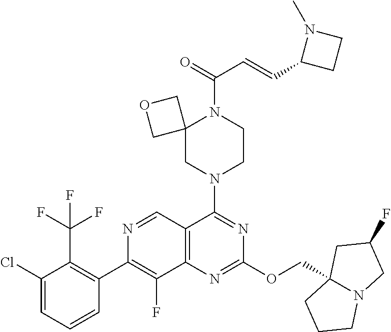 [SMILES: CN1CC[C@@H]1/C=C/C(=O)N1CCN(c2nc(OC[C@@]34CCCN3C[C@H](F)C4)nc3c(F)c(-c4cccc(Cl)c4C(F)(F)F)ncc23)CC12COC2] | + |
| 77 | 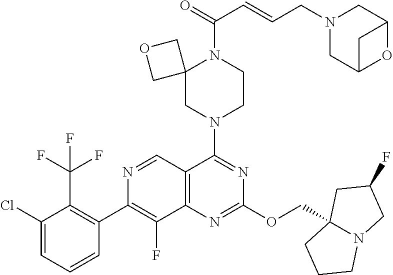 [SMILES: O=C(/C=C/CN1CC2CC(C1)O2)N1CCN(c2nc(OC[C@@]34CCCN3C[C@H](F)C4)nc3c(F)c(-c4cccc(Cl)c4C(F)(F)F)ncc23)CC12COC2] | +++ |
| 78 | 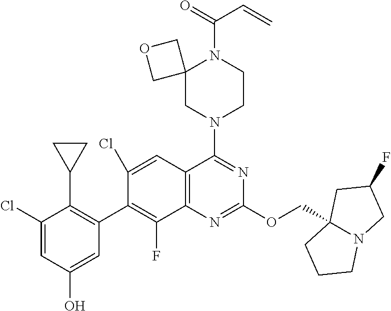 [SMILES: C=CC(=O)N1CCN(c2nc(OC[C@@]34CCCN3C[C@H](F)C4)nc3c(F)c(-c4cc(O)cc(Cl)c4C4CC4)c(Cl)cc23)CC12COC2] | ++++ |
| 79 | 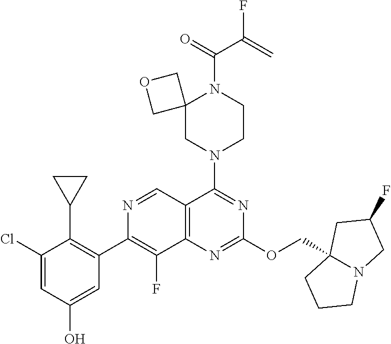 [SMILES: C=C(F)C(=O)N1CCN(c2nc(OC[C@@]34CCCN3C[C@H](F)C4)nc3c(F)c(-c4cc(O)cc(Cl)c4C4CC4)ncc23)CC12COC2] | + |
| 80 | 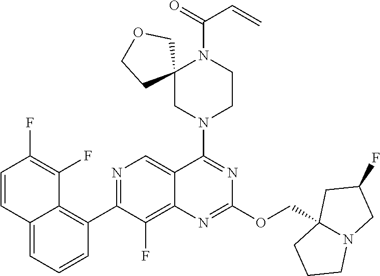 [SMILES: C=CC(=O)N1CCN(c2nc(OC[C@@]34CCCN3C[C@H](F)C4)nc3c(F)c(-c4cccc5ccc(F)c(F)c45)ncc23)C[C@@]12CCOC2] | +++ |
| 81 | 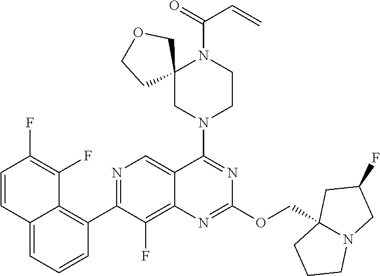 [SMILES: C=CC(=O)N1CCN(c2nc(OC[C@@]34CCCN3C[C@H](F)C4)nc3c(F)c(-c4cccc5ccc(F)c(F)c45)ncc23)C[C@]12CCOC2] | + |
| 82 | 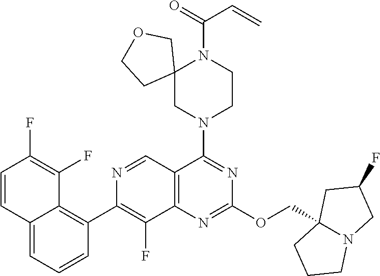 [SMILES: C=CC(=O)N1CCN(c2nc(OC[C@@]34CCCN3C[C@H](F)C4)nc3c(F)c(-c4cccc5ccc(F)c(F)c45)ncc23)CC12CCOC2] | +++ |
| 83 | 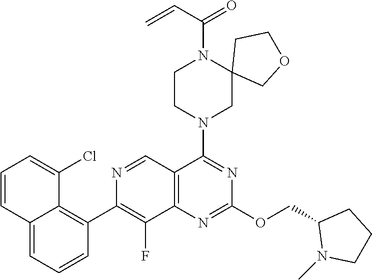 [SMILES: C=CC(=O)N1CCN(c2nc(OC[C@@H]3CCCN3C)nc3c(F)c(-c4cccc5cccc(Cl)c45)ncc23)CC12CCOC2] | + |
| 84 | 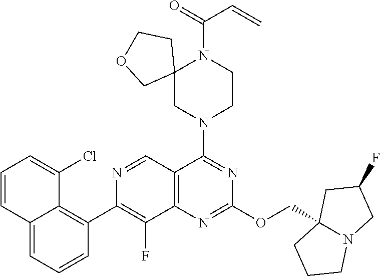 [SMILES: C=CC(=O)N1CCN(c2nc(OC[C@@]34CCCN3C[C@H](F)C4)nc3c(F)c(-c4cccc5cccc(Cl)c45)ncc23)CC12CCOC2] | ++ |
| 85 | 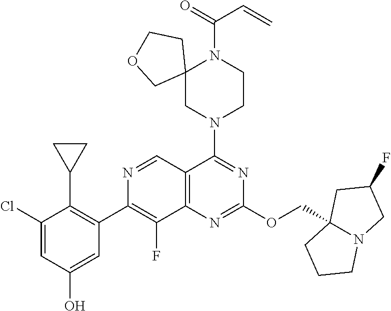 [SMILES: C=CC(=O)N1CCN(c2nc(OC[C@@]34CCCN3C[C@H](F)C4)nc3c(F)c(-c4cc(O)cc(Cl)c4C4CC4)ncc23)CC12CCOC2] | ++++ |
| 86 | 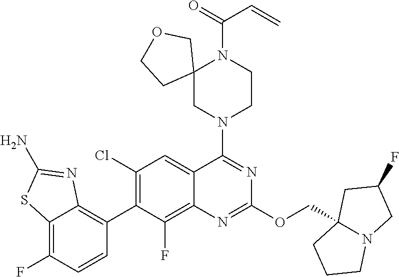 [SMILES: C=CC(=O)N1CCN(c2nc(OC[C@@]34CCCN3C[C@H](F)C4)nc3c(F)c(-c4ccc(F)c5sc(N)nc45)c(Cl)cc23)CC12CCOC2] | ++ |
| 87 | 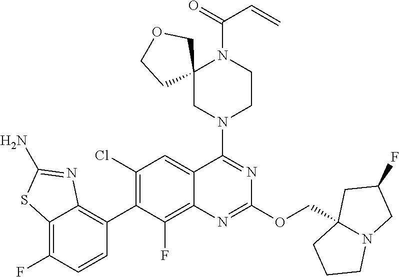 [SMILES: C=CC(=O)N1CCN(c2nc(OC[C@@]34CCCN3C[C@H](F)C4)nc3c(F)c(-c4ccc(F)c5sc(N)nc45)c(Cl)cc23)C[C@]12CCOC2] | + |
| 88 | 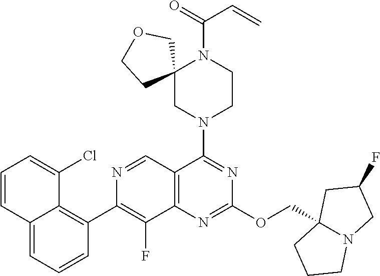 [SMILES: C=CC(=O)N1CCN(c2nc(OC[C@@]34CCCN3C[C@H](F)C4)nc3c(F)c(-c4cccc5cccc(Cl)c45)ncc23)C[C@@]12CCOC2] | ++ |
| 89 | 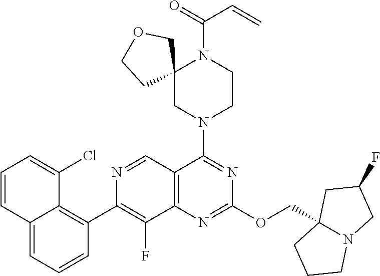 [SMILES: C=CC(=O)N1CCN(c2nc(OC[C@@]34CCCN3C[C@H](F)C4)nc3c(F)c(-c4cccc5cccc(Cl)c45)ncc23)C[C@]12CCOC2] | + |
| 90 | 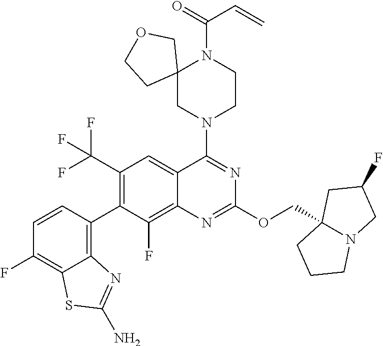 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)CC12CCOC2] | +++ |
| 91 | 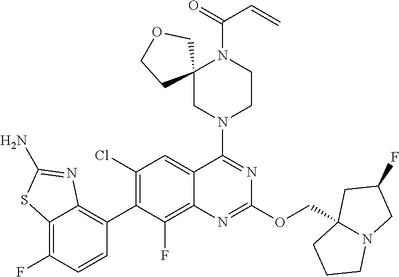 [SMILES: C=CC(=O)N1CCN(c2nc(OC[C@@]34CCCN3C[C@H](F)C4)nc3c(F)c(-c4ccc(F)c5sc(N)nc45)c(Cl)cc23)C[C@@]12CCOC2] | ++ |
| 92 | 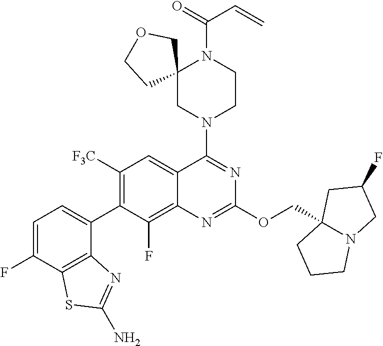 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@]12CCOC2] | +++ |
| 93 | 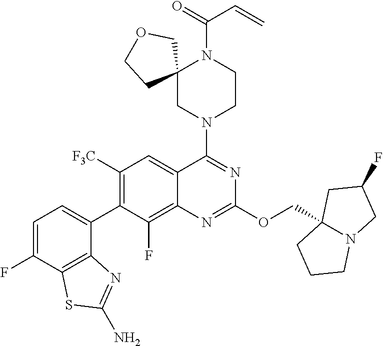 [SMILES: C=CC(=O)N1CCN(c2nc(OC[C@@]34CCCN3C[C@H](F)C4)nc3c(F)c(-c4ccc(F)c5sc(N)nc45)c(C(F)(F)F)cc23)C[C@@]12CCOC2] | + |
| 94 | 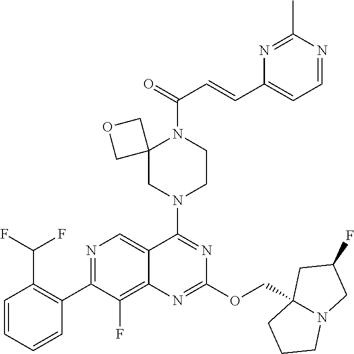 [SMILES: Cc1nccc(/C=C/C(=O)N2CCN(c3nc(OC[C@@]45CCCN4C[C@H](F)C5)nc4c(F)c(-c5ccccc5C(F)F)ncc34)CC23COC3)n1] | + |

IC₅₀ ++++ is less than 10 nM, +++ is 10 to less than 100 nM, ++ is 100 to less than 500 nM, + is greater or equal to 500 nM.

### Example B-2: Inhibition of KRASG12C and PI3Ka Binding

The AlphaScreen technology is used to determine IC₅₀s for compound inhibition of KRAS G12C (present as the Cys-light (C51S, C80L and C₁₁₈S), truncated version comprising amino acids 1-169) and PI3Ka interaction. Compounds are diluted in 100% DMSO and each compound concentration is spotted at 200 nl/well onto low volume, white 384 well plates. The KRAS G12C contains a biotin-AviTag and the PI3Ka, as Ras-binding domain (amino acids 157-300, RBD), is His-tagged. KRAS G12C is preloaded with the GTP analogue Guanosine 5′-[β,γ-imido]triphosphate (GMPPNP). The KRAS G12C is diluted in 25 mM Hepes, pH 7.4, 150 mM NaCl, 5 mM MgCl₂, 0.01% TritonX-100 and 10 μM GMPPNP and added at 10 μl/well to compound-spotted plates resulting in a DMSO concentration of 2%. Plates are incubated for 2 hours. A mixture of RBD and the AlphaScreen streptavidin donor and nickel chelate acceptor beads diluted in 25 mM Hepes, pH 7.4, 150 mM NaCl, 5 mM MgCl₂, 0.01% TritonX-100 and 2% DMSO are then added at 10 μl/well and incubated for 60-90 minutes before the samples are read for emission at 570 nm after excitation of the donor beads at 680 nm. All incubations are performed at room temperature. The final top compound concentration is 50 μM with 1:3 titrations for 10-point dose response curves. Final assay conditions are 1.5 nM KRAS G12C, 100 nM RBD, 1.25 μg/ml of AlphaScreen donor beads and 10 μg/ml AlphaLISA acceptor beads. IC₅₀s are determined using nonlinear regression fit of [inhibitor] vs. response (4 parameters).

A counter assay is also set up to rule out inhibitors of the AlphaScreen technology itself. Compound plates are incubated for 2 hours as above with buffer only. The AlphaScreen beads are added as above except an unrelated biotinylated His-tagged peptide is substituted for the RBD. Samples are read and analyzed as above.

### Example B-3: MCF10A (G12C or G12C-A59G)-KRAS cell viability assay

MCF10A (ATCC, cat. CRL-10317) cells are maintained in MEBM (Lonza, cat. CC-3151) with 1% horse serum (Sigma, cat. H1270), MEGM mammary epithelial cell growth medium SingleQuotsKit (Lonza, cat. CC-4146) and 25 ng/ml Cholera toxin (Sigma, cat. C₈₀₅₂). These cells are transduced with either KRAS G12C or G12C/A59G followed by puromycin selection to generate stably expressing cells. For the cell viability assay, 1000 cells of either MCF10A KRAS G12C or MCF10A G12C/A59G are plated in 384-well spheroid microplate (Corning, cat. 3830). The following day, cells are treated with compounds (10 μM top concentration, 3-fold dilution, and 11 doses). 10 μM Tremetinib (MCE, cat. HY-10999/CS-0060) is used as control. The Tecan: HP D300E is used to dispense the compounds. After five days of incubation, celltiter-glo luminescent assay kit (Promega, cat. G7573) is used according to manufacturer's protocol to measure cellular viability using a BioTek plate reader. The data is then imported to and processed in Dotmatics where EC50s were calculated using the Lavenberg-Marquardt 4 parameters fitting procedure, with difference gradients.

### Example B-4: Treatment of Human Patients

A human patient suffering from a cancer, (e.g., a KRAS mediated cancer, as disclosed herein) can be administered a therapeutically effective dose of a compound disclosed herein (e.g., a compound of Table 1). The treatment can slow down or halt the growth of a tumor, reduce a tumor volume or mass, or eradicate the tumor in the patient.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (I), Formula (II), Formula (III), or Formula (IV) 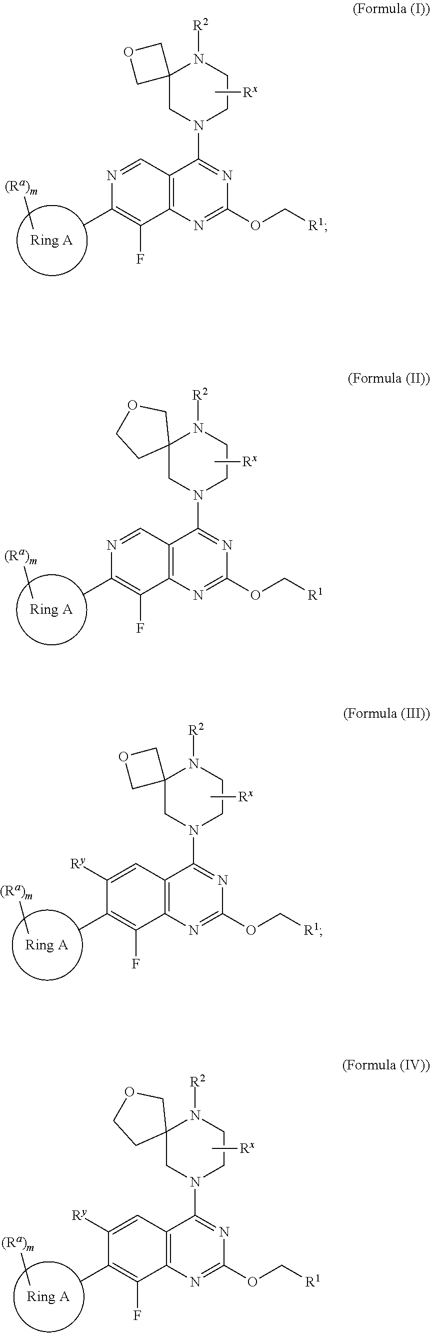
or a salt thereof; and/or an isotopologue thereof; wherein:
Ring A is a 6-10 membered aryl or a 5-10 membered heteroaryl;
each R^{a} is independently selected from the group consisting of halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl;
m is 0, 1, 2 or 3;
R¹ is 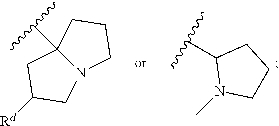
R^{d} is H or F;
R² is 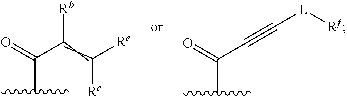
R^{b} is —H or —F;
R^{c} is —H;
R^{e} is —H, —Y—R^{e1}, —Y—R^{e2}, or —Y—R^{e3};
Y is —(C(R^{Y1})(R^{Y2}))ₓ—;
x is 0, 1 or 2 provided that when R^{e} is Y—R^{e3}, x is not 0;
R^{Y1} and R^{Y2} in each occurrence are independently selected from the group consisting of —H and —CH₃;
R^{e1} is a 4-10 membered heterocycle which is substituted with 0, 1, 2, 3 or 4 substituents independently selected from the group consisting of halo, hydroxy, C₁-C₄ alkyl, C₁-C₆ alkoxy, C₁-C₆ alkoxyalkoxy, C₁-C₄ haloalkyl, and C₁-C₄ haloalkoxy;
R^{e2} is a 5-6 membered heteroaryl substituted with 0, 1 or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy, and C₃-C₄ cycloalkyl optionally substituted with one or two instances of fluoro or methyl;
R^{e3} is —NR³¹R³²;
R³¹ and R³² are independently selected from the group consisting of hydrogen, C₁-C₄ alkyl, C₁-C₄ alkoxy, C₁-C₄ haloalkoxy, and C₁-C₄ alkyl substituted with a 3-6 membered heterocycle;
L is —(C(R^{L1})(L^{L2}))_{z}—;
z is 0, 1 or 2;
R^{L1} and R^{L2} in each occurrence are independently selected from the group consisting of —H and —CH₃;
R^{f} is a 4-10 membered heterocycle which is substituted with 0, 1, 2, 3 or 4 substituents independently selected from the group consisting of halo, hydroxy, C₁-C₄ alkyl, C₁-C₆ alkoxy, C₁-C₄ haloalkyl, C₁-C₄ haloalkoxy and C₂-C₃ alkynyl, with the proviso that R^{f} is not substituted with more than one C₂-C₃ alkynyl;
R^{x} is selected from the group consisting of —H and C₁-C₄ alkyl; and
R^{y} is selected from the group consisting of halo and C₁-C₄ haloalkyl.

2. The compound of claim 1, or a salt thereof, and/or an isotopologue thereof, wherein the compound is of Formula (I).

3. The compound of claim 1, or a salt thereof, and/or an isotopologue thereof, wherein the compound is of Formula (II).

4. The compound of claim 1, or a salt thereof, and/or an isotopologue thereof, wherein the compound is of Formula (III).

5. The compound of claim 1, or a salt thereof, and/or an isotopologue thereof, wherein the compound is of Formula (IV).

6. The compound of any one of claims 1-5, or a salt thereof, and/or an isotopologue thereof, wherein Ring A is selected from the group consisting of a 6-10 membered aryl and a 9-10 membered bicyclic heteroaryl having 1, 2 or 3 heteroatoms independently selected from the group consisting of N, O and S.

7. The compound of any one of claims 1-5, or a salt thereof, and/or an isotopologue thereof, wherein Ring A is selected from the group consisting of naphthalenyl, phenyl, benzothiazolyl, isoquinolinyl, indazolyl and pyridinyl.

8. The compound of any one of claims 1-5, or a salt thereof, and/or an isotopologue thereof, wherein Ring A is selected from the group consisting of naphthalen-1-yl and phenyl.

9. The compound of any one of claims 1-8, or a salt thereof, and/or an isotopologue thereof, wherein each R^{a} is independently selected from the group consisting of —F, —Cl, —OH, —NH₂, -Me, -Et, -cyclopropyl, —CF₂H, —CF₃ and —C═CH.

10. The compound of any one of claims 1-5, or a salt thereof, and/or an isotopologue thereof, wherein Ring A is selected from the group consisting of: 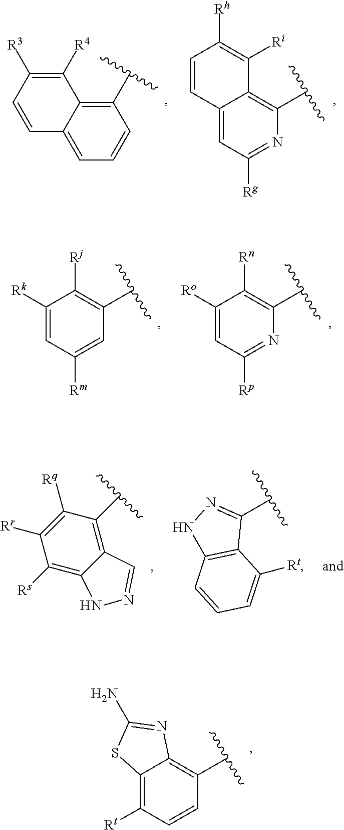
wherein:
each R³, R⁴, R^{h}, Rⁱ, R^{j}, R^{k}, Rⁿ, R^{o}, R^{q}, R^{r} and R^{s} is independently selected from the group consisting of hydrogen, halo, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
each R^{g}, R^{m} and R^{p} is independently selected from the group consisting of hydrogen, halo, —OH, —NH₂, C₁-C₄ alkyl, C₃-C₄ cycloalkyl, C₁-C₄ alkoxy, C₁-C₄ haloalkyl, C₁-C₄ haloalkoxy and C₂-C₃ alkynyl; and
R^{t} is halo.

11. The compound of claim 10, or a salt thereof, and/or an isotopologue thereof, wherein each R³, R⁴, R^{h}, Rⁱ, R^{j}, R^{k}, Rⁿ, R^{o}, R^{q}, R^{r} and R^{s} is independently selected from the group consisting of —H, —F, —Cl, -Me, -Et, -cyclopropyl, —CF₂H, —CF₃ and —C═CH; each R^{g}, R^{m} and R^{p} is independently selected from the group consisting of —H and —OH; and R^{t} is —F or —Cl.

12. The compound of any one of claims 1-5, or a salt thereof, and/or an isotopologue thereof, wherein Ring A is selected from the group consisting of:

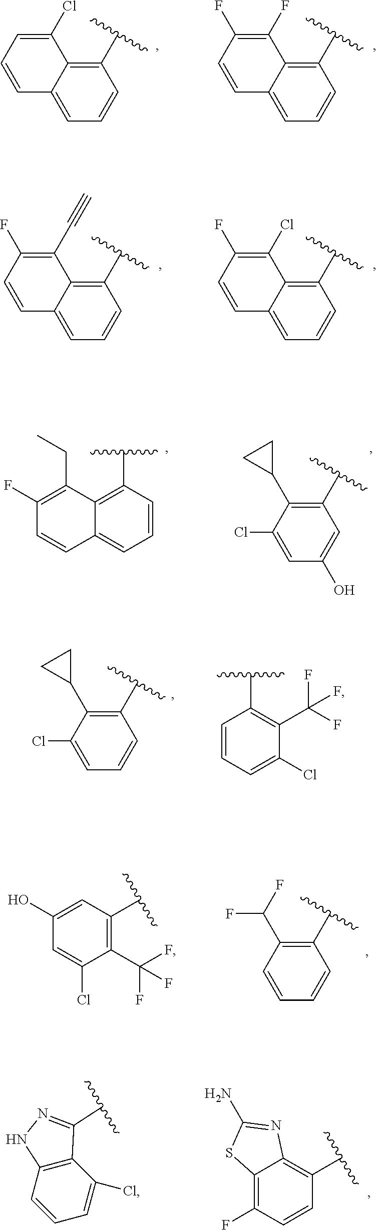

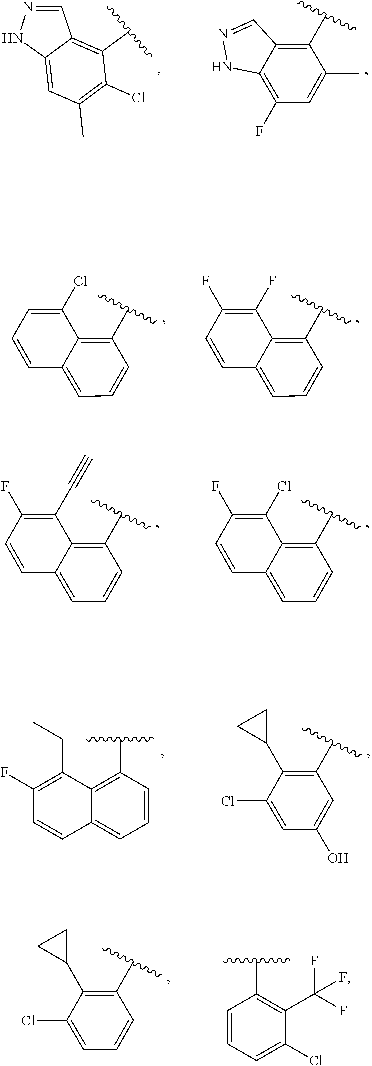

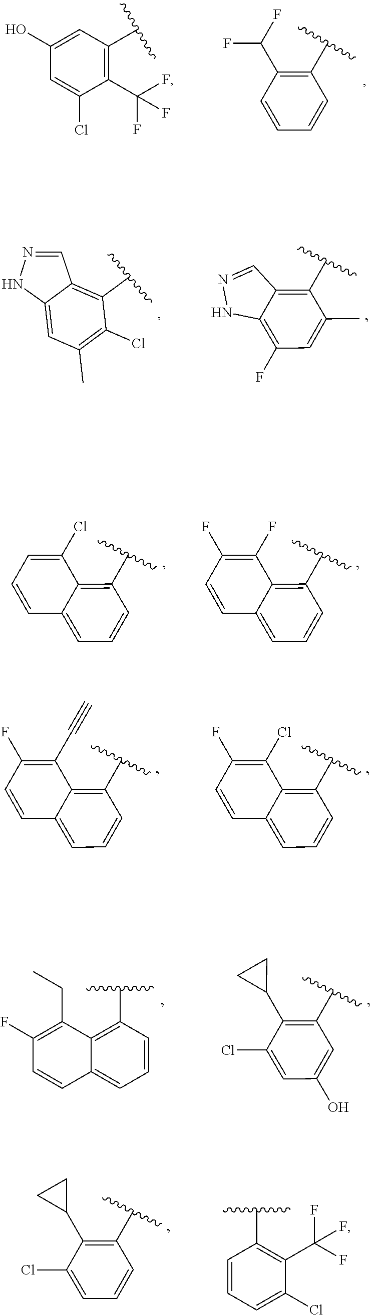

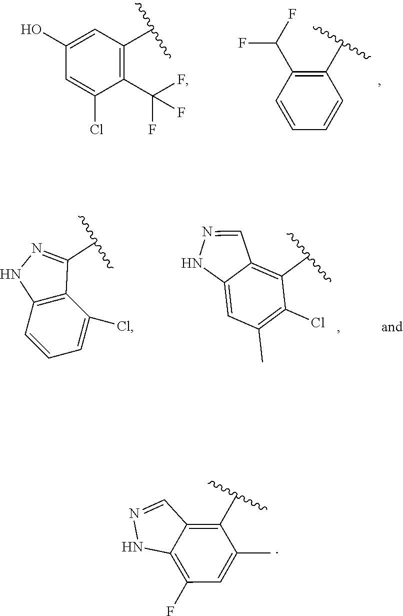 [SMILES: *c1cc(O)cc(Cl)c1C(F)(F)F ; *c1ccccc1C(F)F ; *c1n[nH]c2cccc(Cl)c12 ; *c1c(Cl)c(C)cc2[nH]ncc12 ; *c1c(C)cc(F)c2[nH]ncc12]

13. The compound of any one of claims 1-12, or a salt thereof, and/or an isotopologue thereof, wherein R¹ is selected from the group consisting of

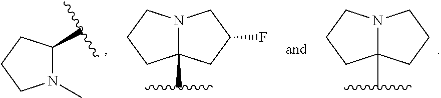

14. The compound of any one of claims 1-13, or a salt thereof, and/or an isotopologue thereof, wherein R² is

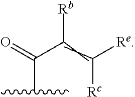

15. The compound of any one of claims 1-14, or a salt thereof, and/or an isotopologue thereof, wherein Y is selected from the group consisting of a bond, —CH₂—, —CH(CH₃)—, —C(CH₃)₂—, —CH₂CH₂—, —CH(CH₃)CH₂— and —CH₂CH(CH₃)—.

16. The compound of any one of claims 1-15, or a salt thereof, and/or an isotopologue thereof, wherein R^{e1} is selected from the group consisting of azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected from the group consisting of halo, hydroxy, C₁-C₄ alkyl, C₁-C₆ alkoxy, C₁-C₆ alkoxyalkoxy, C₁-C₄ haloalkyl and C₁-C₄ haloalkoxy.

17. The compound of any one of claims 1-15, or a salt thereof, and/or an isotopologue thereof, wherein R^{e1} is selected from the group consisting of: 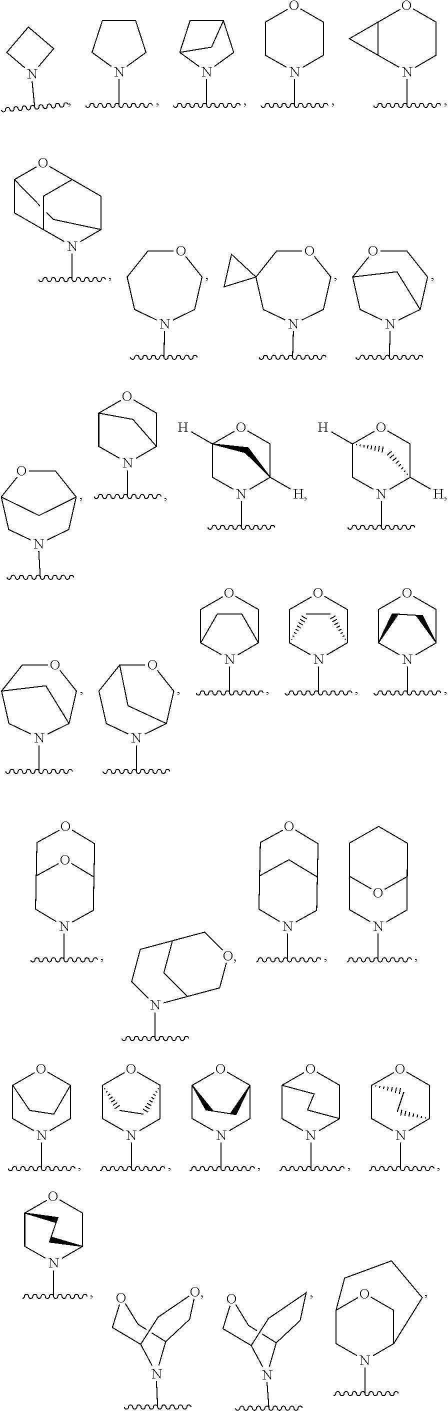 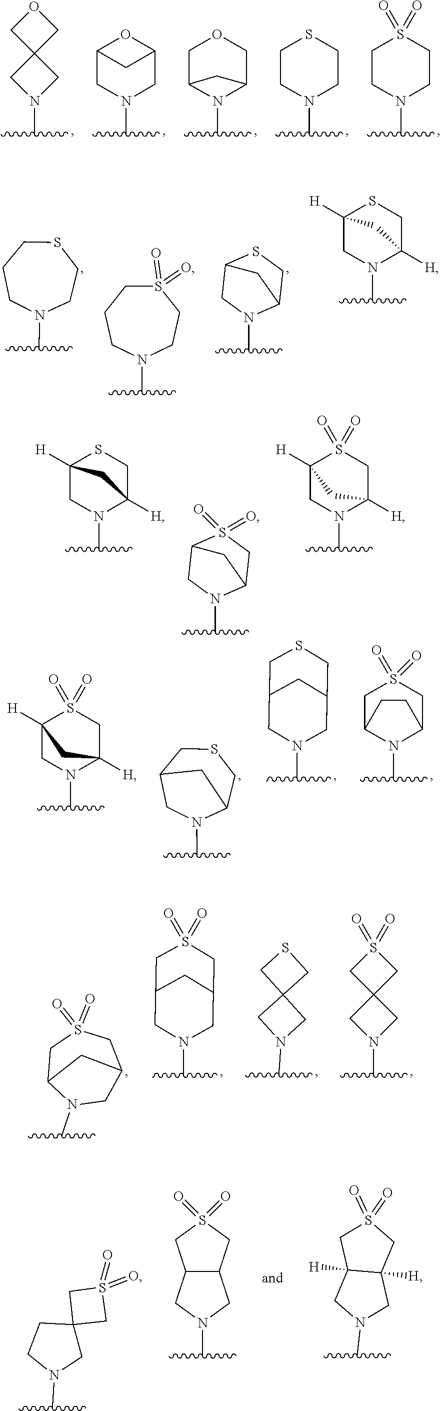
each substituted with 0, 1, 2, 3 or 4 substituents independently selected from the group consisting of halo, hydroxy, C₁-C₄ alkyl, C₁-C₆ alkoxy, C₁-C₆ alkoxyalkoxy, C₁-C₄ haloalkyl and C₁-C₄ haloalkoxy.

18. The compound of any one of claims 1-17, or a salt thereof, and/or an isotopologue thereof, wherein the 4-10 membered heterocycle of R^{e1} is unsubstituted or substituted with 0, 1 or 2 substituents independently selected from the group consisting of —F, —OMe and -Me.

19. The compound of any one of claims 1-15, or a salt thereof, and/or an isotopologue thereof, wherein R^{e1} is selected from the group consisting of:

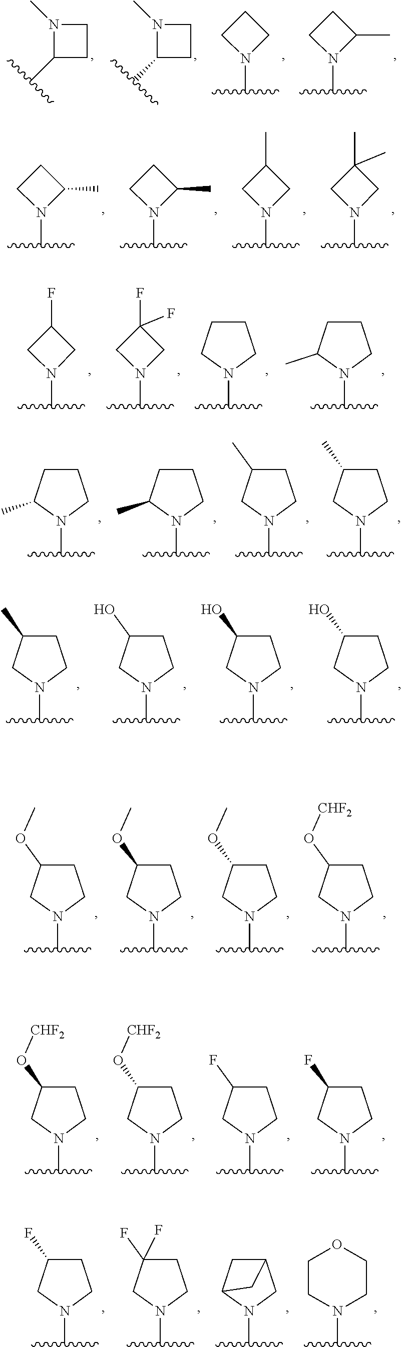

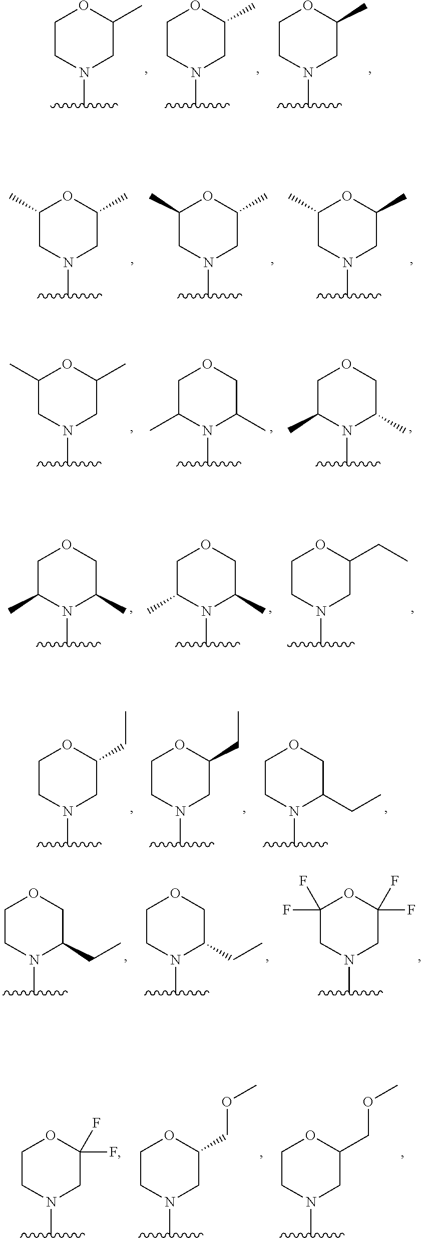

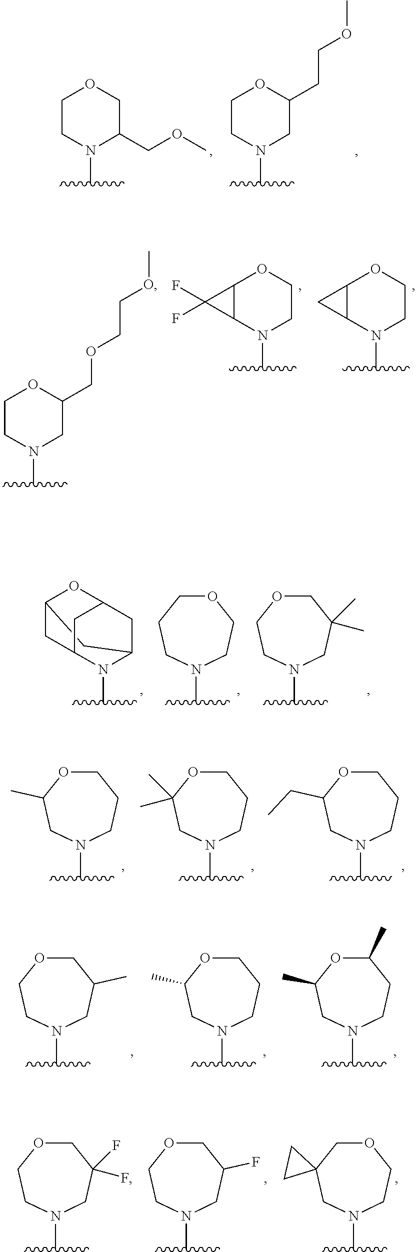

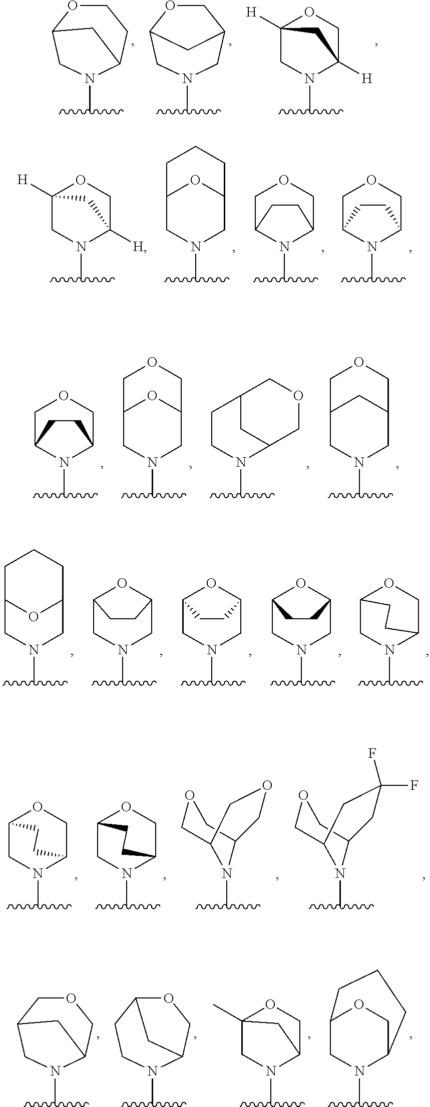

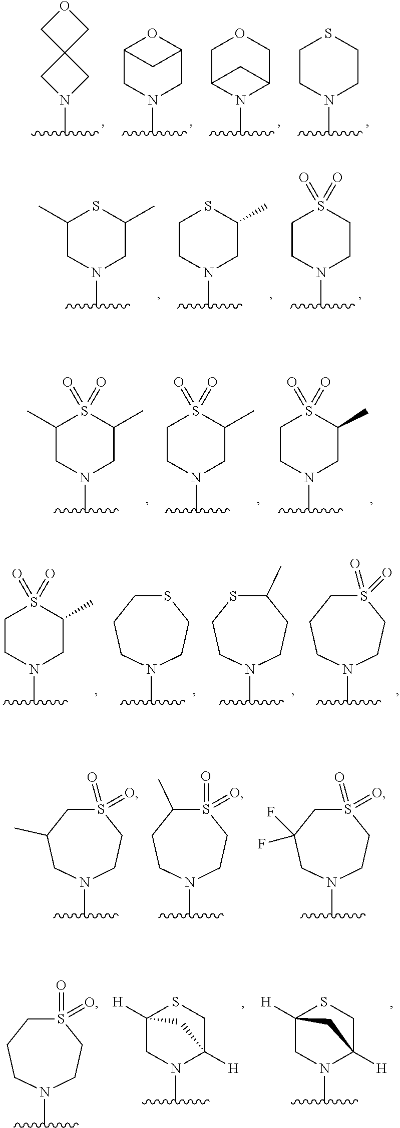

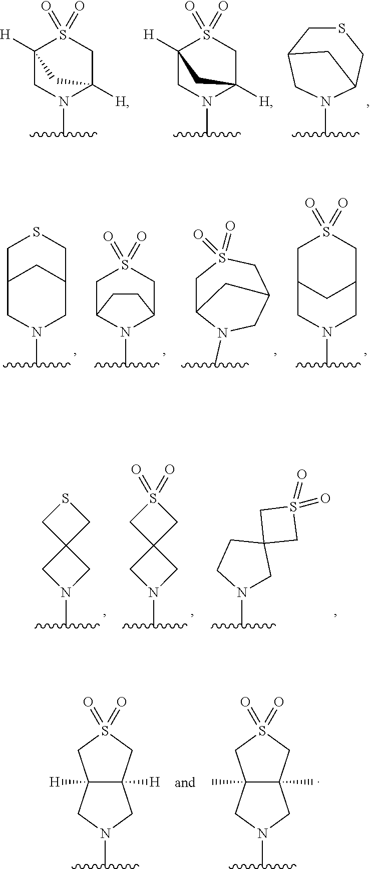

20. The compound of any one of claims 1-14, or a salt thereof, and/or an isotopologue thereof, wherein R^{e} is selected from the group consisting of

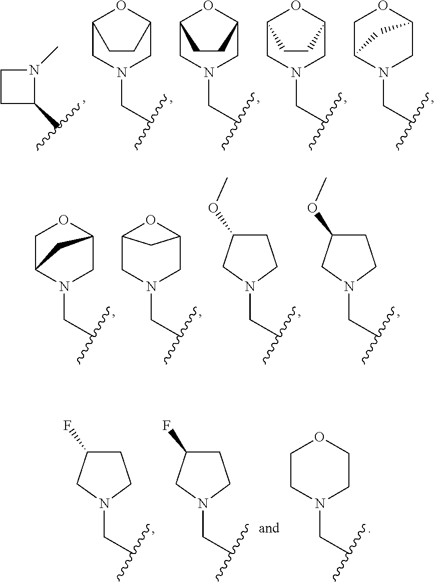

21. The compound of any one of claims 1-15, or a salt thereof, and/or an isotopologue thereof, wherein R^{e2} is selected from the group consisting of pyrimidinyl, pyridazinyl, pyrazinyl, oxazolyl, 1,2,4-oxadiazolyl, 1,3,4-oxadiazolyl, 1H-1,2,4-triazolyl, imidazolyl, 4H-1,2,4-triazolyl, 1,2,4-thiadiazolyl and isoxazolyl, each substituted with 0, 1 or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

22. The compound of any one of claims 1-15, or a salt thereof, and/or an isotopologue thereof, wherein R^{e2} is selected from the group consisting of: 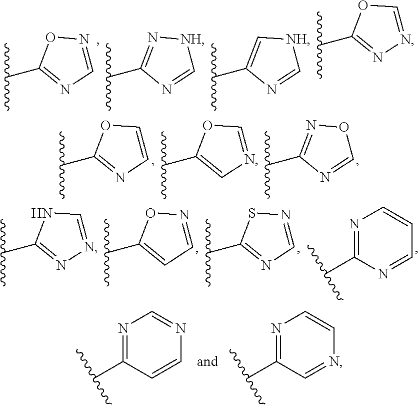
each substituted with 0, 1 or 2 substituents independently selected from the group consisting of halo, C₁-C₄ alkyl, C₁-C₄ hydroxyalkyl, C₁-C₆ alkoxy, C₁-C₄ haloalkyl, C₁-C₄ haloalkoxy and C₃-C₆ cycloalkyl optionally substituted with one or two instances of fluoro or methyl.

23. The compound of any one of claims 1-15, or a salt thereof, and/or an isotopologue thereof, wherein R^{e2} is selected from the group consisting of

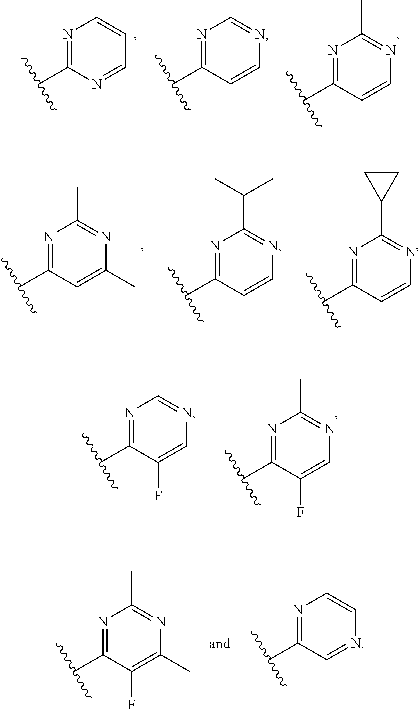

24. The compound of any one of claims 1-15, or a salt thereof, and/or an isotopologue thereof, wherein R³¹ is methyl.

25. The compound of any one of claims 1-15 and 24, or a salt thereof, and/or an isotopologue thereof, wherein R³² is C₁-C₄ alkyl substituted with a heterocycle selected from the group consisting of azetidine, oxetane, pyrrolidine, tetrahydrofuran, piperidine, tetrahydropyran and morpholine.

26. The compound of any one of claims 1-15 and 24, or a salt thereof, and/or an isotopologue thereof, wherein R³² is 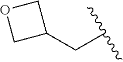 [SMILES: *CC1COC1]
or methyl.

27. The compound of any one of claims 1-15, or a salt thereof, and/or an isotopologue thereof, wherein R^{e3} is selected from the group consisting of

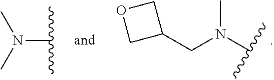

28. The compound of any one of claims 1-15, or a salt thereof, and/or an isotopologue thereof, wherein R^{e} is selected from the group consisting of: —H,

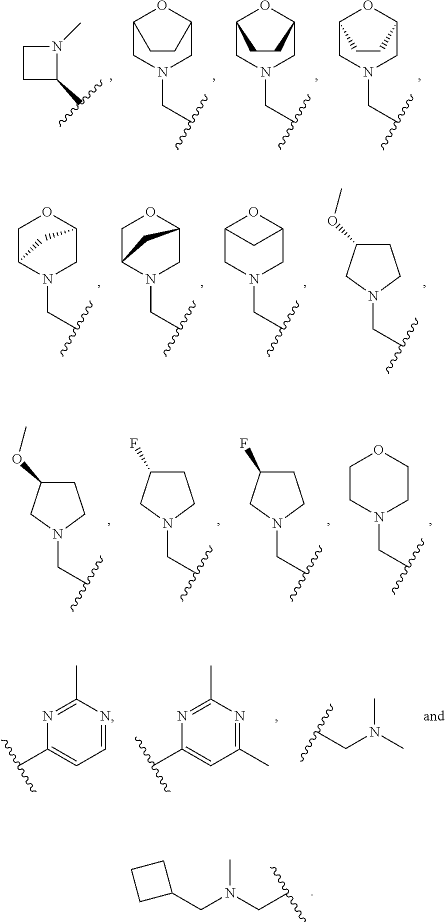

29. The compound of any one of claims 1-13, or a salt thereof, and/or an isotopologue thereof, wherein R² is

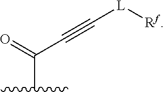

30. The compound of any one of claims 1-13 and 29, or a salt thereof, and/or an isotopologue thereof, wherein L is selected from the group consisting of a bond, —CH₂—, CH(CH₃)—, —C(CH₃)₂—, —CH₂CH₂—, —CH(CH₃)CH₂— and —CH₂CH(CH₃)—.

31. The compound of any one of claims 1-13, 29, and 30, or a salt thereof, and/or an isotopologue thereof, wherein R^{f} is selected from the group consisting of azetidine, pyrrolidine, 2-azabicyclo[2.1.1]hexane, morpholine, 2-oxa-5-azabicyclo[4.1.0]heptane, 1,4-oxazepane, 2-oxa-6-azaadamantane, 5-oxa-8-azaspiro[2.6]nonane, 2-oxa-6-azabicyclo[3.2.1]octane, 6-oxa-3-azabicyclo[3.2.1]octane, 3-oxa-6-azabicyclo[3.2.1]octane, 6-oxa-2-azabicyclo[3.2.1]octane, 2-oxa-5-azabicyclo[2.2.1]heptane, 3-oxa-9-azabicyclo[3.3.1]nonane, 3,7-dioxa-9-azabicyclo[3.3.1]nonane, 3-oxa-7-azabicyclo[3.3.1]nonane, 3,9-dioxa-7-azabicyclo[3.3.1]nonane, 3-oxa-8-azabicyclo[3.2.1]octane, 7-oxa-2-azabicyclo[3.3.1]nonane, 8-oxa-3-azabicyclo[3.2.1]octane, 9-oxa-3-azabicyclo[3.3.1]nonane, 9-oxa-3-azabicyclo[3.3.1]nonane, 2-oxa-6-azaspiro[3.3]heptane, 3-oxa-6-azabicyclo[3.1.1]heptane, 6-oxa-3-azabicyclo[3.1.1]heptane, thiomorpholine, thiomorpholine 1,1-dioxide, 4-thiazepane, 1,4-thiazepane 1,1-dioxide, 3-thia-6-azabicyclo[3.2.1]octane, 3-thia-8-azabicyclo[3.2.1]octane 3,3-dioxide, 3-thia-7-azabicyclo[3.3.1]nonane, 3-thia-6-azabicyclo[3.2.1]octane 3,3-dioxide, 3-thia-7-azabicyclo[3.3.1]nonane 3,3-dioxide, 2-thia-5-azabicyclo[2.2.1]heptane, 2-thia-5-azabicyclo[2.2.1]heptane 2,2-dioxide, 2-thia-6-azaspiro[3.4]octane 2,2-dioxide, 2-thia-6-azaspiro[3.3]heptane 2,2-dioxide, 2-thia-6-azaspiro[3.3]heptane and hexahydro-1H-thieno[3,4-c]pyrrole 2,2-dioxide, each substituted with 0, 1, 2, 3 or 4 substituents independently selected halo, hydroxy, C₁-C₄ alkyl, C₁-C₆ alkoxy, C₁-C₆ alkoxyalkoxy, C₁-C₄ haloalkyl and C₁-C₄ haloalkoxy.

32. The compound of any one of claims 1-13, 29, and 30, or a salt thereof, and/or an isotopologue thereof, wherein R^{f} is selected from the group consisting of: 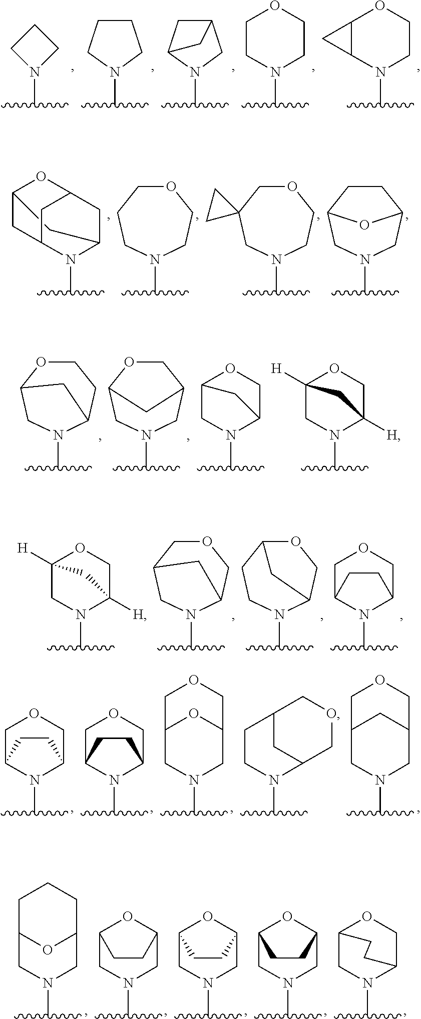 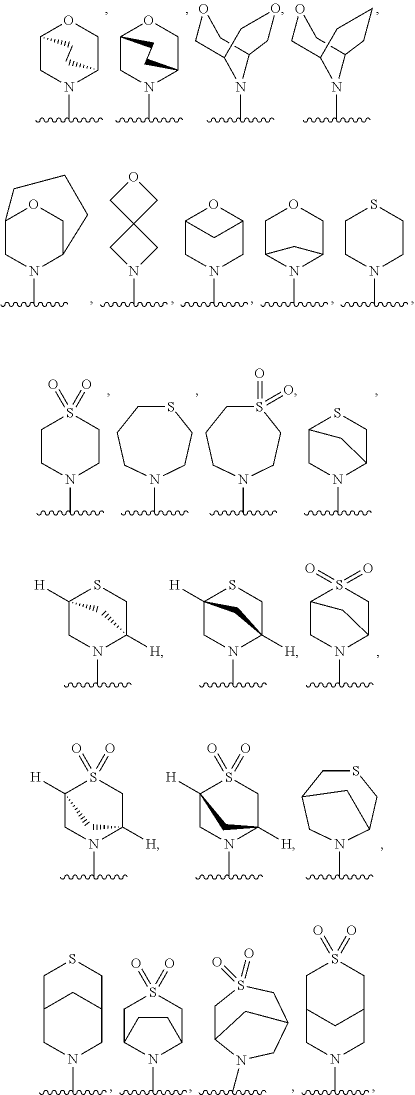 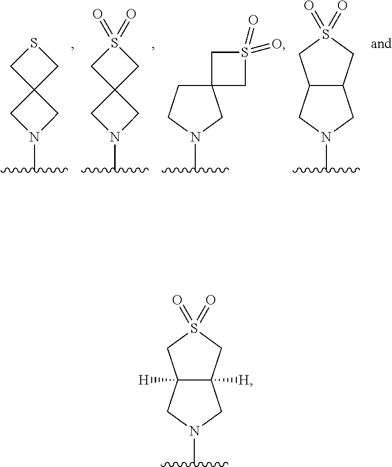
each substituted with 0, 1, 2, 3 or 4 substituents independently selected from the group consisting of halo, hydroxy, C₁-C₄ alkyl, C₁-C₆ alkoxy, C₁-C₆ alkoxyalkoxy, C₁-C₄ haloalkyl and C₁-C₄ haloalkoxy.

33. The compound of any one of claims 1-13 and 29-32, or a salt thereof, and/or an isotopologue thereof, wherein the 4-10 membered heterocycle of R^{f} is unsubstituted or substituted with 0, 1 or 2 substituents independently selected from the group consisting of —F, —OMe and -Me.

34. The compound of any one of claims 1-13, 29, and 30, or a salt thereof, and/or an isotopologue thereof, wherein R^{f} is selected from the group consisting of:

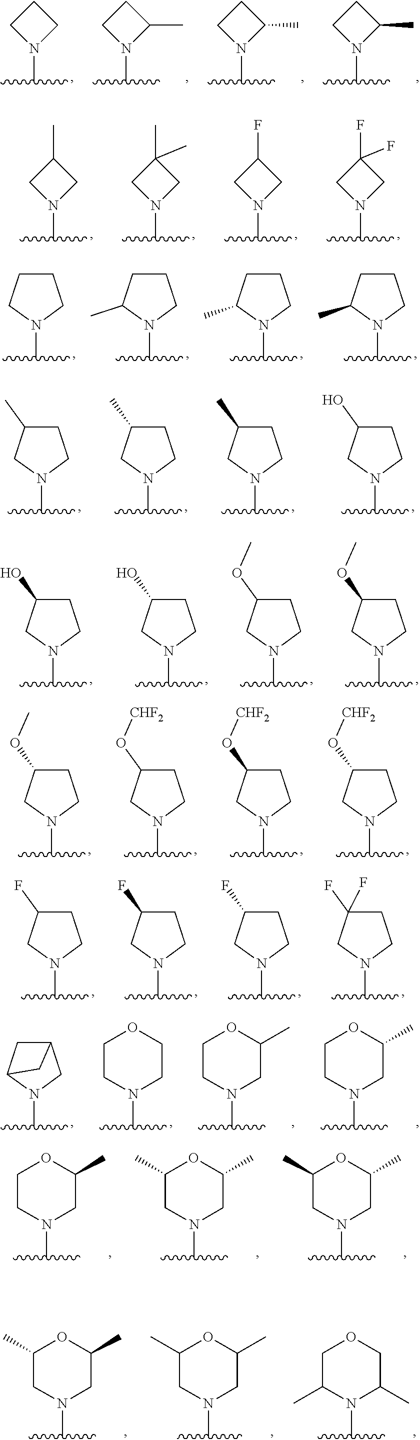

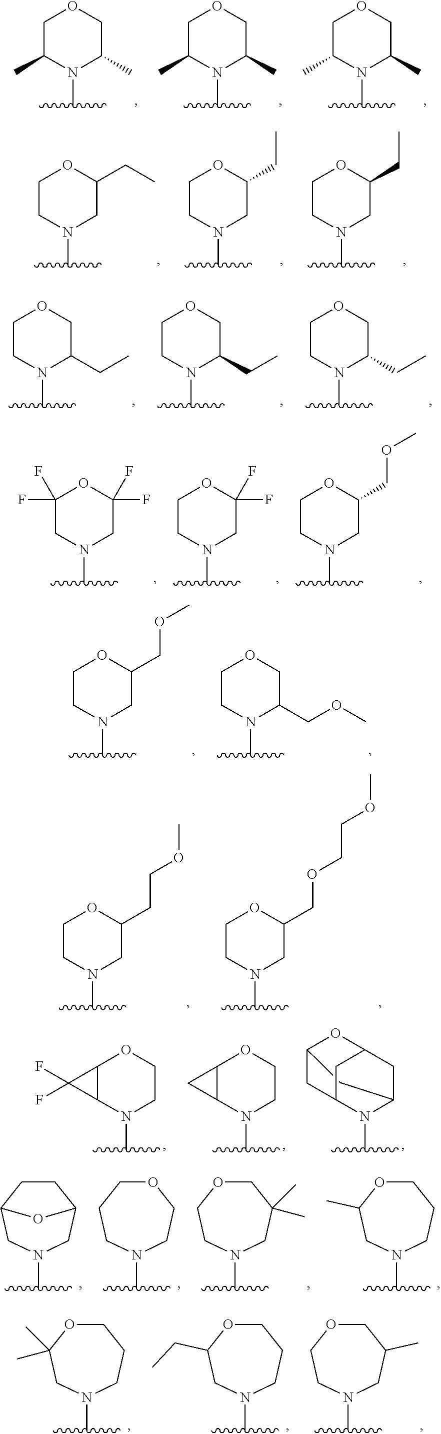

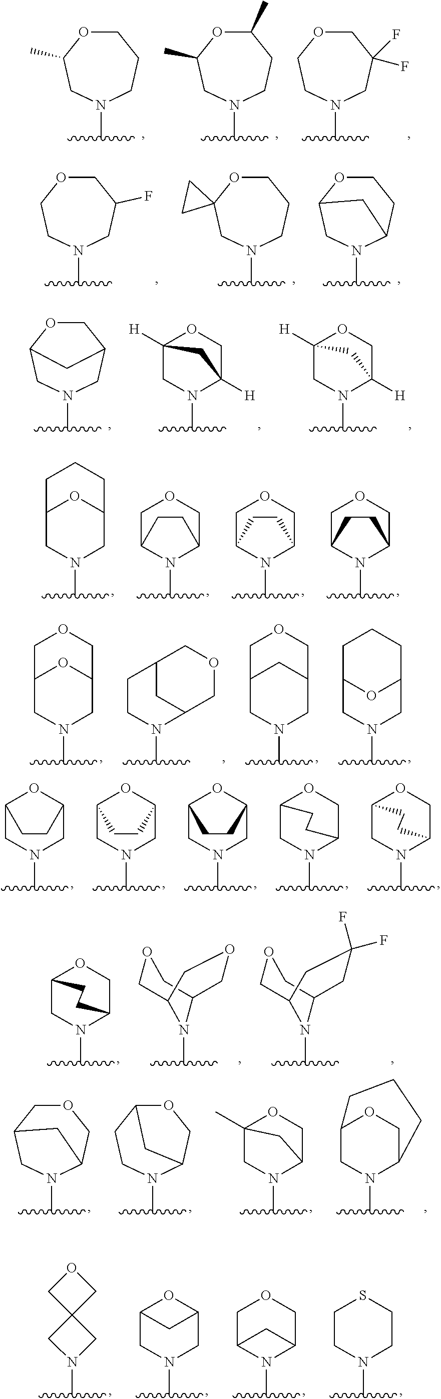

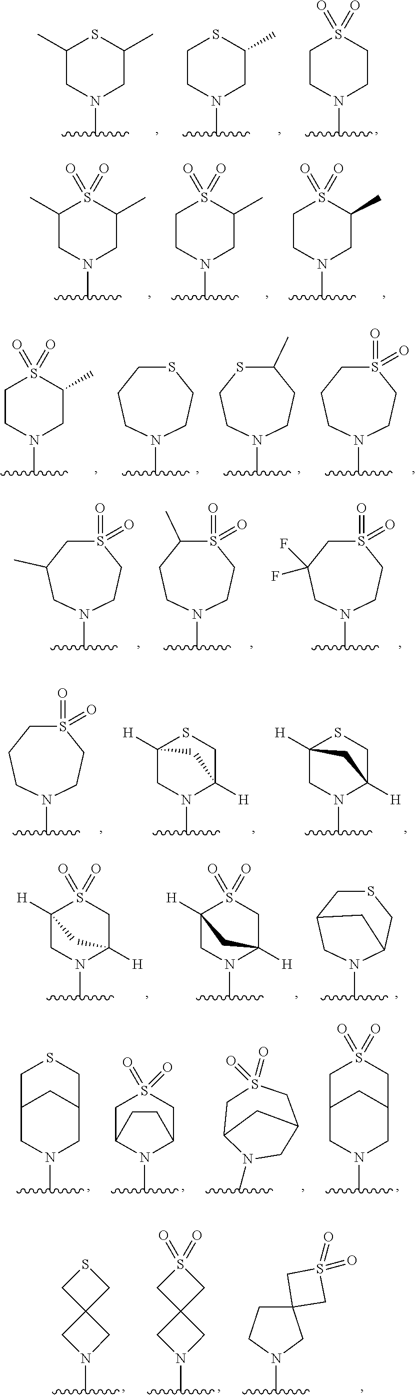

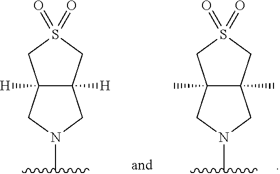

35. The compound of any one of claims 1-13 and 29, or a salt thereof, and/or an isotopologue thereof, wherein L-R^{f} is selected from the group consisting of:

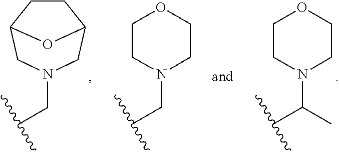

36. The compound of any one of claims 1-13, or a salt thereof, and/or an isotopologue thereof,
wherein R² is selected from the group consisting of:

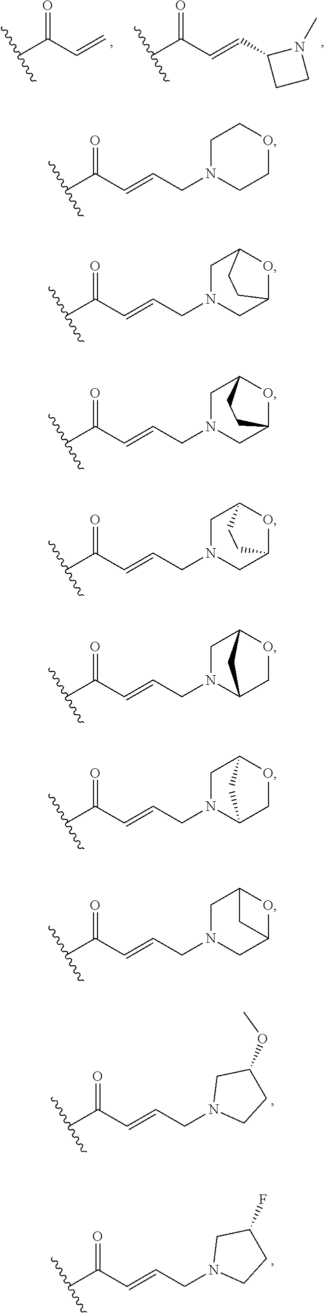

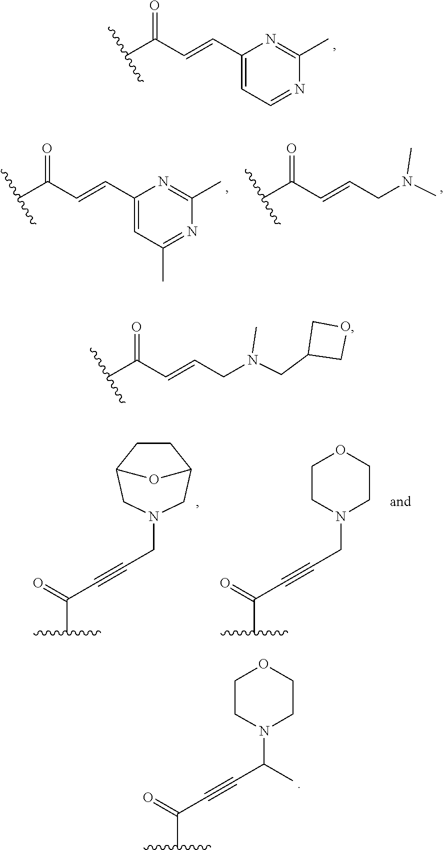

37. The compound of any one of claims 1-36, or a salt thereof, and/or an isotopologue thereof, wherein R^{x} is selected from the group consisting of —H and -Me.

38. The compound of any one of claims 1, 4, and 5-37, or a salt thereof, and/or an isotopologue thereof, wherein R^{y} is selected from the group consisting of —Cl and —CF₃.

39. The compound of claim 1, or a salt thereof, and/or an isotopologue thereof, wherein the compound is selected from Table 1.

40. The compound of any one of claims 1-39, or a salt thereof, and/or an isotopologue thereof, wherein the salt is a pharmaceutically acceptable salt.

41. A pharmaceutical formulation comprising the compound of any one of claims 1-40, or a salt thereof, and/or an isotopologue thereof, and a pharmaceutically acceptable carrier.

42. A method of treating or suppressing cancer comprising: administering a therapeutically effective amount of a compound of any one of claims 1-40, or a salt thereof, and/or an isotopologue thereof, or a pharmaceutical formulation according to claim 41, to a subject in need thereof.

43. The method of claim 42, wherein the cancer is selected from the group consisting of: lung, colorectal, pancreatic, bile duct, thyroid, gall bladder, uterine, mesothelioma, cervical, and bladder cancers.

44. The method of claim 42, wherein the cancer is selected from the group consisting of: glioblastoma multiforme, lower grade glioma, head and neck squamous cell carcinoma, papillary thyroid carcinoma, anaplastic thyroid carcinoma, follicular thyroid carcinoma, lung adenocarcinoma, lung squamous cell carcinoma, breast invasive carcinoma, esophageal carcinoma, stomach adenocarcinoma, small intestine adenocarcinoma, colon adenocarcinoma, rectal adenocarcinoma, liver hepatocellular carcinoma, cholangiocarcinoma, gallbladder carcinoma, pancreatic adenocarcinoma, kidney renal clear cell carcinoma, bladder urothelial carcinoma, prostate adenocarcinoma, ovarian serous cystadenocarcinoma, uterine corpus endometrial carcinoma, cervical squamous carcinoma and endocervical adenocarcinoma, skin cutaneous melanoma, acute lymphoblastic leukemia, acute myeloid leukemia, chronic myeloid leukemia, plasma cellmyeloma, uterine carcinosarcoma, mesothelioma, adrenocortical carcinoma, brain lower grade glioma, diffuse large B-cell lymphoma, esophageal adenocarcinoma, kidney chromophobe, kidney renal papillary cell carcinoma, pheochromocytoma and paraganglioma, sarcoma, testicular germ cell tumors, thymoma, uveal melanoma, metastatic colorectal cancer, bladder cancer, adenoid cystic carcinoma, myelodysplastic, breast cancer, thyroid carcinoma, glioma, esophageal/stomach cancer, pediatric Wilms' tumor, pediatric acute lymphoid leukemia, chronic lymphocytic leukemia, mature B-cellmalignancies, pediatric neuroblastoma, and melanoma.

45. The method of any one of claims 42-44, wherein the cancer is a KRAS G12C mediated cancer.

46. The method of any one of claims 42-44, wherein the subject has been diagnosed as having a KRAS G12C mediated cancer.

47. The method of any one of claims 42-46, wherein the method further comprises administering to the subject a therapeutically effective amount of an additional chemotherapeutic agent.
